(19) United States
(12) Patent Application Publication
(10) Pub. No.: US 2023/0293716 A1
(43) Pub. Date: Sep. 21, 2023

(54) ELASTASE-SUBSTRATE, PEPTIDE LINKER IMMUNOCONJUGATES, AND USES THEREOF

(71) Applicant: BOLT BIOTHERAPEUTICS, INC., Redwood City, CA (US)

(72) Inventors: David DORNAN, Redwood City, CA (US); Romas KUDIRKA, Redwood City, CA (US); Brian SAFINA, Redwood City, CA (US); Matthew ZHOU, Redwood City, CA (US)

(73) Assignee: BOLT BIOTHERAPEUTICS, INC., Redwood City, CA (US)

(21) Appl. No.: 17/923,541

(22) PCT Filed: May 7, 2021
(86) PCT No.: PCT/US2021/031264

§ 371 Date: Nov. 4, 2022

Related U.S. Application Data
(60) Provisional application No. 63/022,069, filed on May 8, 2020.

(51) Int. Cl. A61K 47/68; A61P 35/00

(52) U.S. Cl. CPC A61K 47/6889; A61K 47/6803; A61K 47/6849; A61K 47/6851; A61P 35/00

(57) ABSTRACT

The invention provides immunoconjugates of Formula I comprising a cell-binding agent linked by conjugation to one or more immunostimulatory moieties where the linker is a substrate for elastase. The invention also provides immunostimulant intermediate compositions comprising a reactive functional group. Such intermediate compositions are suitable substrates for formation of the immunoconjugates through a linker or linking moiety. The invention further provides methods of treating cancer with the immunoconjugates.

### CROSS REFERENCE TO RELATED APPLICATIONS

This non-provisional application claims the benefit of priority to U.S. Provisional Application No. 63/022,069, filed 8 May 2020, which is incorporated by reference in its entirety.

### SEQUENCE LISTING

The instant application contains a Sequence Listing which has been submitted electronically in ASCII format and is hereby incorporated by reference in its entirety. Said ASCII copy, created on May 5, 2021, is named 17019_008W01_SL.txt and is 09,056 bytes in size.

### FIELD OF THE INVENTION

The invention relates generally to an immunoconjugate comprising an antibody covalently attached to one or more immunostimulatory moieties by an elastase-substrate, peptide linker.

### BACKGROUND OF THE INVENTION

New compositions and methods for the delivery of antibodies and immunostimulatory adjuvants are needed in order to reach inaccessible tumors and/or to expand treatment options for cancer patients and other subjects.

Human neutrophil elastase (HNE) is a serine protease with destructive proteolytic activity. Elastase is stored in azurophilic granules of neutrophils and released into the extracellular space upon infections or inflammation stimuli. Elastase is also produced in cells of myeloid-lineage including myeloid-derived suppressor cells (MDSC), a heterogenous group of immune cells from myeloid lineage originating from bone marrow stem cells and within the tumor microenvironment. High levels of elastase have been reported in primary tumors and metastasis, where it promotes oncogenic signaling and inhibits tumor suppressors (Starcher, J. Invest. Derm. (1996), 107:159-163). Circulating and infiltrating neutrophils and granulocytic MDSCs correlate with tumor progression and patient survival (Lerman, I. (2018) Steroids 133: 96-101). As a result, elevated neutrophil elastase levels correlate with poor prognosis in different types of solid tumors. Peptide motifs to which elastase binds and cleaves include X-Ala-Ala-Pro-Val (SEQ ID NO: 637) or X-Ala-Ala-Pro-Nva where X is a peptide amino capping group and Nva is norvaline (SEQ ID NO: 638) (US 2002/0193311; US 6855689; WO 2000/069472).

A tumor-targeting, peptidomimetic integrin ligand cyclo(DKP-RGD) was conjugated to the anticancer drug paclitaxel through a Asn-Pro-Val (NPV) tripeptide linker. The Asn-Pro-Val (NPV) tripeptide linker is a substrate of neutrophil-secreted elastase. In vitro linker cleavage assays and cell antiproliferative experiments demonstrated the efficacy of this tumor-targeting conjugate (Dias, A.R.M et al (2019) Chem. Eur. J. 25:1696-1700). The pro-inflammatory environment and the presence of infiltrating cells of the immune system are well-established hallmarks of cancers (Hanahan D. et al (2011) Cell 144:646-674). It is therefore conceivable that elastase-activatable prodrugs may be therapeutically active against a large variety of tumor types.

The stability of antibody-drug conjugates (ADCs) in circulation is dictated by a number of factors, one of which is the susceptibility of the linker to premature cleavage by circulating esterases and proteases. Indeed, proteases such as neutrophil elastase are speculated to play a significant role in both the pharmacokinetics (PK)-exposure and toxicity of several ADCs that have been evaluated in the clinic (Flygare, J.A. et al (2013) Chem Biol Drug Des., 81:113-121.

A pool of 102 unnatural amino acids were incorporated into the S1—S4 binding pockets of human neutrophil elastase as tetrapeptides in a combinatorial library approach to optimize substrate catalytic cleavage efficiency (Kasperkiewicz, P. et al. (2014) Proc. Nat. Acad. Sci. 111:2518-2523). An optimal substrate demonstrated more than three orders of magnitude higher catalytic efficiency and selectivity than commonly used substrates of elastase and revealed the specific presence of active elastase during the process of neutrophil extracellular trap formation. Unnatural amino acids were shown to be much better substrates in terms of specificity and selectivity compared with natural ones (Zervoudi E, et al. (2011) Biochem J 435(2):411-420; Poreba M, et al. (2012) PLoS ONE 7(2):e31938.

### SUMMARY OF THE INVENTION

An aspect of the invention is an immunoconjugate comprising a cell-binding agent covalently attached to one or more immunostimulatory moieties by an elastase-substrate, peptide linker.

Another aspect of the invention is an immunostimulant-elastase substrate, peptide linker compound capable of conjugation with a cell-binding agent.

Another aspect of the invention is a pharmaceutical composition comprising a therapeutically effective amount of the immunoconjugate.

Another aspect of the invention is a method of treatment comprising administering a therapeutically-effective dose of the immunoconjugate to a patient with cancer or an immune-related disorder.

Another aspect of the invention is the use of the immunoconjugate in therapy.

### BRIEF DESCRIPTION OF THE DRAWINGS

FIGS. 1A-D show heavy chain and light chain CDRs of PD-L1 Type A binding agents 1-42.

FIGS. 2A-D show first (HFW1), second (HFW2), third (HFW3), and fourth (HFW4) heavy chain framework region polypeptides of PD-L1 Type A binding agents 1-42.

FIGS. 3A-D show first (LFW1), second (LFW2), third (LFW3), and fourth (LFW4) light chain framework region polypeptides of PD-L1 Type A binding agents 1-42.

FIGS. 4A-D show heavy chain variable region (VH) of PD-L1 Type A binding agents 1-42.

FIGS. 4E-G show light chain variable region (VL) of PD-L1 Type A binding agents 1-42.

FIGS. 5A-B show heavy chain and light chain CDRs of PD-L1 Type B binding agents 1-21.

FIGS. 6A-B show first (HFW1), second (HFW2), third (HFW3), and fourth (HFW4) heavy chain framework region polypeptides of PD-L1 Type B binding agents 1-21.

FIGS. 7A-B show first (LFW1), second (LFW2), third (LFW3), and fourth (LFW4) light chain framework region polypeptides of PD-L1 Type B binding agents 1-21.

FIGS. 8A-B show heavy chain variable region (VH) of PD-L1 Type B binding agents 1-21.

FIGS. 8C-D show light chain variable region (VL) of PD-L1 Type B binding agents 1-21.

FIG. 9 shows a graph of potency in a co-culture of a RAW 264.7 murine macrophage cell line and HCC1954 HER2 expressing tumor cells by a comparison of an elastase cleavable linker (Ala-Pro-Val) immunoconjugate ISAC-1 and a cathepsin B cleavable linker (Val-Cit) immunoconjugate ISAC-2. ISAC-1 has increased potency relative to the cathepsin B cleavable peptide (Val-Cit) ISAC-2 in RAWS only. The Val-Cit linker unit of ISAC-2 is a known cathepsin B substrate. Cells are cultured overnight at a 10:1 effector to target ratio, and mouse TNFa is measured by ELISA as a readout of a proinflammatory response.

### DETAILED DESCRIPTION OF THE INVENTION

Reference will now be made in detail to certain embodiments of the invention, examples of which are illustrated in the accompanying structures and formulas. While the invention will be described in conjunction with the enumerated embodiments, it will be understood that they are not intended to limit the invention to those embodiments. On the contrary, the invention is intended to cover all alternatives, modifications, and equivalents, which may be included within the scope of the invention as defined by the claims.

One skilled in the art will recognize many methods and materials similar or equivalent to those described herein, which could be used in the practice of the present invention. The invention is in no way limited to the methods and materials described.

### DEFINITIONS

The term “immunoconjugate” refers to an antibody construct that is covalently bonded to an immunostimulatory moiety via a linker.

The terms “immunostimulant” and “immunostimulatory” are used equivalently and refer to a moiety, substance or adjuvant capable of eliciting an immune response in a subject exposed to the immunostimulatory moiety or the immunostimulatory compound after in vivo cleavage of the linker. The terms “adjuvant moiety” or “immunostimulatory moiety” refer to an adjuvant that is covalently bonded to a cell-binding agent, such as an antibody construct, through an elastase-substrate, peptide linker, as described herein. The adjuvant moiety can elicit the immune response while bonded to the antibody construct or after cleavage (e.g., enzymatic cleavage) from the antibody construct following administration of an immunoconjugate to the subject. Immunoconjugates allow targeted delivery of an active adjuvant moiety while the target antigen is bound.

The term “pattern-recognition receptor” (PRR) refers to germline-encoded host sensors which detect molecules typical for pathogens and modulate function of the innate immune system (Mahla, RS et al (2013) Frontiers in Immunology 4:248; Kumar, H et al (2011) Intl. Rev. of Immun. 30:16-34; Schroder K et al (2010) Cell 140(6):821-832). PRRs are proteins expressed mainly by cells of the innate immune system such as dendritic cells, macrophages, monocytes, neutrophils and epithelial cells, to identify pathogen-associated molecular patterns (PAMPs) associated with microbial pathogens, and damage-associated molecular patterns (DAMPs) associated with components of host cells released during cell damage or death. PRRs are also called primitive pattern recognition receptors because they evolved before other parts of the immune system, particularly before adaptive immunity. PRRs also mediate the initiation of antigen-specific adaptive immune response and release of inflammatory cytokines. PRRs include but are not limited to: Toll-like receptors (TLRs), STING-like receptors, RIG-I-like receptors (RLRs), NOD-like receptors (NLRs), C-type lectin-like receptors (CLRs), and DNA sensors.

“Adjuvant” refers to an immunostimulatory substance capable of eliciting an immune response in a subject exposed to the adjuvant. The phrase “adjuvant moiety” refers to an adjuvant that is covalently bonded to an antibody construct, e.g., through a linker, as described herein. The adjuvant moiety can elicit the immune response while bonded to the antibody construct or after cleavage (e.g., enzymatic cleavage) from the antibody construct following administration of an immunoconjugate to the subject.

The terms “Toll-like receptor” and “TLR” refer to any member of a family of highly-conserved mammalian proteins which recognizes pathogen-associated molecular patterns and acts as key signaling elements in innate immunity. TLR polypeptides share a characteristic structure that includes an extracellular domain that has leucine-rich repeats, a transmembrane domain, and an intracellular domain that is involved in TLR signaling.

The terms “Toll-like receptor 7” and “TLR7” refer to nucleic acids or polypeptides sharing at least about 70%, about 80%, about 90%, about 95%, about 96%, about 97%, about 98%, about 99%, or more sequence identity to a publicly-available TLR7 sequence, e.g., GenBank accession number AAZ99026 for human TLR7 polypeptide, or GenBank accession number AAK62676 for murine TLR7 polypeptide.

The terms “Toll-like receptor 8” and “TLR8” refer to nucleic acids or polypeptides sharing at least about 70%, about 80%, about 90%, about 95%, about 96%, about 97%, about 98%, about 99%, or more sequence identity to a publicly-available TLR7 sequence, e.g., GenBank accession number AAZ95441 for human TLR8 polypeptide, or GenBank accession number AAK62677 for murine TLR8 polypeptide.

A “TLR agonist” is a substance that binds, directly or indirectly, to a TLR (e.g., TLR7 and/or TLR8) to induce TLR signaling. Any detectable difference in TLR signaling can indicate that an agonist stimulates or activates a TLR. Signaling differences can be manifested, for example, as changes in the expression of target genes, in the phosphorylation of signal transduction components, in the intracellular localization of downstream elements such as nuclear factor-κB (NF-κB), in the association of certain components (such as IL-1 receptor associated kinase (IRAK)) with other proteins or intracellular structures, or in the biochemical activity of components such as kinases (such as mitogen-activated protein kinase (MAPK)).

A “cell-binding agent” refers to a polypeptide that binds to a cell through at least one binding site. Cell-binding agents include antibodies or a fragment of an antibody, peptides and peptidomimetics. Examples of cell-binding agents also include lymphokines, hormones, growth factors, nutrient-transport molecules, or any other cell binding molecule or substance. Conjugates of cell binding agents allow targeted delivery of an active moiety to an effect cell while the antigen is bound to the antibody.

“Antibody” refers to a polypeptide comprising an antigen binding region (including the complementarity determining region (CDRs)) from an immunoglobulin gene or fragments thereof. The term “antibody” specifically encompasses monoclonal antibodies (including full length monoclonal antibodies), polyclonal antibodies, multispecific antibodies (e.g., bispecific antibodies), and antibody fragments that exhibit the desired biological activity. An exemplary immunoglobulin (antibody) structural unit comprises a tetramer. Each tetramer is composed of two identical pairs of polypeptide chains, each pair having one “light” (about 25 kDa) and one “heavy” chain (about 50-70 kDa) connected by disulfide bonds. Each chain is composed of structural domains, which are referred to as immunoglobulin domains. These domains are classified into different categories by size and function, e.g., variable domains or regions on the light and heavy chains (V_{L} and V_{H}, respectively) and constant domains or regions on the light and heavy chains (C_{L} and C_{H}, respectively). The N-terminus of each chain defines a variable region of about 100 to 110 or more amino acids, referred to as the paratope, primarily responsible for antigen recognition, i.e., the antigen binding domain. Light chains are classified as either kappa or lambda. Heavy chains are classified as gamma, mu, alpha, delta, or epsilon, which in turn define the immunoglobulin classes, IgG, IgM, IgA, IgD and IgE, respectively. IgG antibodies are large molecules of about 150 kDa composed of four peptide chains. IgG antibodies contain two identical class γ heavy chains of about 50 kDa and two identical light chains of about 25 kDa, thus a tetrameric quaternary structure. The two heavy chains are linked to each other and to a light chain each by disulfide bonds. The resulting tetramer has two identical halves, which together form the Y-like shape. Each end of the fork contains an identical antigen binding domain. There are four IgG subclasses (IgG1, IgG2, IgG3, and IgG4) in humans, named in order of their abundance in serum (i.e., IgG1 is the most abundant). Typically, the antigen binding domain of an antibody will be most critical in specificity and affinity of binding to cancer cells.

“Antibody construct” refers to an antibody or a fusion protein comprising (i) an antigen binding domain and (ii) an Fc domain.

In some embodiments, the binding agent is an antigen-binding antibody “fragment,” which is a construct that comprises at least an antigen-binding region of an antibody, alone or with other components that together constitute the antigen-binding construct. Many different types of antibody “fragments” are known in the art, including, for instance, (i) a Fab fragment, which is a monovalent fragment consisting of the V_{L}, V_{H}, C_{L}, and CH₁ domains, (ii) a F(ab′)₂ fragment, which is a bivalent fragment comprising two Fab fragments linked by a disulfide bridge at the hinge region, (iii) a Fv fragment consisting of the V_{L} and V_{H} domains of a single arm of an antibody, (iv) a Fab’ fragment, which results from breaking the disulfide bridge of an F(ab′)₂ fragment using mild reducing conditions, (v) a disulfide-stabilized Fv fragment (dsFv), and (vi) a single chain Fv (scFv), which is a monovalent molecule consisting of the two domains of the Fv fragment (i.e., V_{L} and V_{H}) joined by a synthetic linker which enables the two domains to be synthesized as a single polypeptide chain.

The antibody or antibody fragments can be part of a larger construct, for example, a conjugate or fusion construct of the antibody fragment to additional regions. For instance, in some embodiments, the antibody fragment can be fused to an Fc region as described herein. In other embodiments, the antibody fragment (e.g., a Fab or scFv) can be part of a chimeric antigen receptor or chimeric T-cell receptor, for instance, by fusing to a transmembrane domain (optionally with an intervening linker or “stalk” (e.g., hinge region)) and optional intercellular signaling domain. For instance, the antibody fragment can be fused to the gamma and/or delta chains of a t-cell receptor, so as to provide a T-cell receptor like construct that binds PD-L1. In yet another embodiment, the antibody fragment is part of a bispecific T-cell engager (BiTEs) comprising a CD1 or CD3 binding domain and linker.

“Epitope” means any antigenic determinant or epitopic determinant of an antigen to which an antigen binding domain binds (i.e., at the paratope of the antigen binding domain). Antigenic determinants usually consist of chemically active surface groupings of molecules, such as amino acids or sugar side chains, and usually have specific three dimensional structural characteristics, as well as specific charge characteristics.

The terms “Fc receptor” or “FcR” refer to a receptor that binds to the Fc region of an antibody. There are three main classes of Fc receptors: (1) FcγR which bind to IgG, (2) FcαR which binds to IgA, and (3) FcεR which binds to IgE. The FcγR family includes several members, such as FcγI (CD64), FcγRIIA (CD32A), FcγRIIB (CD32B), FcγRIIIA (CD16A), and FcγRIIIB (CD16B). The Fcγ (Fc gamma) receptors differ in their affinity for IgG and also have different affinities for the IgG subclasses (e.g., IgG1, IgG2, IgG3, and IgG4).

Nucleic acid or amino acid sequence “identity,” as referenced herein, can be determined by comparing a nucleic acid or amino acid sequence of interest to a reference nucleic acid or amino acid sequence. The percent identity is the number of nucleotides or amino acid residues that are the same (i.e., that are identical) as between the optimally aligned sequence of interest and the reference sequence divided by the length of the longest sequence (i.e., the length of either the sequence of interest or the reference sequence, whichever is longer). Alignment of sequences and calculation of percent identity can be performed using available software programs. Examples of such programs include CLUSTAL-W, T-Coffee, and ALIGN (for alignment of nucleic acid and amino acid sequences), BLAST programs (e.g., BLAST 2.1, BL2SEQ, BLASTp, BLASTn, and the like) and FASTA programs (e.g., FASTA3x, FASTM, and SSEARCH) (for sequence alignment and sequence similarity searches). Sequence alignment algorithms also are disclosed in, for example, Altschul et al., J. Molecular Biol., 215(3): 403-410 (1990), Beigert et al., Proc. Natl. Acad. Sci. USA, 106(10): 3770-3775 (2009), Durbin et al., eds., Biological Sequence Analysis: Probalistic Models of Proteins and Nucleic Acids, Cambridge University Press, Cambridge, UK (2009), Soding, Bioinformatics, 21(7): 951-960 (2005), Altschul et al., Nucleic Acids Res., 25(17): 3389-3402 (1997), and Gusfield, Algorithms on Strings, Trees and Sequences, Cambridge University Press, Cambridge UK (1997)). Percent (%) identity of sequences can be also calculated, for example, as 100 x [(identical positions)/min(TG_{A}, TG_{B})], where TG_{A} and TG_{B} are the sum of the number of residues and internal gap positions in peptide sequences A and B in the alignment that minimizes TG_{A} and TG_{B}. See, e.g., Russell et al., J. Mol Biol., 244: 332-350 (1994).

The binding agent comprises Ig heavy and light chain variable region polypeptides that together form the antigen binding site. Each of the heavy and light chain variable regions are polypeptides comprising three complementarity determining regions (CDR1, CDR2, and CDR3) connected by framework regions. The binding agent can be any of a variety of types of binding agents known in the art that comprise Ig heavy and light chains. For instance, the binding agent can be an antibody, an antigen-binding antibody “fragment,” or a T-cell receptor.

“Biosimilar” refers to an approved antibody construct that has active properties similar to, for example, a PD-L1-targeting antibody construct previously approved such as atezolizumab (TECENTRIQ™, Genentech, Inc.), durvalumab (IMFINZI™, AstraZeneca), and avelumab (BAVENCIO™, EMD Serono, Pfizer); a HER2-targeting antibody construct previously approved such as trastuzumab (HERCEPTIN™, Genentech, Inc.), and pertuzumab (PERJETA™, Genentech, Inc.); or a CEA-targeting antibody such as labetuzumab (CEA-CIDE™, MN-14, hMN14, Immunomedics) CAS Reg. No. 219649-07-7).

“Biobetter” refers to an approved antibody construct that is an improvement of a previously approved antibody construct, such as atezolizumab, durvalumab, avelumab, trastuzumab, pertuzumab, labetuzumab, or sacituzumab. The biobetter can have one or more modifications (e.g., an altered glycan profile, or a unique epitope) over the previously approved antibody construct.

“Amino acid” refers to any monomeric unit that can be incorporated into a peptide, polypeptide, or protein. Amino acids include naturally-occurring α-amino acids and their stereoisomers, as well as unnatural (non-naturally occurring) amino acids and their stereoisomers. “Stereoisomers” of a given amino acid refer to isomers having the same molecular formula and intramolecular bonds but different three-dimensional arrangements of bonds and atoms (e.g., an _{L}-amino acid and the corresponding D-amino acid). The amino acids can be glycosylated (e.g., N-linked glycans, O-linked glycans, phosphoglycans, C-linked glycans, or glypication) or deglycosylated. Amino acids may be referred to herein by either the commonly known three letter symbols or by the one-letter symbols recommended by the IUPAC-IUB Biochemical Nomenclature Commission.

Naturally-occurring amino acids are those encoded by the genetic code, as well as those amino acids that are later modified, e.g., hydroxyproline, γ-carboxyglutamate, and O-phosphoserine. Naturally-occurring α-amino acids include, without limitation, alanine (Ala), cysteine (Cys), aspartic acid (Asp), glutamic acid (Glu), phenylalanine (Phe), glycine (Gly), histidine (His), isoleucine (Ile), arginine (Arg), lysine (Lys), leucine (Leu), methionine (Met), asparagine (Asn), proline (Pro), glutamine (Gln), serine (Ser), threonine (Thr), valine (Val), tryptophan (Trp), tyrosine (Tyr), and combinations thereof. Stereoisomers of naturally-occurring α-amino acids include, without limitation, D-alanine (D-Ala), D-cysteine (D-Cys), D-aspartic acid (D-Asp), D-glutamic acid (D-Glu), D-phenylalanine (D-Phe), D-histidine (D-His), D-isoleucine (D-Ile), D-arginine (D-Arg), D-lysine (D-Lys), D-leucine (D-Leu), D-methionine (D-Met), D-asparagine (D-Asn), D-proline (D-Pro), D-glutamine (D-Gln), D-serine (D-Ser), D-threonine (D-Thr), D-valine (D-Val), D-tryptophan (D-Trp), D-tyrosine (D-Tyr), and combinations thereof.

Naturally-occurring amino acids include those formed in proteins by post-translational modification, such as citrulline (Cit).

Unnatural (non-naturally occurring) amino acids include, without limitation, amino acid analogs, amino acid mimetics, synthetic amino acids, N-substituted glycines, and N-methyl amino acids in either the L- or D-configuration that function in a manner similar to the naturally-occurring amino acids. For example, “amino acid analogs” can be unnatural amino acids that have the same basic chemical structure as naturally-occurring amino acids (i.e., a carbon that is bonded to a hydrogen, a carboxyl group, an amino group) but have modified side-chain groups or modified peptide backbones, e.g., homoserine, norleucine, methionine sulfoxide, and methionine methyl sulfonium. “Amino acid mimetics” refer to chemical compounds that have a structure that is different from the general chemical structure of an amino acid, but that functions in a manner similar to a naturally-occurring amino acid.

“Linker” refers to a functional group that covalently bonds two or more moieties in a compound or material. For example, the linking moiety can serve to covalently bond an adjuvant moiety to an antibody construct in an immunoconjugate.

“Linking moiety” refers to a functional group that covalently bonds two or more moieties in a compound or material. For example, the linking moiety can serve to covalently bond an adjuvant moiety to an antibody in an immunoconjugate. Useful bonds for connecting linking moieties to proteins and other materials include, but are not limited to, amides, amines, esters, carbamates, ureas, thioethers, thiocarbamates, thiocarbonates, and thioureas.

“Divalent” refers to a chemical moiety that contains two points of attachment for linking two functional groups; polyvalent linking moieties can have additional points of attachment for linking further functional groups. Divalent radicals may be denoted with the suffix “diyl”. For example, divalent linking moieties include divalent polymer moieties such as divalent poly(ethylene glycol), divalent cycloalkyl, divalent heterocycloalkyl, divalent aryl, and divalent heteroaryl group. A “divalent cycloalkyl, heterocycloalkyl, aryl, or heteroaryl group” refers to a cycloalkyl, heterocycloalkyl, aryl, or heteroaryl group having two points of attachment for covalently linking two moieties in a molecule or material. Cycloalkyl, heterocycloalkyl, aryl, or heteroaryl groups can be substituted or unsubstituted. Cycloalkyl, heterocycloalkyl, aryl, or heteroaryl groups can be substituted with one or more groups selected from halo, hydroxy, amino, alkylamino, amido, acyl, nitro, cyano, and alkoxy.

A wavy line (“

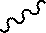

”) represents a point of attachment of the specified chemical moiety. If the specified chemical moiety has two wavy lines (“

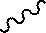

”) present, it will be understood that the chemical moiety can be used bilaterally, i.e., as read from left to right or from right to left. In some embodiments, a specified moiety having two wavy lines (“

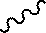

”) present is considered to be used as read from left to right.

“Alkyl” refers to a straight (linear) or branched, saturated, aliphatic radical having the number of carbon atoms indicated. Alkyl can include any number of carbons, for example from one to twelve. Examples of alkyl groups include, but are not limited to, methyl (Me, —CH3), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), 3,3-dimethyl-2-butyl (—CH(CH₃)C(CH₃)₃, 1-heptyl, 1-octyl, and the like. Alkyl groups can be substituted or unsubstituted. “Substituted alkyl” groups can be substituted with one or more groups selected from halo, hydroxy, amino, oxo (═O), alkylamino, amido, acyl, nitro, cyano, and alkoxy.

The term “alkyldiyl” refers to a divalent alkyl radical. Examples of alkyldiyl groups include, but are not limited to, methylene (—CH₂—), ethylene (—CH₂CH₂—), propylene (—CH₂CH₂CH₂—), and the like. An alkyldiyl group may also be referred to as an “alkylene” group.

“Alkenyl” refers to a straight (linear) or branched, unsaturated, aliphatic radical having the number of carbon atoms indicated and at least one carbon-carbon double bond, sp2. Alkenyl can include from two to about 12 or more carbons atoms. Alkenyl groups are radicals having “cis” and “trans” orientations, or alternatively, “E” and “Z” orientations. Examples include, but are not limited to, ethylenyl or vinyl (—CH═CH₂), allyl (—CH₂CH═CH₂). butenyl, pentenyl, and isomers thereof. Alkenyl groups can be substituted or unsubstituted. “Substituted alkenyl” groups can be substituted with one or more groups selected from halo, hydroxy, amino, oxo (═O), alkylamino, amido, acyl, nitro, cyano, and alkoxy.

The terms “alkenylene” or “alkenyldiyl” refer to a linear or branched-chain divalent hydrocarbon radical. Examples include, but are not limited to, ethylenylene or vinylene (—CH═CH—), allyl (—CH₂CH═CH—), and the like.

“Alkynyl” refers to a straight (linear) or branched, unsaturated, aliphatic radical having the number of carbon atoms indicated and at least one carbon-carbon triple bond, sp. Alkynyl can include from two to about 12 or more carbons atoms. For example, C₂-C₆ alkynyl includes, but is not limited to ethynyl (—C═CH), propynyl (propargyl, —CH2C═CH), butynyl, pentynyl, hexynyl, and isomers thereof Alkynyl groups can be substituted or unsubstituted. “Substituted alkynyl” groups can be substituted with one or more groups selected from halo, hydroxy, amino, oxo (=O), alkylamino, amido, acyl, nitro, cyano, and alkoxy.

The term “alkynylene” or “alkynyldiyl” refer to a divalent alkynyl radical.

The terms “carbocycle”, “carbocyclyl”, “carbocyclic ring” and “cycloalkyl” refer to a saturated or partially unsaturated, monocyclic, fused bicyclic, or bridged polycyclic ring assembly containing from 3 to 12 ring atoms, or the number of atoms indicated. Saturated monocyclic carbocyclic rings include, for example, cyclopropyl, cyclobutyl, cyclopentyl, cyclohexyl, and cyclooctyl. Saturated bicyclic and polycyclic carbocyclic rings include, for example, norbornane, [2.2.2] bicyclooctane, decahydronaphthalene and adamantane. Carbocyclic groups can also be partially unsaturated, having one or more double or triple bonds in the ring. Representative carbocyclic groups that are partially unsaturated include, but are not limited to, cyclobutene, cyclopentene, cyclohexene, cyclohexadiene (1,3- and 1,4-isomers), cycloheptene, cycloheptadiene, cyclooctene, cyclooctadiene (1,3-, 1,4- and 1,5-isomers), norbornene, and norbornadiene.

The term “cycloalkyldiyl” refers to a divalent cycloalkyl radical.

“Aryl” refers to a monovalent aromatic hydrocarbon radical of 6-20 carbon atoms (C₆₋ C₂₀) derived by the removal of one hydrogen atom from a single carbon atom of a parent aromatic ring system.. Aryl groups can be monocyclic, fused to form bicyclic or tricyclic groups, or linked by a bond to form a biaryl group. Representative aryl groups include phenyl, naphthyl and biphenyl. Other aryl groups include benzyl, having a methylene linking group. Some aryl groups have from 6 to 12 ring members, such as phenyl, naphthyl or biphenyl. Other aryl groups have from 6 to 10 ring members, such as phenyl or naphthyl.

The terms “arylene” or “aryldiyl” mean a divalent aromatic hydrocarbon radical of 6-20 carbon atoms (C₆-C₂₀) derived by the removal of two hydrogen atom from a two carbon atoms of a parent aromatic ring system. Some aryldiyl groups are represented in the exemplary structures as “Ar”. Aryldiyl includes bicyclic radicals comprising an aromatic ring fused to a saturated, partially unsaturated ring, or aromatic carbocyclic ring. Typical aryldiyl groups include, but are not limited to, radicals derived from benzene (phenyldiyl), substituted benzenes, naphthalene, anthracene, biphenylene, indenylene, indanylene, 1,2-dihydronaphthalene, 1,2,3,4-tetrahydronaphthyl, and the like. Aryldiyl groups are also referred to as “arylene”, and are optionally substituted with one or more substituents described herein.

The terms “heterocycle,” “heterocyclyl” and “heterocyclic ring” are used interchangeably herein and refer to a saturated or a partially unsaturated (i.e., having one or more double and/or triple bonds within the ring) carbocyclic radical of 3 to about 20 ring atoms in which at least one ring atom is a heteroatom selected from nitrogen, oxygen, phosphorus and sulfur, the remaining ring atoms being C, where one or more ring atoms is optionally substituted independently with one or more substituents described below. A heterocycle may be a monocycle having 3 to 7 ring members (2 to 6 carbon atoms and 1 to 4 heteroatoms selected from N, O, P, and S) or a bicycle having 7 to 10 ring members (4 to 9 carbon atoms and 1 to 6 heteroatoms selected from N, O, P, and S), for example: a bicyclo [4,5], [5,5], [5,6], or [6,6] system. Heterocycles are described in Paquette, Leo A.; “Principles of Modern Heterocyclic Chemistry” (W.A. Benjamin, New York, 1968), particularly Chapters 1, 3, 4, 6, 7, and 9; “The Chemistry of Heterocyclic Compounds, A series of Monographs” (John Wiley & Sons, New York, 1950 to present), in particular Volumes 13, 14, 16, 19, and 28; and J. Am. Chem. Soc. (1960) 82:5566. “Heterocyclyl” also includes radicals where heterocycle radicals are fused with a saturated, partially unsaturated ring, or aromatic carbocyclic or heterocyclic ring. Examples of heterocyclic rings include, but are not limited to, morpholin-4-yl, piperidin-1-yl, piperazinyl, piperazin-4-yl-2-one, piperazin-4-yl-3-one, pyrrolidin-1-yl, thiomorpholin-4-yl, S-dioxothiomorpholin-4-yl, azocan-1-yl, azetidin-1-yl, octahydropyrido[1,2-a]pyrazin-2-yl, [1,4]diazepan-1-yl, pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, homopiperazinyl, azetidinyl, oxetanyl, thietanyl, homopiperidinyl, oxepanyl, thiepanyl, oxazepinyl, diazepinyl, thiazepinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinylimidazolinyl, imidazolidinyl, 3-azabicyco[3.1.0]hexanyl, 3-azabicyclo[4.1.0]heptanyl, azabicyclo[2.2.2]hexanyl, 3H-indolyl quinolizinyl and N-pyridyl ureas. Spiro heterocyclyl moieties are also included within the scope of this definition. Examples of spiro heterocyclyl moieties include azaspiro[2.5]octanyl and azaspiro[2.4]heptanyl. Examples of a heterocyclic group wherein 2 ring atoms are substituted with oxo (=O) moieties are pyrimidinonyl and 1,1-dioxo-thiomorpholinyl. The heterocycle groups herein are optionally substituted independently with one or more substituents described herein.

The term “heterocyclyldiyl” refers to a divalent, saturated or a partially unsaturated (i.e., having one or more double and/or triple bonds within the ring) carbocyclic radical of 3 to about 20 ring atoms in which at least one ring atom is a heteroatom selected from nitrogen, oxygen, phosphorus and sulfur, the remaining ring atoms being C, where one or more ring atoms is optionally substituted independently with one or more substituents as described. Examples of 5-membered and 6-membered heterocyclyldiyls include morpholinyldiyl, piperidinyldiyl, piperazinyldiyl, pyrrolidinyldiyl, dioxanyldiyl, thiomorpholinyldiyl, and S-dioxothiomorpholinyldiyl.

The term “heteroaryl” refers to a monovalent aromatic radical of 5-, 6-, or 7-membered rings, and includes fused ring systems (at least one of which is aromatic) of 5-20 atoms, containing one or more heteroatoms independently selected from nitrogen, oxygen, and sulfur. Examples of heteroaryl groups are pyridinyl (including, for example, 2-hydroxypyridinyl), imidazolyl, imidazopyridinyl, pyrimidinyl (including, for example, 4-hydroxypyrimidinyl), pyrazolyl, triazolyl, pyrazinyl, tetrazolyl, furyl, thienyl, isoxazolyl, thiazolyl, oxadiazolyl, oxazolyl, isothiazolyl, pyrrolyl, quinolinyl, isoquinolinyl, tetrahydroisoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. Heteroaryl groups are optionally substituted independently with one or more substituents described herein.

The term “heteroaryldiyl” refers to a divalent aromatic radical of 5-, 6-, or 7-membered rings, and includes fused ring systems (at least one of which is aromatic) of 5-20 atoms, containing one or more heteroatoms independently selected from nitrogen, oxygen, and sulfur. Examples of 5-membered and 6-membered heteroaryldiyls include pyridyldiyl, imidazolyldiyl, pyrimidinyldiyl, pyrazolyldiyl, triazolyldiyl, pyrazinyldiyl, tetrazolyldiyl, furyldiyl, thienyldiyl, isoxazolyldiyldiyl, thiazolyldiyl, oxadiazolyldiyl, oxazolyldiyl, isothiazolyldiyl, and pyrrolyldiyl.

The heterocycle or heteroaryl groups may be carbon (carbon-linked), or nitrogen (nitrogen-linked) bonded where such is possible. By way of example and not limitation, carbon bonded heterocycles or heteroaryls are bonded at position 2, 3, 4, 5, or 6 of a pyridine, position 3, 4, 5, or 6 of a pyridazine, position 2, 4, 5, or 6 of a pyrimidine, position 2, 3, 5, or 6 of a pyrazine, position 2, 3, 4, or 5 of a furan, tetrahydrofuran, thiofuran, thiophene, pyrrole or tetrahydropyrrole, position 2, 4, or 5 of an oxazole, imidazole or thiazole, position 3, 4, or 5 of an isoxazole, pyrazole, or isothiazole, position 2 or 3 of an aziridine, position 2, 3, or 4 of an azetidine, position 2, 3, 4, 5, 6, 7, or 8 of a quinoline or position 1, 3, 4, 5, 6, 7, or 8 of an isoquinoline.

By way of example and not limitation, nitrogen bonded heterocycles or heteroaryls are bonded at position 1 of an aziridine, azetidine, pyrrole, pyrrolidine, 2-pyrroline, 3-pyrroline, imidazole, imidazolidine, 2-imidazoline, 3-imidazoline, pyrazole, pyrazoline, 2-pyrazoline, 3-pyrazoline, piperidine, piperazine, indole, indoline, 1H-indazole, position 2 of a isoindole, or isoindoline, position 4 of a morpholine, and position 9 of a carbazole, or β-carboline.

The terms “halo” and “halogen,” by themselves or as part of another substituent, refer to a fluorine, chlorine, bromine, or iodine atom.

The term “carbonyl,” by itself or as part of another substituent, refers to C(═O) or —C(═O)—, i.e., a carbon atom double-bonded to oxygen and bound to two other groups in the moiety having the carbonyl.

As used herein, the phrase “quaternary ammonium salt” refers to a tertiary amine that has been quaternized with an alkyl substituent (e.g., a C₁-C₄ alkyl such as methyl, ethyl, propyl, or butyl).

The terms “treat,” “treatment,” and “treating” refer to any indicia of success in the treatment or amelioration of an injury, pathology, condition (e.g., cancer), or symptom (e.g., cognitive impairment), including any objective or subjective parameter such as abatement; remission; diminishing of symptoms or making the symptom, injury, pathology, or condition more tolerable to the patient; reduction in the rate of symptom progression; decreasing the frequency or duration of the symptom or condition; or, in some situations, preventing the onset of the symptom. The treatment or amelioration of symptoms can be based on any objective or subjective parameter, including, for example, the result of a physical examination.

The terms “cancer,” “neoplasm,” and “tumor” are used herein to refer to cells which exhibit autonomous, unregulated growth, such that the cells exhibit an aberrant growth phenotype characterized by a significant loss of control over cell proliferation. Cells of interest for detection, analysis, and/or treatment in the context of the invention include cancer cells (e.g., cancer cells from an individual with cancer), malignant cancer cells, pre-metastatic cancer cells, metastatic cancer cells, and non-metastatic cancer cells. Cancers of virtually every tissue are known. The phrase “cancer burden” refers to the quantum of cancer cells or cancer volume in a subject. Reducing cancer burden accordingly refers to reducing the number of cancer cells or the cancer cell volume in a subject. The term “cancer cell” as used herein refers to any cell that is a cancer cell (e.g., from any of the cancers for which an individual can be treated, e.g., isolated from an individual having cancer) or is derived from a cancer cell, e.g., clone of a cancer cell. For example, a cancer cell can be from an established cancer cell line, can be a primary cell isolated from an individual with cancer, can be a progeny cell from a primary cell isolated from an individual with cancer, and the like. In some embodiments, the term can also refer to a portion of a cancer cell, such as a sub-cellular portion, a cell membrane portion, or a cell lysate of a cancer cell. Many types of cancers are known to those of skill in the art, including solid tumors such as carcinomas, sarcomas, glioblastomas, melanomas, lymphomas, and myelomas, and circulating cancers such as leukemias.

As used herein, the term “cancer” includes any form of cancer, including but not limited to, solid tumor cancers (e.g., skin, lung, prostate, breast, gastric, bladder, colon, ovarian, pancreas, kidney, liver, glioblastoma, medulloblastoma, leiomyosarcoma, head & neck squamous cell carcinomas, melanomas, and neuroendocrine) and liquid cancers (e.g., hematological cancers); carcinomas; soft tissue tumors; sarcomas; teratomas; melanomas; leukemias; lymphomas; and brain cancers, including minimal residual disease, and including both primary and metastatic tumors.

“PD-L1 expression” refers to a cell that has a PD-L1 receptor on the cell’s surface. As used herein “PD-L1 overexpression” refers to a cell that has more PD-L1 receptors as compared to corresponding non-cancer cell.

“HER2” refers to the protein human epidermal growth factor receptor 2.

“HER2 expression” refers to a cell that has a HER2 receptor on the cell’s surface. For example, a cell may have from about 20,000 to about 50,000 HER2 receptors on the cell’s surface. As used herein “HER2 overexpression” refers to a cell that has more than about 50,000 HER2 receptors. For example, a cell 2, 5, 10, 100, 1,000, 10,000, 100,000, or 1,000,000 times the number of HER2 receptors as compared to corresponding non-cancer cell (e.g., about 1 or 2 million HER2 receptors). It is estimated that HER2 is overexpressed in about 25% to about 30% of breast cancers.

The “pathology” of cancer includes all phenomena that compromise the well-being of the patient. This includes, without limitation, abnormal or uncontrollable cell growth, metastasis, interference with the normal functioning of neighboring cells, release of cytokines or other secretory products at abnormal levels, suppression or aggravation of inflammatory or immunological response, neoplasia, premalignancy, malignancy, and invasion of surrounding or distant tissues or organs, such as lymph nodes.

As used herein, the phrases “cancer recurrence” and “tumor recurrence,” and grammatical variants thereof, refer to further growth of neoplastic or cancerous cells after diagnosis of cancer. Particularly, recurrence may occur when further cancerous cell growth occurs in the cancerous tissue. “Tumor spread,” similarly, occurs when the cells of a tumor disseminate into local or distant tissues and organs, therefore, tumor spread encompasses tumor metastasis. “Tumor invasion” occurs when the tumor growth spread out locally to compromise the function of involved tissues by compression, destruction, or prevention of normal organ function.

As used herein, the term “metastasis” refers to the growth of a cancerous tumor in an organ or body part, which is not directly connected to the organ of the original cancerous tumor. Metastasis will be understood to include micrometastasis, which is the presence of an undetectable amount of cancerous cells in an organ or body part that is not directly connected to the organ of the original cancerous tumor. Metastasis can also be defined as several steps of a process, such as the departure of cancer cells from an original tumor site, and migration and/or invasion of cancer cells to other parts of the body.

The phrases “effective amount” and “therapeutically effective amount” refer to a dose or amount of a substance such as an immunoconjugate that produces therapeutic effects for which it is administered. The exact dose will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); Goodman & Gilman’s The Pharmacological Basis of Therapeutics, 11^{th} Edition (McGraw-Hill, 2006); and Remington: The Science and Practice of Pharmacy, 22^{nd} Edition, (Pharmaceutical Press, London, 2012)). In the case of cancer, the therapeutically effective amount of the immunoconjugate may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the immunoconjugate may prevent growth and/or kill existing cancer cells, it may be cytostatic and/or cytotoxic. For cancer therapy, efficacy can, for example, be measured by assessing the time to disease progression (TTP) and/or determining the response rate (RR)

“Recipient,” “individual,” “subject,” “host,” and “patient” are used interchangeably and refer to any mammalian subject for whom diagnosis, treatment, or therapy is desired (e.g., humans). “Mammal” for purposes of treatment refers to any animal classified as a mammal, including humans, domestic and farm animals, and zoo, sports, or pet animals, such as dogs, horses, cats, cows, sheep, goats, pigs, camels, etc. In certain embodiments, the mammal is human.

The phrase “synergistic adjuvant” or “synergistic combination” in the context of this invention includes the combination of two immune modulators such as a receptor agonist, cytokine, and adjuvant polypeptide, that in combination elicit a synergistic effect on immunity relative to either administered alone. Particularly, the immunoconjugates disclosed herein comprise synergistic combinations of the claimed adjuvant and antibody construct. These synergistic combinations upon administration elicit a greater effect on immunity, e.g., relative to when the antibody construct or adjuvant is administered in the absence of the other moiety. Further, a decreased amount of the immunoconjugate may be administered (as measured by the total number of antibody constructs or the total number of adjuvants administered as part of the immunoconjugate) compared to when either the antibody construct or adjuvant is administered alone.

As used herein, the term “administering” refers to parenteral, intravenous, intraperitoneal, intramuscular, intratumoral, intralesional, intranasal, or subcutaneous administration, oral administration, administration as a suppository, topical contact, intrathecal administration, or the implantation of a slow-release device, e.g., a mini-osmotic pump, to the subj ect.

The terms “about” and “around,” as used herein to modify a numerical value, indicate a close range surrounding the numerical value. Thus, if “X” is the value, “about X” or “around X” indicates a value of from 0.9X to 1.1X, e.g., from 0.95X to 1.05X or from 0.99X to 1.01X. A reference to “about X” or “around X” specifically indicates at least the values X, 0.95X, 0.96X, 0.97X, 0.98X, 0.99X, 1.01X, 1.02X, 1.03X, 1.04X, and 1.05X. Accordingly, “about X” and “around X” are intended to teach and provide written description support for a claim limitation of, e.g., “0.98X.”

### ANTIBODIES

The immunoconjugate of the invention comprises an antibody. Included in the scope of the embodiments of the invention are functional variants of the antibody constructs or antigen binding domain described herein. The term “functional variant” as used herein refers to an antibody construct having an antigen binding domain with substantial or significant sequence identity or similarity to a parent antibody construct or antigen binding domain, which functional variant retains the biological activity of the antibody construct or antigen binding domain of which it is a variant. Functional variants encompass, for example, those variants of the antibody constructs or antigen binding domain described herein (the parent antibody construct or antigen binding domain) that retain the ability to recognize target cells expressing PD-L1, HER2 or CEA to a similar extent, the same extent, or to a higher extent, as the parent antibody construct or antigen binding domain.

In reference to the antibody construct or antigen binding domain, the functional variant can, for instance, be at least about 30%, about 50%, about 75%, about 80%, about 85%, about 90%, about 91%, about 92%, about 93%, about 94%, about 95%, about 96%, about 97%, about 98%, about 99% or more identical in amino acid sequence to the antibody construct or antigen binding domain.

A functional variant can, for example, comprise the amino acid sequence of the parent antibody construct or antigen binding domain with at least one conservative amino acid substitution. Alternatively, or additionally, the functional variants can comprise the amino acid sequence of the parent antibody construct or antigen binding domain with at least one non-conservative amino acid substitution. In this case, it is preferable for the non-conservative amino acid substitution to not interfere with or inhibit the biological activity of the functional variant. The non-conservative amino acid substitution may enhance the biological activity of the functional variant, such that the biological activity of the functional variant is increased as compared to the parent antibody construct or antigen binding domain.

The antibodies comprising the immunoconjugates of the invention include Fc engineered variants. In some embodiments, the mutations in the Fc region that result in modulated binding to one or more Fc receptors can include one or more of the following mutations: SD (S239D), SDIE (S239D/I332E), SE (S267E), SELF (S267E/L328F), SDIE (S239D/I332E), SDIEAL (S239D/I332E/A330L), GA (G236A), ALIE (A330L/I332E), GASDALIE (G236A/S239D/A330L/I332E), V9 (G237D/P238D/P271G/A330R), and V11 (G237D/P238D/H268D/P271G/A330R), and/or one or more mutations at the following amino acids: E345R, E233, G237, P238, H268, P271, L328 and A330. Additional Fc region modifications for modulating Fc receptor binding are described in, for example, US 2016/0145350; US 7416726; and US 5624821, which are hereby incorporated by reference in their entireties herein.

The antibodies comprising the immunoconjugates of the invention include glycan variants, such as afucosylation. In some embodiments, the Fc region of the binding agents are modified to have an altered glycosylation pattern of the Fc region compared to the native non-modified Fc region.

Amino acid substitutions of the inventive antibody constructs or antigen binding domains are preferably conservative amino acid substitutions. Conservative amino acid substitutions are known in the art, and include amino acid substitutions in which one amino acid having certain physical and/or chemical properties is exchanged for another amino acid that has the same or similar chemical or physical properties. For instance, the conservative amino acid substitution can be an acidic/negatively charged polar amino acid substituted for another acidic/negatively charged polar amino acid (e.g., Asp or Glu), an amino acid with a nonpolar side chain substituted for another amino acid with a nonpolar side chain (e.g., Ala, Gly, Val, Ile, Leu, Met, Phe, Pro, Trp, Cys, Val, etc.), a basic/positively charged polar amino acid substituted for another basic/positively charged polar amino acid (e.g., Lys, His, Arg, etc.), an uncharged amino acid with a polar side chain substituted for another uncharged amino acid with a polar side chain (e.g., Asn, Gln, Ser, Thr, Tyr, etc.), an amino acid with a beta-branched side-chain substituted for another amino acid with a beta-branched side-chain (e.g., Ile, Thr, and Val), an amino acid with an aromatic side-chain substituted for another amino acid with an aromatic side chain (e.g., His, Phe, Trp, and Tyr), etc.

The antibody construct or antigen binding domain can consist essentially of the specified amino acid sequence or sequences described herein, such that other components, e.g., other amino acids, do not materially change the biological activity of the antibody construct or antigen binding domain functional variant.

In some embodiments, the antibodies in the immunoconjugates contain a modified Fc region, wherein the modification modulates the binding of the Fc region to one or more Fc receptors.

In some embodiments, the antibodies in the immunoconjugates (e.g., antibodies conjugated to at least two adjuvant moieties) contain one or more modifications (e.g., amino acid insertion, deletion, and/or substitution) in the Fc region that results in modulated binding (e.g., increased binding or decreased binding) to one or more Fc receptors (e.g., FcγRI (CD64), FcγRIIA (CD32A), FcγRIIB (CD32B), FcγRIIIA (CD16a), and/or FcγRIIIB (CD16b)) as compared to the native antibody lacking the mutation in the Fc region. In some embodiments, the antibodies in the immunoconjugates contain one or more modifications (e.g., amino acid insertion, deletion, and/or substitution) in the Fc region that reduce the binding of the Fc region of the antibody to FcγRIIB. In some embodiments, the antibodies in the immunoconjugates contain one or more modifications (e.g., amino acid insertion, deletion, and/or substitution) in the Fc region of the antibody that reduce the binding of the antibody to FcγRIIB while maintaining the same binding or having increased binding to FcγRI (CD64), FcγRIIA (CD32A), and/or FcRγIIIA (CD16a) as compared to the native antibody lacking the mutation in the Fc region. In some embodiments, the antibodies in the immunoconjugates contain one of more modifications in the Fc region that increase the binding of the Fc region of the antibody to FcγRIIB.

In some embodiments, the modulated binding is provided by mutations in the Fc region of the antibody relative to the native Fc region of the antibody. The mutations can be in a CH2 domain, a CH3 domain, or a combination thereof. A “native Fc region” is synonymous with a “wild-type Fc region” and comprises an amino acid sequence that is identical to the amino acid sequence of an Fc region found in nature or identical to the amino acid sequence of the Fc region found in the native antibody (e.g., cetuximab). Native sequence human Fc regions include a native sequence human IgG1 Fc region, native sequence human IgG2 Fc region, native sequence human IgG3 Fc region, and native sequence human IgG4 Fc region, as well as naturally occurring variants thereof. Native sequence Fc includes the various allotypes of Fcs (Jefferis et al., (2009) mAbs, 1(4):332-338).

In some embodiments, the mutations in the Fc region that result in modulated binding to one or more Fc receptors can include one or more of the following mutations: SD (S239D), SDIE (S239D/I332E), SE (S267E), SELF (S267E/L328F), SDIE (S239D/I332E), SDIEAL (S239D/I332E/A330L), GA (G236A), ALIE (A330L/I332E), GASDALIE (G236A/S239D/A330L/I332E), V9 (G237D/P238D/P271G/A330R), and V11 (G237D/P238D/H268D/P271G/A330R), and/or one or more mutations at the following amino acids: E233, G237, P238, H268, P271, L328 and A330. Additional Fc region modifications for modulating Fc receptor binding are described in, for example, US 2016/0145350 and US 7416726 and US 5624821, which are hereby incorporated by reference in their entireties.

In some embodiments, the Fc region of the antibodies of the immunoconjugates are modified to have an altered glycosylation pattern of the Fc region compared to the native non-modified Fc region.

Human immunoglobulin is glycosylated at the Asn297 residue in the Cγ2 domain of each heavy chain. This N-linked oligosaccharide is composed of a core heptasaccharide, N-acetylglucosamine4Mannose3 (GlcNAc4Man3). Removal of the heptasaccharide with endoglycosidase or PNGase F is known to lead to conformational changes in the antibody Fc region, which can significantly reduce antibody-binding affinity to activating FcγR and lead to decreased effector function. The core heptasaccharide is often decorated with galactose, bisecting GlcNAc, fucose, or sialic acid, which differentially impacts Fc binding to activating and inhibitory FcγR. Additionally, it has been demonstrated that α2,6-sialyation enhances anti-inflammatory activity in vivo, while defucosylation leads to improved FcγRIIIa binding and a 10-fold increase in antibody-dependent cellular cytotoxicity and antibody-dependent phagocytosis. Specific glycosylation patterns, therefore, can be used to control inflammatory effector functions.

In some embodiments, the modification to alter the glycosylation pattern is a mutation. For example, a substitution at Asn297. In some embodiments, Asn297 is mutated to glutamine (N297Q). Methods for controlling immune response with antibodies that modulate FcγR-regulated signaling are described, for example, in US 7416726; US 2007/0014795; and US 2008/0286819, which are hereby incorporated by reference in their entireties.

In some embodiments, the antibodies of the immunoconjugates are modified to contain an engineered Fab region with a non-naturally occurring glycosylation pattern. For example, hybridomas can be genetically engineered to secrete afucosylated mAb, desialylated mAb or deglycosylated Fc with specific mutations that enable increased FcRγIIIa binding and effector function. In some embodiments, the antibodies of the immunoconjugates are engineered to be afucosylated.

In some embodiments, the antibody construct further comprises an Fc domain. In certain embodiments, the antibody construct is an antibody. In certain embodiments, the antibody construct is a fusion protein. The antigen binding domain can be a single-chain variable region fragment (scFv). A single-chain variable region fragment (scFv), which is a truncated Fab fragment including the variable (V) domain of an antibody heavy chain linked to a V domain of a light antibody chain via a synthetic peptide, can be generated using routine recombinant DNA technology techniques. Similarly, disulfide-stabilized variable region fragments (dsFv) can be prepared by recombinant DNA technology. The antibody construct or antigen binding domain may comprise one or more variable regions (e.g., two variable regions) of an antigen binding domain of an anti-PD-L1 antibody, an anti-HER2 antibody, or an anti-CEA antibody, each variable region comprising a CDR1, a CDR2, and a CDR3.

In some embodiments, the antibodies in the immunoconjugates contain a modified Fc region, wherein the modification modulates the binding of the Fc region to one or more Fc receptors.

In some embodiments, the entire Fc region of an antibody in the immunoconjugates is exchanged with a different Fc region, so that the Fab region of the antibody is conjugated to a non-native Fc region. For example, the Fab region of cetuximab, which normally comprises an IgG1 Fc region, can be conjugated to IgG2, IgG3, IgG4, or IgA, or the Fab region of nivolumab, which normally comprises an IgG4 Fc region, can be conjugated to IgG1, IgG2, IgG3, IgA1, or IgG2. In some embodiments, the Fc modified antibody with a non-native Fc domain also comprises one or more amino acid modification, such as the S228P mutation within the IgG4 Fc, that modulate the stability of the Fc domain described. In some embodiments, the Fc modified antibody with a non-native Fc domain also comprises one or more amino acid modifications described herein that modulate Fc binding to FcR.

In some embodiments, the modifications that modulate the binding of the Fc region to FcR do not alter the binding of the Fab region of the antibody to its antigen when compared to the native non-modified antibody. In other embodiments, the modifications that modulate the binding of the Fc region to FcR also increase the binding of the Fab region of the antibody to its antigen when compared to the native non-modified antibody.

In an exemplary embodiment, the immunoconjugates of the invention comprise an antibody construct that comprises an antigen binding domain that specifically recognizes and binds PD-L1.

In an exemplary embodiment, the immunoconjugates of the invention comprise an antibody construct that comprises an antigen binding domain that specifically recognizes and binds PD-L1.

Programmed Death-Ligand 1 (PD-L1, cluster of differentiation 274, CD274, B7-homolog 1, or B7-H1) belongs to the B7 protein superfamily, and is a ligand of programmed cell death protein 1 (PD-1, PDCD1, cluster of differentiation 279, or CD279). PD-L1 can also interact with B7.1 (CD80) and such interaction is believed to inhibit T cell priming. The PD-L1/PD-1 axis plays a large role in suppressing the adaptive immune response. More specifically, it is believed that engagement of PD-L1 with its receptor, PD-1, delivers a signal that inhibits activation and proliferation of T-cells. Agents that bind to PD-L1 and prevent the ligand from binding to the PD-1 receptor prevent this immunosuppression, and can, therefore, enhance an immune response when desired, such as for the treatment of cancers, or infections. PD-L1/PD-1 pathway also contributes to preventing autoimmunity and therefore agonistic agents against PD-L1 or agents that deliver immune inhibitory payloads may help treatment of autoimmune disorders.

Several antibodies targeting PD-L1 have been developed for the treatment of cancer, including atezolizumab (TECENTRIQ™), durvalumab (IMFINZI™), and avelumab (BAVENCIO™). Nevertheless, there continues to be a need for new PD-L1-binding agents, including agents that bind PD-L1 with high affinity and effectively prevent PD-L1/PD-1 signaling and agents that can deliver therapeutic payloads to PD-L1 expressing cells. In addition, there is a need for new PD-L1-binding agents to treat autoimmune disorders and infections.

A method is provided of delivering an aminobenzazepine derivative payload to a cell expressing PD-L1 comprising administering to the cell, or mammal comprising the cell, an immunoconjugate comprising an anti-PD-L1 antibody covalently attached to a linker which is covalently attached to one or more aminobenzazepine moieties.

Also provided is a method for enhancing or reducing or inhibiting an immune response in a mammal, and a method for treating a disease, disorder, or condition in a mammal that is responsive to PD-L1 inhibition, which methods comprise administering a PD-L1 immunoconjugate thereof, to the mammal.

The invention provides a PD-L1 binding agent comprising an immunoglobulin heavy chain variable region polypeptide and an immunoglobulin light chain variable region polypeptide.

The PD-L1 binding agent specifically binds PD-L1. The binding specificity of the agent allows for targeting PD-L1 expressing cells, for instance, to deliver therapeutic payloads to such cells.

In some embodiments, the PD-L1 binding agent (Type A or Type B) binds to human PD-L1, for example, a protein comprising SEQ ID NO: 307. However, binding agents that bind to any PD-L1 homolog or paralog also are encompassed. In some embodiments, the PD-L1 protein comprises at least about 70%, about 75%, about 80%, about 85%, about 90%, about 91%, about 92%, about 93%, about 94%, about 95%, about 96%, about 97%, about 98%, about 99%, or more sequence identity to SEQ ID NO: 307. In some embodiments, the binding agent binds human PD-L1 and cynomolgus PD-L1; or human, cynomolgus and mouse PD-L1.

MRIFAVFIFMTYWHLLNAFTVTVPKDLYWEYGSNMTIECKFPVEKQLDLA
ALIVYWEMEDKNIIQFVHGEEDLKVQHSSYRQRARLLKDQLSLGNAALQI
TDVKLQDAGVYRCMISYGGADYKRITVKVNAPYNKINQRILWDPVTSEHE
LTCQAEGYPKAEVIWTSSDHQVLSGKTTTTNSKREEKLFNVTSTLRINTT
TNEIFYCTFRRLDPEENHTAELVIPELPLAHPPNERTHLVILGAILLCLG
VALTFIFRLRKGRMMDVKKCGIQDTNSKKQSDTHLEET SEQ ID NO:
307

In some embodiments, the PD-L1 binding agent binds PD-L1 without substantially inhibiting or preventing PD-L1 from binding to its receptor, PD-1. However, in other embodiments, the PD-L1 binding agent can completely or partially block (inhibit or prevent) binding of PD-L1 to its receptor, PD-1, such that the antibody can be used to inhibit PD-L1/PD-1 signaling (e.g., for therapeutic purposes).

The antibody or antigen-binding antibody fragment can be monospecific for PD-L1, or can be bispecific or multi-specific. For instance, in bivalent or multivalent antibodies or antibody fragments, the binding domains can be different targeting different epitopes of the same antigen or targeting different antigens. Methods of constructing multivalent binding constructs are known in the art. Bispecific and multispecific antibodies are known in the art. Furthermore, a diabody, triabody, or tetrabody can be provided, which is a dimer, trimer, or tetramer of polypeptide chains each comprising a V_{H} connected to a V_{L} by a peptide linker that is too short to allow pairing between the V_{H} and V_{L} on the same polypeptide chain, thereby driving the pairing between the complementary domains on different V_{H} -V_{L} polypeptide chains to generate a multimeric molecule having two, three, or four functional antigen binding sites. Also, bis-scFv fragments, which are small scFv fragments with two different variable domains can be generated to produce bispecific bis-scFv fragments capable of binding two different epitopes. Fab dimers (Fab2) and Fab trimers (Fab3) can be produced using genetic engineering methods to create multispecific constructs based on Fab fragments.

The PD-L1-binding agent also can be an antibody conjugate. In this respect, the PD-L1-binding agent can be a conjugate of (1) an antibody, an alternative scaffold, or fragments thereof, and (2) a protein or non-protein moiety. For example, the PD-L1 binding agent can be conjugated to a peptide, a fluorescent molecule, chemotherapeutic or other cytotoxic payload, immune-activating or immune-suppressive agent.

The PD-L1-binding agent can be, or can be obtained from, a human antibody, a non-human antibody, a humanized antibody, or a chimeric antibody, or corresponding antibody fragments. A “chimeric” antibody is an antibody or fragment thereof typically comprising human constant regions and non-human variable regions. A “humanized” antibody is a monoclonal antibody typically comprising a human antibody scaffold but with non-human origin amino acids or sequences in at least one CDR (e.g., 1, 2, 3, 4, 5, or all six CDRs).

### PD-L1-Binding Agents - Type A

Provided herein are PD-L1 binding agents comprising an immunoglobulin heavy chain variable region polypeptide and an immunoglobulin light chain variable region polypeptide. In some embodiments, the PD-L1 binding agents (Type A) comprise an immunoglobulin heavy chain variable region of any one of SEQ ID NOs: 223-264, or at least the CDRs thereof; and an immunoglobulin light chain variable region of any one of SEQ ID NOs: 265-306 or at least the CDRs thereof. In other embodiments, the PD-L1 binding agents (Type A) comprise an immunoglobulin heavy chain variable region polypeptide with an amino acid sequence that is at least 90% identical to any one of SEQ ID NOs: 223-264, and an immunoglobulin light chain variable region polypeptide with an amino acid sequence that is at least 90% identical to any one of SEQ ID NOs: 265-306. In yet other embodiments, the PD-L1 binding agent (Type A), the immunoglobulin heavy chain variable region polypeptide comprises a complementarity determining region 1 (HCDR1) comprising any one of SEQ ID NOs: 1-23, a complementarity determining region 2 (HCDR2) comprising any one of SEQ ID NOs: 24-57, and a complementarity determining region 3 (HCDR3) comprising any one of SEQ ID NOs: 58-95; and/or the immunoglobulin light chain variable region polypeptide comprises a complementarity determining region 1 (LCDR1) comprising any one of SEQ ID NOs: 96-128, a complementarity determining region 2 (LCDR2) comprising any one of SEQ ID NOs: 129-151, and a complementarity determining region 3 (LCDR3) comprising any one of SEQ ID NOs: 152-155. Also provided are nucleic acids encoding the PD-L1 binding agents, or the individual heavy and light chains thereof; vectors and cells comprising the nucleic acids; and compositions comprising the binding agents or nucleic acids.

Furthermore, in some embodiments, the PD-L1 binding agents (Type A) provided herein cause cellular internalization of PD-L1 or the PD-L1/PD-L1 binding agent complex upon binding to PD-L1 on the cell surface. Without wishing to be bound by any particular theory or mechanism of action, it is believed that the PD-L1 binding agents according to this embodiment cause PD-L1 internalization upon binding, and remain bound to PD-L1 during internalization resulting in internalization of the binding agent along with PD-L1. Cellular internalization of PD-L1 and bound PD-L1 binding agent can be determined by any suitable method, such as assaying for persistence on the cell surface and/or detection of internalized antibodies. In some embodiments, the PD-L1 binding agent internalizes strongly enough that at least about 25% (e.g., at least about 35%, at least about 50%, at least about 75%, or at least about 90%) of the PD-L1 binding agent that binds PD-L1 on the cell surface is internalized (e.g., using a surface persistence assay, about 75% or less, about 65% or less, about 50% or less, about 75% or less or about 10% or less of PD-L1 binding agent molecules bound to PD-L1 on the cell surface at the beginning of the assay remain bound at the end of the assay).

In an embodiment, the PD-L1 binding agent (Type A) comprises an immunoglobulin heavy chain variable region of any one of SEQ ID NOs: 223-264, a sequence that is at least about 90%, at least about 91%, at least about 92%, at least about 93%, at least about 94%, at least about 95%, at least about 96%, at least about 97%, at least about 98%, or at least about 99% identical to SEQ ID NOs: 223-264, or at least the CDRs thereof; and/or an immunoglobulin light chain variable region of any one of SEQ ID NOs: 265-306, a sequence that is at least about 90%, at least about 91%, at least about 92%, at least about 93%, at least about 94%, at least about 95%, at least about 96%, at least about 97%, at least about 98%, or at least about 99% identical to SEQ ID NOs: 265-306, or at least the CDRs thereof.

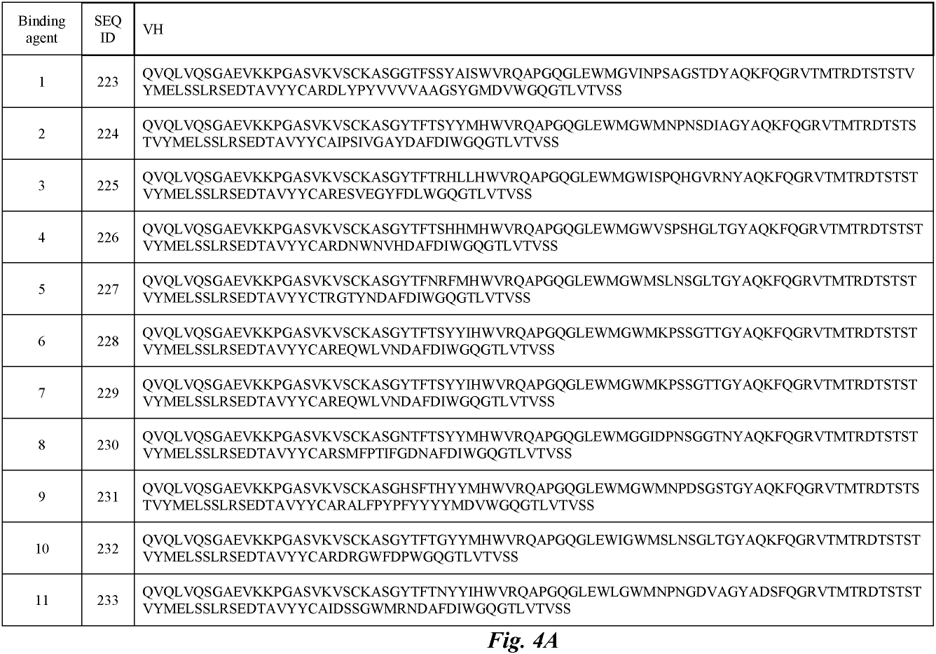

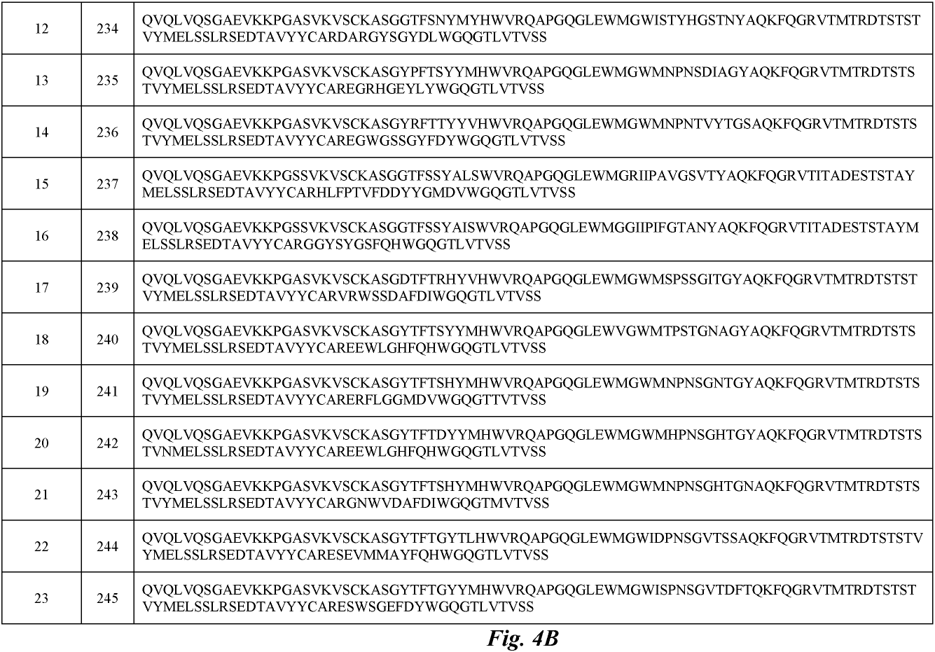

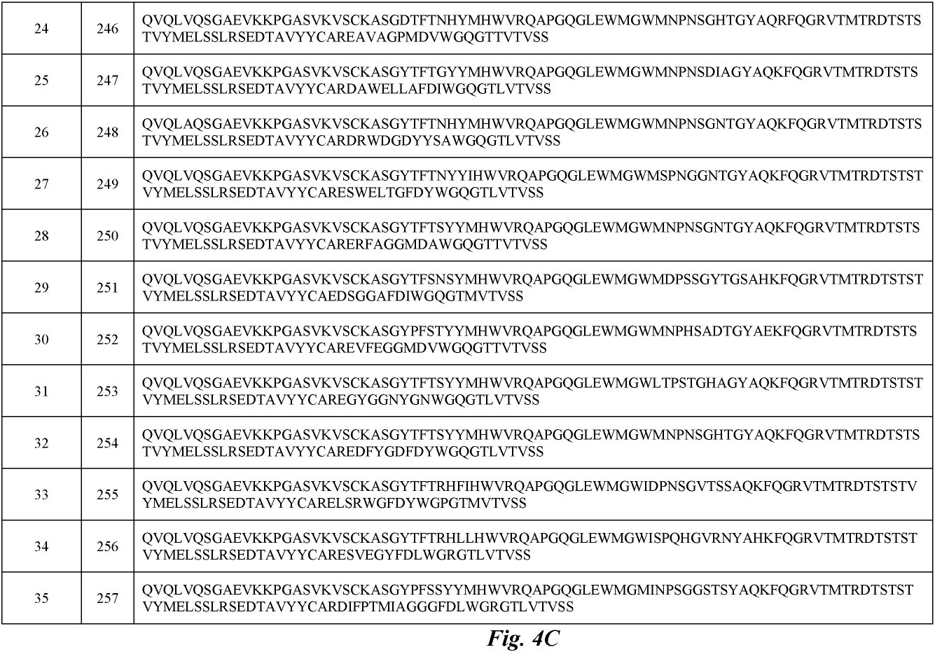

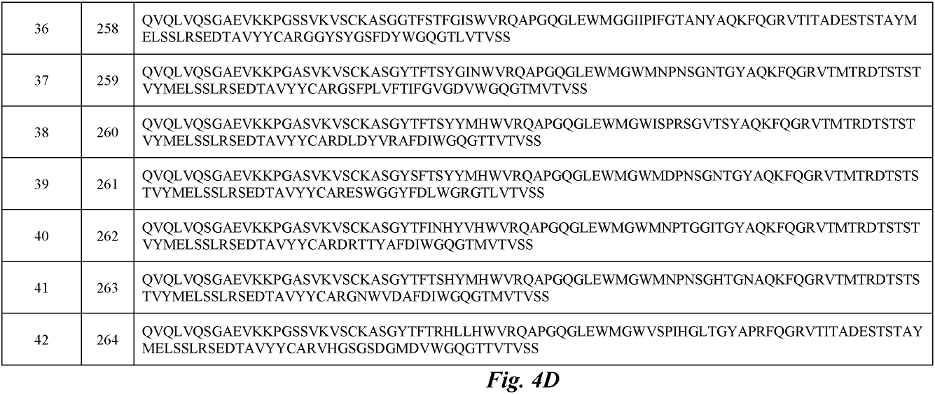

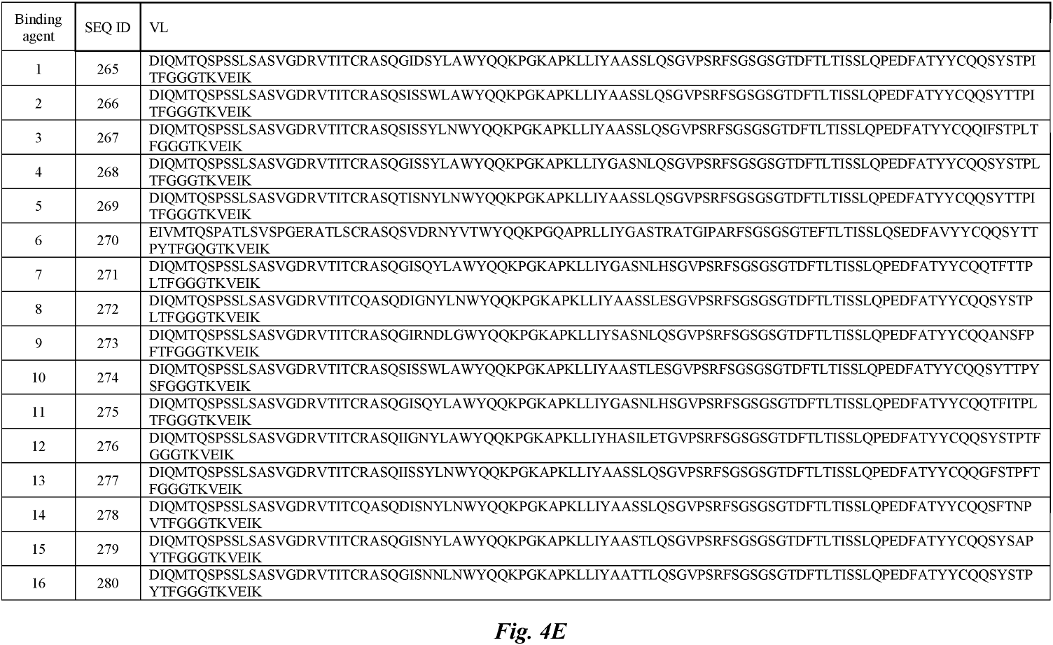

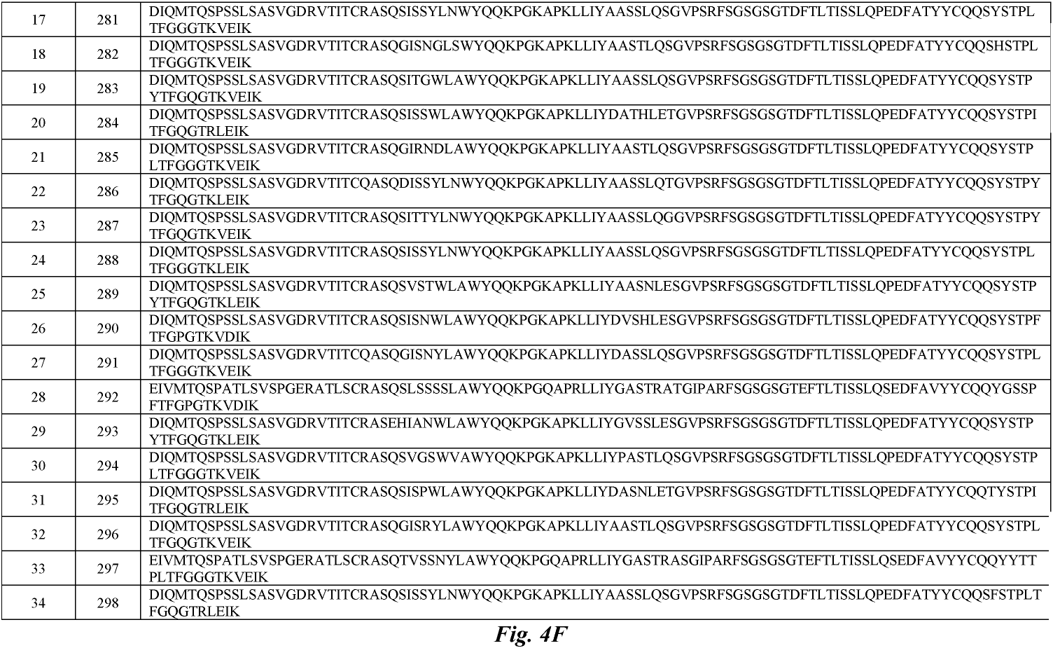

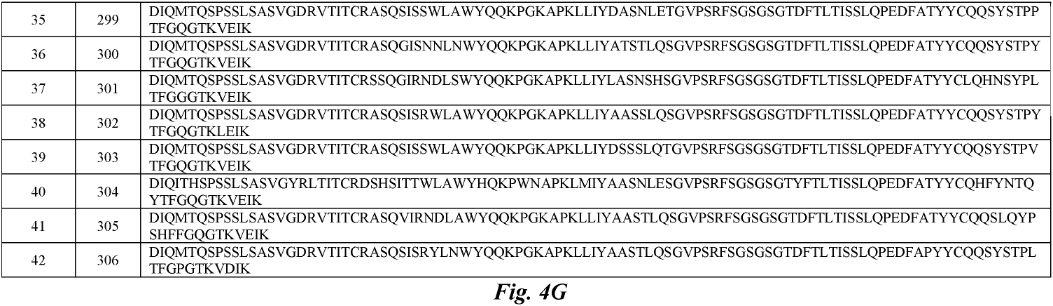

By way of further illustration, the PD-L1 binding agent (Type A) can comprise:
(1) an immunoglobulin heavy chain variable region of SEQ ID NO: 223, or at least the CDRs thereof, and/or an immunoglobulin light chain variable region of SEQ ID NO: 265, or at least the CDRs thereof;
(2) an immunoglobulin heavy chain variable region of SEQ ID NO: 224, or at least the CDRs thereof, and/or an immunoglobulin light chain variable region of SEQ ID NO: 266, or at least the CDRs thereof;
(3) an immunoglobulin heavy chain variable region of SEQ ID NO: 225, or at least the CDRs thereof, and/or an immunoglobulin light chain variable region of SEQ ID NO: 267, or at least the CDRs thereof;
(4) an immunoglobulin heavy chain variable region of SEQ ID NO: 226, or at least the CDRs thereof, and/or an immunoglobulin light chain variable region of SEQ ID NO: 268, or at least the CDRs thereof;
(5) an immunoglobulin heavy chain variable region of SEQ ID NO: 227, or at least the CDRs thereof, and/or an immunoglobulin light chain variable region of SEQ ID NO: 269, or at least the CDRs thereof;
(6) an immunoglobulin heavy chain variable region of SEQ ID NO: 228, or at least the CDRs thereof, and/or an immunoglobulin light chain variable region of SEQ ID NO: 270, or at least the CDRs thereof;
(7) an immunoglobulin heavy chain variable region of SEQ ID NO: 229, or at least the CDRs thereof, and/or an immunoglobulin light chain variable region of SEQ ID NO: 271, or at least the CDRs thereof;
(8) an immunoglobulin heavy chain variable region of SEQ ID NO: 230, or at least the CDRs thereof, and/or an immunoglobulin light chain variable region of SEQ ID NO: 272, or at least the CDRs thereof;
(9) an immunoglobulin heavy chain variable region of SEQ ID NO: 231, or at least the CDRs thereof, and/or an immunoglobulin light chain variable region of SEQ ID NO: 273, or at least the CDRs thereof;
(10) an immunoglobulin heavy chain variable region of SEQ ID NO: 232, or at least the CDRs thereof, and/or an immunoglobulin light chain variable region of SEQ ID NO: 274, or at least the CDRs thereof;
(11) an immunoglobulin heavy chain variable region of SEQ ID NO: 233, or at least the CDRs thereof, and/or an immunoglobulin light chain variable region of SEQ ID NO: 275, or at least the CDRs thereof;
(12) an immunoglobulin heavy chain variable region of SEQ ID NO: 234, or at least the CDRs thereof, and/or an immunoglobulin light chain variable region of SEQ ID NO: 276, or at least the CDRs thereof;
(13) an immunoglobulin heavy chain variable region of SEQ ID NO: 235, or at least the CDRs thereof, and/or an immunoglobulin light chain variable region of SEQ ID NO: 277, or at least the CDRs thereof;
(14) an immunoglobulin heavy chain variable region of SEQ ID NO: 236, or at least the CDRs thereof, and/or an immunoglobulin light chain variable region of SEQ ID NO: 278, or at least the CDRs thereof;
(15) an immunoglobulin heavy chain variable region of SEQ ID NO: 237, or at least the CDRs thereof, and/or an immunoglobulin light chain variable region of SEQ ID NO: 279, or at least the CDRs thereof;
(16) an immunoglobulin heavy chain variable region of SEQ ID NO: 238, or at least the CDRs thereof, and/or an immunoglobulin light chain variable region of SEQ ID NO: 280, or at least the CDRs thereof;
(17) an immunoglobulin heavy chain variable region of SEQ ID NO: 239, or at least the CDRs thereof, and/or an immunoglobulin light chain variable region of SEQ ID NO: 281, or at least the CDRs thereof;
(18) an immunoglobulin heavy chain variable region of SEQ ID NO: 240, or at least the CDRs thereof, and/or an immunoglobulin light chain variable region of SEQ ID NO: 282, or at least the CDRs thereof;
(19) an immunoglobulin heavy chain variable region of SEQ ID NO: 241, or at least the CDRs thereof, and/or an immunoglobulin light chain variable region of SEQ ID NO: 283, or at least the CDRs thereof;
(20) an immunoglobulin heavy chain variable region of SEQ ID NO: 242, or at least the CDRs thereof, and/or an immunoglobulin light chain variable region of SEQ ID NO: 284, or at least the CDRs thereof;
(21) an immunoglobulin heavy chain variable region of SEQ ID NO: 243, or at least the CDRs thereof, and/or an immunoglobulin light chain variable region of SEQ ID NO: 285, or at least the CDRs thereof;
(22) an immunoglobulin heavy chain variable region of SEQ ID NO: 244, or at least the CDRs thereof, and/or an immunoglobulin light chain variable region of SEQ ID NO: 286, or at least the CDRs thereof;
(23) an immunoglobulin heavy chain variable region of SEQ ID NO: 245, or at least the CDRs thereof, and/or an immunoglobulin light chain variable region of SEQ ID NO: 287, or at least the CDRs thereof;
(24) an immunoglobulin heavy chain variable region of SEQ ID NO: 246, or at least the CDRs thereof, and/or an immunoglobulin light chain variable region of SEQ ID NO: 288, or at least the CDRs thereof;
(25) an immunoglobulin heavy chain variable region of SEQ ID NO: 247, or at least the CDRs thereof, and/or an immunoglobulin light chain variable region of SEQ ID NO: 289, or at least the CDRs thereof;
(26) an immunoglobulin heavy chain variable region of SEQ ID NO: 248, or at least the CDRs thereof, and/or an immunoglobulin light chain variable region of SEQ ID NO: 290, or at least the CDRs thereof;
(27) an immunoglobulin heavy chain variable region of SEQ ID NO: 249, or at least the CDRs thereof, and/or an immunoglobulin light chain variable region of SEQ ID NO: 291, or at least the CDRs thereof;
(28) an immunoglobulin heavy chain variable region of SEQ ID NO: 250, or at least the CDRs thereof, and/or an immunoglobulin light chain variable region of SEQ ID NO: 292, or at least the CDRs thereof;
(29) an immunoglobulin heavy chain variable region of SEQ ID NO: 251, or at least the CDRs thereof, and/or an immunoglobulin light chain variable region of SEQ ID NO: 293, or at least the CDRs thereof;
(30) an immunoglobulin heavy chain variable region of SEQ ID NO: 252, or at least the CDRs thereof, and/or an immunoglobulin light chain variable region of SEQ ID NO: 294, or at least the CDRs thereof;
(31) an immunoglobulin heavy chain variable region of SEQ ID NO: 253, or at least the CDRs thereof, and/or an immunoglobulin light chain variable region of SEQ ID NO: 295, or at least the CDRs thereof;
(32) an immunoglobulin heavy chain variable region of SEQ ID NO: 254, or at least the CDRs thereof, and/or an immunoglobulin light chain variable region of SEQ ID NO: 296, or at least the CDRs thereof;
(33) an immunoglobulin heavy chain variable region of SEQ ID NO: 255, or at least the CDRs thereof, and/or an immunoglobulin light chain variable region of SEQ ID NO: 297, or at least the CDRs thereof;
(34) an immunoglobulin heavy chain variable region of SEQ ID NO: 256, or at least the CDRs thereof, and/or an immunoglobulin light chain variable region of SEQ ID NO: 298, or at least the CDRs thereof;
(35) an immunoglobulin heavy chain variable region of SEQ ID NO: 257, or at least the CDRs thereof, and/or an immunoglobulin light chain variable region of SEQ ID NO: 299, or at least the CDRs thereof;
(36) an immunoglobulin heavy chain variable region of SEQ ID NO: 258, or at least the CDRs thereof, and/or an immunoglobulin light chain variable region of SEQ ID NO: 300, or at least the CDRs thereof;
(37) an immunoglobulin heavy chain variable region of SEQ ID NO: 259, or at least the CDRs thereof, and/or an immunoglobulin light chain variable region of SEQ ID NO: 301, or at least the CDRs thereof;
(38) an immunoglobulin heavy chain variable region of SEQ ID NO: 260, or at least the CDRs thereof, and/or an immunoglobulin light chain variable region of SEQ ID NO: 302, or at least the CDRs thereof;
(39) an immunoglobulin heavy chain variable region of SEQ ID NO: 261, or at least the CDRs thereof, and/or an immunoglobulin light chain variable region of SEQ ID NO: 303, or at least the CDRs thereof;
(40) an immunoglobulin heavy chain variable region of SEQ ID NO: 262, or at least the CDRs thereof, and/or an immunoglobulin light chain variable region of SEQ ID NO: 304, or at least the CDRs thereof;
(41) an immunoglobulin heavy chain variable region of SEQ ID NO: 263, or at least the CDRs thereof, and/or an immunoglobulin light chain variable region of SEQ ID NO: 305, or at least the CDRs thereof;
(42) an immunoglobulin heavy chain variable region of SEQ ID NO: 164, or at least the CDRs thereof, and/or an immunoglobulin light chain variable region of SEQ ID NO: 306, or at least the CDRs thereof; and/or
(43) an immunoglobulin heavy chain variable region of FIGS. 4A-D and/or an immunoglobulin light chain variable region of FIGS. 4E-G, or at least the CDRs thereof.

The CDRs of a given heavy or light chain Ig sequence can be determined in accordance with any of the various known Ig numbering schemes (e.g., Kabat, Chothia, Martin (Enhanced Chothia), IGMT, AbM). In certain embodiments, the PD-L1 binding agent (Type A) comprises one or more of the following CDRs:
a HCDR1 comprising or consisting of any one of SEQ ID NOs: 1-23 or a sequence that is at least about 90%, at least about 91%, at least about 92%, at least about 93%, at least about 94%, at least about 95%, at least about 96%, at least about 97%, at least about 98%, or at least about 99% identical to SEQ ID NOs: 1-23;
a HCDR2 comprising or consisting of any one of SEQ ID NOs: 24-57 or a sequence that is at least about 90%, at least about 91%, at least about 92%, at least about 93%, at least about 94%, at least about 95%, at least about 96%, at least about 97%, at least about 98%, or at least about 99% identical to SEQ ID NOs: 24-57; and
a HCDR3 comprising or consisting of any one of SEQ ID NOs: 58-95 or a sequence that is at least about 90%, at least about 91%, at least about 92%, at least about 93%, at least about 94%, at least about 95%, at least about 96%, at least about 97%, at least about 98%, or at least about 99% identical to SEQ ID NOs: 58-95; and/or the immunoglobulin light chain polypeptide comprises
a LCDR1 comprising or consisting of any one of SEQ ID NOs: 96-128 or a sequence that is at least about 90%, at least about 91%, at least about 92%, at least about 93%, at least about 94%, at least about 95%, at least about 96%, at least about 97%, at least about 98%, or at least about 99% identical to SEQ ID NOs: 96-128;
a LCDR2 comprising or consisting of any one of SEQ ID NOs: 129-151 or a sequence that is at least about 90%, at least about 91%, at least about 92%, at least about 93%, at least about 94%, at least about 95%, at least about 96%, at least about 97%, at least about 98%, or at least about 99% identical to SEQ ID NOs: 129-151; and
a LCDR3 comprising or consisting of any one of SEQ ID NOs: 152-155 or a sequence that is at least about 90%, at least about 91%, at least about 92%, at least about 93%, at least about 94%, at least about 95%, at least about 96%, at least about 97%, at least about 98%, or at least about 99% identical to SEQ ID NOs: 152-155.

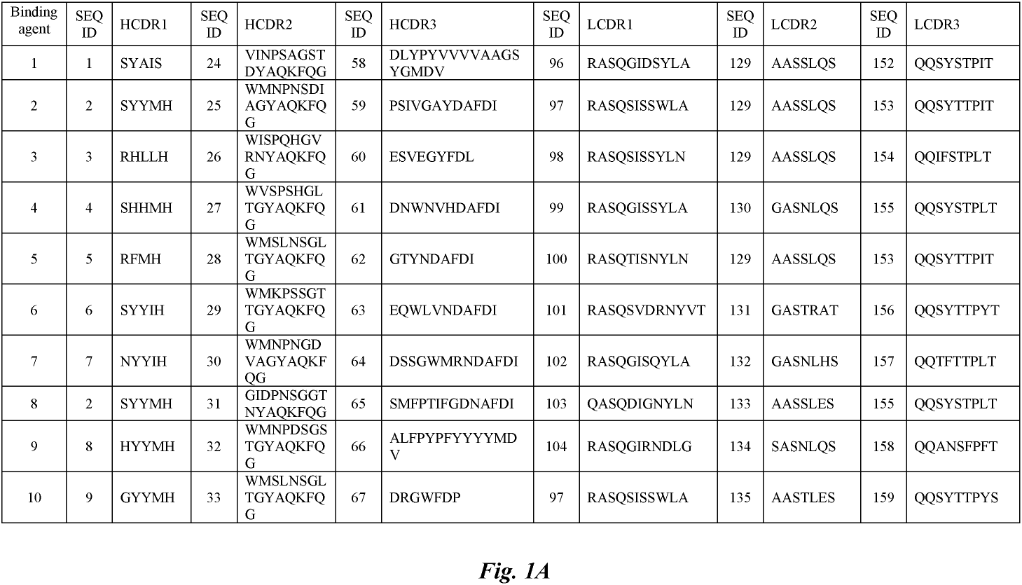

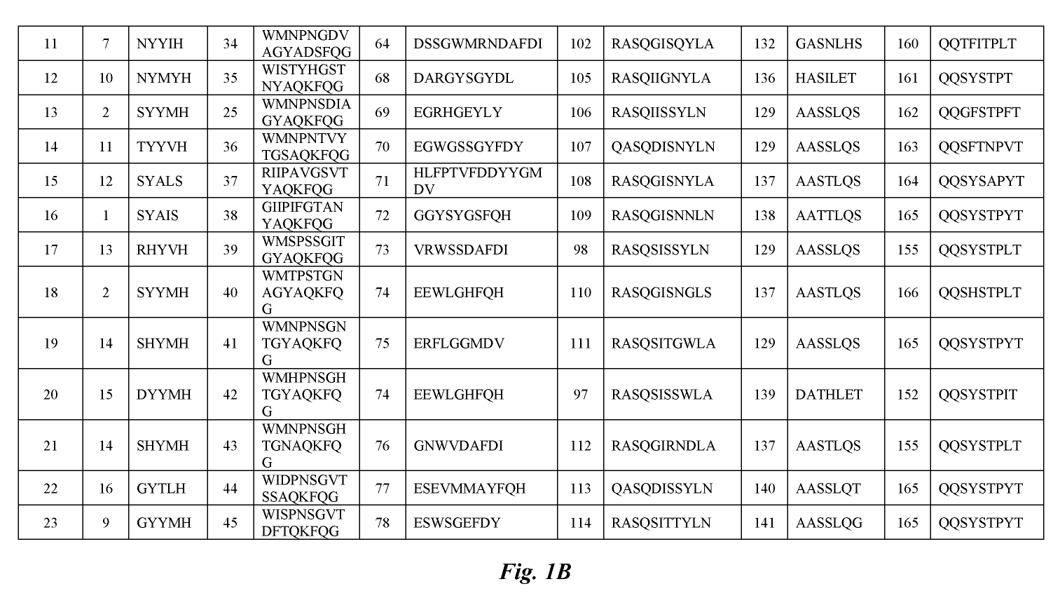

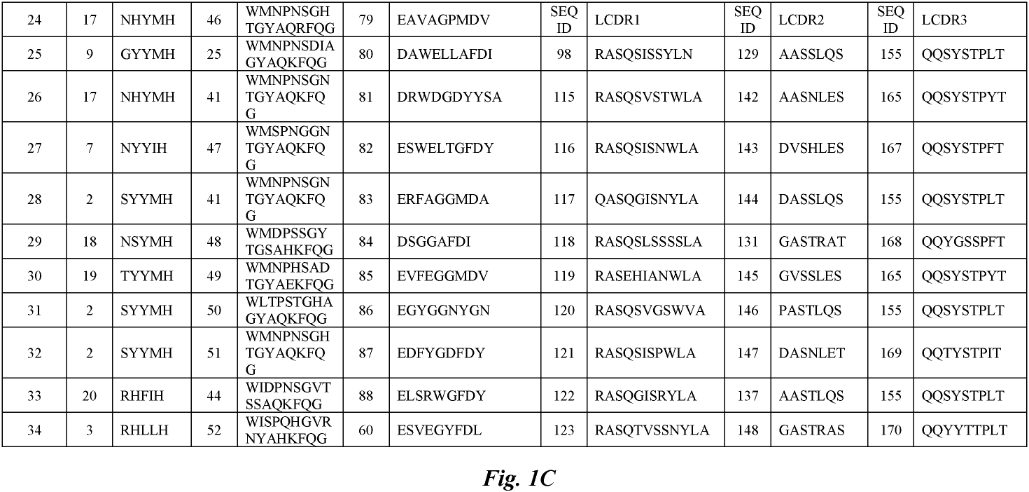

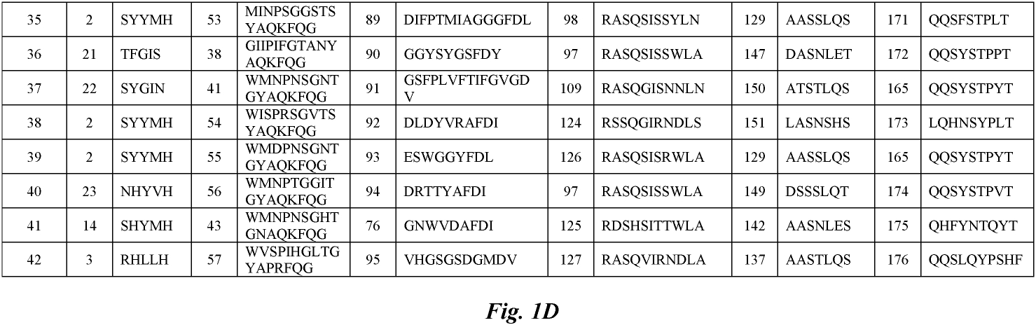

In particular embodiments, the binding agent (Type A) comprises an immunoglobulin heavy chain polypeptide and an immunoglobulin light chain polypeptide, wherein:
(1) the immunoglobulin heavy chain polypeptide comprises a HCDR1 comprising or consisting of SEQ ID NO: 1, a HCDR2 comprising or consisting of SEQ ID NO: 24, and a HCDR3 comprising or consisting of SEQ ID NO: 58; and/or the immunoglobulin light chain polypeptide comprises a LCDR1 comprising or consisting of SEQ ID NO: 96, a LCDR2 comprising or consisting of SEQ ID NO: 129, and a LCDR3 comprising or consisting of SEQ ID NO: 152;
(2) the immunoglobulin heavy chain polypeptide comprises a HCDR1 comprising or consisting of SEQ ID NO: 2, a HCDR2 comprising or consisting of SEQ ID NO: 25, and a HCDR3 comprising or consisting of SEQ ID NO: 59; and/or the immunoglobulin light chain polypeptide comprises a LCDR1 comprising or consisting of SEQ ID NO: 97, a LCDR2 comprising or consisting of SEQ ID NO: 129, and a LCDR3 comprising or consisting of SEQ ID NO: 153;
(3) the immunoglobulin heavy chain polypeptide comprises a HCDR1 comprising or consisting of SEQ ID NO: 3, a HCDR2 comprising or consisting of SEQ ID NO: 26, and a HCDR3 comprising or consisting of SEQ ID NO: 60; and/or the immunoglobulin light chain polypeptide comprises a LCDR1 comprising or consisting of SEQ ID NO: 98, a LCDR2 comprising or consisting of SEQ ID NO: 129, and a LCDR3 comprising or consisting of SEQ ID NO: 154;
(4) the immunoglobulin heavy chain polypeptide comprises a HCDR1 comprising or consisting of SEQ ID NO: 4, a HCDR2 comprising or consisting of SEQ ID NO: 27, and a HCDR3 comprising or consisting of SEQ ID NO: 61; and/or the immunoglobulin light chain polypeptide comprises a LCDR1 comprising or consisting of SEQ ID NO: 99, a LCDR2 comprising or consisting of SEQ ID NO: 130, and a LCDR3 comprising or consisting of SEQ ID NO: 155;
(5) the immunoglobulin heavy chain polypeptide comprises a HCDR1 comprising or consisting of SEQ ID NO: 5, a HCDR2 comprising or consisting of SEQ ID NO: 28, and a HCDR3 comprising or consisting of SEQ ID NO: 62; and/or the immunoglobulin light chain polypeptide comprises a LCDR1 comprising or consisting of SEQ ID NO: 100, a LCDR2 comprising or consisting of SEQ ID NO: 129, and a LCDR3 comprising or consisting of SEQ ID NO: 153;
(6) the immunoglobulin heavy chain polypeptide comprises a HCDR1 comprising or consisting of SEQ ID NO: 6, a HCDR2 comprising or consisting of SEQ ID NO: 29, and a HCDR3 comprising or consisting of SEQ ID NO: 63; and/or the immunoglobulin light chain polypeptide comprises a LCDR1 comprising or consisting of SEQ ID NO: 101, a LCDR2 comprising or consisting of SEQ ID NO: 131, and a LCDR3 comprising or consisting of SEQ ID NO: 156;
(7) the immunoglobulin heavy chain polypeptide comprises a HCDR1 comprising or consisting of SEQ ID NO: 7, a HCDR2 comprising or consisting of SEQ ID NO: 30, and a HCDR3 comprising or consisting of SEQ ID NO: 64; and/or the immunoglobulin light chain polypeptide comprises a LCDR1 comprising or consisting of SEQ ID NO: 102, a LCDR2 comprising or consisting of SEQ ID NO: 132, and a LCDR3 comprising or consisting of SEQ ID NO: 157;
(8) the immunoglobulin heavy chain polypeptide comprises a HCDR1 comprising or consisting of SEQ ID NO: 2, a HCDR2 comprising or consisting of SEQ ID NO: 31, and a HCDR3 comprising or consisting of SEQ ID NO: 65; and/or the immunoglobulin light chain polypeptide comprises a LCDR1 comprising or consisting of SEQ ID NO: 103, a LCDR2 comprising or consisting of SEQ ID NO: 133, and a LCDR3 comprising or consisting of SEQ ID NO: 155;
(9) the immunoglobulin heavy chain polypeptide comprises a HCDR1 comprising or consisting of SEQ ID NO: 8, a HCDR2 comprising or consisting of SEQ ID NO: 32, and a HCDR3 comprising or consisting of SEQ ID NO: 66; and/or the immunoglobulin light chain polypeptide comprises a LCDR1 comprising or consisting of SEQ ID NO: 104, a LCDR2 comprising or consisting of SEQ ID NO: 134, and a LCDR3 comprising or consisting of SEQ ID NO: 158;
(10) the immunoglobulin heavy chain polypeptide comprises a HCDR1 comprising or consisting of SEQ ID NO: 9, a HCDR2 comprising or consisting of SEQ ID NO: 33, and a HCDR3 comprising or consisting of SEQ ID NO: 67; and/or the immunoglobulin light chain polypeptide comprises a LCDR1 comprising or consisting of SEQ ID NO: 97, a LCDR2 comprising or consisting of SEQ ID NO: 135, and a LCDR3 comprising or consisting of SEQ ID NO: 159;
(11) the immunoglobulin heavy chain polypeptide comprises a HCDR1 comprising or consisting of SEQ ID NO: 7, a HCDR2 comprising or consisting of SEQ ID NO: 34, and a HCDR3 comprising or consisting of SEQ ID NO: 64; and/or the immunoglobulin light chain polypeptide comprises a LCDR1 comprising or consisting of SEQ ID NO: 102, a LCDR2 comprising or consisting of SEQ ID NO: 132, and a LCDR3 comprising or consisting of SEQ ID NO: 160;
(12) the immunoglobulin heavy chain polypeptide comprises a HCDR1 comprising or consisting of SEQ ID NO: 10, a HCDR2 comprising or consisting of SEQ ID NO: 35, and a HCDR3 comprising or consisting of SEQ ID NO: 68; and/or the immunoglobulin light chain polypeptide comprises a LCDR1 comprising or consisting of SEQ ID NO: 105, a LCDR2 comprising or consisting of SEQ ID NO: 136, and a LCDR3 comprising or consisting of SEQ ID NO: 161;
(13) the immunoglobulin heavy chain polypeptide comprises a HCDR1 comprising or consisting of SEQ ID NO: 2, a HCDR2 comprising or consisting of SEQ ID NO: 25, and a HCDR3 comprising or consisting of SEQ ID NO: 69; and/or the immunoglobulin light chain polypeptide comprises a LCDR1 comprising or consisting of SEQ ID NO: 106, a LCDR2 comprising or consisting of SEQ ID NO: 129, and a LCDR3 comprising or consisting of SEQ ID NO: 162;
(14) the immunoglobulin heavy chain polypeptide comprises a HCDR1 comprising or consisting of SEQ ID NO: 11, a HCDR2 comprising or consisting of SEQ ID NO: 36, and a HCDR3 comprising or consisting of SEQ ID NO: 70; and/or the immunoglobulin light chain polypeptide comprises a LCDR1 comprising or consisting of SEQ ID NO: 107, a LCDR2 comprising or consisting of SEQ ID NO: 129, and a LCDR3 comprising or consisting of SEQ ID NO: 163;
(15) the immunoglobulin heavy chain polypeptide comprises a HCDR1 comprising or consisting of SEQ ID NO: 12, a HCDR2 comprising or consisting of SEQ ID NO: 37, and a HCDR3 comprising or consisting of SEQ ID NO: 71; and/or the immunoglobulin light chain polypeptide comprises a LCDR1 comprising or consisting of SEQ ID NO: 108, a LCDR2 comprising or consisting of SEQ ID NO: 137, and a LCDR3 comprising or consisting of SEQ ID NO: 164;
(16) the immunoglobulin heavy chain polypeptide comprises a HCDR1 comprising or consisting of SEQ ID NO: 1, a HCDR2 comprising or consisting of SEQ ID NO: 38, and a HCDR3 comprising or consisting of SEQ ID NO: 72; and/or the immunoglobulin light chain polypeptide comprises a LCDR1 comprising or consisting of SEQ ID NO: 109, a LCDR2 comprising or consisting of SEQ ID NO: 138, and a LCDR3 comprising or consisting of SEQ ID NO: 165;
(17) the immunoglobulin heavy chain polypeptide comprises a HCDR1 comprising or consisting of SEQ ID NO: 13, a HCDR2 comprising or consisting of SEQ ID NO: 39, and a HCDR3 comprising or consisting of SEQ ID NO: 73; and/or the immunoglobulin light chain polypeptide comprises a LCDR1 comprising or consisting of SEQ ID NO: 98, a LCDR2 comprising or consisting of SEQ ID NO: 129, and a LCDR3 comprising or consisting of SEQ ID NO: 155;
(18) the immunoglobulin heavy chain polypeptide comprises a HCDR1 comprising or consisting of SEQ ID NO: 2, a HCDR2 comprising or consisting of SEQ ID NO: 40, and a HCDR3 comprising or consisting of SEQ ID NO: 74; and/or the immunoglobulin light chain polypeptide comprises a LCDR1 comprising or consisting of SEQ ID NO: 110, a LCDR2 comprising or consisting of SEQ ID NO: 137, and a LCDR3 comprising or consisting of SEQ ID NO: 166;
(19) the immunoglobulin heavy chain polypeptide comprises a HCDR1 comprising or consisting of SEQ ID NO: 14, a HCDR2 comprising or consisting of SEQ ID NO: 41, and a HCDR3 comprising or consisting of SEQ ID NO: 75; and/or the immunoglobulin light chain polypeptide comprises a LCDR1 comprising or consisting of SEQ ID NO: 111, a LCDR2 comprising or consisting of SEQ ID NO: 129, and a LCDR3 comprising or consisting of SEQ ID NO: 165;
(20) the immunoglobulin heavy chain polypeptide comprises a HCDR1 comprising or consisting of SEQ ID NO: 15, a HCDR2 comprising or consisting of SEQ ID NO: 42, and a HCDR3 comprising or consisting of SEQ ID NO: 74; and/or the immunoglobulin light chain polypeptide comprises a LCDR1 comprising or consisting of SEQ ID NO: 97, a LCDR2 comprising or consisting of SEQ ID NO: 139, and a LCDR3 comprising or consisting of SEQ ID NO: 152;
(21) the immunoglobulin heavy chain polypeptide comprises a HCDR1 comprising or consisting of SEQ ID NO: 14, a HCDR2 comprising or consisting of SEQ ID NO: 43, and a HCDR3 comprising or consisting of SEQ ID NO: 76; and/or the immunoglobulin light chain polypeptide comprises a LCDR1 comprising or consisting of SEQ ID NO: 112, a LCDR2 comprising or consisting of SEQ ID NO: 137, and a LCDR3 comprising or consisting of SEQ ID NO: 155;
(22) the immunoglobulin heavy chain polypeptide comprises a HCDR1 comprising or consisting of SEQ ID NO: 16, a HCDR2 comprising or consisting of SEQ ID NO: 44, and a HCDR3 comprising or consisting of SEQ ID NO: 77; and/or the immunoglobulin light chain polypeptide comprises a LCDR1 comprising or consisting of SEQ ID NO: 113, a LCDR2 comprising or consisting of SEQ ID NO: 140, and a LCDR3 comprising or consisting of SEQ ID NO: 165;
(23) the immunoglobulin heavy chain polypeptide comprises a HCDR1 comprising or consisting of SEQ ID NO: 9, a HCDR2 comprising or consisting of SEQ ID NO: 45, and a HCDR3 comprising or consisting of SEQ ID NO: 78; and/or the immunoglobulin light chain polypeptide comprises a LCDR1 comprising or consisting of SEQ ID NO: 114, a LCDR2 comprising or consisting of SEQ ID NO: 141, and a LCDR3 comprising or consisting of SEQ ID NO: 165;
(24) the immunoglobulin heavy chain polypeptide comprises a HCDR1 comprising or consisting of SEQ ID NO: 17, a HCDR2 comprising or consisting of SEQ ID NO: 46, and a HCDR3 comprising or consisting of SEQ ID NO: 79; and/or the immunoglobulin light chain polypeptide comprises a LCDR1 comprising or consisting of SEQ ID NO: 98, a LCDR2 comprising or consisting of SEQ ID NO: 129, and a LCDR3 comprising or consisting of SEQ ID NO: 155;
(25) the immunoglobulin heavy chain polypeptide comprises a HCDR1 comprising or consisting of SEQ ID NO: 9, a HCDR2 comprising or consisting of SEQ ID NO: 25, and a HCDR3 comprising or consisting of SEQ ID NO: 80; and/or the immunoglobulin light chain polypeptide comprises a LCDR1 comprising or consisting of SEQ ID NO: 115, a LCDR2 comprising or consisting of SEQ ID NO: 142, and a LCDR3 comprising or consisting of SEQ ID NO: 165;
(26) the immunoglobulin heavy chain polypeptide comprises a HCDR1 comprising or consisting of SEQ ID NO: 17, a HCDR2 comprising or consisting of SEQ ID NO: 41, and a HCDR3 comprising or consisting of SEQ ID NO: 81; and/or the immunoglobulin light chain polypeptide comprises a LCDR1 comprising or consisting of SEQ ID NO: 116, a LCDR2 comprising or consisting of SEQ ID NO: 143, and a LCDR3 comprising or consisting of SEQ ID NO: 167;
(27) the immunoglobulin heavy chain polypeptide comprises a HCDR1 comprising or consisting of SEQ ID NO: 7, a HCDR2 comprising or consisting of SEQ ID NO: 47, and a HCDR3 comprising or consisting of SEQ ID NO: 82; and/or the immunoglobulin light chain polypeptide comprises a LCDR1 comprising or consisting of SEQ ID NO: 117, a LCDR2 comprising or consisting of SEQ ID NO: 144, and a LCDR3 comprising or consisting of SEQ ID NO: 155;
(28) the immunoglobulin heavy chain polypeptide comprises a HCDR1 comprising or consisting of SEQ ID NO: 2, a HCDR2 comprising or consisting of SEQ ID NO: 41, and a HCDR3 comprising or consisting of SEQ ID NO: 83; and/or the immunoglobulin light chain polypeptide comprises a LCDR1 comprising or consisting of SEQ ID NO: 118, a LCDR2 comprising or consisting of SEQ ID NO: 131, and a LCDR3 comprising or consisting of SEQ ID NO: 168;
(29) the immunoglobulin heavy chain polypeptide comprises a HCDR1 comprising or consisting of SEQ ID NO: 18, a HCDR2 comprising or consisting of SEQ ID NO: 48, and a HCDR3 comprising or consisting of SEQ ID NO: 84; and/or the immunoglobulin light chain polypeptide comprises a LCDR1 comprising or consisting of SEQ ID NO: 119, a LCDR2 comprising or consisting of SEQ ID NO: 145, and a LCDR3 comprising or consisting of SEQ ID NO: 165;
(30) the immunoglobulin heavy chain polypeptide comprises a HCDR1 comprising or consisting of SEQ ID NO: 19, a HCDR2 comprising or consisting of SEQ ID NO: 49, and a HCDR3 comprising or consisting of SEQ ID NO: 85; and/or the immunoglobulin light chain polypeptide comprises a LCDR1 comprising or consisting of SEQ ID NO: 120, a LCDR2 comprising or consisting of SEQ ID NO: 146, and a LCDR3 comprising or consisting of SEQ ID NO: 155;
(31) the immunoglobulin heavy chain polypeptide comprises a HCDR1 comprising or consisting of SEQ ID NO: 2, a HCDR2 comprising or consisting of SEQ ID NO: 50, and a HCDR3 comprising or consisting of SEQ ID NO: 86; and/or the immunoglobulin light chain polypeptide comprises a LCDR1 comprising or consisting of SEQ ID NO: 121, a LCDR2 comprising or consisting of SEQ ID NO: 147, and a LCDR3 comprising or consisting of SEQ ID NO: 169;
(32) the immunoglobulin heavy chain polypeptide comprises a HCDR1 comprising or consisting of SEQ ID NO: 2, a HCDR2 comprising or consisting of SEQ ID NO: 51, and a HCDR3 comprising or consisting of SEQ ID NO: 87; and/or the immunoglobulin light chain polypeptide comprises a LCDR1 comprising or consisting of SEQ ID NO: 122, a LCDR2 comprising or consisting of SEQ ID NO: 137, and a LCDR3 comprising or consisting of SEQ ID NO: 155;
(33) the immunoglobulin heavy chain polypeptide comprises a HCDR1 comprising or consisting of SEQ ID NO: 20, a HCDR2 comprising or consisting of SEQ ID NO: 44, and a HCDR3 comprising or consisting of SEQ ID NO: 88; and/or the immunoglobulin light chain polypeptide comprises a LCDR1 comprising or consisting of SEQ ID NO: 123, a LCDR2 comprising or consisting of SEQ ID NO: 148, and a LCDR3 comprising or consisting of SEQ ID NO: 170;
(34) the immunoglobulin heavy chain polypeptide comprises a HCDR1 comprising or consisting of SEQ ID NO: 3, a HCDR2 comprising or consisting of SEQ ID NO: 52, and a HCDR3 comprising or consisting of SEQ ID NO: 60; and/or the immunoglobulin light chain polypeptide comprises a LCDR1 comprising or consisting of SEQ ID NO: 98, a LCDR2 comprising or consisting of SEQ ID NO: 129, and a LCDR3 comprising or consisting of SEQ ID NO: 171;
(35) the immunoglobulin heavy chain polypeptide comprises a HCDR1 comprising or consisting of SEQ ID NO: 2, a HCDR2 comprising or consisting of SEQ ID NO: 53, and a HCDR3 comprising or consisting of SEQ ID NO: 89; and/or the immunoglobulin light chain polypeptide comprises a LCDR1 comprising or consisting of SEQ ID NO: 97, a LCDR2 comprising or consisting of SEQ ID NO: 147, and a LCDR3 comprising or consisting of SEQ ID NO: 172;
(36) the immunoglobulin heavy chain polypeptide comprises a HCDR1 comprising or consisting of SEQ ID NO: 21, a HCDR2 comprising or consisting of SEQ ID NO: 38, and a HCDR3 comprising or consisting of SEQ ID NO: 90; and/or the immunoglobulin light chain polypeptide comprises a LCDR1 comprising or consisting of SEQ ID NO: 109, a LCDR2 comprising or consisting of SEQ ID NO: 150, and a LCDR3 comprising or consisting of SEQ ID NO: 165;
(37) the immunoglobulin heavy chain polypeptide comprises a HCDR1 comprising or consisting of SEQ ID NO: 22, a HCDR2 comprising or consisting of SEQ ID NO: 41, and a HCDR3 comprising or consisting of SEQ ID NO: 91; and/or the immunoglobulin light chain polypeptide comprises a LCDR1 comprising or consisting of SEQ ID NO: 124, a LCDR2 comprising or consisting of SEQ ID NO: 151, and a LCDR3 comprising or consisting of SEQ ID NO: 173;
(38) the immunoglobulin heavy chain polypeptide comprises a HCDR1 comprising or consisting of SEQ ID NO: 2, a HCDR2 comprising or consisting of SEQ ID NO: 54, and a HCDR3 comprising or consisting of SEQ ID NO: 92; and/or the immunoglobulin light chain polypeptide comprises a LCDR1 comprising or consisting of SEQ ID NO: 126, a LCDR2 comprising or consisting of SEQ ID NO: 129, and a LCDR3 comprising or consisting of SEQ ID NO: 165;
(39) the immunoglobulin heavy chain polypeptide comprises a HCDR1 comprising or consisting of SEQ ID NO: 2, a HCDR2 comprising or consisting of SEQ ID NO: 55, and a HCDR3 comprising or consisting of SEQ ID NO: 93; and/or the immunoglobulin light chain polypeptide comprises a LCDR1 comprising or consisting of SEQ ID NO: 97, a LCDR2 comprising or consisting of SEQ ID NO: 149, and a LCDR3 comprising or consisting of SEQ ID NO: 174;
(40) the immunoglobulin heavy chain polypeptide comprises a HCDR1 comprising or consisting of SEQ ID NO: 23, a HCDR2 comprising or consisting of SEQ ID NO: 56, and a HCDR3 comprising or consisting of SEQ ID NO: 94; and/or the immunoglobulin light chain polypeptide comprises a LCDR1 comprising or consisting of SEQ ID NO: 125, a LCDR2 comprising or consisting of SEQ ID NO: 142, and a LCDR3 comprising or consisting of SEQ ID NO: 175;
(41) the immunoglobulin heavy chain polypeptide comprises a HCDR1 comprising or consisting of SEQ ID NO: 14, a HCDR2 comprising or consisting of SEQ ID NO: 43, and a HCDR3 comprising or consisting of SEQ ID NO: 76; and/or the immunoglobulin light chain polypeptide comprises a LCDR1 comprising or consisting of SEQ ID NO: 127, a LCDR2 comprising or consisting of SEQ ID NO: 137, and a LCDR3 comprising or consisting of SEQ ID NO: 176;
(42) the immunoglobulin heavy chain polypeptide comprises a HCDR1 comprising or consisting of SEQ ID NO: 3, a HCDR2 comprising or consisting of SEQ ID NO: 57, and a HCDR3 comprising or consisting of SEQ ID NO: 95; and/or the immunoglobulin light chain polypeptide comprises a LCDR1 comprising or consisting of SEQ ID NO: 128, a LCDR2 comprising or consisting of SEQ ID NO: 137, and a LCDR3 comprising or consisting of SEQ ID NO: 155; and/or
(43) the immunoglobulin heavy chain polypeptide and light chain polypeptide comprises any combination of the CDRs listed in FIGS. 1A-D of PD-L1 Type A binding agents 1-42

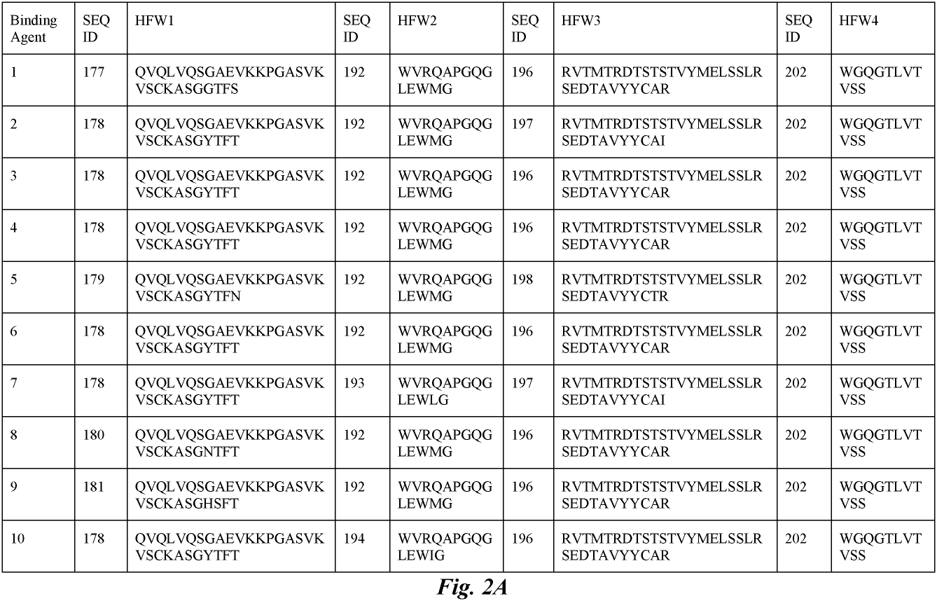

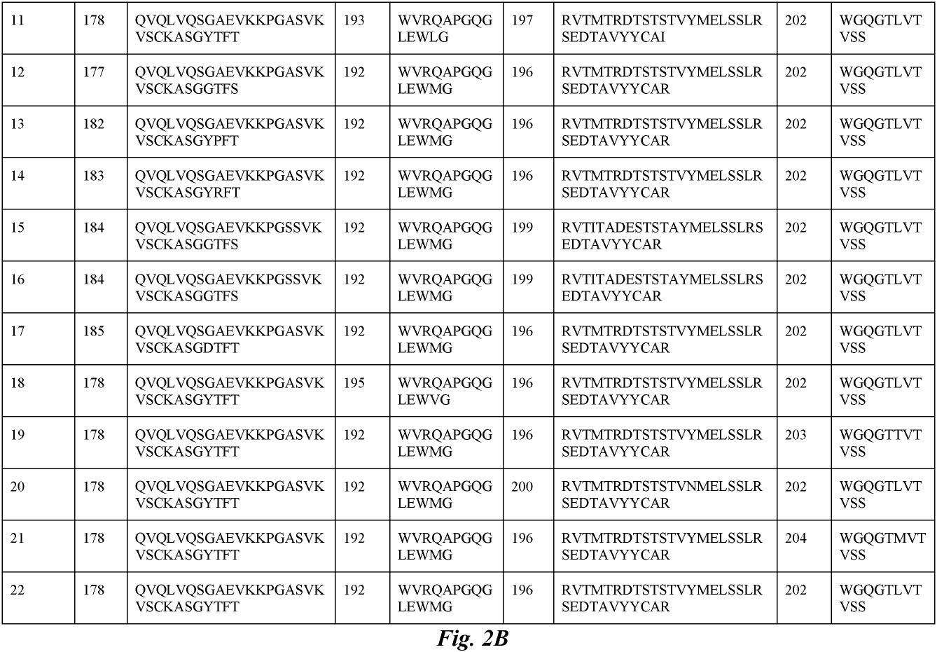

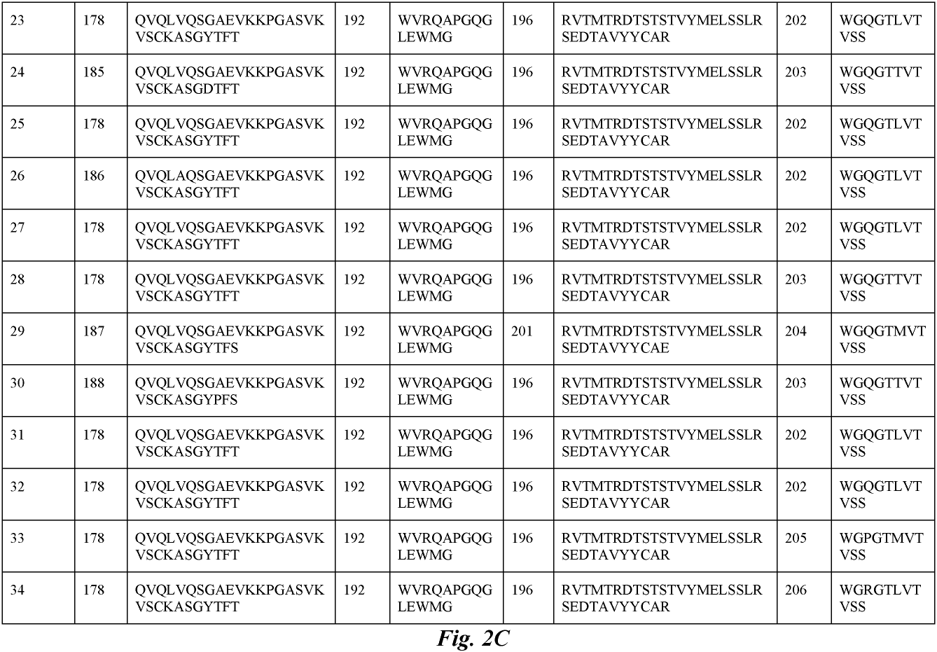

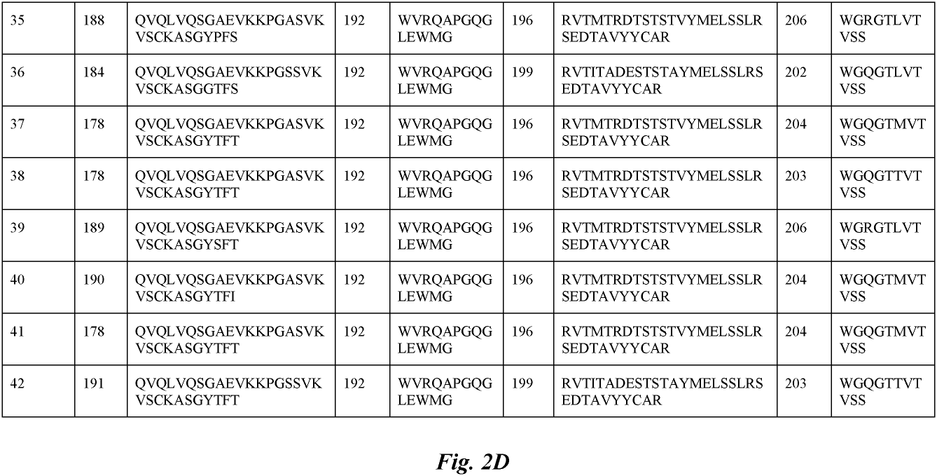

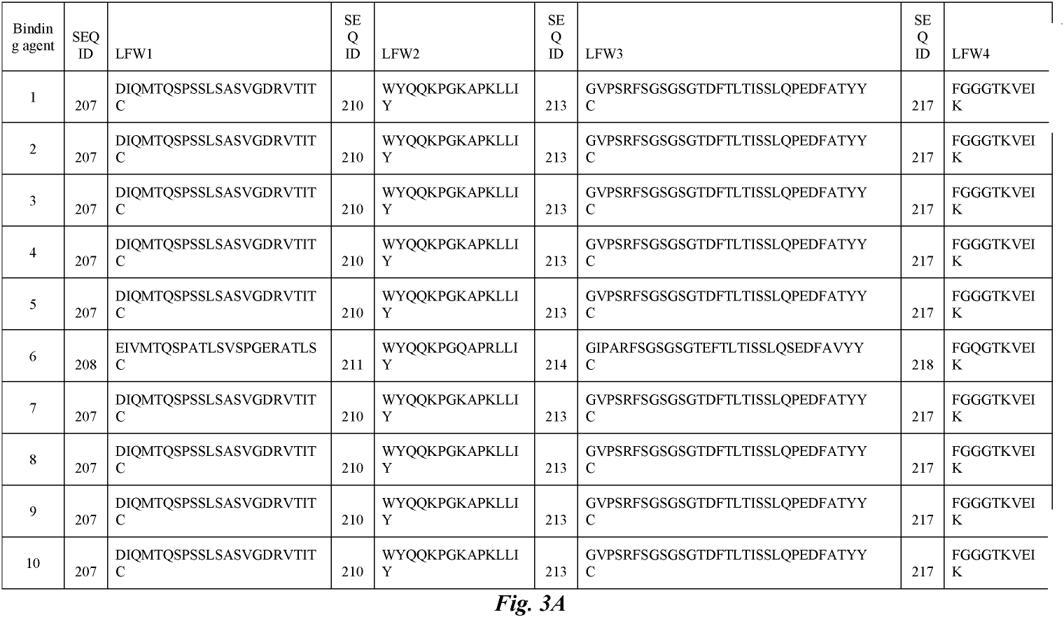

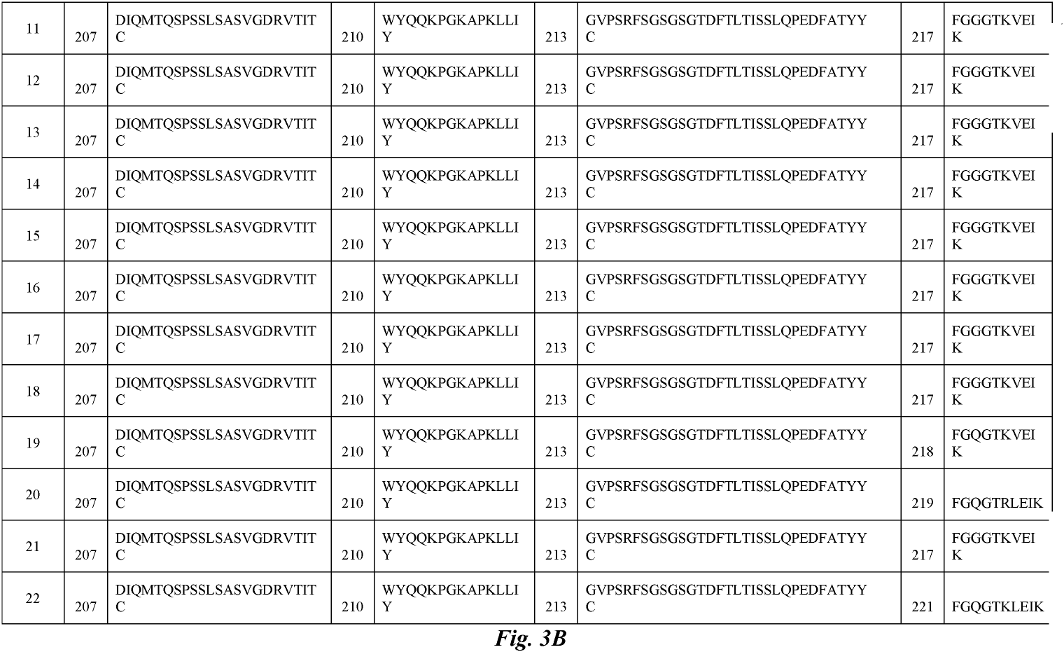

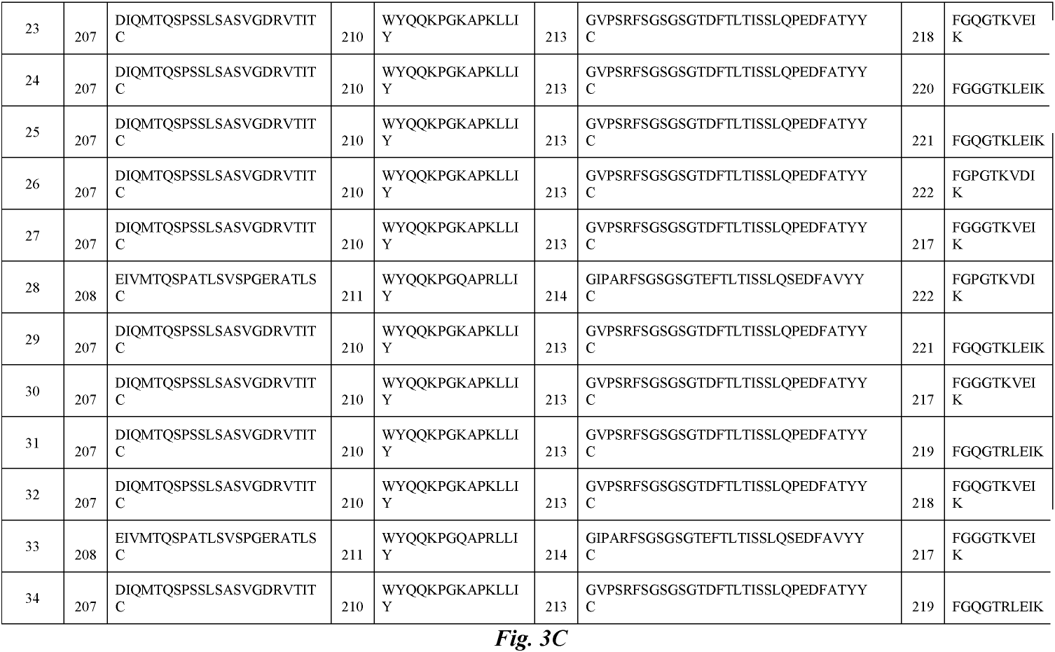

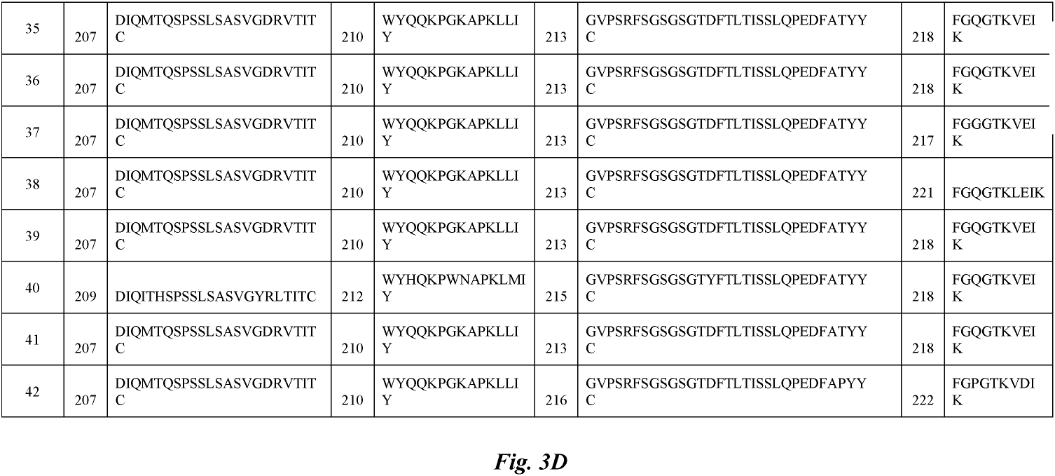

In particular embodiments, the binding agent comprises an immunoglobulin heavy chain polypeptide and an immunoglobulin light chain polypeptide, wherein the immunoglobulin heavy chain polypeptide comprises a first framework region, a second framework region, a third framework region, and/or a fourth framework region; and/or the immunoglobulin light chain polypeptide comprises a first framework region, a second framework region, a third framework region, and/or a fourth framework region; and/or the immunoglobulin heavy chain polypeptide and light chain polypeptide comprises any combination of the framework regions listed in FIGS. 2A-D and FIGS. 3A-D, respectively.

### PD-L1-Binding Agents - Type B

Provided herein are PD-L1 binding agents (Type B) comprising an immunoglobulin heavy chain variable region polypeptide and an immunoglobulin light chain variable region polypeptide. In some embodiments, the PD-L1 binding agents (Type B) comprise an immunoglobulin heavy chain variable region of any one of SEQ ID NOs: 430-450, or at least the CDRs thereof; and an immunoglobulin light chain variable region of any one of SEQ ID NOs: 451-471, or at least the CDRs thereof. In other embodiments, the PD-L1 binding agents comprise an immunoglobulin heavy chain variable region polypeptide with an amino acid sequence that is at least 90% identical to any one of SEQ ID NOs: 430-450, and an immunoglobulin light chain variable region polypeptide with an amino acid sequence that is at least 90% identical to any one of SEQ ID NOs: 451-471. In yet other embodiments, the PD-L1 binding agent, the immunoglobulin heavy chain variable region polypeptide comprises a complementarity determining region 1 (HCDR1) comprising any one of SEQ ID NOs: 308-321, a complementarity determining region 2 (HCDR2) comprising any one of SEQ ID NOs: 322-338, and a complementarity determining region 3 (HCDR3) comprising any one of SEQ ID NOs: 339-359; and/or the immunoglobulin light chain variable region polypeptide comprises a complementarity determining region 1 (LCDR1) comprising any one of SEQ ID NOs: 360-374, a complementarity determining region 2 (LCDR2) comprising any one of SEQ ID NOs: 375-386, and a complementarity determining region 3 (LCDR3) comprising any one of SEQ ID NOs: 387-398. Also provided are nucleic acids encoding the PD-L1 binding agents, or the individual heavy and light chains thereof; vectors and cells comprising the nucleic acids; and compositions comprising the binding agents or nucleic acids.

In an embodiment, the PD-L1 binding agent (Type B) comprises an immunoglobulin heavy chain variable region of any one of SEQ ID NOs: 430-450, a sequence that is at least about 90%, at least about 91%, at least about 92%, at least about 93%, at least about 94%, at least about 95%, at least about 96%, at least about 97%, at least about 98%, or at least about 99% identical to SEQ ID NOs: 430-450, or at least the CDRs thereof; and/or an immunoglobulin light chain variable region of any one of SEQ ID NOs: 451-471, a sequence that is at least about 90%, at least about 91%, at least about 92%, at least about 93%, at least about 94%, at least about 95%, at least about 96%, at least about 97%, at least about 98%, or at least about 99% identical to SEQ ID NOs: 451-471, or at least the CDRs thereof.

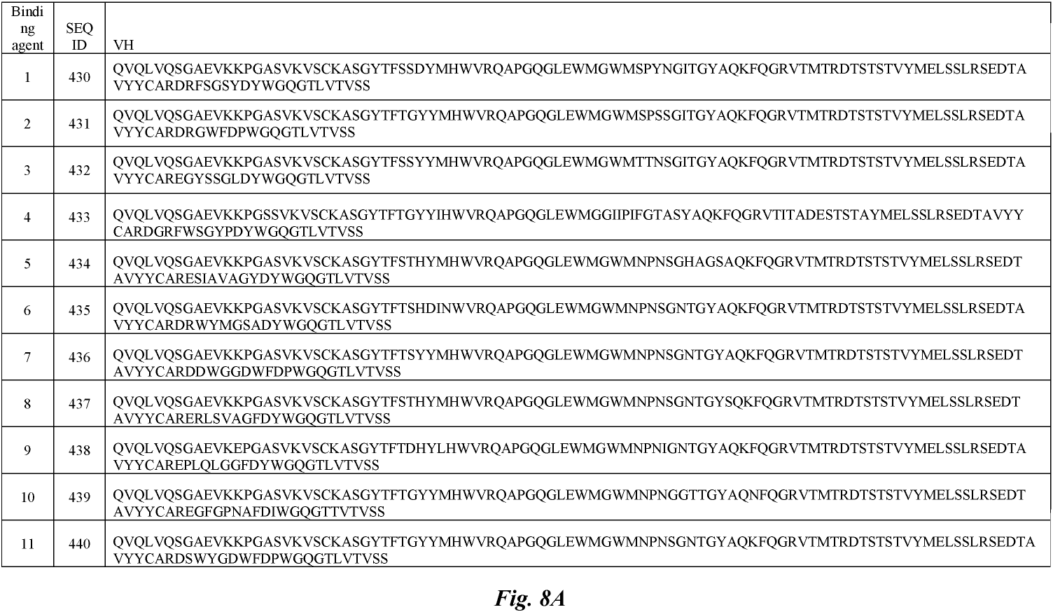

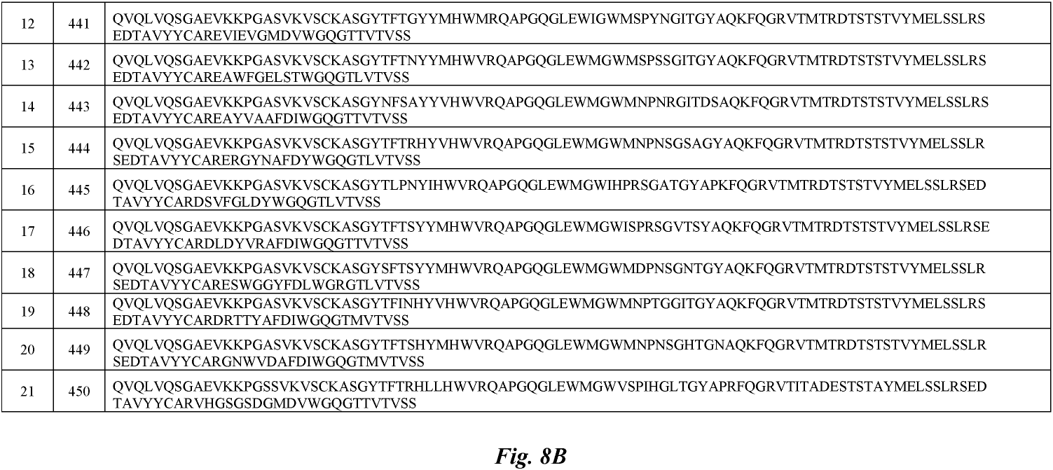

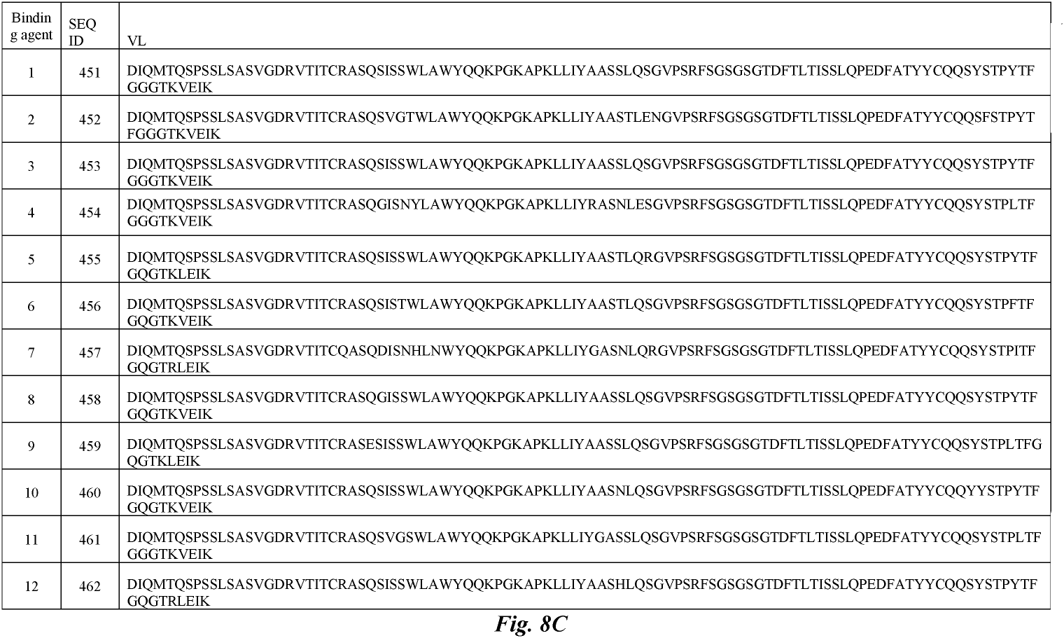

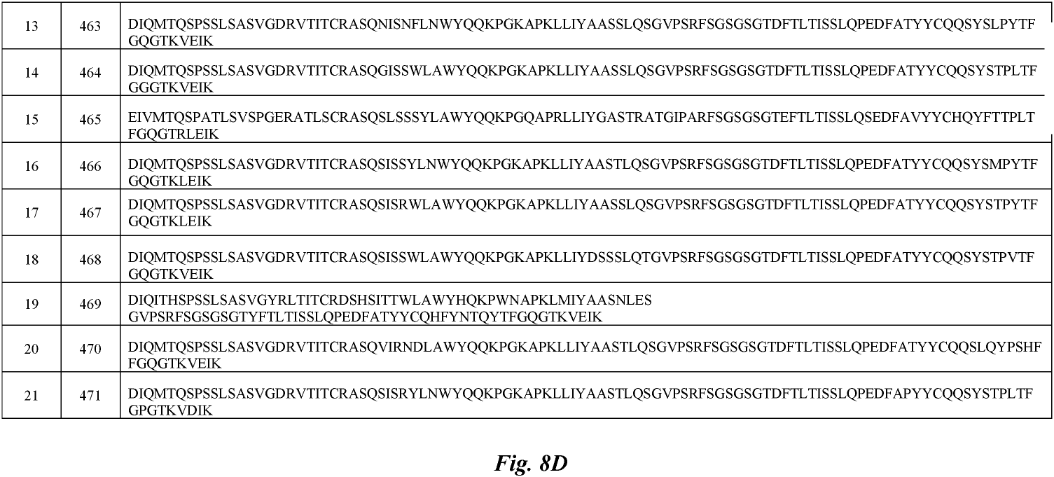

By way of further illustration, the PD-L1 binding agent (Type B) can comprise:
(1) an immunoglobulin heavy chain variable region of SEQ ID NO: 429, or at least the CDRs thereof, and/or an immunoglobulin light chain variable region of SEQ ID NO: 450, or at least the CDRs thereof;
(2) an immunoglobulin heavy chain variable region of SEQ ID NO: 430, or at least the CDRs thereof, and/or an immunoglobulin light chain variable region of SEQ ID NO: 451, or at least the CDRs thereof;
(3) an immunoglobulin heavy chain variable region of SEQ ID NO: 431, or at least the CDRs thereof, and/or an immunoglobulin light chain variable region of SEQ ID NO: 452, or at least the CDRs thereof;
(4) an immunoglobulin heavy chain variable region of SEQ ID NO: 432, or at least the CDRs thereof, and/or an immunoglobulin light chain variable region of SEQ ID NO: 453, or at least the CDRs thereof;
(5) an immunoglobulin heavy chain variable region of SEQ ID NO: 433, or at least the CDRs thereof, and/or an immunoglobulin light chain variable region of SEQ ID NO: 454, or at least the CDRs thereof;
(6) an immunoglobulin heavy chain variable region of SEQ ID NO: 434, or at least the CDRs thereof, and/or an immunoglobulin light chain variable region of SEQ ID NO: 455, or at least the CDRs thereof;
(7) an immunoglobulin heavy chain variable region of SEQ ID NO: 435, or at least the CDRs thereof, and/or an immunoglobulin light chain variable region of SEQ ID NO: 456, or at least the CDRs thereof;
(8) an immunoglobulin heavy chain variable region of SEQ ID NO: 436, or at least the CDRs thereof, and/or an immunoglobulin light chain variable region of SEQ ID NO: 457, or at least the CDRs thereof;
(9) an immunoglobulin heavy chain variable region of SEQ ID NO: 437, or at least the CDRs thereof, and/or an immunoglobulin light chain variable region of SEQ ID NO: 458, or at least the CDRs thereof;
(10) an immunoglobulin heavy chain variable region of SEQ ID NO: 438, or at least the CDRs thereof, and/or an immunoglobulin light chain variable region of SEQ ID NO: 459, or at least the CDRs thereof;
(11) an immunoglobulin heavy chain variable region of SEQ ID NO: 439, or at least the CDRs thereof, and/or an immunoglobulin light chain variable region of SEQ ID NO: 460, or at least the CDRs thereof;
(12) an immunoglobulin heavy chain variable region of SEQ ID NO: 440, or at least the CDRs thereof, and/or an immunoglobulin light chain variable region of SEQ ID NO: 461, or at least the CDRs thereof;
(13) an immunoglobulin heavy chain variable region of SEQ ID NO: 441, or at least the CDRs thereof, and/or an immunoglobulin light chain variable region of SEQ ID NO: 462, or at least the CDRs thereof;
(14) an immunoglobulin heavy chain variable region of SEQ ID NO: 442, or at least the CDRs thereof, and/or an immunoglobulin light chain variable region of SEQ ID NO: 463, or at least the CDRs thereof;
(15) an immunoglobulin heavy chain variable region of SEQ ID NO: 443, or at least the CDRs thereof, and/or an immunoglobulin light chain variable region of SEQ ID NO: 464, or at least the CDRs thereof;
(16) an immunoglobulin heavy chain variable region of SEQ ID NO: 444, or at least the CDRs thereof, and/or an immunoglobulin light chain variable region of SEQ ID NO: 465, or at least the CDRs thereof;
(17) an immunoglobulin heavy chain variable region of SEQ ID NO: 445, or at least the CDRs thereof, and/or an immunoglobulin light chain variable region of SEQ ID NO: 466, or at least the CDRs thereof;
(18) an immunoglobulin heavy chain variable region of SEQ ID NO: 446, or at least the CDRs thereof, and/or an immunoglobulin light chain variable region of SEQ ID NO: 467, or at least the CDRs thereof;
(19) an immunoglobulin heavy chain variable region of SEQ ID NO: 447, or at least the CDRs thereof, and/or an immunoglobulin light chain variable region of SEQ ID NO: 468, or at least the CDRs thereof;
(20) an immunoglobulin heavy chain variable region of SEQ ID NO: 448, or at least the CDRs thereof, and/or an immunoglobulin light chain variable region of SEQ ID NO: 469, or at least the CDRs thereof; and/or
(21) an immunoglobulin heavy chain variable region of SEQ ID NO: 449, or at least the CDRs thereof, and/or an immunoglobulin light chain variable region of SEQ ID NO: 470, or at least the CDRs thereof; and/or
(22) an immunoglobulin heavy chain variable region of FIGS. 8A-B and/or an immunoglobulin light chain variable region of FIGS. 8C-D, or at least the CDRs thereof.

The CDRs of a given heavy or light chain Ig sequence can be determined in accordance with any of the various known Ig numbering schemes (e.g., Kabat, Chothia, Martin (Enhanced Chothia), IGMT, AbM). In certain embodiments, the PD-L1 binding agent comprises one or more of the following CDRs:
a HCDR1 comprising or consisting of any one of SEQ ID NOs: 308-321 or a sequence that is at least about 90%, at least about 91%, at least about 92%, at least about 93%, at least about 94%, at least about 95%, at least about 96%, at least about 97%, at least about 98%, or at least about 99% identical to SEQ ID NOs: 308-321;
a HCDR2 comprising or consisting of any one of SEQ ID NOs: 322-338 or a sequence that is at least about 90%, at least about 91%, at least about 92%, at least about 93%, at least about 94%, at least about 95%, at least about 96%, at least about 97%, at least about 98%, or at least about 99% identical to SEQ ID NOs: 322-338; and
a HCDR3 comprising or consisting of any one of SEQ ID NOs: 339-359 or a sequence that is at least about 90%, at least about 91%, at least about 92%, at least about 93%, at least about 94%, at least about 95%, at least about 96%, at least about 97%, at least about 98%, or at least about 99% identical to SEQ ID NOs: 339-359; and/or the immunoglobulin light chain polypeptide comprises
a LCDR1 comprising or consisting of any one of SEQ ID NOs: 360-374 or a sequence that is at least about 90%, at least about 91%, at least about 92%, at least about 93%, at least about 94%, at least about 95%, at least about 96%, at least about 97%, at least about 98%, or at least about 99% identical to SEQ ID NOs: 360-374;
a LCDR2 comprising or consisting of any one of SEQ ID NOs: 375-386 or a sequence that is at least about 90%, at least about 91%, at least about 92%, at least about 93%, at least about 94%, at least about 95%, at least about 96%, at least about 97%, at least about 98%, or at least about 99% identical to SEQ ID NOs: 375-386; and
a LCDR3 comprising or consisting of any one of SEQ ID NOs: 387-398 or a sequence that is at least about 90%, at least about 91%, at least about 92%, at least about 93%, at least about 94%, at least about 95%, at least about 96%, at least about 97%, at least about 98%, or at least about 99% identical to SEQ ID NOs: 387-398.

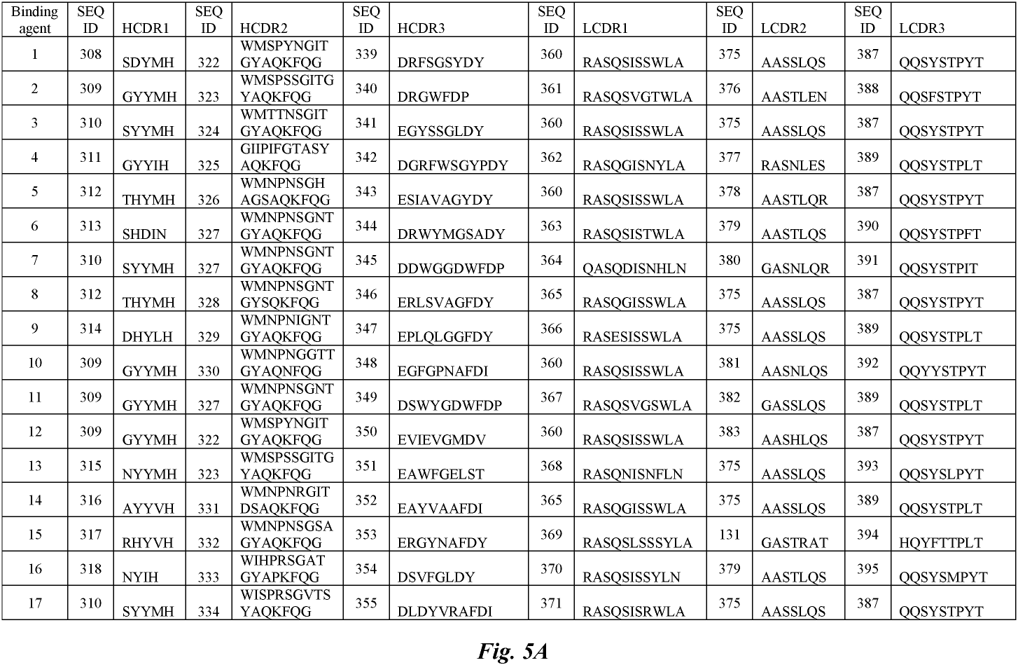

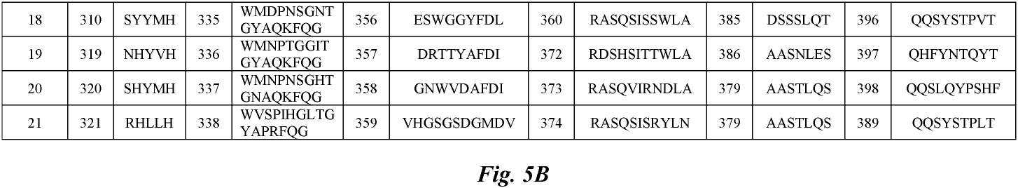

In particular embodiments, the binding agent comprises an immunoglobulin heavy chain polypeptide and an immunoglobulin light chain polypeptide, wherein:
(1) the immunoglobulin heavy chain polypeptide comprises a HCDR1 comprising or consisting of SEQ ID NO: 308, a HCDR2 comprising or consisting of SEQ ID NO: 322, and a HCDR3 comprising or consisting of SEQ ID NO: 339; and/or the immunoglobulin light chain polypeptide comprises a LCDR1 comprising or consisting of SEQ ID NO: 360, a LCDR2 comprising or consisting of SEQ ID NO: 375, and a LCDR3 comprising or consisting of SEQ ID NO: 387;
(2) the immunoglobulin heavy chain polypeptide comprises a HCDR1 comprising or consisting of SEQ ID NO: 309, a HCDR2 comprising or consisting of SEQ ID NO: 323, and a HCDR3 comprising or consisting of SEQ ID NO: 340; and/or the immunoglobulin light chain polypeptide comprises a LCDR1 comprising or consisting of SEQ ID NO: 361, a LCDR2 comprising or consisting of SEQ ID NO: 376, and a LCDR3 comprising or consisting of SEQ ID NO: 388;
(3) the immunoglobulin heavy chain polypeptide comprises a HCDR1 comprising or consisting of SEQ ID NO: 310, a HCDR2 comprising or consisting of SEQ ID NO: 324, and a HCDR3 comprising or consisting of SEQ ID NO: 341; and/or the immunoglobulin light chain polypeptide comprises a LCDR1 comprising or consisting of SEQ ID NO: 360, a LCDR2 comprising or consisting of SEQ ID NO: 375, and a LCDR3 comprising or consisting of SEQ ID NO: 387;
(4) the immunoglobulin heavy chain polypeptide comprises a HCDR1 comprising or consisting of SEQ ID NO: 311, a HCDR2 comprising or consisting of SEQ ID NO: 325, and a HCDR3 comprising or consisting of SEQ ID NO: 342; and/or the immunoglobulin light chain polypeptide comprises a LCDR1 comprising or consisting of SEQ ID NO: 362, a LCDR2 comprising or consisting of SEQ ID NO: 377, and a LCDR3 comprising or consisting of SEQ ID NO: 389;
(5) the immunoglobulin heavy chain polypeptide comprises a HCDR1 comprising or consisting of SEQ ID NO: 312, a HCDR2 comprising or consisting of SEQ ID NO: 326, and a HCDR3 comprising or consisting of SEQ ID NO: 343; and/or the immunoglobulin light chain polypeptide comprises a LCDR1 comprising or consisting of SEQ ID NO: 360, a LCDR2 comprising or consisting of SEQ ID NO: 378, and a LCDR3 comprising or consisting of SEQ ID NO: 387;
(6) the immunoglobulin heavy chain polypeptide comprises a HCDR1 comprising or consisting of SEQ ID NO: 313, a HCDR2 comprising or consisting of SEQ ID NO: 327, and a HCDR3 comprising or consisting of SEQ ID NO: 344; and/or the immunoglobulin light chain polypeptide comprises a LCDR1 comprising or consisting of SEQ ID NO: 363, a LCDR2 comprising or consisting of SEQ ID NO: 379, and a LCDR3 comprising or consisting of SEQ ID NO: 390;
(7) the immunoglobulin heavy chain polypeptide comprises a HCDR1 comprising or consisting of SEQ ID NO: 314, a HCDR2 comprising or consisting of SEQ ID NO: 327, and a HCDR3 comprising or consisting of SEQ ID NO: 345; and/or the immunoglobulin light chain polypeptide comprises a LCDR1 comprising or consisting of SEQ ID NO: 364, a LCDR2 comprising or consisting of SEQ ID NO: 380, and a LCDR3 comprising or consisting of SEQ ID NO: 391;
(8) the immunoglobulin heavy chain polypeptide comprises a HCDR1 comprising or consisting of SEQ ID NO: 312, a HCDR2 comprising or consisting of SEQ ID NO: 328, and a HCDR3 comprising or consisting of SEQ ID NO: 346; and/or the immunoglobulin light chain polypeptide comprises a LCDR1 comprising or consisting of SEQ ID NO: 365, a LCDR2 comprising or consisting of SEQ ID NO: 375, and a LCDR3 comprising or consisting of SEQ ID NO: 387;
(9) the immunoglobulin heavy chain polypeptide comprises a HCDR1 comprising or consisting of SEQ ID NO: 314, a HCDR2 comprising or consisting of SEQ ID NO: 329, and a HCDR3 comprising or consisting of SEQ ID NO: 347; and/or the immunoglobulin light chain polypeptide comprises a LCDR1 comprising or consisting of SEQ ID NO: 366, a LCDR2 comprising or consisting of SEQ ID NO: 375, and a LCDR3 comprising or consisting of SEQ ID NO: 389;
(10) the immunoglobulin heavy chain polypeptide comprises a HCDR1 comprising or consisting of SEQ ID NO: 309, a HCDR2 comprising or consisting of SEQ ID NO: 330, and a HCDR3 comprising or consisting of SEQ ID NO: 348; and/or the immunoglobulin light chain polypeptide comprises a LCDR1 comprising or consisting of SEQ ID NO: 360, a LCDR2 comprising or consisting of SEQ ID NO: 381, and a LCDR3 comprising or consisting of SEQ ID NO: 392;
(11) the immunoglobulin heavy chain polypeptide comprises a HCDR1 comprising or consisting of SEQ ID NO: 309, a HCDR2 comprising or consisting of SEQ ID NO: 327, and a HCDR3 comprising or consisting of SEQ ID NO: 349; and/or the immunoglobulin light chain polypeptide comprises a LCDR1 comprising or consisting of SEQ ID NO: 367, a LCDR2 comprising or consisting of SEQ ID NO: 382, and a LCDR3 comprising or consisting of SEQ ID NO: 389;
(12) the immunoglobulin heavy chain polypeptide comprises a HCDR1 comprising or consisting of SEQ ID NO: 309, a HCDR2 comprising or consisting of SEQ ID NO: 322, and a HCDR3 comprising or consisting of SEQ ID NO: 350; and/or the immunoglobulin light chain polypeptide comprises a LCDR1 comprising or consisting of SEQ ID NO: 360, a LCDR2 comprising or consisting of SEQ ID NO: 383, and a LCDR3 comprising or consisting of SEQ ID NO: 387;
(13) the immunoglobulin heavy chain polypeptide comprises a HCDR1 comprising or consisting of SEQ ID NO: 315, a HCDR2 comprising or consisting of SEQ ID NO: 323, and a HCDR3 comprising or consisting of SEQ ID NO: 351; and/or the immunoglobulin light chain polypeptide comprises a LCDR1 comprising or consisting of SEQ ID NO: 368, a LCDR2 comprising or consisting of SEQ ID NO: 375, and a LCDR3 comprising or consisting of SEQ ID NO: 393;
(14) the immunoglobulin heavy chain polypeptide comprises a HCDR1 comprising or consisting of SEQ ID NO:316, a HCDR2 comprising or consisting of SEQ ID NO: 331, and a HCDR3 comprising or consisting of SEQ ID NO: 352; and/or the immunoglobulin light chain polypeptide comprises a LCDR1 comprising or consisting of SEQ ID NO: 365, a LCDR2 comprising or consisting of SEQ ID NO: 375, and a LCDR3 comprising or consisting of SEQ ID NO: 389;
(15) the immunoglobulin heavy chain polypeptide comprises a HCDR1 comprising or consisting of SEQ ID NO: 317, a HCDR2 comprising or consisting of SEQ ID NO: 332, and a HCDR3 comprising or consisting of SEQ ID NO: 353; and/or the immunoglobulin light chain polypeptide comprises a LCDR1 comprising or consisting of SEQ ID NO: 369, a LCDR2 comprising or consisting of SEQ ID NO: 384, and a LCDR3 comprising or consisting of SEQ ID NO: 394;
(16) the immunoglobulin heavy chain polypeptide comprises a HCDR1 comprising or consisting of SEQ ID NO: 318, a HCDR2 comprising or consisting of SEQ ID NO: 333, and a HCDR3 comprising or consisting of SEQ ID NO: 354; and/or the immunoglobulin light chain polypeptide comprises a LCDR1 comprising or consisting of SEQ ID NO: 370, a LCDR2 comprising or consisting of SEQ ID NO: 379, and a LCDR3 comprising or consisting of SEQ ID NO: 395;
(17) the immunoglobulin heavy chain polypeptide comprises a HCDR1 comprising or consisting of SEQ ID NO:310, a HCDR2 comprising or consisting of SEQ ID NO: 334, and a HCDR3 comprising or consisting of SEQ ID NO: 355; and/or the immunoglobulin light chain polypeptide comprises a LCDR1 comprising or consisting of SEQ ID NO: 371, a LCDR2 comprising or consisting of SEQ ID NO: 375, and a LCDR3 comprising or consisting of SEQ ID NO: 387;
(18) the immunoglobulin heavy chain polypeptide comprises a HCDR1 comprising or consisting of SEQ ID NO:310, a HCDR2 comprising or consisting of SEQ ID NO: 335, and a HCDR3 comprising or consisting of SEQ ID NO: 356; and/or the immunoglobulin light chain polypeptide comprises a LCDR1 comprising or consisting of SEQ ID NO: 360, a LCDR2 comprising or consisting of SEQ ID NO: 385, and a LCDR3 comprising or consisting of SEQ ID NO: 396;
(19) the immunoglobulin heavy chain polypeptide comprises a HCDR1 comprising or consisting of SEQ ID NO: 319, a HCDR2 comprising or consisting of SEQ ID NO: 336, and a HCDR3 comprising or consisting of SEQ ID NO: 357; and/or the immunoglobulin light chain polypeptide comprises a LCDR1 comprising or consisting of SEQ ID NO: 372, a LCDR2 comprising or consisting of SEQ ID NO: 386, and a LCDR3 comprising or consisting of SEQ ID NO: 397;
(20) the immunoglobulin heavy chain polypeptide comprises a HCDR1 comprising or consisting of SEQ ID NO: 320, a HCDR2 comprising or consisting of SEQ ID NO: 337, and a HCDR3 comprising or consisting of SEQ ID NO: 358; and/or the immunoglobulin light chain polypeptide comprises a LCDR1 comprising or consisting of SEQ ID NO: 373, a LCDR2 comprising or consisting of SEQ ID NO: 379, and a LCDR3 comprising or consisting of SEQ ID NO: 398;
(21) the immunoglobulin heavy chain polypeptide comprises a HCDR1 comprising or consisting of SEQ ID NO: 321, a HCDR2 comprising or consisting of SEQ ID NO: 338, and a HCDR3 comprising or consisting of SEQ ID NO: 359; and/or the immunoglobulin light chain polypeptide comprises a LCDR1 comprising or consisting of SEQ ID NO: 374, a LCDR2 comprising or consisting of SEQ ID NO: 379, and a LCDR3 comprising or consisting of SEQ ID NO: 389; and/or
(22) the immunoglobulin heavy chain polypeptide and light chain polypeptide comprises any combination of the CDRs listed in FIGS. 5A-B (Type B).

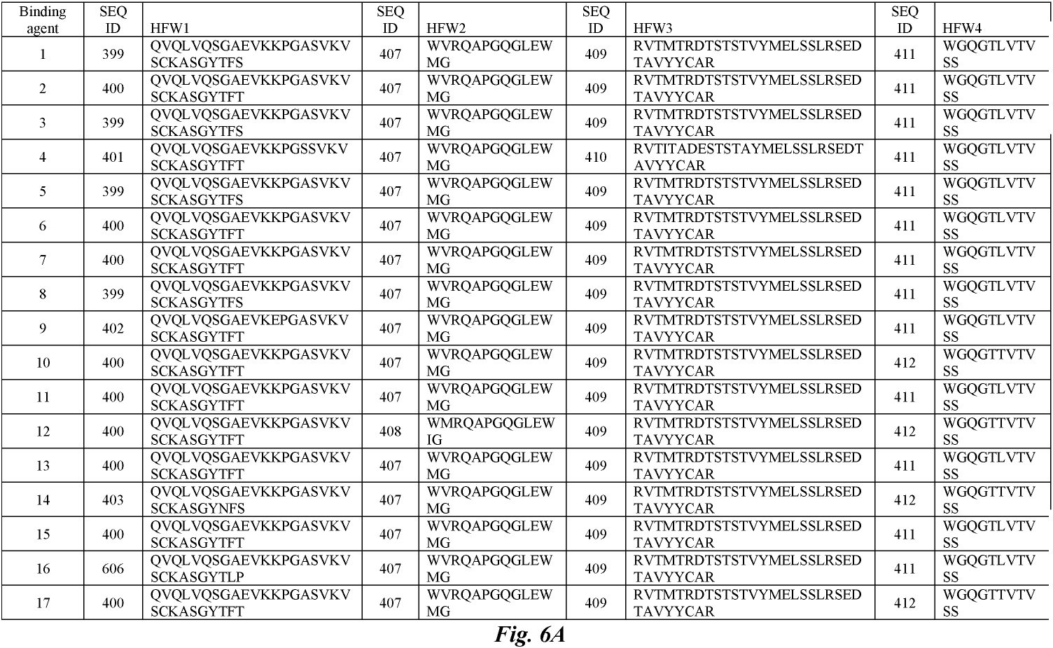

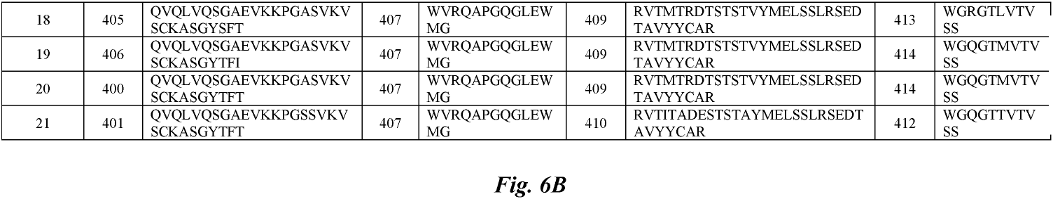

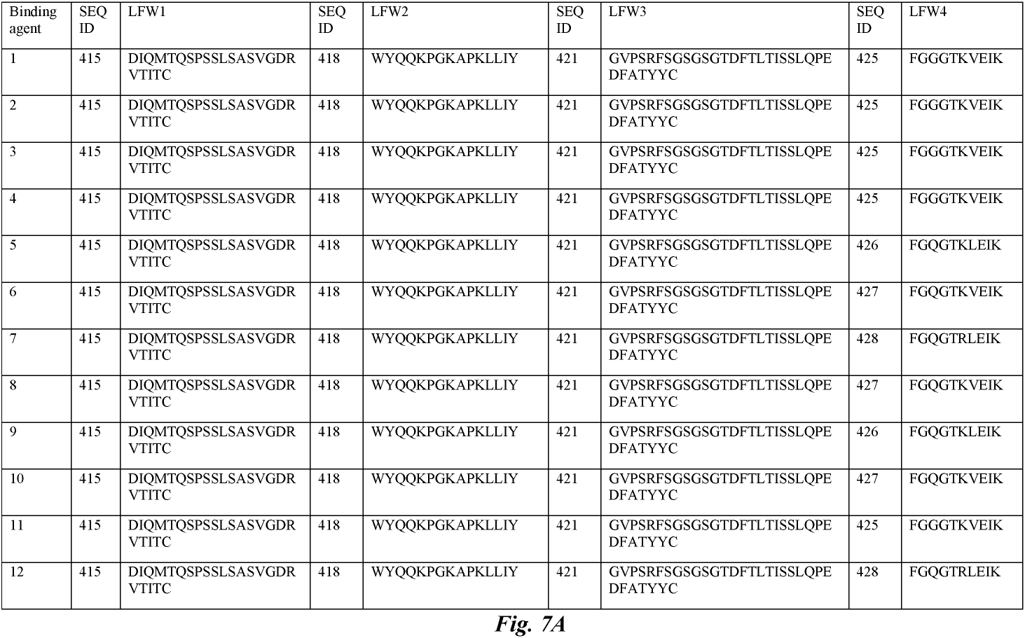

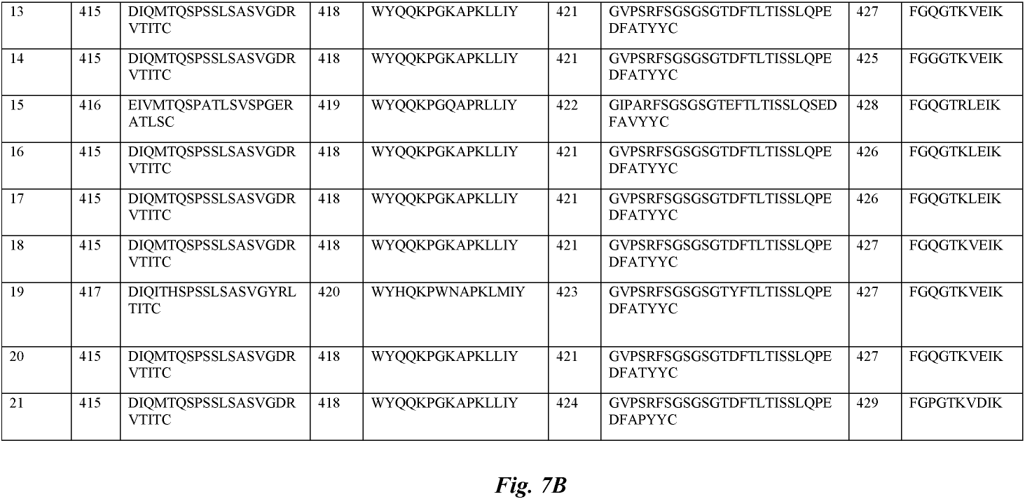

In particular embodiments, the binding agent comprises an immunoglobulin heavy chain polypeptide and an immunoglobulin light chain polypeptide, wherein the immunoglobulin heavy chain polypeptide comprises a first framework region, a second framework region, a third framework region, and/or a fourth framework region; and/or the immunoglobulin light chain polypeptide comprises a first framework region, a second framework region, a third framework region, and/or a fourth framework region; and/or the immunoglobulin heavy chain polypeptide and light chain polypeptide comprises any combination of the framework regions listed in FIGS. 6A-B and/or FIGS. 7A-B (Type B), respectively.

In an exemplary embodiment, the immunoconjugates of the invention comprise an antibody construct that comprises an antigen binding domain that specifically recognizes and binds HER2.

In certain embodiments, immunoconjugates of the invention comprise anti-HER2 antibodies. In one embodiment of the invention, an anti-HER2 antibody of an immunoconjugate of the invention comprises a humanized anti-HER2 antibody, e.g., huMAb4D5-1, huMAb4D5-2, huMAb4D5-3, huMAb4D5-4, huMAb4D5-5, huMAb4D5-6, huMAb4D5-7 and huMAb4D5-8, as described in Table 3 of US 5821337, which is specifically incorporated by reference herein. Those antibodies contain human framework regions with the complementarity-determining regions of a murine antibody (4D5) that binds to HER2. The humanized antibody huMAb4D5-8 is also referred to as trastuzumab, commercially available under the tradename HERCEPTIN™ (Genentech, Inc.).

Trastuzumab (CAS 180288-69-1, HERCEPTIN®, huMAb4D5-8, rhuMAb HER2, Genentech) is a recombinant DNA-derived, IgG1 kappa, monoclonal antibody that is a humanized version of a murine anti-HER2 antibody (4D5) that selectively binds with high affinity in a cell-based assay (Kd = 5 nM) to the extracellular domain of HER2 (US 5677171; US 5821337; US 6054297; US 6165464; US 6339142; US 6407213; US 6639055; US 6719971; US 6800738; US 7074404; Coussens et al (1985) Science 230:1132-9; Slamon et al (1989) Science 244:707-12; Slamon et al (2001) New Engl. J. Med. 344:783-792).

In an embodiment of the invention, the antibody construct or antigen binding domain comprises the CDR regions of trastuzumab. In an embodiment of the invention, the anti-HER2 antibody further comprises the framework regions of the trastuzumab. In an embodiment of the invention, the anti-HER2 antibody further comprises one or both variable regions of trastuzumab.

In another embodiment of the invention, an anti-HER2 antibody of an immunoconjugate of the invention comprises a humanized anti-HER2 antibody, e.g., humanized 2C4, as described in US 7862817. An exemplary humanized 2C4 antibody is pertuzumab (CAS Reg. No. 380610-27-5), PERJETA™ (Genentech, Inc.). Pertuzumab is a HER dimerization inhibitor (HDI) and functions to inhibit the ability of HER2 to form active heterodimers or homodimers with other HER receptors (such as EGFR/HER1, HER2, HER3 and HER4). See, for example, Harari and Yarden, Oncogene 19:6102-14 (2000); Yarden and Sliwkowski. Nat Rev Mol Cell Biol 2:127-37 (2001); Sliwkowski Nat Struct Biol 10:158-9 (2003); Cho et al. Nature 421:756-60 (2003); and Malik et al. Pro Am Soc Cancer Res 44:176-7 (2003). PERJETA™ is approved for the treatment of breast cancer.

In an embodiment of the invention, the antibody construct or antigen binding domain comprises the CDR regions of pertuzumab. In an embodiment of the invention, the anti-HER2 antibody further comprises the framework regions of the pertuzumab. In an embodiment of the invention, the anti-HER2 antibody further comprises one or both variable regions of pertuzumab.

In an exemplary embodiment, the immunoconjugates of the invention comprise an antibody construct that comprises an antigen binding domain that specifically recognizes and binds Caprin-1 (Ellis JA, Luzio JP (1995) J Biol Chem. 270(35):20717-23; Wang B, et al (2005) J Immunol. 175 (7):4274-82; Solomon S, et al (2007) Mol Cell Biol. 27(6):2324-42). Caprin-1 is also known as GPIAP1, GPIP137, GRIP137, M11S1, RNG105, p137GPI, and cell cycle associated protein 1.

Cytoplasmic activation/proliferation-associated protein-1 (caprin-1) is an RNA-binding protein that participates in the regulation of cell cycle control-associated genes. Caprin-1 selectively binds to c-Myc and cyclin D2 mRNAs, which accelerates cell progression through the G₁ phase into the S phase, enhances cell viability and promotes cell growth, indicating that it may serve an important role in tumorigenesis (Wang B, et al (2005) J Immunol. 175:4274-4282). Caprin-1 acts alone or in combination with other RNA-binding proteins, such as RasGAP SH3-domain-binding protein 1 and fragile X mental retardation protein. In the tumorigenesis process, caprin-1 primarily functions by activating cell proliferation and upregulating the expression of immune checkpoint proteins. Through the formation of stress granules, caprin-1 is also involved in the process by which tumor cells adapt to adverse conditions, which contributes to radiation and chemotherapy resistance. Given its role in various clinical malignancies, caprin-1 holds the potential to be used as a biomarker and a target for the development of novel therapeutics (Yang, Z-S, et al (2019) Oncology Letters 18:15-21).

Antibodies that target caprin-1 for treatment and detection have been described (WO 2011/096519; WO 2013/125654; WO 2013/125636; WO 2013/125640; WO 2013/125630; WO 2013/018889; WO 2013/018891; WO 2013/018883; WO 2013/018892; WO 2014/014082; WO 2014/014086; WO 2015/020212; WO 2018/079740).

In an exemplary embodiment, the immunoconjugates of the invention comprise an antibody construct that comprises an antigen binding domain that specifically recognizes and binds CEA.

Elevated expression of carcinoembryonic antigen (CEA, CD66e, CEACAM5) has been implicated in various biological aspects of neoplasia, especially tumor cell adhesion, metastasis, the blocking of cellular immune mechanisms, and having antiapoptosis functions. CEA is also used as a blood marker for many carcinomas. Labetuzumab (CEA-CIDE™, Immunomedics, CAS Reg. No. 219649-07-7), also known as MN-14 and hMN14, is a humanized IgG1 monoclonal antibody and has been studied for the treatment of colorectal cancer (Blumenthal, R. et al (2005) Cancer Immunology Immunotherapy 54(4):315-327). Labetuzumab conjugated to a camptothecin analog (labetuzumab govitecan, IMMU-130) targets carcinoembryonic antigen-related cell adhesion mol. 5 (CEACAM5) and is being studied in patients with relapsed or refractory metastatic colorectal cancer (Sharkey, R. et al, (2018), Molecular Cancer Therapeutics 17(1):196-203; Cardillo, T. et al (2018) Molecular Cancer Therapeutics 17(1):150-160).

In an embodiment of the invention, the CEA-targeting antibody construct or antigen binding domain comprises the Variable light chain (VL kappa) of hMN-14/labetuzumab SEQ ID NO. 472 (US 6676924).

DIQLTQSPSSLSASVGDRVTITCKASQDVGTSVAWYQQKPGKAPKLLIYW
TSTRHTGVPSRFSGSGSGTDFTFTISSLQPEDIATYYCQQYSLYRSFGQG
TKVEIK SEQ ID NO. 472

In an embodiment of the invention, the CEA-targeting antibody construct or antigen binding domain comprises the light chain CDR (complementarity determining region) or light chain framework (LFR) sequences of hMN-14/labetuzumab SEQ ID NO. 473-479 (US 6676924).

| Region | Sequence Fragment | Residues | Length | SEQ ID NO. |
|---|---|---|---|---|
| LFR1 | DIQLTQSPSSLSASVGDRVTITC | 1 - 23 | 23 | 473 |
| CDR-L1 | KASQDVGTSVA | 24 - 34 | 11 | 474 |
| LFR2 | WYQQKPGKAPKLLIY | 35 - 49 | 15 | 475 |
| CDR-L2 | WTSTRHT | 50 - 56 | 7 | 476 |
| LFR3 | GVPSRFSGSGSGTDFTFTISSLQPEDIATYYC | 57 - 88 | 32 | 477 |
| CDR-L3 | QQYSLYRS | 89 - 96 | 8 | 478 |
| LFR4 | FGQGTKVEIK | 97- 106 | 10 | 479 |

In an embodiment of the invention, the CEA-targeting antibody construct or antigen binding domain comprises the Variable heavy chain (VH) of hMN-14/labetuzumab SEQ ID NO. 480 (US 6676924).

EVQLVESGGGVVQPGRSLRLSCSSSGFDFTTYWMSWVRQAPGKGLEWVAE
IHPDSSTINYAPSLKDRFTISRDNSKNTLFLQMDSLRPEDTGVYFCASLY
FGFPWFAYWGQGTPVTVSS SEQ ID NO. 480

In an embodiment of the invention, the CEA-targeting antibody construct or antigen binding domain comprises the heavy chain CDR (complementarity determining region) or heavy chain framework (HFR) sequences of hMN-14/labetuzumab SEQ ID NO. 481-487 (US 6676924).

| Region | Sequence Fragment | Residues | Length | SEQ ID NO. |
|---|---|---|---|---|
| HFR1 | EVQLVESGGGVVQPGRSLRLSCSSSGFDFT | 1 - 30 | 30 | 481 |
| CDR-H1 | TYWMS | 31 - 35 | 5 | 482 |
| HFR2 | WVRQARGKGLEWVA | 36 - 49 | 14 | 483 |
| CDR-H2 | EIHPDSSTINYAPSLKD | 50 - 66 | 17 | 484 |
| HFR3 | RFTISRDNSKNTLFLQMDSLRPEDTGVYFCAS | 67 - 98 | 32 | 485 |
| CDR-H3 | LYFGFPWFAY | 99 - 108 | 10 | 486 |
| HFR4 | WGQGTPVTVSS | 109 - 119 | 11 | 487 |

In an embodiment of the invention, the CEA-targeting antibody construct or antigen binding domain comprises the Variable light chain (VL kappa) of hPR1A3 SEQ ID NO. 488 (US 8642742).

DIQMTQSPSSLSASVGDRVTITCKASAAVGTYVAWYQQKPGKAPKLLIYS
ASYRKRGVPSRFSGSGSGTDFTLTISSLQPEDFATYYCHQYYTYPLFTFG
QGTKLEIK SEQ ID NO. 488

In an embodiment of the invention, the CEA-targeting antibody construct or antigen binding domain comprises the light chain CDR (complementarity determining region) or light chain framework (LFR) sequences of hPR1A3 SEQ ID NO. 489-495 (US 8642742).

| Region | Sequence Fragment | Residues | Length | SEQ ID NO. |
|---|---|---|---|---|
| LFR1 | DIQMTQSPSSLSASVGDRVTITC | 1 - 23 | 23 | 489 |
| CDR-L1 | KASAAVGTYVA | 24 - 34 | 11 | 490 |
| LFR2 | WYQQKPGKAPKLLIY | 35 - 49 | 15 | 491 |
| CDR-L2 | SASYRKR | 50 - 56 | 7 | 492 |
| LFR3 | GVPSRFSGSGSGTDFTLTISSLQPEDFATYYC | 57 - 88 | 32 | 493 |
| CDR-L3 | HQYYTYPLFT | 89 - 98 | 10 | 494 |
| LFR4 | FGQGTKLEIK | 99 - 108 | 10 | 495 |

In an embodiment of the invention, the CEA-targeting antibody construct or antigen binding domain comprises the heavy chain CDR (complementarity determining region) or heavy chain framework (HFR) sequences of hPR1A3 SEQ ID NO. 496-502 (US 8642742).

| Region | Sequence Fragment | Residues | Length | SEQ ID NO. |
|---|---|---|---|---|
| HFR1 | QVQLVQSGAEVKKPGASVKVSCKASGYTFT | 1 - 30 | 30 | 496 |
| CDR-H1 | EFGMN | 31 - 35 | 5 | 497 |
| HFR2 | WVRQAPGQGLEWMG | 36 - 49 | 14 | 498 |
| CDR-H2 | WINTKTGEATYVEEFKG | 50 - 66 | 17 | 499 |
| HFR3 | RVTFTTDTSTSTAYMELRSDDTAVYYCAR | 67 - 98 | 32 | 500 |
| CDR-H3 | WDFAYYVEAMDY | 99 - 110 | 12 | 501 |
| HFR4 | WGQGTTVTVSS | 111 - 121 | 11 | 502 |

In an embodiment of the invention, the CEA-targeting antibody construct or antigen binding domain comprises the Variable light chain (VL kappa) of hMFE-23 SEQ ID NO. 503 (US 723288).

ENVLTQSPSSMSASVGDRVNIACSASSSVSYMHWFQQKPGKSPKLWIYST
SNLASGVPSRFSGSGSGTDYSLTISSMQPEDAATYYCQQRSSYPLTFGGG
TKLEIK SEQ ID NO. 503

In an embodiment of the invention, the CEA-targeting antibody construct or antigen binding domain comprises the light chain CDR (complementarity determining region) or light chain framework (LFR) sequences of hMFE-23 SEQ ID NO. 504-510 (US 723288).

| Region | Sequence Fragment | Residues | Length | SEQ ID NO. |
|---|---|---|---|---|
| LFR1 | ENVLTQSPSSMSASVGDRVIAC | 1 - 23 | 23 | 504 |
| CDR-L1 | SASSSVSYMH | 24 - 33 | 10 | 505 |
| LFR2 | WFQQKPGKSPKLWIY | 34 - 48 | 15 | 506 |
| CDR-L2 | STSNLAS | 49 - 55 | 7 | 507 |
| LFR3 | GVPSRFSGSGSGTDYSLTISSMQPEDAATYYC | 56 - 87 | 32 | 508 |
| CDR-L3 | QQRSSYPLT | 88 - 96 | 9 | 509 |
| LFR4 | FGGGTKLEIK | 97 - 106 | 10 | 510 |

In an embodiment of the invention, the CEA-targeting antibody construct or antigen binding domain comprises the Variable heavy chain (VH) of hMFE-23 SEQ ID NO. 511 (US 723288).

QVKLEQSGAEVVKPGASVKLSCKASGFNIKDSYMHWLRQGPGQRLEWIGW
IDPENGDTEYAPKFQGKATFTTDTSANTAYLGLSSLRPEDTAVYYCNEGT
PTGPYYFDYWGQGTLVTVSS SEQ ID NO. 511

In an embodiment of the invention, the CEA-targeting antibody construct or antigen binding domain comprises the heavy chain CDR (complementarity determining region) or heavy chain framework (HFR) sequences of hMFE-23 SEQ ID NO. 512-518 (US 723288).

| Region | Sequence Fragment | Residues | Length | SEQ ID NO. |
|---|---|---|---|---|
| HFR 1 | QVKLEQSGAEVVKPGASVKLSCKASGFNIK | 1 - 30 | 30 | 512 |
| CDR-H1 | DSYMH | 31 - 35 | 5 | 513 |
| HFR2 | WLRQGPGQRLEWIG | 36 - 49 | 14 | 514 |
| CDR-H2 | WIDPENGDTEYAPKFQG | 50 - 66 | 17 | 515 |
| HFR3 | KATFTTDTSANTAYLGLSSLRREDTAVYYCNE | 67 - 98 | 32 | 516 |
| CDR-H3 | GTPTGPYYFDY | 99 - 109 | 11 | 517 |
| HFR4 | WGQGTLVTVSS | 110 - 120 | 11 | 518 |

In an embodiment of the invention, the CEA-targeting antibody construct or antigen binding domain comprises the Variable light chain (VL kappa) of SM3E SEQ ID NO. 519 (US 723288).

ENVLTQSPSSMSVSVGDRVTIACSASSSVPYMHWLQQKPGKSPKLLIYLT
SNLASGVPSRFSGSGSGTDYSLTISSVQPEDAATYYCQQRSSYPLTFGGG
TKLEIK SEQ ID NO. 519

In an embodiment of the invention, the CEA-targeting antibody construct or antigen binding domain comprises the light chain CDR (complementarity determining region) or light chain framework (LFR) sequences of SM3E SEQ ID NO. 520-526 (US 723288).

| Region | Sequence Fragment | Residues | Length | SEQ ID NO. |
|---|---|---|---|---|
| LFR1 | ENVLTQSPSSMSVSVGDRVTIAC | 1 - 23 | 23 | 520 |
| CDR-L1 | SASSSVPYMH | 24 - 33 | 10 | 521 |
| LFR2 | WLQQKPGKSPKLLIY | 34 - 48 | 15 | 522 |
| CDR-L2 | LTSNLAS | 49 - 55 | 7 | 523 |
| LFR3 | GVPSRFSGSGSGTDYSLTISSVQPEDAATYYC | 56 - 87 | 32 | 524 |
| CDR-L3 | QQRSSYPLT | 88 - 96 | 9 | 525 |
| LFR4 | FGGGTKLEIK | 97 - 106 | 10 | 526 |

In an embodiment of the invention, the CEA-targeting antibody construct or antigen binding domain comprises the Variable heavy chain (VH) of SM3E SEQ ID NO. 527 (US 723288).

QVKLEQSGAEVVKPGASVKLSCKASGFNIKDSYMHWLRQGPGQRLEWIGW
IDPENGDTEYAPKFQGKATFTTDTSANTAYLGLSSLRPEDTAVYYCNEGT
PTGPYYFDYWGQGTLVTVSS SEQ ID NO. 527

In an embodiment of the invention, the CEA-targeting antibody construct or antigen binding domain comprises the heavy chain CDR (complementarity determining region) or heavy chain framework (HFR) sequences of SM3E SEQ ID NO. 528-534 (US 723288).

| Region | Sequence Fragment | Residues | Length | SEQ ID NO. |
|---|---|---|---|---|
| HFR1 | QVKLEQSGAEVVKPGASVKLSCKASGFNIK | 1 - 30 | 30 | 528 |
| CDR-H1 | DSYMH | 31 - 35 | 5 | 529 |
| HFR2 | WLRQGPGQRLEWIG | 36 - 49 | 14 | 530 |
| CDR-H2 | WIDPENGDTEYAPKFQG | 50 - 66 | 17 | 531 |
| HFR3 | KATFTTDTSANTAYLGLSSLRPEDTAVYYCNE | 67 - 98 | 32 | 532 |
| CDR-H3 | GTPTGPYYFDY | 99 - 109 | 11 | 533 |
| HFR4 | WGQGTLVTVSS | 110 - 120 | 11 | 534 |

In an embodiment of the invention, the CEA-targeting antibody construct or antigen binding domain comprises the light chain CDR (complementarity determining region) or light chain framework (LFR) sequences of NP-4/arcitumomab SEQ ID NO. 535-541.

| Region | Sequence Fragment | Residues | Length | SEQ ID NO. |
|---|---|---|---|---|
| LFR1 | QTVLSQSPAILSASPGEKVTMTC | 1 - 23 | 23 | 535 |
| CDR-L1 | RASSSVTYIH | 24 - 33 | 10 | 536 |
| LFR2 | WYQQKPGSSPKSWIY | 34 - 48 | 15 | 537 |
| CDR-L2 | ATSNLAS | 49 - 55 | 7 | 538 |
| LFR3 | GVPARFSGSGSGTSYSLTISRVEAEDAATYYC | 56 - 87 | 32 | 539 |
| CDR-L3 | QHWSSKPPT | 88 - 96 | 3 | 540 |
| LFR4 | FGGGTKLEIK | 97 - 106 | 10 | 541 |

In an embodiment of the invention, the CEA-targeting antibody construct or antigen binding domain comprises the Variable heavy chain (VH) of NP-4/arcitumomab SEQ ID NO. 542.

EVKLVESGGGLVQPGGSLRLSCATSGFTFTDYYMNWVRQPPGKALEWLGF
IGNKANGYTTEYSASVKGRFTISRDKSQSILYLQMNTLRAEDSATYYCTR
DRGLRFYFDYWGQGTTLTVSS SEQ ID NO. 542.

In an embodiment of the invention, the CEA-targeting antibody construct or antigen binding domain comprises the heavy chain CDR (complementarity determining region) or heavy chain framework (HFR) sequences of NP-4 SEQ ID NO. 543-549.

| Region | Sequence Fragment | Residues | Length | SEQ ID NO. |
|---|---|---|---|---|
| HFR1 | EVKLVESGGGLVQPGGSLRLSCATSGFTFT | 1 - 30 | 30 | 543 |
| CDR-H1 | DYYMN | 31 - 35 | 5 | 544 |
| HFR2 | WVRQPPGKALEWLG | 36 - 49 | 14 | 545 |
| CDR-H2 | FIGNKANGYTTEYSASVKG | 50 - 68 | 19 | 546 |
| HFR3 | RFTISRDKSQSILYLQMNTLRAEDSATYYCTR | 69 - 100 | 32 | 547 |
| CDR-H3 | DRGLRFYFDY | 101 - 110 | 10 | 548 |
| HFR4 | WGQGTTLTVSS | 111 - 121 | 11 | 549 |

In an embodiment of the invention, the CEA-targeting antibody construct or antigen binding domain comprises the Variable light chain (VL kappa) of M5A/hT84.66 SEQ ID NO. 550 (US 7776330).

DIQLTQSPSSLSASVGDRVTITCRAGESVDIFGVGFLHWYQQKPGKAPKL
LIYRASNLESGVPSRFSGSGSRTDFTLTISSLQPEDFATYYCQQTNEDPY
TFGQGTKVEIK SEQ ID NO. 550

In an embodiment of the invention, the CEA-targeting antibody construct or antigen binding domain comprises the light chain CDR (complementarity determining region) or light chain framework (LFR) sequences of M5A/hT84.66 SEQ ID NO. 551-557 (US 7776330).

| Region | Sequence Fragment | Residues | Length | SEQ ID NO. |
|---|---|---|---|---|
| LFR1 | DIQLTQSPSSLSASVGDRVTITC | 1 - 23 | 23 | 551 |
| CDR-L1 | RAGESVDIFGVGFLH | 24 - 38 | 15 | 552 |
| LFR2 | WYQQKPGKAPKLLIY | 39 - 53 | 15 | 553 |
| CDR-L2 | RASNLES | 54 - 60 | 7 | 554 |
| LFR3 | GVPSRFSGSGSRTDFTLTISSLQPEDFATYYC | 61 - 92 | 32 | 555 |
| CDR-L3 | QQTNEDPYT | 93 - 101 | 9 | 556 |
| LFR4 | FGQGTKVEIK | 102 - 111 | 10 | 557 |

In an embodiment of the invention, the CEA-targeting antibody construct or antigen binding domain comprises the Variable heavy chain (VH) of M5A/hT84.66 SEQ ID NO. 558 (US 7776330).

EVQLVESGGGLVQPGGSLRLSCAASGFNIKDTYMHWVRQAPGKGLEWVAR
IDPANGNSKYADSVKGRFTISADTSKNTAYLQMNSLRAEDTAVYYCAPFG
YYVSDYAMAYWGQGTLVTVSS SEQ ID NO. 558

In an embodiment of the invention, the CEA-targeting antibody construct or antigen binding domain comprises the heavy chain CDR (complementarity determining region) or heavy chain framework (HFR) sequences of M5A/hT84.66 SEQ ID NO. 559-565 (US 7776330).

| Region | Sequence Fragment | Residues | Length | SEQ ID NO. |
|---|---|---|---|---|
| HFR1 | EVQLVESGGGLVQPGGSLRLSCAASGFNIK | 1 - 30 | 30 | 559 |
| CDR-H1 | DTYMH | 31 - 35 | 5 | 560 |
| HFR2 | WVRQAPGKGLEWVA | 36 - 49 | 14 | 561 |
| CDR-H2 | RIDPANGNSKYADSVKG | 50 - 66 | 17 | 562 |
| HFR3 | RFTISADTSKNTAYLQMNSLRAEDTAVYYCAP | 67 - 98 | 32 | 563 |
| CDR-H3 | FGYYVSDYAMAY | 99 - 110 | 12 | 564 |
| HFR4 | WGQGTLVTVSS | 111 - 121 | 11 | 565 |

In an embodiment of the invention, the CEA-targeting antibody construct or antigen binding domain comprises the Variable light chain (VL kappa) of hAb2-3 SEQ ID NO. 566 (US 9617345).

DIQMTQSPASLSASVGDRVTITCRASENIFSYLAWYQQKPGKSPKLLVYN
TRTLAEGVPSRFSGSGSGTDFSLTISSLQPEDFATYYCQHHYGTPFTFGS
GTKLEIK SEQ ID NO. 566

In an embodiment of the invention, the CEA-targeting antibody construct or antigen binding domain comprises the light chain CDR (complementarity determining region) or light chain framework (LFR) sequences of hAb2-3 SEQ ID NO. 567-573 (US 9617345).

| Region | Sequence Fragment | Residues | Length | SEQ ID NO. |
|---|---|---|---|---|
| LFR1 | DIQMTQSPASLSASVGDRVTITC | 1 - 23 | 23 | 567 |
| CDR-L1 | RASENIFSYLA | 24 - 34 | 11 | 568 |
| LFR2 | WYQQKPGKSPKLLVY | 35 - 49 | 15 | 569 |
| CDR-L2 | NTRTLAE | 50- 56 | 7 | 570 |
| LFR3 | GVPSRFSGSGSGTDFSLTISSLQPEDFATYYC | 57 - 88 | 32 | 571 |
| CDR-L3 | QHHYGTPFT | 89 - 97 | 9 | 572 |
| LFR4 | FGSGTKLEIK | 98 - 107 | 10 | 573 |

In an embodiment of the invention, the CEA-targeting antibody construct or antigen binding domain comprises the Variable heavy chain (VH) of SEQ ID NO. 574 (US 9617345).

EVQLQESGPGLVKPGGSLSLSCAASGFVFSSYDMSWVRQTPERGLEWVAY
ISSGGGITYAPSTVKGRFTVSRDNAKNTLYLQMNSLTSEDTAVYYCAAHY
FGSSGPFAYWGQGTLVTVSS SEQ ID NO. 574

In an embodiment of the invention, the CEA-targeting antibody construct or antigen binding domain comprises the heavy chain CDR (complementarity determining region) or heavy chain framework (HFR) sequences of hAb2-3 SEQ ID NO. 575-581.

| Region | Sequence Fragment | Residues | Length | SEQ ID NO. |
|---|---|---|---|---|
| HFR1 | EVQLQESGPGLVKPGGSLSLSCAASGFVFS | 1 - 30 | 30 | 575 |
| CDR-H1 | SYDMS | 31 - 35 | 5 | 576 |
| HFR2 | WVRQTPERGLEWVA | 36 - 49 | 14 | 577 |
| CDR-H2 | YISSGGGITYAPSTVKG | 50 - 66 | 17 | 578 |
| HFR3 | RFTVSRDNAKNTLYLQMNSLTSEDTAVYYCAA | 67 - 98 | 32 | 579 |
| CDR-H3 | HYFGSSGPFAY | 99 - 109 | 11 | 580 |
| HFR4 | WGQGTLVTVSS | 110 - 120 | 11 | 581 |

In an embodiment of the invention, the CEA-targeting antibody construct or antigen binding domain comprises the Variable light chain (VL kappa) of A240VL-B9VH/AMG-211 SEQ ID NO. 582 (US 9982063).

QAVLTQPASLSASPGASASLTCTLRRGINVGAYSIYWYQQKPGSPPQYLL
RYKSDSDKQQGSGVSSRFSASKDASANAGILLISGLQSEDEADYYCMIWH
SGASAVFGGGTKLTVL SEQ ID NO. 582

In an embodiment of the invention, the CEA-targeting antibody construct or antigen binding domain comprises the light chain CDR (complementarity determining region) or light chain framework (LFR) sequences of A240VL-B9VH/AMG-211 SEQ ID NO. 583-589 (US 9982063).

| Region | Sequence Fragment | Residues | Length | SEQ ID NO. |
|---|---|---|---|---|
| LFR1 | QAVLTQPASLSASPGASASLTC | 1 - 22 | 22 | 583 |
| CDR-L1 | TLRRGINVGAYSIY | 23 - 36 | 14 | 584 |
| LFR2 | WYQQKPGSPPQYLLR | 37 - 51 | 15 | 585 |
| CDR-L2 | YKSDSDKQQGS | 52 - 62 | 11 | 586 |
| LFR3 | GVSSRFSASKDASANAGILLISGLQSEDEADYYC | 63 - 96 | 34 | 587 |
| CDR-L3 | MIWHSGASAV | 97 - 106 | 10 | 588 |
| LFR4 | FGGGTKLTVL | 107 - 116 | 10 | 589 |

In an embodiment of the invention, the CEA-targeting antibody construct or antigen binding domain comprises the Variable heavy chain (VH) of B9VH SEQ ID NO. 590 (US 9982063).

EVQLVESGGGLVQPGRSLRLSCAASGFTVSSYWMHWVRQAPGKGLEWVGF
IRNKANGGTTEYAASVKGRFTISRDDSKNTLYLQMNSLRAEDTAVYYCAR
DRGLRFYFDYWGQGTTVTVSS SEQ ID NO. 590

In an embodiment of the invention, the CEA-targeting antibody construct or antigen binding domain comprises the heavy chain CDR (complementarity determining region) or heavy chain framework (HFR) sequences of SEQ ID NO. 591-597 (US 9982063).

| Region | Sequence Fragment | Residues | Length | SEQ ID NO. |
|---|---|---|---|---|
| HFR1 | EVQLVESGGGLVQPGRSLRLSCAASGFTVS | 1 - 30 | 30 | 591 |
| CDR-H1 | SYWMH | 31 - 35 | 5 | 592 |
| HFR2 | WVRQAPGKGLEWVG | 36 - 49 | 14 | 593 |
| CDR-H2 | FIRNKANGGTTEYAASVKG | 50 - 68 | 19 | 594 |
| HFR3 | RFTISRDDSKNTLYLQMNSLRAEDTAVYYCAR | 69 - 100 | 32 | 595 |
| CDR-H3 | DRGLRFYFDY | 101 - 110 | 10 | 596 |
| HFR4 | WGQGTTVTVSS | 111 - 121 | 11 | 597 |

In an embodiment of the invention, the CEA-targeting antibody construct or antigen binding domain comprises the Variable heavy chain (VH) of E12VH SEQ ID NO. 598 (US 9982063).

EVQLVESGGGLVQPGRSLRLSCAASGFTVSSYWMHWVRQAPGKGLEWVGF
ILNKANGGTTEYAASVKGRFTISRDDSKNTLYLQMNSLRAEDTAVYYCAR
DRGLRFYFDYWGQGTTVTVSS SEQ ID NO. 598

In an embodiment of the invention, the CEA-targeting antibody construct or antigen binding domain comprises the heavy chain CDR (complementarity determining region) or heavy chain framework (HFR) sequences of SEQ ID NO. 599-605 (US 9982063).

| Region | Sequence Fragment | Residues | Length | SEQ ID NO. |
|---|---|---|---|---|
| HFR1 | EVQLVESGGGLVQPGPSLRLSCAASGFTVS | 1 - 30 | 30 | 599 |
| CDR-H1 | SYVMH | 31 - 35 | 5 | 600 |
| HFR2 | WVRQAPGKGLEWVG | 36 - 49 | 14 | 601 |
| CDR-H2 | FILNKANGGTTEYAASVKG | 50 - 68 | 19 | 602 |
| HFR3 | RFTISRDDSKNTLYLQMNSLRAEDTAVYYCAR | 69 - 100 | 32 | 603 |
| CDR-H3 | DRGLRFYFDY | 101 - 110 | 10 | 604 |
| HFR4 | WGQGTTVTVSS | 111 - 121 | 11 | 605 |

In an exemplary embodiment, the immunoconjugates of the invention comprise an antibody construct that comprises an antigen binding domain that specifically recognizes and binds Trop2. Tumor-associated calcium signal transducer 2 (TROP-2) is a transmembrane glycoprotein encoded by the TACSTD2 gene (Linnenbach AJ, et al (1993) Mol Cell Biol. 13(3): 1507-15; Calabrese G, et al (2001) Cytogenet Cell Genet. 92(1-2): 164-5). Trop2 is an intracellular calcium signal transducer that is differentially expressed in many cancers and signals cells for self-renewal, proliferation, invasion, and survival. Trop2 is considered a stem cell marker and is expressed in many normal tissues, though in contrast, it is overexpressed in many cancers (Ohmachi T, et al., (2006) Clin. Cancer Res., 12(10), 3057-3063; Muhlmann G, et al., (2009) J. Clin. Pathol., 62(2), 152-158; Fong D, et al., (2008) Br. J. Cancer, 99(8), 1290-1295; Fong D, et al., (2008) Mod. Pathol., 21(2), 186-191; Ning S, et al., (2013) Neurol. Sci., 34(10), 1745-1750). Overexpression of Trop2 is of prognostic significance. Several ligands have been proposed that interact with Trop2. Trop2 signals the cells via different pathways and it is transcriptionally regulated by a complex network of several transcription factors.

Human Trop2 (TACSTD2: tumor-associated calcium signal transducer 2, GA733-1, EGP-1, M1S1; hereinafter, referred to as hTrop2) is a single-pass transmembrane type 1 cell membrane protein consisting of 323 amino acid residues. While the presence of a cell membrane protein involved in immune resistance, which is common to human trophoblasts and cancer cells (Faulk W P, et al., Proc. Natl. Acad. Sci. 75(4): 1947-1951 (1978)), has previously been suggested, an antigen molecule recognized by a monoclonal antibody against a cell membrane protein in a human choriocarcinoma cell line was identified and designated as Trop2 as one of the molecules expressed in human trophoblasts (Lipinski M, et al., Proc. Natl. Acad. Sci. 78(8), 5147-5150 (1981)). This molecule was also designated as tumor antigen GA733-1 recognized by a mouse monoclonal antibody GA733 (Linnenbach A J, et al., Proc. Natl. Acad. Sci. 86(1), 27-31 (1989)) obtained by immunization with a gastric cancer cell line or an epithelial glycoprotein (EGP-1; Basu A, et al., Int. J. Cancer, 62 (4), 472-479 (1995)) recognized by a mouse monoclonal antibody RS7-3G11 obtained by immunization with non-small cell lung cancer cells. In 1995, however, the Trop2 gene was cloned, and all of these molecules were confirmed to be identical molecules (Fornaro M, et al., Int. J. Cancer, 62(5), 610-618 (1995)). The DNA sequence and amino acid sequence of hTrop2 are available on a public database and can be referred to, for example, under Accession Nos. NM_002353 and NP_002344 (NCBI).

In response to such information suggesting the association with cancer, a plurality of anti-hTrop2 antibodies have been established so far and studied for their antitumor effects. Among these antibodies, there is disclosed, for example, an unconjugated antibody that exhibits in itself antitumor activity in nude mouse xenograft models (WO 2008/144891; WO 2011/145744; WO 2011/155579; WO 2013/077458) as well as an antibody that exhibits antitumor activity as ADC with a cytotoxic drug (WO 2003/074566; WO 2011/068845; WO 2013/068946; US 7999083). However, the strength or coverage of their activity is still insufficient, and there are unsatisfied medical needs for hTrop2 as a therapeutic target.

Trop2 expression in cancer cells has been correlated with drug resistance. Several strategies target Trop2 on cancer cells that include antibodies, antibody fusion proteins, chemical inhibitors, nanoparticles, etc. The in vitro studies and pre-clinical studies, using these various therapeutic treatments, have resulted in significant inhibition of tumor cell growth both in vitro and in vivo in mice. Clinical studies have explored the potential application of Trop2 as both a prognostic biomarker and as a therapeutic target to reverse resistance.

Sacituzumab govitecan (TRODELVY®, Immunomedics, IMMU-132), an antibody-drug conjugate comprising a Trop2-directed antibody linked to a topoisomerase inhibitor drug, is indicated for the treatment of metastatic triple-negative breast cancer (mTNBC) in adult patients that have received at least two prior therapies. The Trop2 antibody in sacituzumab govitecan is conjugated to SN-38, the active metabolite of irinotecan (US 2016/0297890; WO 2015/098099).

In an embodiment of the invention, the Trop2-targeting antibody construct or antigen binding domain comprises the light chain CDR (complementarity determining region) sequences of hRS7 (humanized RS7), SEQ ID NO. 607-609 (US 7238785, incorporated by reference herein).

| Region | CDR Sequence Fragment | SEQ ID NO. |
|---|---|---|
| CDR-L1 | KASQDVSIAVA | 607 |
| CDR-L2 | SASYRYT | 608 |
| CDR-L3 | QQHYITPLT | 609 |

In an embodiment of the invention, the Trop2-targeting antibody construct or antigen binding domain comprises the heavy chain CDR (complementarity determining region) sequences of hRS7 (humanized RS7), SEQ ID NO. 610-612 (US 7238785; US 9797907; US 9382329; WO 2020/142659, each incorporated by reference herein).

| Region | CDR Sequence Fragment | SEQ ID NO. |
|---|---|---|
| CDR-H1 | NYGMN | 610 |
| CDR-H2 | WINTYTGEPTYTDDFKG | 611 |
| CDR-H3 | GGFGSSYWYFDV | 612 |

In an embodiment of the invention, the Trop2-targeting antibody construct or antigen binding domain comprises the light chain CDR (complementarity determining region) sequences of AR47A6.4.2, SEQ ID NO. 607-609 (US 7420040, incorporated by reference herein).

| Region | CDR Sequence Fragment | SEQ ID NO. |
|---|---|---|
| CDR-L1 | KASQDVSIAVA | 607 |
| CDR-L2 | SASYRYT | 608 |
| CDR-L3 | QQHYITPLT | 609 |

In an embodiment of the invention, the Trop2-targeting antibody construct or antigen binding domain comprises the heavy chain CDR (complementarity determining region) sequences of AR47A6.4.2, SEQ ID NO. 610, 613, 614 (US 7420040, incorporated by reference herein).

| Region | CDR Sequence Fragment | SEQ ID NO. |
|---|---|---|
| CDR-H1 | NYGMN | 610 |
| CDR-H2 | WINTKTGEPTYAEEFKG | 613 |
| CDR-H3 | GGYGSSYWYFDV | 614 |

In an embodiment of the invention, the Trop2-targeting antibody construct or antigen binding domain comprises the light chain CDR (complementarity determining region) sequences of humanized KM4097, SEQ ID NO. 615-617 (US 2012/0237518, incorporated by reference herein).

| Region | CDR Sequence Fragment | SEQ ID NO. |
|---|---|---|
| CDR-L1 | KSSQSLLNSGNQQNYLA | 615 |
| CDR-L2 | GASTRES | 616 |
| CDR-L3 | QSDHIYPYT | 617 |

In an embodiment of the invention, the Trop2-targeting antibody construct or antigen binding domain comprises the heavy chain CDR (complementarity determining region) sequences of humanized KM4097, SEQ ID NO. 618-620 (US 2012/0237518, incorporated by reference herein).

| Region | CDR Sequence Fragment | SEQ ID NO. |
|---|---|---|
| CDR-H1 | IYWLG | 618 |
| CDR-H2 | NIFPGSAYINYNEKFKG | 619 |
| CDR-H3 | EGSNSGY | 620 |

In an embodiment of the invention, the Trop2-targeting antibody construct or antigen binding domain comprises the light chain CDR (complementarity determining region) sequences of HTINA1-HIL1, SEQ ID NO. 608, 609, 621 (US 10,227,417, incorporated by reference herein).

| Region | CDR Sequence Fragment | SEQ ID NO. |
|---|---|---|
| CDR-L1 | KASQDVSTAVA | 621 |
| CDR-L2 | SASYRYT | 608 |
| CDR-L3 | QQHYITPLT | 609 |

In an embodiment of the invention, the Trop2-targeting antibody construct or antigen binding domain comprises the heavy chain CDR (complementarity determining region) sequences of HTINA1-HIL1, SEQ ID NO. 622-624 (US 10,227,417, incorporated by reference herein).

| Region | CDR Sequence Fragment | SEQ ID NO. |
|---|---|---|
| CDR-H1 | TAGMQ | 622 |
| CDR-H2 | WINTHSGVPKYAEDFKG | 623 |
| CDR-H3 | SGFGSSYWYFDV | 624 |

In an embodiment of the invention, the Trop2-targeting antibody construct or antigen binding domain comprises the light chain CDR (complementarity determining region) sequence of HTINA1-HIL1, SEQ ID NO. 625-627 (US 8871908, incorporated by reference herein).

| Region | CDR Sequence Fragment | SEQ ID NO. |
|---|---|---|
| CDR-L1 | RASKSVSTS (X1) YSYMH where X1 is G, L, or N | 625 |
| CDR-L2 | LASNLES | 626 |
| CDR-L3 | QHSRELPYT | 627 |

In an embodiment of the invention, the Trop2-targeting antibody construct or antigen binding domain comprises the heavy chain CDR (complementarity determining region) sequences of HTINA1-HIL1, SEQ ID NO. 628-633 (US 8871908, incorporated by reference herein).

| Region | CDR Sequence Fragment | SEQ ID NO. |
|---|---|---|
| CDR-H1 | SYGVH | 628 |
| CDR-H1 | GGSISSY | 629 |
| CDR-H1 | GGSISSYGVH | 630 |
| CDR-H2 | VIWT (X1) G (X2) TDYNSALM (X3) where X1 is G or S; X2 is S or V; X3 is S or G | 631 |
| CDR-H2 | WT (X1) G (X2) where X1 is G or S; X2 is S or V | 632 |
| CDR-H3 | DGDYDRYTMDY | 633 |

In an embodiment of the invention, the Trop2-targeting antibody construct or antigen binding domain comprises the light chain CDR (complementarity determining region) sequences SEQ ID NO. 626, 627, 634 of hTINA1-H1L1, (US 8871908, incorporated by reference herein).

| Region | CDR Sequence Fragment | SEQ ID NO. |
|---|---|---|
| CDR-L1 | RASKSVSTSGYSYMH | 634 |
| CDR-L2 | LASNLES | 626 |
| CDR-L3 | QHSRELPYT | 627 |

In an embodiment of the invention, the Trop2-targeting antibody construct or antigen binding domain comprises the heavy chain CDR (complementarity determining region) sequences SEQ ID NO. 628-630, 633, 635, 636 of HTINA1-HIL1, (US 8871908, incorporated by reference herein).

| Region | CDR Sequence Fragment | SEQ ID NO. |
|---|---|---|
| CDR-H1 | SYGVH | 628 |
| CDR-H1 | GGSISSY | 629 |
| CDR-H1 | GGSISSYGVH | 630 |
| CDR-H2 | VIWTSGVTDYNSALMG | 635 |
| CDR-H2 | WTSGV | 636 |
| CDR-H3 | DGDYDRYTMDY | 633 |

In some embodiments, the antibody construct further comprises an Fc domain. In certain embodiments, the antibody construct is an antibody. In certain embodiments, the antibody construct is a fusion protein. The antigen binding domain can be a single-chain variable region fragment (scFv). A single-chain variable region fragment (scFv), which is a truncated Fab fragment including the variable (V) domain of an antibody heavy chain linked to a V domain of a light antibody chain via a synthetic peptide, can be generated using routine recombinant DNA technology techniques. Similarly, disulfide-stabilized variable region fragments (dsFv) can be prepared by recombinant DNA technology. The antibody construct or antigen binding domain may comprise one or more variable regions (e.g., two variable regions) of an antigen binding domain of an anti-Trop2 antibody, each variable region comprising a CDR1, a CDR2, and a CDR3 .

In some embodiments, the Fc region is modified by inclusion of a transforming growth factor beta 1 (TGFβ1) receptor, or a fragment thereof, that is capable of binding TGFβ1. For example, the receptor can be TGFβ receptor II (TGFβRII). In some embodiments, theTGFβ receptor is a human TGFβreceptor. In some embodiments, the IgG has a C-terminal fusion to a TGFβRII extracellular domain, ECD (US 9676863). An “Fc linker” may be used to attach the IgG to the TGFβRII extracellular domain. The Fc linker may be a short, flexible peptide that allows for the proper three-dimensional folding of the molecule while maintaining the binding-specificity to the targets. In some embodiments, the N-terminus of the TGFβ receptor is fused to the Fc of the antibody construct (with or without an Fc linker). In some embodiments, the C-terminus of the antibody construct heavy chain is fused to the TGFβ receptor (with or without an Fc linker). In some embodiments, the C-terminal lysine residue of the antibody construct heavy chain is mutated to alanine.

In some embodiments, the antibodies in the immunoconjugates are glycosylated.

In some embodiments, the antibodies in the immunoconjugates is a cysteine-engineered antibody which provides for site-specific conjugation of an adjuvant, label, or drug moiety to the antibody through cysteine substitutions at sites where the engineered cysteines are available for conjugation but do not perturb immunoglobulin folding and assembly or alter antigen binding and effector functions (Junutula, et al., 2008b Nature Biotech., 26(8):925-932; Dornan et al. (2009) Blood 114(13):2721-2729; US 7521541; US 7723485; US 2012/0121615; WO 2009/052249). A “cysteine engineered antibody” or “cysteine engineered antibody variant” is an antibody in which one or more residues of an antibody are substituted with cysteine residues. Cysteine-engineered antibodies can be conjugated to the thienoazepine adjuvant moiety as a thienoazepine-linker compound with uniform stoichiometry (e.g., up to two thienoazepine moieties per antibody in an antibody that has a single engineered cysteine site).

In some embodiments, cysteine-engineered antibodies used to prepare the immunoconjugates of Table 3 have a cysteine residue introduced at the 149-lysine site of the light chain (LC K149C). In other embodiments, the cysteine-engineered antibodies have a cysteine residue introduced at the 118-alanine site (EU numbering) of the heavy chain (HC A118C). This site is alternatively numbered 121 by Sequential numbering or 114 by Kabat numbering. In other embodiments, the cysteine-engineered antibodies have a cysteine residue introduced in the light chain at G64C or R142C according to Kabat numbering, or in the heavy chain at D101C, V184C or T205C according to Kabat numbering.

### IMMUNOSTIMULATORY COMPOUNDS

The immunoconjugate of the invention comprises an immunostimulatory moiety. After cleavage of the elastase-substrate, peptide linker from the cell-binding agent, the active immunostimulatory compound can interact with and/or modulate a receptor to elicit an immune response. Such receptors include, but are not limited to: (1) various toll-like receptors, TLR (Javaid, N. et al (2019) Pharmaceutics 11(9), 441); (2) STING, STING1 (Ramanjulu, J.M. et al (2018 ) Nature 564:439--443; Barber, G.N. (2015) Nature RevImmunol 15:760-770; US 2019/0300513; ); (3) NOD2 (Negroni, A. et al (2018) J. Inflamm. Res. 11:49-60; Coulombe, F. et al (2009) J. Exp. Med. 206(8): 1709-1716; WO 2017/156152); (4) RIG-1, DDX58 (Elion, D.L. et al (2018) Oncotarget 9(48):29007-29017; Kohlway, A. (2013) EMBO Rep 14:772-779; WO 2015/172099); and (5) NLRP3 (Mangan, M. et al (2018) Nature Reviews Drug Discovery 17:588-606).

Immunostimulatory moieties may interact with and/or modulate a pattern-recognition receptor (PRR). PRRs include but are not limited to: Toll-like receptors (TLRs), STING-like receptors, RIG-I-like receptors (RLRs), NOD-like receptors (NLRs), C-type lectin-like receptors (CLRs), and DNA sensors.

In one embodiment, the immunostimulatory moiety is a TLR agonist. TLRs are type-I transmembrane proteins that are responsible for the initiation of innate immune responses in vertebrates. TLRs recognize a variety of pathogen-associated molecular patterns from bacteria, viruses, and fungi and act as a first line of defense against invading pathogens. TLRs elicit overlapping yet distinct biological responses due to differences in cellular expression and in the signaling pathways that they initiate. Once engaged (e.g., by a natural stimulus or a synthetic TLR agonist), TLRs initiate a signal transduction cascade leading to activation of nuclear factor-κB (NF-κB) via the adapter protein myeloid differentiation primary response gene 88 (MyD88) and recruitment of the IL-1 receptor associated kinase (IRAK). Phosphorylation of IRAK then leads to recruitment of TNF-receptor associated factor 6 (TRAF6), which results in the phosphorylation of the NF-κB inhibitor I-κB. As a result, NF-κB enters the cell nucleus and initiates transcription of genes whose promoters contain NF-κB binding sites, such as cytokines. Additional modes of regulation for TLR signaling include TIR-domain containing adapter-inducing interferon-β (TRIF)-dependent induction of TNF-receptor associated factor 6 (TRAF6) and activation of MyD88 independent pathways via TRIF and TRAF3, leading to the phosphorylation of interferon response factor three (IRF3). Similarly, the MyD88 dependent pathway also activates several IRF family members, including IRF5 and IRF7 whereas the TRIF dependent pathway also activates the NF-κB pathway.

Typically, the TLR agonist described herein is a TLR7 and/or TLR8 agonist. TLR7 and TLR8 are both expressed in monocytes and dendritic cells. In humans, TLR7 is also expressed in plasmacytoid dendritic cells (pDCs) and B cells. TLR8 is expressed mostly in cells of myeloid origin, i.e., monocytes, granulocytes, and myeloid dendritic cells. TLR7 and TLR8 are capable of detecting the presence of “foreign” single-stranded RNA within a cell, as a means to respond to viral invasion. Treatment of TLR8-expressing cells, with TLR8 agonists can result in production of high levels of IL-12, IFN-γ, IL-1, TNF-α, IL-6, and other inflammatory cytokines. Similarly, stimulation of TLR7-expressing cells, such as pDCs, with TLR7 agonists can result in production of high levels of IFN-α and other inflammatory cytokines. TLR7/TLR8 engagement and resulting cytokine production can activate dendritic cells and other antigen-presenting cells, driving diverse innate and acquired immune response mechanisms leading to tumor destruction.

Exemplary TLR ⅞ agonists include an amino-imidazoquinoline of formulas a-d:

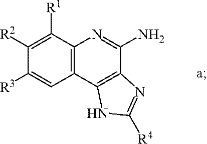

an aminoquinoline of formula b:

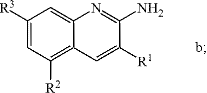

an amino-benzazepine of formula c:

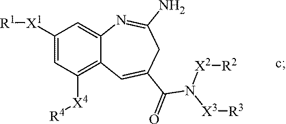

an amino-thienoazepine of formula d:

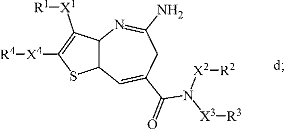

amino-pyrazoloazepines of formulas e and f:

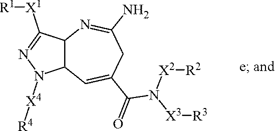

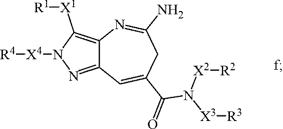

wherein substituents X¹⁻⁴ and R¹⁻⁴ are described herein.

An exemplary amino-benzazepine immunostimulatory TLR moiety has the structure:

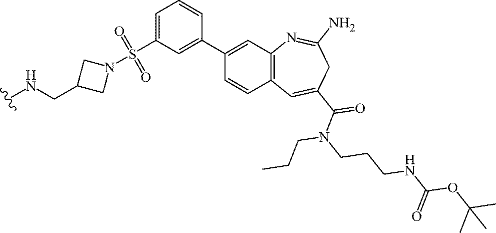

where the wavy line indicates the site of attachment to the elastase-substrate, peptide linker, L.

### ELASTASE-SUBSTRATE, PEPTIDE LINKERS

The invention includes a linking unit, i.e. L or linker, between the cell-binding agent and the immunostimulatory moiety. The linker is a peptide radical based on a linear sequence of specific amino acid residues which can be selectively cleaved by a tumor-associated elastase enzyme or an enzyme with elastase-like activity. The peptide radical may be about two to about twelve amino acids. Cleavage of a bond within the elastase-substrate, peptide linker by elastase releases an active form of the immunostimulatory moiety. This leads to an increase in the tissue specificity of the conjugates according to the invention and thus to an additional decrease of toxicity of the conjugates according to the invention in other tissue types.

The linker provides sufficient stability of the immunoconjugate in biological media, e.g. culture medium or serum and, at the same time, the desired intracellular action within tumor tissue as a result of its specific enzymatic or hydrolytic cleavability with release of the immunostimulatory moiety, i.e. “payload”.

The enzymatic activity of elastase can catalyze cleavage of a covalent bond of the immunoconjugate under physiological conditions. The enzymatic activity being the expression product of cells associated with tumor tissue. The enzymatic activity on the cleavage site of the targeting peptide converts the immunoconjugate to an active immunostimulatory drug free of targeting peptide and linking group. The cleavage site may be specifically recognized by elastase. Elastase may catalyze the cleavage of a specific peptidic bond between the C-terminal amino acid residue of the specific peptide and the immunostimulatory moiety of the immunoconjugate.

Specific cleavage of the immunoconjugates of the invention takes advantage of the presence of tumor infiltrating cells of the immune system and leukocyte-secreted enzymes, to promote the activation of an anticancer drug at the tumor site.

In one embodiment, the elastase-substrate, peptide linker (EsPEP) has the formula:

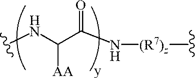

where AA is independently selected from a natural or unnatural amino acid side chain, or one or more of AA, and an adjacent nitrogen atom form a 5-membered ring proline amino acid, and the wavy line indicates a point of attachment;
R⁷ is selected from the group consisting of C₆-C₂₀ aryldiyl and C₁-C₂₀ heteroaryldiyl, substituted with a group selected from —CH₂O—C(═O)—, —CH₂O—, —CH₂—, —CH₂N(R⁸)—, and —CH(R⁸)O—C(═O)—, where R⁸ is H or C₁-C₆ alkyl;
y is an integer from 2 to 12; and
z is 0 or 1.

In exemplary embodiments, EsPEP is a tripeptide and has the formulas:

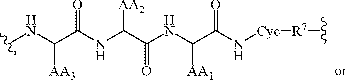

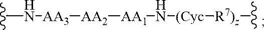

or EsPEP is a tetrapeptide and has the formulas:

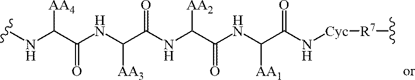

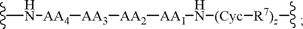

where AA₁, AA₂ and AA₃ are independently selected from a natural or unnatural amino acid side chain, or one or more of AA₁, AA₂, AA₃, AA₄, and an adjacent nitrogen atom form a 5-membered ring proline amino acid, and the wavy line indicates a point of attachment.

Cyc is selected from C₆-C₂₀ aryldiyl and C₁-C₂₀ heteroaryldiyl, optionally substituted with one or more groups selected from F, Cl, NO₂, —OH, —OCH₃, and a glucuronic acid having the structure:

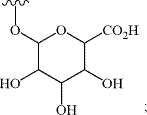

R⁷ is selected from the group consisting of —CH(R⁸)O—, —CH₂—, —CH₂N(R⁸)—, and —CH(R⁸)O—C(═O)—, where R⁸ is selected from H, C₁-C₆ alkyl, C(═O)—C₁-C₆ alkyl, and —C(═O)N(R⁹)₂, where R⁹ is independently selected from the group consisting of H, C₁-C₁₂ alkyl, and —(CH₂CH₂O)n—(CH₂)ₘ—OH, where m is an integer from 1 to 5, and n is an integer from 2 to 50, or two R⁹ groups together form a 5- or 6-membered heterocyclyl ring;
z is 0 or 1.

In an exemplary embodiment, EsPEP is a tripeptide wherein AA₁ is methyl, AA₂ forms proline, and AA₃ is isopropyl.

In an exemplary embodiment, EsPEP is a tetrapeptide wherein
AA₁ is selected from the group consisting of Abu, Ala, and Val;
AA₂ is selected from the group consisting of Nle(O-Bzl), Oic and Pro;
AA₃ is selected from the group consisting of Ala and Met(O)₂; and
AA₄ is selected from the group consisting of Oic, Arg(NO₂), Bpa, and Nle(O-Bzl).

In an exemplary embodiment, EsPEP is comprised of amino acid residues of amino acids selected from the group consisting of:

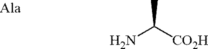

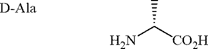

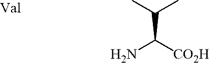

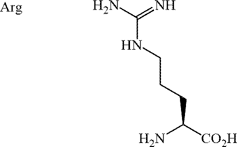 [SMILES: N=C(N)NCCC[C@H](N)C(=O)O ; "Arg"]

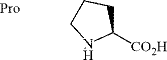

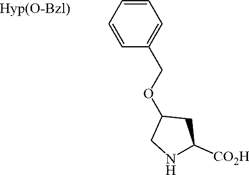

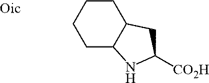

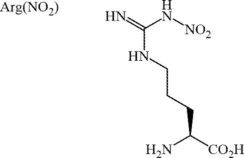 [SMILES: "Arg(NO2)" ; N=C(NCCC[C@H](N)C(=O)O)N[N+](=O)[O-]]

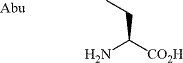

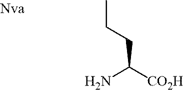

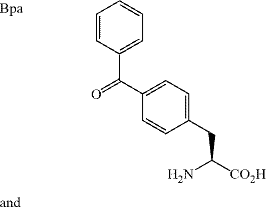

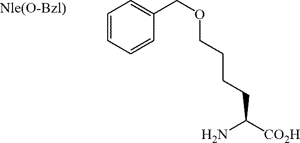 [SMILES: "Nle(O-Bzl)" ; N[C@@H](CCCCOCc1ccccc1)C(=O)O]

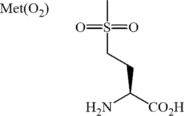

In an exemplary embodiment, EsPEP is selected from the group consisting of Ala-Pro-Val, Asn-Pro-Val, Ala-Ala-Val, Ala-Ala-Pro-Ala (SEQ ID NO: 639), Ala-Ala-Pro-Val (SEQ ID NO: 640), and Ala-Ala-Pro-Nva (SEQ ID NO: 641).

In an exemplary embodiment, EsPEP has the formula:

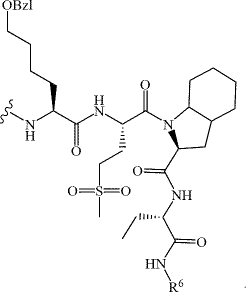

In an exemplary embodiment, EsPEP has the formula:

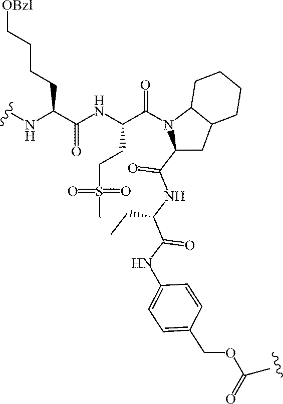

### IMMUNOSTIMULANT-ELASTASE SUBSTRATE, PEPTIDE LINKER COMPOUNDS

The immunoconjugates of the invention are prepared by conjugation of a cell-binding agent with an immunostimulant-elastase substrate, peptide linker compound. Immunostimulant-elastase substrate, peptide linker compounds comprise an immunostimulatory moiety covalently attached to a linker unit. The linker units comprise an elastase-substrate, peptide unit and functional groups and subunits which affect stability, permeability, solubility, and other pharmacokinetic, safety, and efficacy properties of the immunoconjugates. The linker unit includes a reactive functional group which reacts, i.e. conjugates, with a reactive functional group of the antibody. For example, a nucleophilic group such as a lysine side chain amino of the antibody reacts with an electrophilic reactive functional group of the immunostimulant-elastase substrate, peptide linker compound to form the immunoconjugate. A cysteine thiol of the cell-binding agent, e.g. an antibody, reacts with a maleimide or bromoacetamide group of the immunostimulant-elastase substrate, peptide linker compound to form the immunoconjugate.

Electrophilic reactive functional groups suitable for the immunostimulant-elastase substrate, peptide linker compounds include, but are not limited to, N-hydroxysuccinimidyl (NHS) esters and N-hydroxysulfosuccinimidyl (sulfo-NHS) esters (amine reactive); carbodiimides (amine and carboxyl reactive); hydroxymethyl phosphines (amine reactive); maleimides (thiol reactive); halogenated acetamides such as N-iodoacetamides (thiol reactive); aryl azides (primary amine reactive); fluorinated aryl azides (reactive via carbon-hydrogen (C—H) insertion); pentafluorophenyl (PFP) esters (amine reactive); tetrafluorophenyl (TFP) esters (amine reactive); tetrafluorophenyl, sulfonate (sulfo-TFP) esters, imidoesters (amine reactive); isocyanates (hydroxyl reactive); vinyl sulfones (thiol, amine, and hydroxyl reactive); pyridyl disulfides (thiol reactive); and benzophenone derivatives (reactive via C—H bond insertion). Further reagents include, but are not limited, to those described in Hermanson, Bioconjugate Techniques 2^{nd} Edition, Academic Press, 2008.

The invention provides solutions to the limitations and challenges to the design, preparation and use of immunoconjugates. Some linkers may be labile in the blood stream, thereby releasing unacceptable amounts of the adjuvant/drug prior to internalization in a target cell (Khot, A. et al (2015) Bioanalysis 7(13):1633-1648). Other linkers may provide stability in the bloodstream, but intracellular release effectiveness may be negatively impacted. Linkers that provide for desired intracellular release typically have poor stability in the bloodstream. Alternatively stated, bloodstream stability and intracellular release are typically inversely related. In addition, in standard conjugation processes, the amount of adjuvant/drug moiety loaded on the antibody, i.e. drug loading, the amount of aggregate that is formed in the conjugation reaction, and the yield of final purified conjugate that can be obtained are interrelated. For example, aggregate formation is generally positively correlated to the number of equivalents of adjuvant/drug moiety and derivatives thereof conjugated to the antibody. Under high drug loading, formed aggregates must be removed for therapeutic applications. As a result, drug loading-mediated aggregate formation decreases immunoconjugate yield and can render process scale-up difficult.

Exemplary embodiments of immunostimulant-elastase substrate, peptide linker compounds are selected from formulas IIa-f:

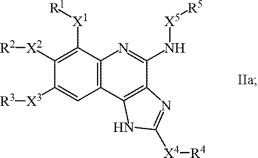

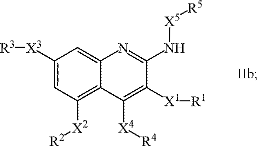

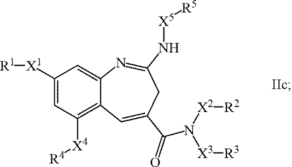

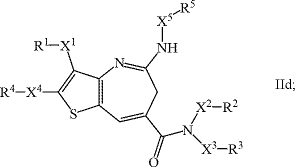

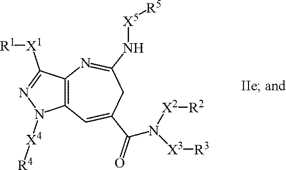

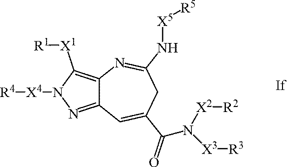

wherein R¹, R², R³, R⁴, and R⁵ are independently selected from the group consisting of H, C₁-C₁₂ alkyl, C₂-C₆ alkenyl, C₂-C₆ alkynyl, C₃-C₁₂ carbocyclyl, C₆-C₂₀ aryl, C₂-C₉ heterocyclyl, and C₁-C₂₀ heteroaryl, where alkyl, alkenyl, alkynyl, carbocyclyl, aryl, heterocyclyl, and heteroaryl are independently and optionally substituted with one or more groups selected from:
-(C₁-C₁₂ alkyldiyl)—N(R⁶)—*;
-(C₁-C₁₂ alkyldiyl)-N(R⁶)₂;
-(C₁-C₁₂ alkyldiyl)-OR⁶;
-(C₃-C₁₂ carbocyclyl);
-(C₃-C₁₂ carbocyclyl)-*;
-(C₃-C₁₂ carbocyclyl)-(C₁-C₁₂ alkyldiyl)—NR⁶—*;
-(C₃-C₁₂ carbocyclyl)-(C₁-C₁₂ alkyldiyl)-N(R⁶)₂;
-(C₃-C₁₂ carbocyclyl)-NR⁵-C(=NR^{6a})NR⁶-*;
-(C6-C₂₀ aryl);
-(C6-C₂₀ aryldiyl)-*;
-(C6-C₂₀ aryldiyl)—N(R⁶)—*;
-(C6-C₂₀ aryldiyl)-(C₁-C₁₂ alkyldiyl)—N(R⁶)—*;
-(C6-C₂₀ aryldiyl)-(C₁-C₁₂ alkyldiyl)-(C₂-C₂o heterocyclyldiyl)-*;
-(C6-C₂₀ aryldiyl)-(C₁-C₁₂ alkyldiyl)-N(R⁶)₂;
-(C6-C₂₀ aryldiyl)-(C₁-C₁₂ alkyldiyl)-NR⁶-C(=NR^{6a})N(R⁶)-*;
-(C₂-C₂₀ heterocyclyl);
-(C₂-C₂₀ heterocyclyl)-*;
-(C₂-C₉ heterocyclyl)-(C₁-C₁₂ alkyldiyl)—NR⁶—*;
-(C₂-C₉ heterocyclyl)-(C₁-C₁₂ alkyldiyl)-N(R⁶)₂;
-(C₂-C₉ heterocyclyl)—C(═O)—(C₁-C₁₂ alkyldiyl)—N(R⁶)—*;
-(C₂-C₉ heterocyclyl)-NR⁵-C(=NR^{6a})NR⁶-*;
-(C₂-C₉ heterocyclyl)-NR⁶-(C₆-C₂₀ aryldiyl)-(C₁-C₁₂ alkyldiyl)—N(R⁶)—*;
-(C₂-C₉ heterocyclyl)-(C₆-C₂₀ aryldiyl)-*;
-(C₁-C₂₀ heteroaryl);
-(C₁-C₂₀ heteroaryldiyl)-*;
-(C₁-C₂₀ heteroaryldiyl)-(C₁-C₁₂ alkyldiyl)—N(R⁶)—*;
-(C₁-C₂₀ heteroaryldiyl)-(C₁-C₁₂ alkyldiyl)-N(R⁶)₂;
-(C₁-C₂₀ heteroaryldiyl)-NR⁶-C(=NR^{6a})N(R⁶)-*;
-(C₁-C₂₀ heteroaryldiyl)—N(R⁶)C(═O)^{_}(C₁-C₁₂ alkyldiyl)—N(R⁶)—*;
—C(═O)—*;
—C(═O)—(C₁-C₁₂ alkyldiyl)—N(R⁶)—*;
—C(═O)—(C₂-C₂₀ heterocyclyldiyl)-*;
—C(═O)N(R⁶)₂;
—C(═O)N(R⁶)—*;
—C(═O)N(R⁶)—(C₁-C₁₂ alkyldiyl)—N(R⁶)C(═O)R⁵;
—C(═O)N(R⁶)—(C₁-C₁₂ alkyldiyl)—N(R⁶)C(═O)N(R⁶)₂;
—C(═O)NR⁶—(C₁-C₁₂ alkyldiyl)-N(R⁶)CO₂R⁶;
—C(═O)NR⁶—(C₁-C₁₂ alkyldiyl)-N(R⁶)C(=NR^{6a})N(R⁶)₂;
—C(═O)NR⁶—(C₁-C₁₂ alkyldiyl)-NR⁶C(=NR^{6a})R⁶;
—C(═O)NR⁶—(C₁—Ca alkyldiyl)-NR⁶(C₂-C₅ heteroaryl);
—C(═O)NR⁶—(C₁-C₂₀heteroaryldiyl)—N(R⁶)—*;
—C(═O)NR⁶—(C₁-C₂₀ heteroaryldiyl)-*;
-C(=O)NR^{6_}(C₁-C₂₀ heteroaryldiyl)-(C₁-C₁₂ alkyldiyl)-N(R⁶)₂;
-C(=O)NR^{6_}(C₁-C₂₀ heteroaryldiyl)-(C₂-C₂₀ heterocyclyldiyl)—C(═O)NR⁶—(C₁-C₁₂ alkyldiyl)—NR⁶—*;
—N(R⁶)₂;
—N(R⁶)—*;
—N(R⁶)C(═O)R⁶;
—N(R⁶)C(═O)—*;
—N(R⁶)C(═O)N(R⁶)₂;
—N(R⁶)C(═O)N(R⁶)—*;
—N(R⁶)CO₂R⁶;
—N(R⁶)CO₂(R⁶)—*;
-NR⁶C(=NR^{6a})N(R⁶)₂;
-NR⁶C(=NR^{6a})N(R⁶)-*;
-NR⁶C(=NR^{6a})R⁶_{;}
—N(R⁶)C(═O)—(C₁-C₁₂ alkyldiyl)—N(R⁶)—*;
—N(R⁶)—(C₂-C₅ heteroaryl);
—N(R⁶)—S(═O)z—(C₁-C₁₂ alkyl);
—O—(C₁-C₁₂ alkyl);
—O—(C₁-C₁₂ alkyldiyl)-N(R⁶)₂;
—O—(C₁-C₁₂ alkyldiyl)—N(R⁶)—*;
—OC(═O)N(R⁶)₂;
—OC(═O)N(R⁶)—*;
—S(═O)₂—(C₂-C₂₀ heterocyclyldiyl)-*;
—S(═O)₂—(C₂-C₂₀ heterocyclyldiyl)-(C₁-C₁₂ alkyldiyl)-N(R⁶)₂;
—S(═O)₂—(C₂-C₂₀ heterocyclyldiyl)-(C₁-C₁₂ alkyldiyl)—NR⁶—*; and
—S(═O)₂—(C₂-C₂₀ heterocyclyldiyl)-(C₁-C₁₂ alkyldiyl)-OH;
or R² and R³ of formulas Ic or Id together form a 5- or 6-membered heterocyclyl ring;
X¹, X², X³, X⁴, and X⁵ are independently selected from the group consisting of a bond, C(═O), C(═O)N(R⁶), O, N(R⁶), S, S(O)₂, and S(O)₂N(R⁶);
R⁶ is selected from the group consisting of H, C₆-C₂₀ aryl, C₆-C₂₀ aryldiyl, C₁-C₁₂ alkyl, and C₁-C₁₂ alkyldiyl, or two R⁶ groups together form a 5- or 6-membered heterocyclyl ring;
R^{6a} is selected from the group consisting of C₆-C₂₀ aryl and C₁-C₂₀ heteroaryl;
where the asterisk * indicates the attachment site of L, and where one of R¹, R², R³, R⁴ and R⁵ is attached to L;
L is selected from the group consisting of:
Q—C(═O)—(PEG)—C(═O)—(EsPEP)—;
Q—C(═O)—(PEG)—C(═O)—(EsPEP)—N(R⁶)—(C₁-C₁₂ alkyldiyl)-;
Q—C(═O)—(PEG)—C(═O)—(EsPEP)—N(R⁶)^{_}(C₁-C₁₂ alkyldiyl)—N(R⁶)C(═O)—(C₂-C₅ monoheterocyclyldiyl)-;
Q—C(═O)—(PEG)—N(R⁶)—(PEG)—C(═O)—(EsPEP)—;
Q—C(═O)—(PEG)—N⁺(R⁶)₂—(PEG)—C(═O)—(EsPEP)—;
Q—C(═O)—(PEG)—C(═O)—N(R⁶)CH(AA₁)C(═O)—(PEG)—C(═O)—(EsPEP)—;
Q—C(═O)—(C₁-C₁₂ alkyldiyl)—C(═O)—(EsPEP)—;
Q—C(═O)—(C₁-C₁₂alkyldiyl)—C(═O)—(EsPEP)—N(R⁶)—(C₁-C₁₂alkyldiyl)-;
Q—C(═O)—(C₁-C₁₂ alkyldiyl)—C(═O)—(EsPEP)—N(R⁶)—(C₁-C₁₂ alkyldiyl)—N(R⁶)—C(═O);
Q—C(═O)—(C₁-C₁₂ alkyldiyl)—C(═O)—(EsPEP)—N(R⁶)—(C₁-C₁₂ alkyldiyl)—N(R⁶)C(═O)—(C₂-C₅ monoheterocyclyldiyl)-;
Q—(CH₂)ₘ₋C(═O)N(R⁶)—PEG—C(═O)—(EsPEP)—;
Q—(CH₂)ₘ—C(═O)—(EsPEP)—N(R⁶)—(C₁-C₁₂ alkyldiyl)-;
Q—(CH₂)ₘC(═O)—(EsPEP)—N(R6)—(C1-C₁₂ alkyldiyl)N(R⁶)C(═O)—; and
Q—(CH₂)ₘ—C(═O)—(EsPEP)—N(R⁶)^{_}(C₁-C₁₂alkyldiyl)—N(R⁶)C(═O)—(C₂-C₅ monoheterocyclyldiyl)-;
PEG has the formula: —(CH₂CH₂O)n—(CH₂)ₘ—; m is an integer from 1 to 5, and n is an integer from 2 to 50;
EsPEP is an elastase-substrate, peptide linker unit comprising 2 to 12 amino acid residues; and
Q is selected from the group consisting of N-hydroxysuccinimidyl, N-hydroxysulfosuccinimidyl, maleimide, and phenoxy substituted with one or more groups independently selected from F, Cl, NO₂, and SO₃ ;
alkyl, alkyldiyl, alkenyl, alkenyldiyl, alkynyl, alkynyldiyl, aryl, aryldiyl, carbocyclyl, carbocyclyldiyl, heterocyclyl, heterocyclyldiyl, heteroaryl, and heteroaryldiyl are independently and optionally substituted with one or more groups independently selected from F, Cl, Br, I, —CN, —CH₃, —CH₂CH₃, —CH═CH₂, —C≡CH, —C≡CCH₃, —CH₂CH₂CH₃, —CH(CH₃)₂, —CH₂CH(CH₃)₂, —CH₂OH, —CH₂OCH₃, —CH₂CH₂OH, —C(CH₃)₂OH, —CH(OH)CH(CH₃)₂, —C(CH₃)₂CH₂OH, —CH₂CH₂SO₂CH₃, —CH₂OP(O)(OH)₂, —CH₂F, —CHF₂, —CF₃, —CH₂CF₃, —CH₂CHF₂, —CH(CH₃)CN, —C(CH₃)₂CN, —CH₂CN, —CH₂NH₂, —CH₂NHSO₂CH₃, —CH₂NHCH₃, —CH₂N(CH₃)₂, —CO₂H, —COCH₃, —CO₂CH₃, —CO₂C(CH₃)₃, —COCH(OH)CH₃, —CONH₂, —CONHCH₃, —CON(CH₃)₂, —C(CH₃)₂CONH₂, —NH₂, —NHCH₃, —N(CH₃)₂, —NHCOCH₃, —N(CH₃)COCH₃, —NHS(O)₂CH₃, —N(CH₃)C(CH₃)₂CONH₂, —N(CH₃)CH₂CH₂S(O)₂CH₃, —NHC(═NH)H, —NHC(═NH)CH₃, —NHC(═NH)NH₂, —NHC(═O)NH₂, —NO₂, ═O, —OH, —OCH₃, —OCH₂CH₃, —OCH₂CH₂OCH₃, —OCH₂CH₂OH, —OCH₂CH₂N(CH₃)₂, —O(CH₂CH₂O)n—(CH₂)ₘCO₂H, —O(CH₂CH₂O)nH, —OP(O)(OH)₂, —S(O)₂N(CH₃)₂, —SCH₃, —S(O)₂CH₃, and —S(O)₃H.

An exemplary embodiment of an immunostimulant-elastase substrate, peptide linker compound has the formula:

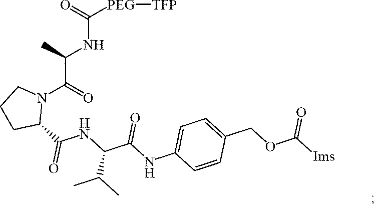

where TFP is 2,3,5,6-tetrafluorophenoxy.

An exemplary embodiment of an immunostimulant-elastase substrate, peptide linker compound includes wherein PEG has the formula: —(CH₂CH₂0)₂₅—(CH₂)₂— or —(CH₂CH₂O)₁₀—(CH₂)₂—.

Exemplary embodiments of immunostimulant-elastase substrate, peptide linker compounds comprise a structure selected from IIe-h:

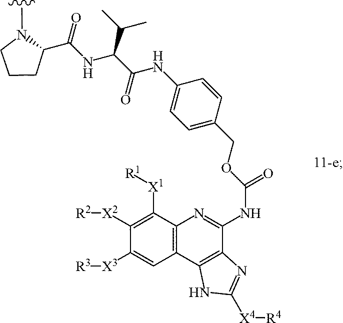

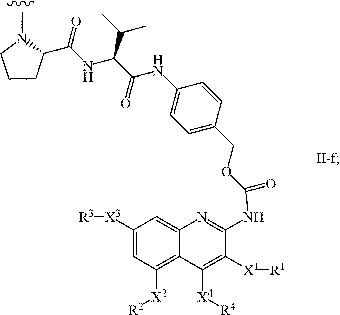

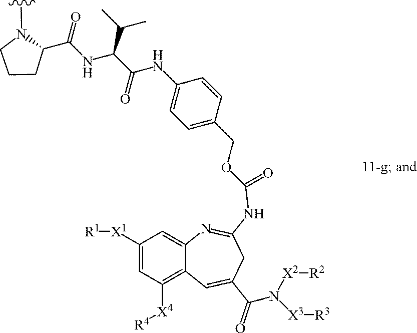

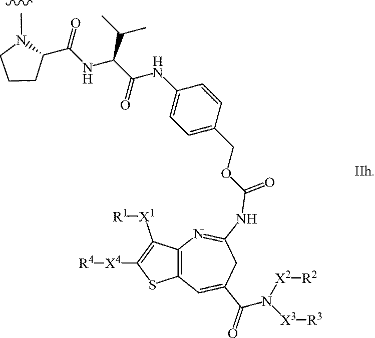

where the wavy line indicates the attachment through L to the antibody.

Exemplary embodiments of immunostimulant-elastase substrate, peptide linker compounds comprise a structure selected from IIi-1:

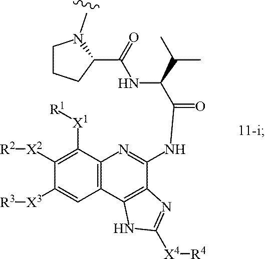

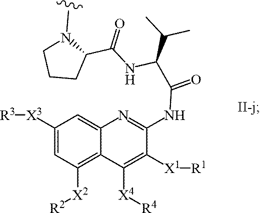

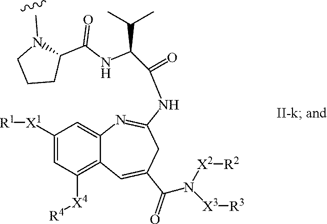

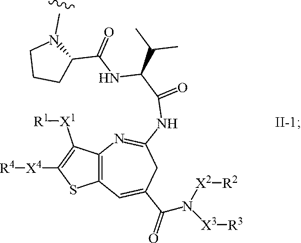

where the wavy line indicates the attachment through L to the antibody.

The invention includes all reasonable combinations, and permutations of the features, of the Formula II embodiments.

An exemplary embodiment of the immunostimulant-elastase substrate, peptide linker compound of Formula II is selected from Tables 1a and 1b. Each compound was synthesized and purified by the methods in the Examples provided herein, characterized by mass spectrometry, and shown to have the mass indicated. When conjugated to an antibody, the compounds of Tables 1a and 1b demonstrate surprising and unexpected properties which may predict useful therapeutic activity to treat cancer and other disorders.

TABLE 1a

Immunostimulant-elastase substrate, peptide linker compound of Formula II and intermediates thereof

| II No. | Structure | MW |
|---|---|---|
| II-1 | 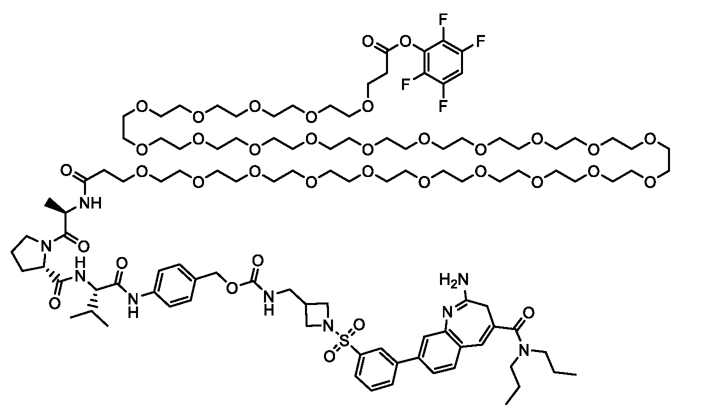 [SMILES: CCCN(CCCNC(=O)OC(C)(C)C)C(=O)C1=Cc2ccc(-c3cccc(S(=O)(=O)N4CC(CNC(=O)OCc5ccc(NC(=O)[C@@H](NC(=O)[C@@H]6CCCN6C(=O)CCOCCOCCOCCOCCOCCOCCOCCOCCOCCOCCC(=O)Oc6c(F)c(F)cc(F)c6F)C(C)C)cc5)C4)c3)cc2N=C(N)C1] | 2275.6 |
| II-2 | 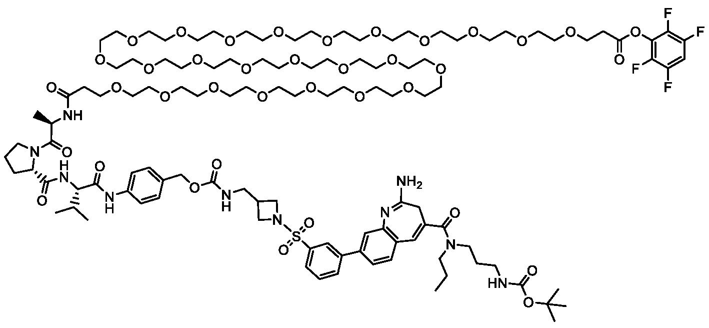 [SMILES: CCCN(CCC)C(=O)C1=Cc2sc(CN3CCN(C(=O)OCc4ccc(NC(=O)[C@@H](NC(=O)[C@@H]5CCCN5C(=O)[C@@H](C)NC(=O)OCC5c6ccccc6-c6ccccc65)C(C)C)cc4)CC3)cc2N=C(N)C1] | 2390.7 |
| II-3 | 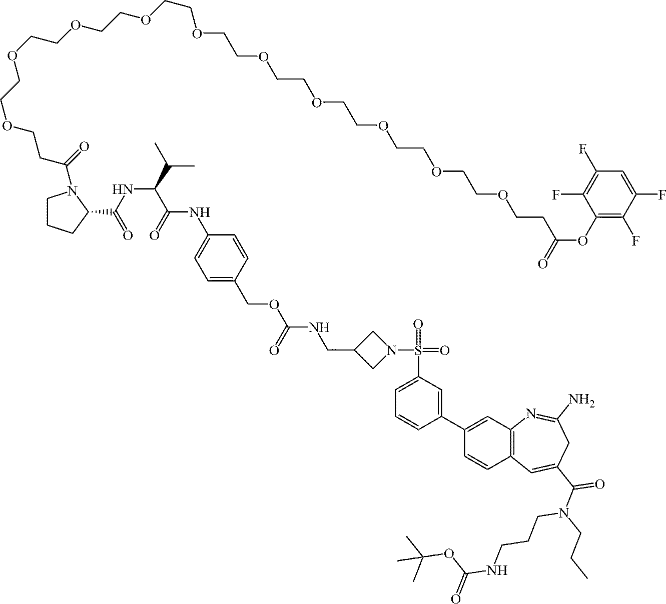 [SMILES: CCCC[C@@H](CCO)Nc1nc(NC(=O)OCc2ccc(NC(=O)[C@@H](NC(=O)[C@@H]3CCCN3C(=O)[C@@H](C)NC(=O)OCC3c4ccccc4-c4ccccc43)C(C)C)cc2)nc2ccccc12] | 1658.9 |
| II-4 | 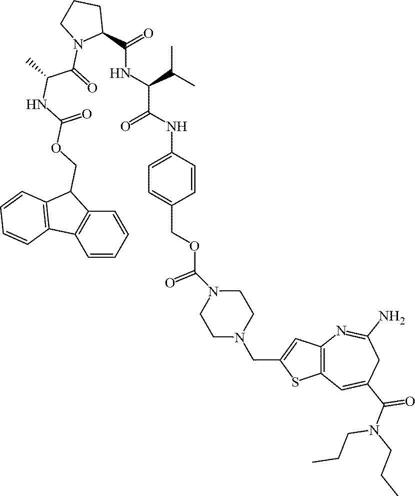 [SMILES: CCCN(CCCNC(=O)CC(C)(C)C)C(=O)C1=Cc2sc(CN3CCN(C(=O)OCc4ccc(NC(=O)[C@@H](NC(=O)[C@@H]5CCCN5C(=O)[C@@H](C)NC(=O)OCC5c6ccccc6-c6ccccc65)C(C)C)cc4)CC3)cc2N=C(N)C1] | 1028.3 |
| II-5 | 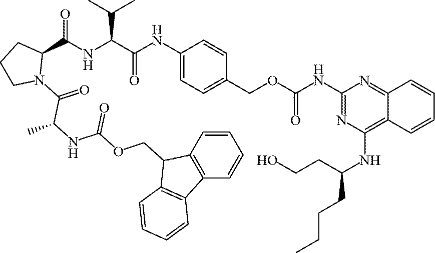 [SMILES: CCn1nc(C)cc1C(=O)/N=c1\[nH]c2cc(C(N)=O)cc(OC)c2n1C/C=C/Cn1/c(=N/C(=O)c2cc(C)nn2CC)[nH]c2cc(C(N)=O)cc(OCCCN3CCN(C(=O)OCc4ccc(NC(=O)[C@@H](NC(=O)[C@@H]5CCCN5C(=O)[C@H](C)NC(=O)CCOCCOCCOCCOCCOCCOCCOCCOCCOCCOCCNC(=O)CN5C(=O)C=CC5=O)C(C)C)cc4)CC3)c21] | 913.1 |
| II-6 | 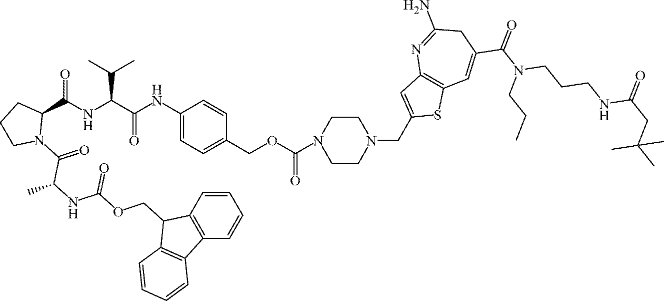 | 1141.4 |

TABLE 1b

STING agonist immunostimulant-elastase substrate, peptide linker compound

| No. | Structure | MW |
|---|---|---|
| II-7 | 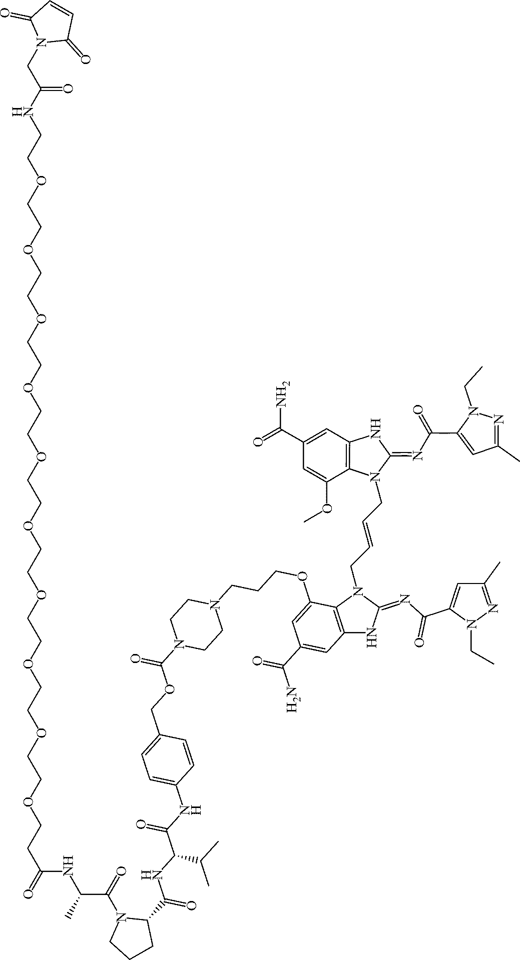 | 1914.2 |

### IMMUNOCONJUGATES

Exemplary embodiments of immunoconjugates comprise a cell-binding agent covalently attached to one or more immunostimulatory moieties by an elastase-substrate, peptide linker, having the Formula I:

Targeting properties of the cell-binding agent may drive the immunoconjugate accumulation at a tumor site, where the proinflammatory stimuli result in the recruitment of tumor-infiltrating leukocytes, such as neutrophils. The activation of the latter promotes the release of elastase, which triggers the payload release in the tumor microenvironment. This mode of activation may possess potential therapeutic benefits, since the free payload would diffuse in the tumor mass, and act against a large variety of cells (e.g., antigen-negative cancer cells, endothelial and other cancer-associated host cells) leading to a localized damage (Li, F. et al (2016) Cancer Res. 76:2710-2719). Lipophilic immunostimulatory moieties may be most suited for this strategy, as the membrane permeability may facilitate biological activity by the so-called “bystander effect.”

The invention includes an immunoconjugates comprising a cell-binding agent covalently attached to one or more immunostimulatory moieties by an elastase-substrate, peptide linker.

An exemplary embodiment of the immunoconjugate includes wherein the cell-binding agent is an antibody.

The antibody may be an antibody construct that has an antigen binding domain that binds PD-L1. The antibody may be selected from the group consisting of atezolizumab, durvalumab, and avelumab, or a biosimilar or a biobetter thereof.

The antibody may be an antibody construct that has an antigen binding domain that binds HER2. The antibody may be selected from the group consisting of trastuzumab and pertuzumab, or a biosimilar or a biobetter thereof.

The antibody may be an antibody construct that has an antigen binding domain that binds CEA. The antibody may be labetuzumab, or a biosimilar or a biobetter thereof.

An exemplary embodiment of the immunoconjugate includes wherein the one or more immunostimulatory moieties is a pattern-recognition receptor.

An exemplary embodiment of the immunoconjugate includes wherein the one or more immunostimulatory moieties interact with or modulate a receptor selected from the group consisting of TLR, STING, NOD2, RIG-1, and NLRP3.

An exemplary embodiment of the immunoconjugate has Formula I:

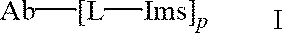

or a pharmaceutically acceptable salt thereof, wherein:
Ab is the antibody;
L is the linker comprising an elastase-substrate, peptide linker unit;
Ims is the immunostimulatory moiety; and
p is an integer from 1 to 8.

An exemplary embodiment of the immunoconjugate includes wherein Ims is selected from formulas Ia-f:

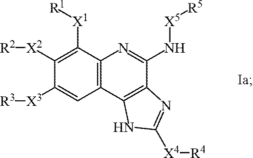

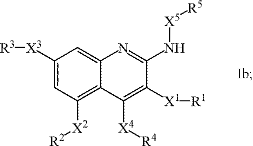

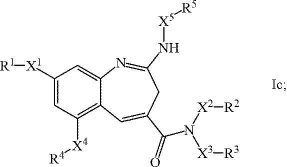

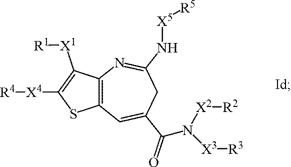

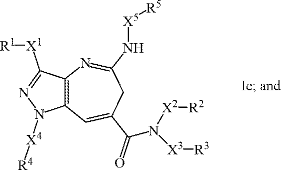

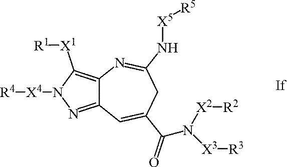

wherein R¹, R², R³, R⁴, and R⁵ are independently selected from the group consisting of H, C₁-C₁₂ alkyl, C₂-C₆ alkenyl, C₂-C₆ alkynyl, C₃-C₁₂ carbocyclyl, C₆-C₂₀ aryl, C₂-C₉ heterocyclyl, and C₁-C₂₀ heteroaryl, where alkyl, alkenyl, alkynyl, carbocyclyl, aryl, heterocyclyl, and heteroaryl are independently and optionally substituted with one or more groups selected from:
   -(C₁-C₁₂ alkyldiyl)—N(R⁶)—*;
   -(C₁-C₁₂ alkyldiyl)-N(R⁶)₂;
   -(C₁-C₁₂ alkyldiyl)-OR⁶;
   -(C₃-C₁₂ carbocyclyl);
   -(C₃-C₁₂ carbocyclyl)-*;
   -(C₃-C₁₂ carbocyclyl)-(C₁-C₁₂ alkyldiyl)—NR⁶—*;
   -(C₃-C₁₂ carbocyclyl)-(C₁-C₁₂ alkyldiyl)-N(R⁶)₂;
   -(C₃-C₁₂ carbocyclyl)-NR⁵-C(=NR^{6a})NR⁶-*;
   -(C₆-C₂₀ aryl);
   -(C₆-C₂₀ aryldiyl)-*;
   -(C₆-C₂₀ aryldiyl)—N(R⁶)—*;
   -(C₆-C₂₀ aryldiyl)-(C₁-C₁₂ alkyldiyl)—N(R⁶)—*;
   -(C₆-C₂₀ aryldiyl)-(C₁-C₁₂ alkyldiyl)-(C₂-C₂₀ heterocyclyldiyl)-*;
   -(C₆-C₂₀ aryldiyl)-(C₁-C₁₂ alkyldiyl)-N(R⁶)₂;
   -(C₆-C₂₀ aryldiyl)-(C₁-C₁₂ alkyldiyl)—NR⁶—C(═NR^{6a})N(R⁶)—*;
   -(C₂-C₂₀ heterocyclyl);
   -(C₂-C₂₀ heterocyclyl)-*;
   -(C₂-C₉ heterocyclyl)-(C₁-C₁₂ alkyldiyl)—NR⁶—*;
   -(C₂-C₉ heterocyclyl)-(C₁-C₁₂ alkyldiyl)-N(R⁶)₂;
   -(C₂-C₉ heterocyclyl)—C(═O)—(C₁-C₁₂ alkyldiyl)—N(R⁶)—*;
   -(C₂-C₉ heterocyclyl)-NR⁵-C(=NR^{6a})NR⁶-*;
   -(C₂-C₉ heterocyclyl)-NR⁶-(C₆-C₂₀ aryldiyl)-(C₁-C₁₂ alkyldiyl)—N(R⁶)—*;
   -(C₂-C₉ heterocyc1yl)-(C₆-C₂₀ aryldiyl)-*;
   -(C₁-C₂₀ heteroaryl);
   -(C₁-C₂₀ heteroaryldiyl)-*;
   -(C₁-C₂₀ heteroaryldiyl)-(C₁-C₁₂ alkyldiyl)—N(R⁶)—*;
   -(C₁-C₂₀ heteroaryldiyl)-(C₁-C₁₂ alkyldiyl)-N(R⁶)₂;
   -(C₁-C₂₀ heteroaryldiyl)-NR⁶-C(=NR^{6a})N(R⁶)-*;
   -(C₁-C₂₀ heteroaryldiyl)—N(R⁶)C(═O)—(C₁-C₁₂ alkyldiyl)—N(R⁶)—*;
   —C(═O)—*;
   —C(═O)—(C ₁-C₁₂ alkyldiyl)—N(R⁶)—*;
   —C(═O)—(C₂-C₂₀ heterocyclyldiyl)-*;
   —C(═O)N(R⁶)₂;
   —C(═O)N(R⁶)—*;
   —C( =0)N(R⁶)-(C₁-C₁₂ alkyldiyl)—N(R⁶)C(═O)R⁵;
   —C( =0)N(R⁶)-(C₁-C₁₂ alkyldiyl)—N(R⁶)C( ═O)N(R ⁶)₂;
   —C(═0)NR^{6_}(C₁-C₁₂ alkyldiyl)-N(R⁶)CO₂R⁶;
   -C( =O)NR^{6_}(C1-C₁₂ alkyldiyl)-N(R ⁶)C(=NR ^{6a})N(R ⁶)₂;
   —C(═O)NR⁶—(C₁-C₁₂ alkyldiyl)-NR⁶C(=NR^{6a})R⁶;
   —C(═O)NR⁶—(C₁-C₈ alkyldiyl)-NR⁶(C₂-C₅ heteroaryl);
   —C(═O)NR⁶—(C₁-C₂₀ heteroaryldiyl)—N(R⁶)—*;
   —C(═O)NR⁶—(C₁-C₂₀ heteroaryldiyl)-*;
   —C(═O)NR⁶—(C₁-C₂₀ heteroaryldiyl)-(C₁-C₁₂ alkyldiyl)-N(R⁶)₂;
   —C(═O)NR⁶—(C₁-C₂₀ heteroaryldiyl)-(C₂-C₂₀ heterocyclyldiyl)—C(═O)NR⁶—(C₁-C₁₂ alkyldiyl)—NR⁶—*;
   —N(R⁶)₂;
   —N(R⁶)—*;
   —N(R⁶)C(═O)R⁶;
   —N(R⁶)C(═O)—*;
   —N(R⁶)C(═O)N(R⁶)z;
   —N(R⁶)C(═O)N(R⁶)—*;
   —N(R⁶)CO₂R⁶;
   —N(R⁶)CO₂(R⁶)—*;
   —NR⁶C(═NR^{6a})N(R⁶)₂;
   —NR⁶C(═NR^{6a})N(R⁶)—*;
   —NR⁶C(═NR^{6a})R⁶;
   —N(R⁶)C(═O)—(C₁-C₁₂ alkyldiyl)—N(R⁶)—*;
   —N(R⁶)—(C₂-C₅ heteroaryl);
   —N(R⁶)—S(═O)₂—(C₁-C₁₂ alkyl);
   —O—(C₁-C₁₂ alkyl);
   —O—(C₁-C₁₂ alkyldiyl)-N(R⁶)₂;
   —O—(C₁-C₁₂ alkyldiyl)—N(R⁶)—*;
   —OC(═O)N(R⁶)₂;
   —OC(═O)N(R⁶)—*;
   —S(═O)₂—(C₂-C₂₀ heterocyclyldiyl)-*;
   —S(═O)₂—(C₂-C₂₀ heterocyclyldiyl)-(C₁-C₁₂ alkyldiyl)-N(R⁶)₂;
   —S(═O)₂—(C₂-C₂₀ heterocyclyldiyl)-(C₁-C₁₂ alkyldiyl)—NR⁶—*; and
   —S(═O)₂—(C₂-C₂₀ heterocyclyldiyl)-(C₁-C₁₂ alkyldiyl)-OH;
   or R² and R³ of formulas Ic or Id together form a 5- or 6-membered heterocyclyl ring;
   X¹, X², X³, X⁴, and X⁵ are independently selected from the group consisting of a bond, C(═O), C(═O)N(R⁶), O, N(R⁶), S, S(O)₂, and S(O)₂N(R⁶);
   R⁶ is selected from the group consisting of H, C₆-C₂₀ aryl, C₆-C₂₀ aryldiyl, C₁-C₁₂ alkyl, and C₁-C₁₂ alkyldiyl, or two R⁶ groups together form a 5- or 6-membered heterocyclyl ring;
   R^{6a} is selected from the group consisting of C₆-C₂₀ aryl and C₁-C₂₀ heteroaryl;
   where the asterisk * indicates the attachment site of L, and where one of R¹, R², R³, R⁴ and R⁵ is attached to L; and
   alkyl, alkyldiyl, alkenyl, alkenyldiyl, alkynyl, alkynyldiyl, aryl, aryldiyl, carbocyclyl, carbocyclyldiyl, heterocyclyl, heterocyclyldiyl, heteroaryl, and heteroaryldiyl are independently and optionally substituted with one or more groups independently selected from F, Cl, Br, I, —CN, —CH₃, —CH₂CH₃, —CH═CH₂, —C≡CH, —C≡CCH₃, —CH₂CH₂CH₃, —CH(CH₃)₂, —CH₂CH(CH₃)₂, —CH₂OH, —CH₂OCH₃, —CH₂CH₂OH, —C(CH₃)₂OH, —CH(OH)CH(CH₃)₂, —C(CH₃)₂CH₂OH, —CH₂CH₂SO₂CH₃, —CH₂OP(O)(OH)₂, —CH₂F, —CHF₂, —CF₃, —CH₂CF₃, —CH₂CHF₂, —CH(CH₃)CN, —C(CH₃)₂CN, —CH₂CN, —CH₂NH₂, —CH₂NHSO₂CH₃, —CH₂NHCH₃, —CH₂N(CH₃)₂, —CO₂H, —COCH₃, —CO₂CH₃, —CO₂C(CH₃)₃, —COCH(OH)CH₃, —CONH₂, —CONHCH₃, —CON(CH₃)₂, —C(CH₃)₂CONH₂, —NH₂, —NHCH₃, —N(CH₃)₂, —NHCOCH3, —N(CH₃)COCH₃, —NHS(O)₂CH₃, —N(CH₃)C(CH₃)₂CONH₂, —N(CH₃)CH₂CH₂S(O)₂CH₃, —NHC(═NH)H, —NHC(═NH)CH₃, —NHC(═NH)NH₂, —NHC(═O)NH₂, —NO₂, ═O, —OH, —OCH₃, —OCH₂CH₃, —OCH₂CH₂OCH₃, —OCH₂CH₂OH, —OCH₂CH₂N(CH₃)₂, —O(CH₂CH₂O)ₙ—(CH₂)ₘCO₂H, —O(CH₂CH₂O)nH, —OP(O)(OH)₂, —S(O)₂N(CH₃)₂, —SCH₃, —S(O)₂CH₃, and —S(O)₃H.

An exemplary embodiment of the immunoconjugate includes wherein one of R¹, R², R³, R⁴ and R⁵ is selected from the formulas:

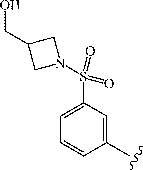

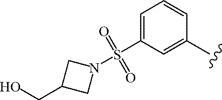

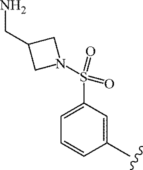

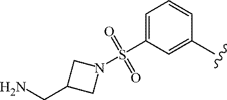

An exemplary embodiment of the immunoconjugate includes wherein Ims has formula Ig:

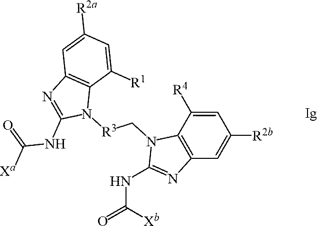

wherein X^{a} and X^{b} are independently selected from a five-membered heteroaryl;
R¹ is selected from the group consisting of F, Cl, Br, I, —CN, —OH, and —O—(C₁-C₆ alkyldiyl).
R^{2a} and R^{2b} are independently selected from —C(═O)N(R⁵)₂;
R³ is selected from C₁-C₆ alkyldiyl, -(C₁-C₃ alkyldiyl)-O-(C₁-C₃ alkyldiyl)-, C₂-C₆ alkenyldiyl and C₂-C₆ alkynyldiyl, optionally substituted with one or more groups selected from F, Cl, —OH, —OCH₃, —OCH₂CH₃, —OCH₂CH₂OCH₃, —OCH₂CH₂OH, —OCH₂CH₂N(CH₃)₂;
R⁴ is selected from the group consisting of:
   -(C₁-C₁₂ alkyldiyl)—N(R⁵)—*;
   -(C₁-C₁₂ alkyldiyl)-(C₂-C₂₀ heterocyclyldiyl)-*;
   —O—(C₁-C₁₂ alkyldiyl)—N(R⁵)—*;
   —O—(C₁-C₁₂ alkyldiyl)-(C₂-C₂₀ heterocyclyldiyl)-*;
   —O—(C₁-C₁₂ alkyldiyl)-(C₂-C₂₀ heterocyclyldiyl)—N(R⁵)—*;
   —OC(═O)N(R⁵)—*;
   —N(R⁵)—(C₁-C₁₂ alkyldiyl)—N(R⁵)—*;
   —N(R⁵)—(C₁-C₁₂ alkyldiyl)-(C₂-C₂₀ heterocyclyldiyl)-*;
   —C(═O)N(R⁵)—*;
   -(C₂-C₂₀ heterocyclyldiyl)-*;
   —S(═O)₂—(C₂-C₂₀ heterocyclyldiyl)-*;
   —S(═O)₂—(C₂-C₂₀ heterocyclyldiyl)-(C₁-C₁₂ alkyldiyl)—NR⁵—*;
where the asterisk * indicates the attachment site of L;
R⁵ is independently H or C₁-C₆ alkyl, or two R⁵ groups together form a 5- or 6-membered heterocyclyl ring; and
alkyl, alkyldiyl, alkenyl, alkenyldiyl, alkynyl, alkynyldiyl, aryl, aryldiyl, carbocyclyl, carbocyclyldiyl, heterocyclyl, heterocyclyldiyl, heteroaryl, and heteroaryldiyl are independently and optionally substituted with one or more groups independently selected from F, Cl, Br, I, —CN, —CH₃, —CH₂CH₃, —CH═CH₂, —C≡CH, —C≡CCH₃, —CH₂CH₂CH₃, —CH(CH₃)₂, —CH₂CH(CH₃)₂, —CH₂OH, —CH₂OCH₃, —CH₂CH₂OH, —C(CH₃)₂OH, —CH(OH)CH(CH₃)₂, —C(CH₃)₂CH₂OH, —CH₂CH₂SO₂CH₃, —CH₂OP(O)(OH)₂, —CH₂F, —CHF₂, —CF₃, —CH₂CF₃, —CH₂CHF₂, —CH(CH₃)CN, —C(CH₃)₂CN, —CH₂CN, —CH₂NH₂, —CH₂NHSO₂CH₃, —CH₂NHCH₃, —CH₂N(CH₃)₂, —CO₂H, —COCH₃, —CO₂CH₃, —CO₂C(CH₃)₃, —COCH(OH)CH₃, —CONH₂, —CONHCH₃, —CON(CH₃)₂, —C(CH₃)₂CONH₂, —NH₂, —NHCH₃, —N(CH₃)₂, —NHCOCH3, —N(CH₃)COCH₃, —NHS(O)₂CH₃, —N(CH₃)C(CH₃)₂CONH₂, —N(CH₃)CH₂CH₂S(O)₂CH₃, —NHC(═NH)H, —NHC(═NH)CH₃, —NHC(═NH)NH₂, —NHC(═O)NH₂, —NO₂, ═O, —OH, —OCH₃, —OCH₂CH₃, —OCH₂CH₂OCH₃, —OCH₂CH₂OH, —OCH₂CH₂N(CH₃)₂, —O(CH₂CH₂O)n—(CH₂)ₘCO₂H, —O(CH₂CH₂O)nH, —OCH₂F, —OCHF₂, —OCF₃, —OP(O)(OH)₂, —S(O)₂N(CH₃)₂, —SCH₃, —S(O)₂CH₃, and —S(O)₃H.

An exemplary embodiment of formula Ig includes wherein wherein X^{a} and X^{b} are independently selected from the group consisting of imidazolyl, pyrazolyl, triazolyl, tetrazolyl, furyl, thienyl, isoxazolyl, thiazolyl, oxadiazolyl, oxazolyl, isothiazolyl, pyrrolyl, oxadiazolyl, and thiadiazolyl.

An exemplary embodiment of formula Ig includes wherein wherein X^{a} and X^{b} are each pyrazolyl, substituted with one or more C₁-C₁₂ alkyl groups.

An exemplary embodiment of formula Ig includes wherein wherein R¹ is selected from the group consisting of —OCH₃, —OCH₂CH₃, —OCH₂CH₂OCH₃, —OCH₂CH₂OH, and —OCH₂CH₂N(CH₃)₂

An exemplary embodiment of formula Ig includes wherein R¹ is —OCH₃.

An exemplary embodiment of formula Ig includes wherein R¹ is F.

An exemplary embodiment of formula Ig includes wherein R^{2a} and R^{2b} are each —C(═O)NH₂.

An exemplary embodiment of formula Ig includes wherein R³ is selected from —CH₂CH₂—, —CH═CH—, and —C≡C—.

An exemplary embodiment of formula Ig includes wherein R³ is C₂-C₄ alkenyldiyl, substituted with one or more groups selected from F, —OH, and —OCH₃.

An exemplary embodiment of formula Ig includes wherein R⁴ is —O—(C₁-C₁₂ alkyldiyl)-(C₂-C₂₀ heterocyclyldiyl)-*.

An exemplary embodiment of formula Ig includes wherein C₁-C₁₂ alkyldiyl is propyldiyl and C₂-C₂₀ heterocyclyldiyl is piperidiyl.

An exemplary embodiment of the immunoconjugate includes wherein L is selected from the group consisting of:
—C(═O)—(PEG)—C(═O)—(EsPEP)—;
—C(═O)—(PEG)—C(═O)—(EsPEP)—N(R⁶)—;
—C(═O)—(PEG)—C(═O)—(EsPEP)—N(R⁶)—(C₁-C₁₂ alkyldiyl)-;
—C(═O)—(PEG)—C(═O)—(EsPEP)—N(R⁶)—(C₁-C₁₂ alkyldiyl)—N(R⁶)C(═O)—(C₂-C₅ monoheterocyclyldiyl)-;
—C(═O)—(PEG)—N(R⁶)—(PEG)—C(═O)—(EsPEP)—;
—C(═O)—(PEG)—N⁺(R⁶)₂—(PEG)—C(═O)—(EsPEP)—;
—C(═O)—(PEG)—C(═O)—N(R⁶)CH(AA₁)C(═O)—(PEG)—C(═O)—(EsPEP)—;
—C(═O)—(C₁-C₁₂ alkyldiyl)—C(═O)—(EsPEP)—;
—C(═O)—(C₁-C₁₂ alkyldiyl)—C(═O)—(EsPEP)—N(R⁶)—(C₁-C₁₂ alkyldiyl)-;
—C(═O)—(C₁-C₁₂alkyldiyl)—C(═O)—(EsPEP)—N(R⁶)—(C₁-C₁₂alkyldiyl)—N(R⁶)—C(═O);
—C(═O)—(C₁-C₁₂alkyldiyl)—C(═O)—(EsPEP)—N(R⁶)—(C₁-C₁₂alkyldiyl)—N(R⁶)C(═O)—(C₂-C₅ monoheterocyclyldiyl)-;
-succinimidyl—(CH₂)m—C(═O)N(R⁶)—PEG—C(═O)—(EsPEP)—;
-succinimidyl—(CH₂)m—C(═O)—(EsPEP)—N(R⁶)—(C₁-C₁₂ alkyldiyl)-;
-succinimidyl—(CH₂)ₘ—C(═O)—(EsPEP)—N(R⁶)—(C₁-C₁₂alkyldiyl)N(R⁶)C(═O)—; and
-(succinimidyl)—(CH₂)ₘ—C(═O)—(EsPEP)—N(R⁶)—(C₁-C₁₂alkyldiyl)—N(R⁶)C(═O)—(C₂-C₅ monoheterocyclyldiyl)-;
PEG has the formula: —(CH₂CH₂O)ₙ—(CH₂)ₘ—; m is an integer from 1 to 5, and n is an integer from 2 to 50;
EsPEP is the elastase-substrate, peptide linker unit comprising 2 to 12 amino acid residues; and
R⁶ is selected from the group consisting of H, C₆-C₂₀ aryl, C₆-C₂₀ aryldiyl, C₁-C₁₂ alkyl, and C₁-C₁₂ alkyldiyl, or two R⁶ groups together form a 5- or 6-membered heterocyclyl ring;
alkyl, alkyldiyl, alkenyl, alkenyldiyl, alkynyl, alkynyldiyl, aryl, aryldiyl, carbocyclyl, carbocyclyldiyl, heterocyclyl, heterocyclyldiyl, heteroaryl, and heteroaryldiyl are independently and optionally substituted with one or more groups independently selected from F, Cl, Br, I, —CN, —CH₃, —CH₂CH₃, —CH═CH₂, —C≡CH, —C≡CCH₃, —CH₂CH₂CH₃, —CH(CH₃)₂, —CH₂CH(CH₃)₂, —CH₂OH, —CH₂OCH₃, —CH₂CH₂OH, —C(CH₃)₂OH, —CH(OH)CH(CH₃)₂, —C(CH₃)₂CH₂OH, —CH₂CH₂SO₂CH₃, —CH₂OP(O)(OH)₂, —CH₂F, —CHF₂, —CF₃, —CH₂CF₃, —CH₂CHF₂, —CH(CH₃)CN, —C(CH₃)₂CN, —CH₂CN, —CH₂NH₂, —CH₂NHSO₂CH₃, —CH₂NHCH₃, —CH₂N(CH₃)₂, —CO₂H, —COCH₃, —CO₂CH₃, —CO₂C(CH₃)₃, —COCH(OH)CH₃, —CONH₂, —CONHCH₃, —CON(CH₃)₂, —C(CH₃)₂CONH₂, —NH₂, —NHCH3, —N(CH₃)₂, —NHCOCH₃, —N(CH₃)COCH₃, —NHS(O)₂CH₃, —N(CH₃)C(CH₃)₂CONH₂, —N(CH₃)CH₂CH₂S(O)₂CH₃, —NHC(═NH)H, —NHC(═NH)CH₃, —NHC(═NH)NH₂, —NHC(═O)NH₂, —NO₂, ═O, —OH, —OCH3, —OCH₂CH₃, —OCH₂CH₂OCH₃, —OCH₂CH₂OH, —OCH₂CH₂N(CH₃)₂, —O(CH₂CH₂O)ₙ—(CH₂)ₘCO₂H, —O(CH₂CH₂O)nH, —OP(O)(OH)₂, —S(O)₂N(CH₃)₂, —SCH₃, —S(O)₂CH₃, and —S(O)₃H.

An exemplary embodiment of the immunoconjugate includes wherein EsPEP has the formula:

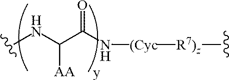

where AA is independently selected from a natural or unnatural amino acid side chain, or one or more of AA, and an adjacent nitrogen atom form a 5-membered ring proline amino acid, and the wavy line indicates a point of attachment;
Cyc is selected from C₆-C₂₀ aryldiyl and C₁-C₂₀ heteroaryldiyl, optionally substituted with one or more groups selected from F, Cl, NO₂, —OH, —OCH₃, and a glucuronic acid having the structure:

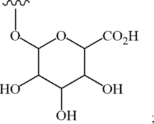

R⁷ is selected from the group consisting of -CH(R⁸)O- -CH₂-, —CH₂N(R⁸)—, and —CH(R⁸)O—C(═O)—, where R⁸ is selected from H, C₁-C₆ alkyl, C(═O)—C₁-C₆ alkyl, and —C(═O)N(R⁹)₂, where R⁹ is independently selected from the group consisting of H, C₁-C₁₂ alkyl, and —(CH₂CH₂O)ₙ—(CH₂)ₘ—OH, where m is an integer from 1 to 5, and n is an integer from 2 to 50, or two R⁹ groups together form a 5- or 6-membered heterocyclyl ring;
y is an integer from 2 to 12; and
z is 0 or 1.

An exemplary embodiment of the immunoconjugate includes wherein y is selected from 2, 3, and 4.

An exemplary embodiment of the immunoconjugate wherein EsPEP is a tripeptide having the formula:

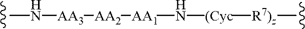

where AA₁, AA₂ and AA₃ are independently selected from a natural or unnatural amino acid, and the wavy line indicates a point of attachment;
Cyc is selected from C₆-C₂₀ aryldiyl and C₁-C₂₀ heteroaryldiyl, optionally substituted with one or more groups selected from F, Cl, NO₂, —OH, —OCH₃, and a glucuronic acid having the structure:

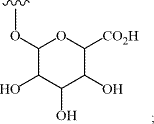

R⁷ is selected from the group consisting of —CH(R⁸)O—, —CH₂—, —CH₂N(R⁸)—, and —CH(R⁸)O—C(═O)—, where R⁸ is selected from H, C₁-C₆ alkyl, C(═O)—C₁-C₆ alkyl, and —C(═O)N(R⁹)₂, where R⁹ is independently selected from the group consisting of H, C₁-C₁₂ alkyl, and —(CH₂CH₂O)ₙ—(CH₂)ₘ—OH, where m is an integer from 1 to 5, and n is an integer from 2 to 50, or two R⁹ groups together form a 5- or 6-membered heterocyclyl ring; and
z is 0 or 1.

An exemplary embodiment of the immunoconjugate includes wherein AA₁ is methyl, AA₂ forms proline, and AA₃ is isopropyl.

An exemplary embodiment of the immunoconjugate includes wherein EsPEP has the formula:

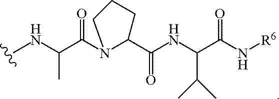

An exemplary embodiment of the immunoconjugate includes wherein EsPEP is selected from the formulas:

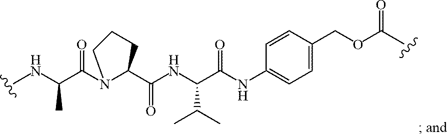

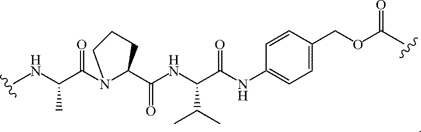

An exemplary embodiment of the immunoconjugate includes wherein L is:

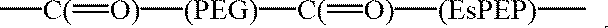

An exemplary embodiment of the immunoconjugate includes wherein PEG is:

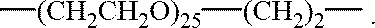

An exemplary embodiment of the immunoconjugate has the formula:

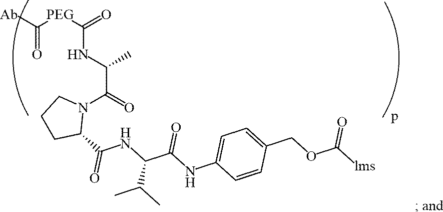

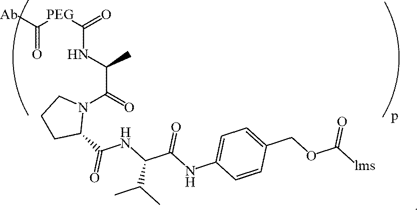

An exemplary embodiment of the immunoconjugate includes wherein Ims has formula IIc:

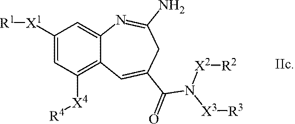

Exemplary embodiments of the immunoconjugate includes wherein Ims have the structures:

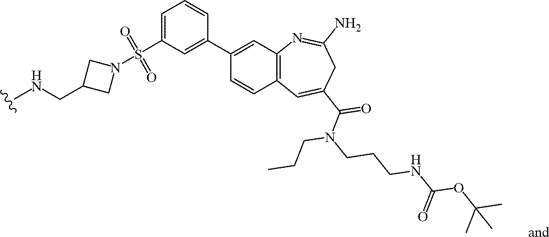

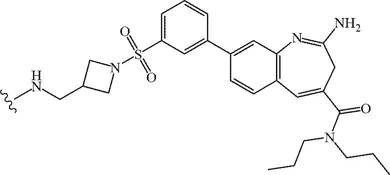

where the wavy line indicates the site of attachment to the linker.

An exemplary embodiment of the immunoconjugate includes wherein EsPEP is a tetrapeptide having the formula:

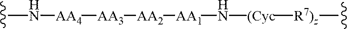

where AA₁, AA₂, AA₃ and AA₄ are independently selected from a natural or unnatural amino acid, and the wavy line indicates a point of attachment;
Cyc is selected from C₆-C₂₀ aryldiyl and C₁-C₂₀ heteroaryldiyl, optionally substituted with one or more groups selected from F, Cl, NO₂, —OH, —OCH₃, and a glucuronic acid having the structure:

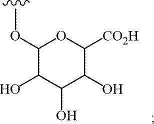

R⁷ is selected from the group consisting of -CH(R⁸)O- -CH₂-, —CH₂N(R⁸)—, and —CH(R⁸)O—C(═O)—, where R⁸ is selected from H, C₁-C₆ alkyl, C(═O)—C₁-C₆ alkyl, and —C(═O)N(R⁹)₂, where R⁹ is independently selected from the group consisting of H, C₁-C₁₂ alkyl, and —(CH₂CH₂O)ₙ—(CH₂)ₘ—OH, where m is an integer from 1 to 5, and n is an integer from 2 to 50, or two R⁹ groups together form a 5- or 6-membered heterocyclyl ring; and
z is 0 or 1.

An exemplary embodiment of the immunoconjugate includes wherein
AA₁ is selected from the group consisting of Abu, Ala, and Val;
AA₂ is selected from the group consisting of Nle(O-Bzl), Oic and Pro;
AA₃ is selected from the group consisting of Ala and Met(O)₂; and
AA₄ is selected from the group consisting of Oic, Arg(NO₂), Bpa, and Nle(O-Bzl).

An exemplary embodiment of the immunoconjugate includes wherein EsPEP has the formula:

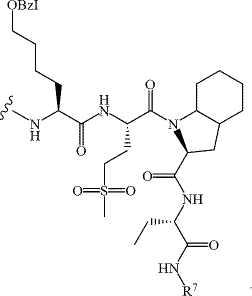

An exemplary embodiment of the immunoconjugate includes wherein EsPEP has the formula:

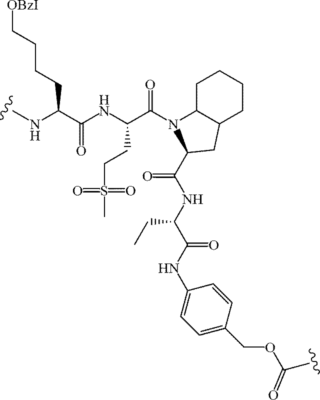

An exemplary embodiment of the immunoconjugate includes a structure selected from IIe-h:

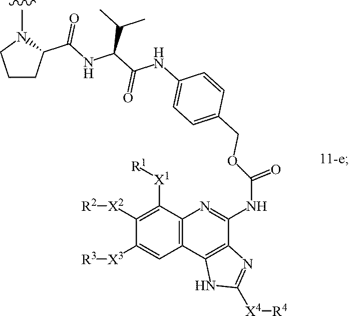

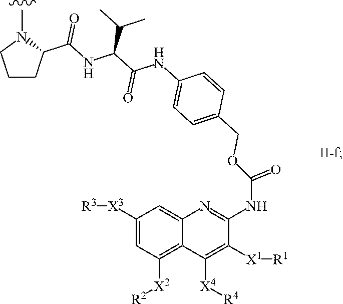

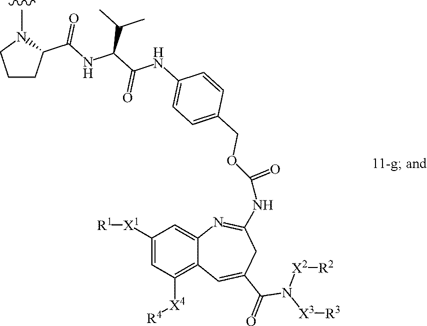

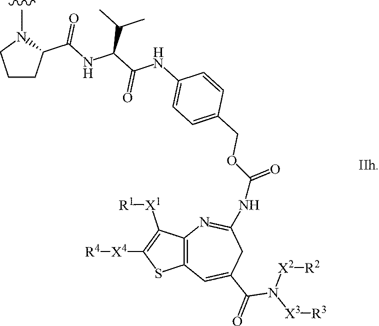

where the wavy line indicates the attachment through L to the antibody.

An exemplary embodiment of the immunoconjugate includes a structure selected from IIi-1:

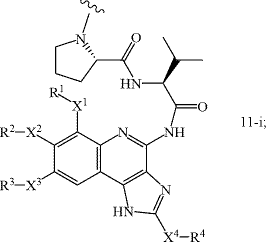

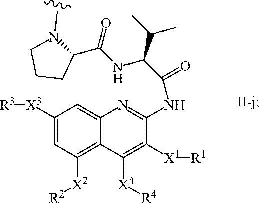

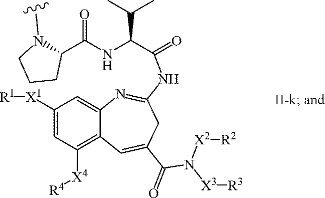

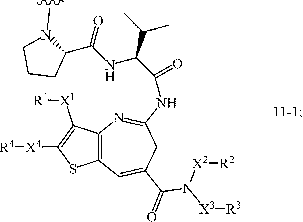

where the wavy line indicates the attachment through L to the antibody.

An exemplary embodiment of the immunoconjugate includes wherein R² and R³ are each C₁-C₈ alkyl.

An exemplary embodiment of the immunoconjugate includes wherein R² and R³ are each —CH₂CH₂CH₃.

An exemplary embodiment of the immunoconjugate includes wherein X² and X³ are each a bond, and R² or R³ is —O—(C₁-C₁₂ alkyl).

An exemplary embodiment of the immunoconjugate includes wherein R² or R³ is —OCH₂CH₃.

An exemplary embodiment of the immunoconjugate includes wherein the elastase-substrate, peptide linker is cleaved by elastase.

Exemplary embodiments of the immunoconjugate have the structures:

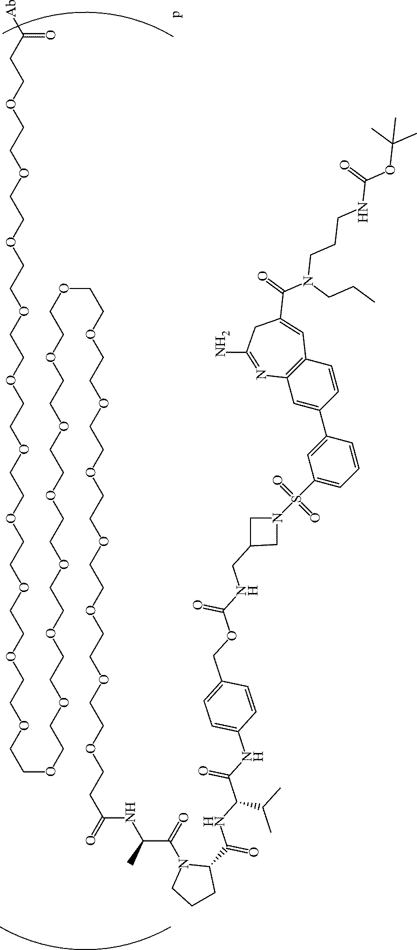

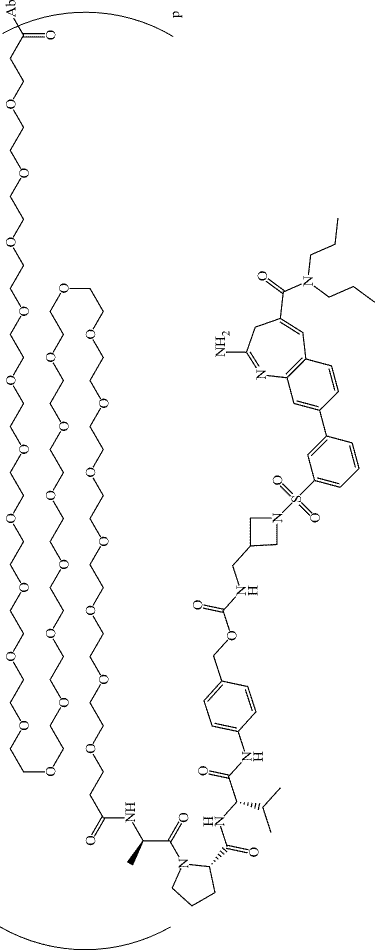

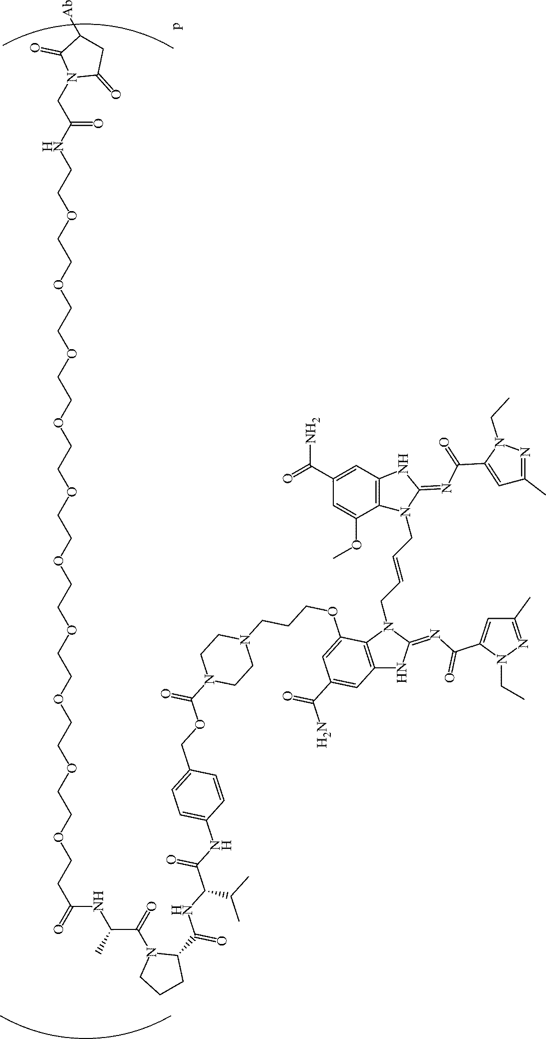

The invention includes all reasonable combinations, and permutations of the features, of the Formula I embodiments.

Drug loading is represented by p, the number of immunostimulatory moieties per antibody in an immunoconjugate of Formula I. Drug (immunostimulant) loading may range from 1 to about 8 drug moieties (D) per antibody. Immunoconjugates of Formula I include mixtures or collections of antibodies conjugated with a range of drug moieties, from 1 to about 8. In some embodiments, the number of drug moieties that can be conjugated to an antibody is limited by the number of reactive or available amino acid side chain residues such as lysine and cysteine. In some embodiments, free cysteine residues are introduced into the antibody amino acid sequence by the methods described herein. In such aspects, p may be 1, 2, 3, 4, 5, 6, 7, or 8, and ranges thereof, such as from 1 to 8 or from 2 to 5. In any such aspect, p and n are equal (i.e., p = n = 1, 2, 3, 4, 5, 6, 7, or 8, or some range there between). Exemplary immunoconjugates of Formula I include, but are not limited to, antibodies that have 1, 2, 3, or 4 engineered cysteine amino acids (Lyon, R. et al. (2012) Methods in Enzym. 502:123-138). In some embodiments, one or more free cysteine residues are already present in an antibody forming intrachain disulfide bonds, without the use of engineering, in which case the existing free cysteine residues may be used to conjugate the antibody to a drug. In some embodiments, an antibody is exposed to reducing conditions prior to conjugation of the antibody in order to generate one or more free cysteine residues.

For some immunoconjugates, p may be limited by the number of attachment sites on the antibody. For example, where the attachment is a cysteine thiol, as in certain exemplary embodiments described herein, an antibody may have only one or a limited number of cysteine thiol groups, or may have only one or a limited number of sufficiently reactive thiol groups, to which the drug may be attached. In other embodiments, one or more lysine amino groups in the antibody may be available and reactive for conjugation with an immunostimulant-linker compound of Formula II. In certain embodiments, higher drug loading, e.g. p >5, may cause aggregation, insolubility, toxicity, or loss of cellular permeability of certain antibody-drug conjugates. In certain embodiments, the average drug loading for an immunoconjugate ranges from 1 to about 8; from about 2 to about 6; or from about 3 to about 5. In certain embodiments, an antibody is subjected to denaturing conditions to reveal reactive nucleophilic groups such as lysine or cysteine.

The loading (drug/antibody ratio) of an immunoconjugate may be controlled in different ways, and for example, by: (i) limiting the molar excess of the immunostimulant-linker intermediate compound relative to antibody, (ii) limiting the conjugation reaction time or temperature, and (iii) partial or limiting reductive denaturing conditions for optimized antibody reactivity.

Where more than one nucleophilic group of the antibody reacts with a drug, then the resulting product is a mixture of immunoconjugate compounds with a distribution of one or more drug moieties attached to an antibody. The average number of drugs per antibody may be calculated from the mixture by a dual ELISA antibody assay, which is specific for antibody and specific for the drug. Individual immunoconjugate molecules may be identified in the mixture by mass spectroscopy and separated by HPLC, e.g. hydrophobic interaction chromatography (see, e.g., McDonagh et al. (2006) Prot. Engr. Design & Selection 19(7):299-307; Hamblett et al. (2004) Clin. Cancer Res. 10:7063-7070; Hamblett, K.J., et al. “Effect of drug loading on the pharmacology, pharmacokinetics, and toxicity of an anti-CD30 antibody-drug conjugate,” Abstract No. 624, American Association for Cancer Research, 2004 Annual Meeting, March 27-31, 2004, Proceedings of the AACR, Volume 45, March 2004; Alley, S.C., et al. “Controlling the location of drug attachment in antibody-drug conjugates,” Abstract No. 627, American Association for Cancer Research, 2004 Annual Meeting, March 27-31, 2004, Proceedings of the AACR, Volume 45, March 2004). In certain embodiments, a homogeneous immunoconjugate with a single loading value may be isolated from the conjugation mixture by electrophoresis or chromatography.

### BIOLOGICAL ACTIVITY OF IMMUNOCONJUGATES

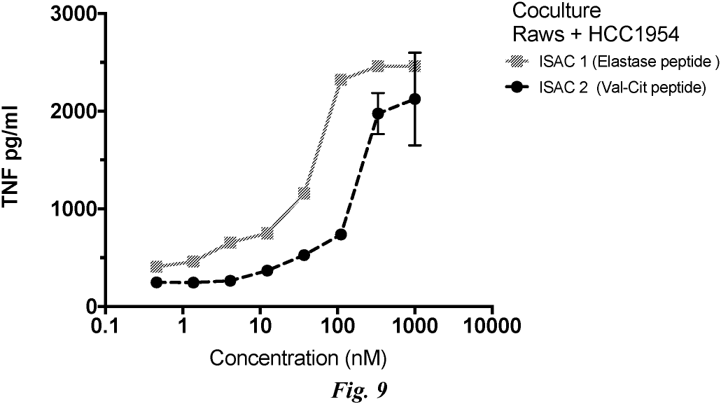

FIG. 9 shows a graph measuring potency as measured by TNFa production in a co-culture experiment with RAW 264.7 murine macrophage cells and HCC1954 HER2 expressing tumor cells. This experiment compared an elastase cleavable linker (Ala-Pro-Val) immunoconjugate ISAC-1, and a cathepsin B cleavable linker (Val-Cit) immunoconjugate ISAC-2. The antibody of ISAC-1 and ISAC-2 is anti-HER2 trastuzumab. The Val-Cit linker unit of ISAC-2 is a known cathepsin B substrate. Cells were cultured overnight at a 10:1 effector (macrophage) to target (HCC1954 tumor cell) ratio, and mouse TNFa was measured by ELISA as a readout of a proinflammatory response. The data demonstrated that ISAC-1 has increased potency relative to the cathepsin B cleavable peptide (Val-Cit) ISAC-2.

The RAW 264.7 murine macrophage cell line was cultured according to vendor protocols (Invivogen) and Example 203.

### PHARMACEUTICAL COMPOSITIONS OF IMMUNOCONJUGATES

The invention provides a composition, e.g., a pharmaceutically or pharmacologically acceptable composition or formulation, comprising a plurality of immunoconjugates as described herein and optionally a carrier therefor, e.g., a pharmaceutically or pharmacologically acceptable carrier. The immunoconjugates can be the same or different in the composition, i.e., the composition can comprise immunoconjugates that have the same number of immunostimulants linked to the same positions on the antibody construct and/or immunoconjugates that have the same number of immunostimulants linked to different positions on the antibody construct, that have different numbers of immunostimulants linked to the same positions on the antibody construct, or that have different numbers of immunostimulants linked to different positions on the antibody construct.

In an exemplary embodiment, a pharmaceutical composition comprises a therapeutically effective amount of the immunoconjugate and one or more pharmaceutically acceptable diluent, vehicle, carrier or excipient.

In an exemplary embodiment, a composition comprising the immunoconjugate compounds comprises a mixture of the immunoconjugate compounds, wherein the average drug (immunostimulatory moieties) loading per antibody in the mixture of immunoconjugate compounds is about 2 to about 5.

A composition of immunoconjugates of the invention can have an average adjuvant to antibody construct ratio (DAR) of about 0.4 to about 10. A skilled artisan will recognize that the number of immunostimulatory moieties conjugated to the antibody construct may vary from immunoconjugate to immunoconjugate in a composition comprising multiple immunoconjugates of the invention and thus the adjuvant to antibody construct (e.g., antibody) ratio can be measured as an average which may be referred to as the drug to antibody ratio (DAR). The adjuvant to antibody construct (e.g., antibody) ratio can be assessed by any suitable means, many of which are known in the art.

The average number of adjuvant moieties per antibody (DAR) in preparations of immunoconjugates from conjugation reactions may be characterized by conventional means such as mass spectrometry, ELISA assay, and HPLC. The quantitative distribution of immunoconjugates in a composition in terms of p may also be determined. In some instances, separation, purification, and characterization of homogeneous immunoconjugates where p is a certain value from immunoconjugates with other drug loadings may be achieved by means such as reverse phase HPLC or electrophoresis.

In some embodiments, the composition further comprises one or more pharmaceutically or pharmacologically acceptable excipients. For example, the immunoconjugates of the invention can be formulated for parenteral administration, such as IV administration or administration into a body cavity or lumen of an organ. Alternatively, the immunoconjugates can be injected intra-tumorally. Compositions for injection will commonly comprise a solution of the immunoconjugate dissolved in a pharmaceutically acceptable carrier. Among the acceptable vehicles and solvents that can be employed are water and an isotonic solution of one or more salts such as sodium chloride, e.g., Ringer’s solution. In addition, sterile fixed oils can conventionally be employed as a solvent or suspending medium. For this purpose, any bland fixed oil can be employed, including synthetic monoglycerides or diglycerides. In addition, fatty acids such as oleic acid can likewise be used in the preparation of inj ectables. These compositions desirably are sterile and generally free of undesirable matter. These compositions can be sterilized by conventional, well known sterilization techniques. The compositions can contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like.

The composition can contain any suitable concentration of the immunoconjugate. The concentration of the immunoconjugate in the composition can vary widely, and will be selected primarily based on fluid volumes, viscosities, body weight, and the like, in accordance with the particular mode of administration selected and the patient’s needs. In certain embodiments, the concentration of an immunoconjugate in a solution formulation for injection will range from about 0.1% (w/w) to about 10% (w/w).

### METHOD OF TREATING CANCER WITH IMMUNOCONJUGATES

The invention provides a method for treating cancer. The method includes administering a therapeutically effective amount of an immunoconjugate as described herein (e.g., as a composition as described herein) to a subject in need thereof, e.g., a subject that has cancer and is in need of treatment for the cancer. The method includes administering a therapeutically effective amount of an immunoconjugate (IC).

In certain embodiments, the immunoconjugate compounds of the invention include those with immunostimulatory activity. The immunoconjugates of the invention selectively deliver an effective dose of an immunostimulatory drug to tumor tissue, whereby greater selectivity (i.e., a lower efficacious dose) may be achieved while increasing the therapeutic index (“therapeutic window”) relative to the unconjugated immunostimulatory drug.

In an exemplary embodiment, the invention provides a pharmaceutical composition for use in therapy.

The invention provides a method of treatment comprising administering a therapeutically-effective dose of the immunoconjugate to a patient with an immune-related disorder. The elastase-substrate, peptide linker of the immunoconjugate may be cleaved by elastase.

It is contemplated that the immunoconjugate of the present invention may be used to treat various hyperproliferative diseases or disorders, e.g. characterized by the overexpression of a tumor antigen. Exemplary hyperproliferative disorders include benign or malignant solid tumors and hematological disorders such as leukemia and lymphoid malignancies.

In another aspect, an immunoconjugate for use as a medicament is provided. In certain embodiments, the invention provides an immunoconjugate for use in a method of treating an individual comprising administering to the individual an effective amount of the immunoconjugate. In one such embodiment, the method further comprises administering to the individual an effective amount of at least one additional therapeutic agent, e.g., as described herein.

In a further aspect, the invention provides for the use of an immunoconjugate in the manufacture or preparation of a medicament. In one embodiment, the medicament is for treatment of cancer, the method comprising administering to an individual having cancer an effective amount of the medicament. In one such embodiment, the method further comprises administering to the individual an effective amount of at least one additional therapeutic agent, e.g., as described herein.

Carcinomas are malignancies that originate in the epithelial tissues. Epithelial cells cover the external surface of the body, line the internal cavities, and form the lining of glandular tissues. Examples of carcinomas include, but are not limited to, adenocarcinoma (cancer that begins in glandular (secretory) cells such as cancers of the breast, pancreas, lung, prostate, stomach, gastroesophageal junction, and colon) adrenocortical carcinoma; hepatocellular carcinoma; renal cell carcinoma; ovarian carcinoma; carcinoma in situ; ductal carcinoma; carcinoma of the breast; basal cell carcinoma; squamous cell carcinoma; transitional cell carcinoma; colon carcinoma; nasopharyngeal carcinoma; multilocular cystic renal cell carcinoma; oat cell carcinoma; large cell lung carcinoma; small cell lung carcinoma; non-small cell lung carcinoma; and the like. Carcinomas may be found in prostrate, pancreas, colon, brain (usually as secondary metastases), lung, breast, and skin. In some embodiments, methods for treating non-small cell lung carcinoma include administering an immunoconjugate containing an antibody construct that is capable of binding PD-L1 (e.g., atezolizumab, durvalumab, avelumab, biosimilars thereof, or biobetters thereof). In some embodiments, methods for treating breast cancer include administering an immunoconjugate containing an antibody construct that is capable of binding PD-L1 (e.g., atezolizumab, durvalumab, avelumab, biosimilars thereof, or biobetters thereof). In some embodiments, methods for treating triple-negative breast cancer include administering an immunoconjugate containing an antibody construct that is capable of binding PD-L1 (e.g., atezolizumab, durvalumab, avelumab, biosimilars thereof, or biobetters thereof).

Soft tissue tumors are a highly diverse group of rare tumors that are derived from connective tissue. Examples of soft tissue tumors include, but are not limited to, alveolar soft part sarcoma; angiomatoid fibrous histiocytoma; chondromyoxid fibroma; skeletal chondrosarcoma; extraskeletal myxoid chondrosarcoma; clear cell sarcoma; desmoplastic small round-cell tumor; dermatofibrosarcoma protuberans; endometrial stromal tumor; Ewing’s sarcoma; fibromatosis (Desmoid); fibrosarcoma, infantile; gastrointestinal stromal tumor; bone giant cell tumor; tenosynovial giant cell tumor; inflammatory myofibroblastic tumor; uterine leiomyoma; leiomyosarcoma; lipoblastoma; typical lipoma; spindle cell or pleomorphic lipoma; atypical lipoma; chondroid lipoma; well-differentiated liposarcoma; myxoid/round cell liposarcoma; pleomorphic liposarcoma; myxoid malignant fibrous histiocytoma; high-grade malignant fibrous histiocytoma; myxofibrosarcoma; malignant peripheral nerve sheath tumor; mesothelioma; neuroblastoma; osteochondroma; osteosarcoma; primitive neuroectodermal tumor; alveolar rhabdomyosarcoma; embryonal rhabdomyosarcoma; benign or malignant schwannoma; synovial sarcoma; Evan’s tumor; nodular fasciitis; desmoid-type fibromatosis; solitary fibrous tumor; dermatofibrosarcoma protuberans (DFSP); angiosarcoma; epithelioid hemangioendothelioma; tenosynovial giant cell tumor (TGCT); pigmented villonodular synovitis (PVNS); fibrous dysplasia; myxofibrosarcoma; fibrosarcoma; synovial sarcoma; malignant peripheral nerve sheath tumor; neurofibroma; pleomorphic adenoma of soft tissue; and neoplasias derived from fibroblasts, myofibroblasts, histiocytes, vascular cells/endothelial cells, and nerve sheath cells.

A sarcoma is a rare type of cancer that arises in cells of mesenchymal origin, e.g., in bone or in the soft tissues of the body, including cartilage, fat, muscle, blood vessels, fibrous tissue, or other connective or supportive tissue. Different types of sarcoma are based on where the cancer forms. For example, osteosarcoma forms in bone, liposarcoma forms in fat, and rhabdomyosarcoma forms in muscle. Examples of sarcomas include, but are not limited to, askin’s tumor; sarcoma botryoides; chondrosarcoma; ewing’s sarcoma; malignant hemangioendothelioma; malignant schwannoma; osteosarcoma; and soft tissue sarcomas (e.g., alveolar soft part sarcoma; angiosarcoma; cystosarcoma phyllodesdermatofibrosarcoma protuberans (DFSP); desmoid tumor; desmoplastic small round cell tumor; epithelioid sarcoma; extraskeletal chondrosarcoma; extraskeletal osteosarcoma; fibrosarcoma; gastrointestinal stromal tumor (GIST); hemangiopericytoma; hemangiosarcoma (more commonly referred to as “angiosarcoma”); kaposi’s sarcoma; leiomyosarcoma; liposarcoma; lymphangiosarcoma; malignant peripheral nerve sheath tumor (MPNST); neurofibrosarcoma; synovial sarcoma; and undifferentiated pleomorphic sarcoma).

A teratoma is a type of germ cell tumor that may contain several different types of tissue (e.g., can include tissues derived from any and/or all of the three germ layers: endoderm, mesoderm, and ectoderm), including, for example, hair, muscle, and bone. Teratomas occur most often in the ovaries in women, the testicles in men, and the tailbone in children.

Melanoma is a form of cancer that begins in melanocytes (cells that make the pigment melanin). Melanoma may begin in a mole (skin melanoma), but can also begin in other pigmented tissues, such as in the eye or in the intestines.

Merkel cell carcinoma is a rare type of skin cancer that usually appears as a flesh-colored or bluish-red nodule on the face, head or neck. Merkel cell carcinoma is also called neuroendocrine carcinoma of the skin. In some embodiments, methods for treating Merkel cell carcinoma include administering an immunoconjugate containing an antibody construct that is capable of binding PD-L1 (e.g., atezolizumab, durvalumab, avelumab, biosimilars thereof, or biobetters thereof). In some embodiments, the Merkel cell carcinoma has metastasized when administration occurs.

Leukemias are cancers that start in blood-forming tissue, such as the bone marrow, and cause large numbers of abnormal blood cells to be produced and enter the bloodstream. For example, leukemias can originate in bone marrow-derived cells that normally mature in the bloodstream. Leukemias are named for how quickly the disease develops and progresses (e.g., acute versus chronic) and for the type of white blood cell that is affected (e.g., myeloid versus lymphoid). Myeloid leukemias are also called myelogenous or myeloblastic leukemias. Lymphoid leukemias are also called lymphoblastic or lymphocytic leukemia. Lymphoid leukemia cells may collect in the lymph nodes, which can become swollen. Examples of leukemias include, but are not limited to, Acute myeloid leukemia (AML), Acute lymphoblastic leukemia (ALL), Chronic myeloid leukemia (CML), and Chronic lymphocytic leukemia (CLL).

Lymphomas are cancers that begin in cells of the immune system. For example, lymphomas can originate in bone marrow-derived cells that normally mature in the lymphatic system. There are two basic categories of lymphomas. One category of lymphoma is Hodgkin lymphoma (HL), which is marked by the presence of a type of cell called the Reed-Sternberg cell. There are currently 6 recognized types of HL. Examples of Hodgkin lymphomas include nodular sclerosis classical Hodgkin lymphoma (CHL), mixed cellularity CHL, lymphocyte-depletion CHL, lymphocyte-rich CHL, and nodular lymphocyte predominant HL.

The other category of lymphoma is non-Hodgkin lymphomas (NHL), which includes a large, diverse group of cancers of immune system cells. Non-Hodgkin lymphomas can be further divided into cancers that have an indolent (slow-growing) course and those that have an aggressive (fast-growing) course. There are currently 61 recognized types of NHL. Examples of non-Hodgkin lymphomas include, but are not limited to, AIDS-related Lymphomas, anaplastic large-cell lymphoma, angioimmunoblastic lymphoma, blastic NK-cell lymphoma, Burkitt’s lymphoma, Burkitt-like lymphoma (small non-cleaved cell lymphoma), chronic lymphocytic leukemia/small lymphocytic lymphoma, cutaneous T-Cell lymphoma, diffuse large B-Cell lymphoma, enteropathy-type T-Cell lymphoma, follicular lymphoma, hepatosplenic gamma-delta T-Cell lymphomas, T-Cell leukemias, lymphoblastic lymphoma, mantle cell lymphoma, marginal zone lymphoma, nasal T-Cell lymphoma, pediatric lymphoma, peripheral T-Cell lymphomas, primary central nervous system lymphoma, transformed lymphomas, treatment-related T-Cell lymphomas, and Waldenstrom’s macroglobulinemia.

Brain cancers include any cancer of the brain tissues. Examples of brain cancers include, but are not limited to, gliomas (e.g., glioblastomas, astrocytomas, oligodendrogliomas, ependymomas, and the like), meningiomas, pituitary adenomas, and vestibular schwannomas, primitive neuroectodermal tumors (medulloblastomas).

Immunoconjugates of the invention can be used either alone or in combination with other agents in a therapy. For instance, an immunoconjugate may be co-administered with at least one additional therapeutic agent, such as a chemotherapeutic agent. Such combination therapies encompass combined administration (where two or more therapeutic agents are included in the same or separate formulations), and separate administration, in which case, administration of the immunoconjugate can occur prior to, simultaneously, and/or following, administration of the additional therapeutic agent and/or adjuvant. Immunoconjugates can also be used in combination with radiation therapy.

The immunoconjugates of the invention (and any additional therapeutic agent) can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein.

Atezolizumab, durvalumab, avelumab, biosimilars thereof, and biobetters thereof are known to be useful in the treatment of cancer, particularly breast cancer, especially triple negative (test negative for estrogen receptors, progesterone receptors, and excess HER2 protein) breast cancer, bladder cancer, and Merkel cell carcinoma. The immunoconjugate described herein can be used to treat the same types of cancers as atezolizumab, durvalumab, avelumab, biosimilars thereof, and biobetters thereof, particularly breast cancer, especially triple negative (test negative for estrogen receptors, progesterone receptors, and excess HER2 protein) breast cancer, bladder cancer, and Merkel cell carcinoma.

The immunoconjugate is administered to a subject in need thereof in any therapeutically effective amount using any suitable dosing regimen, such as the dosing regimens utilized for atezolizumab, durvalumab, avelumab, biosimilars thereof, and biobetters thereof. For example, the methods can include administering the immunoconjugate to provide a dose of from about 100 ng/kg to about 50 mg/kg to the subject. The immunoconjugate dose can range from about 5 mg/kg to about 50 mg/kg, from about 10 µg/kg to about 5 mg/kg, or from about 100 µg/kg to about 1 mg/kg. The immunoconjugate dose can be about 100, 200, 300, 400, or 500 µg/kg. The immunoconjugate dose can be about 1, 2, 3, 4, 5, 6, 7, 8, 9, or 10 mg/kg. The immunoconjugate dose can also be outside of these ranges, depending on the particular conjugate as well as the type and severity of the cancer being treated. Frequency of administration can range from a single dose to multiple doses per week, or more frequently. In some embodiments, the immunoconjugate is administered from about once per month to about five times per week. In some embodiments, the immunoconjugate is administered once per week.

In another aspect, the invention provides a method for preventing cancer. The method comprises administering a therapeutically effective amount of an immunoconjugate (e.g., as a composition as described above) to a subject. In certain embodiments, the subject is susceptible to a certain cancer to be prevented. For example, the methods can include administering the immunoconjugate to provide a dose of from about 100 ng/kg to about 50 mg/kg to the subject. The immunoconjugate dose can range from about 5 mg/kg to about 50 mg/kg, from about 10 µg/kg to about 5 mg/kg, or from about 100 µg/kg to about 1 mg/kg. The immunoconjugate dose can be about 100, 200, 300, 400, or 500 µg/kg. The immunoconjugate dose can be about 1, 2, 3, 4, 5, 6, 7, 8, 9, or 10 mg/kg. The immunoconjugate dose can also be outside of these ranges, depending on the particular conjugate as well as the type and severity of the cancer being treated. Frequency of administration can range from a single dose to multiple doses per week, or more frequently. In some embodiments, the immunoconjugate is administered from about once per month to about five times per week. In some embodiments, the immunoconjugate is administered once per week.

Some embodiments of the invention provide methods for treating cancer as described above, wherein the cancer is breast cancer. Breast cancer can originate from different areas in the breast, and a number of different types of breast cancer have been characterized. For example, the immunoconjugates of the invention can be used for treating ductal carcinoma in situ; invasive ductal carcinoma (e.g., tubular carcinoma; medullary carcinoma; mucinous carcinoma; papillary carcinoma; or cribriform carcinoma of the breast); lobular carcinoma in situ; invasive lobular carcinoma; inflammatory breast cancer; and other forms of breast cancer such as triple negative (test negative for estrogen receptors, progesterone receptors, and excess HER2 protein) breast cancer. In some embodiments, methods for treating breast cancer include administering an immunoconjugate containing an antibody construct that is capable of binding HER2 (e.g. trastuzumab, pertuzumab, biosimilars, or biobetters thereof ) and PD-L1 (e.g., atezolizumab, durvalumab, avelumab, biosimilars, or biobetters thereof). In some embodiments, methods for treating colon cancer lung cancer, renal cancer, pancreatic cancer, gastric cancer, and esophageal cancer include administering an immunoconjugate containing an antibody construct that is capable of binding CEA, or tumors over-expressing CEA (e.g. labetuzumab, biosimilars, or biobetters thereof).

In some embodiments, the cancer is susceptible to a pro-inflammatory response induced by TLR7 and/or TLR8.

### EXAMPLES

### Preparation of Immunostimulatory Compounds and Intermediates

### Example 1 Synthesis of 2,3,5,6-tetrafluorophenyl (R)-1-((S)-2-(((S)-1-((4-(((((1-((3-(2-Amino-4-(Dipropylcarbamoyl)-3h-Benzo[B]Azepin-8-Yl)Phenyl)Sulfonyl)Azetidin-3-Yl)Methyl)Carbamoyl)Oxy)Methyl)Phenyl)Amino)-3-Methyl-1-Oxobutan-2-Yl)Carbamoyl)Pyrrolidin-1-Yl)-2-Methyl-1,4-Dioxo-7,10,13,16,19,22,25,28,31,34,37,40,43,46,49,52,55,58,61,64,67,70,73,76,79-penTacosaoxa-3-Azadooctacontan-82-Oate, II-1

### Preparation of O2-(2,5-Dioxopyrrolidin-1-Yl) O1-(9H-Fluoren-9-Ylmethyl) (2S)-Pyrrolidine-1,2-Dicarboxylate, II-1b

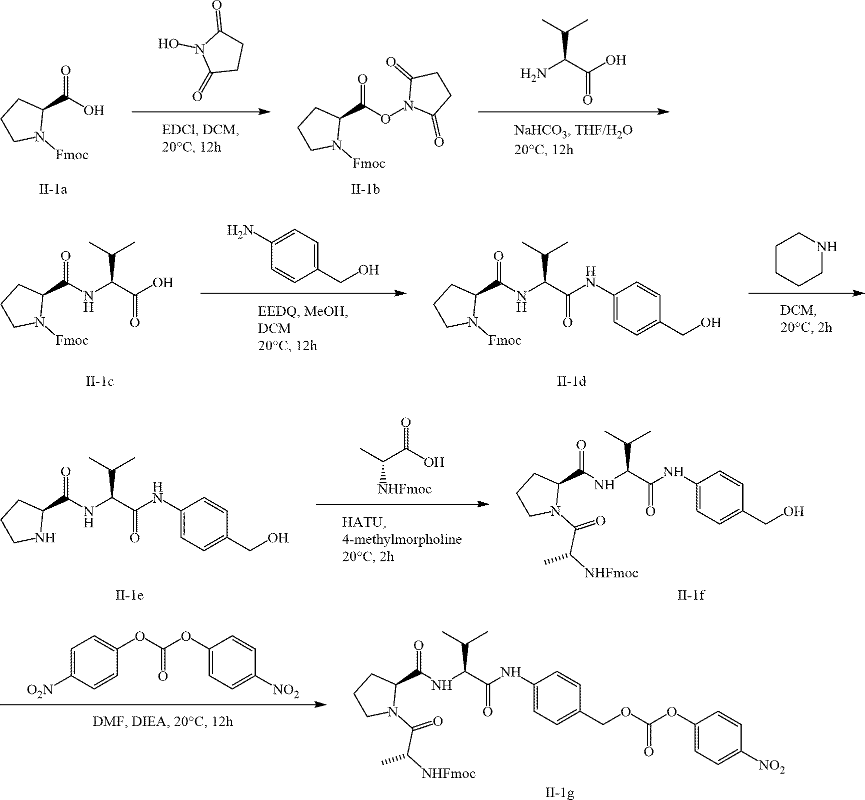

To a solution of (2S)-1-(9H-fluoren-9-ylmethoxycarbonyl)pyrrolidine-2-carboxylic acid, II-1a (15 g, 44.5 mmol, 1.0 eq) in DCM (200 mL) was added 1-hydroxypyrrolidine-2,5-dione (5.12 g, 44.5 mmol, 1.0 eq) and 1-ethyl-3-(3-dimethylaminopropyl)carbodiimide hydrochloride, EDCI (10.2 g, 53.44 mmol, 1.2 eq). The mixture was stirred at 20° C. for 12 h and then washed by saturated aqueous solution of NaHCO₃ (70 mL × 3). The organic layer was dried over Na₂SO₄ and concentrated to give 11-1b (17.5 g, 40.28 mmol, 90.60% yield) as a white solid.

### Preparation of (2S)-2-[[(2S)-1-(9H-Fluoren-9-Ylmethoxycarbonyl)Pyrrolidine-2-Carbonyl]Amino]-3-Methyl-Butanoic Acid, II-1c

To a solution of (2S)-2-amino-3-methyl-butanoic acid (4.95 g, 42.3 mmol, 1.05 eq) in THF (200 mL) was added NaHCO₃ (3.55 g, 42.3 mmol, 1.64 mL, 1.05 eq) in H₂O (50 mL) and II-1b (17.5 g, 40.28 mmol, 1.0 eq) and it was stirred at 20° C. for 12 h. The mixture was extracted with methyl, tert-butylether, MTBE (2 × 100 mL) (discarded). The pH of aqueous layer was adjusted to 5-6 with HCl (6 M) and extracted with EtOAc (3 × 200 ml). The combined organic layer was dried over Na₂SO₄ and concentrated to give II-1c (15 g, 34.36 mmol, 85.31% yield) as white solid. ¹H NMR (MeOD, 400 MHz) δ7.80 (d, J = 7.2 Hz, 2H), 7.70-7.54 (m, 2H), 7.43-7.28 (m, 4H), 4.49-4.15 (m, 5H), 3.69-3.38 (m, 2H), 2.42-2.01 (m, 3H), 2.00-1.82 (m, 2H), 1.01-0.86 (m, 6H)

### Preparation of 9h-Fluoren-9-Ylmethyl(2s)-2-[[(1s)-1-[[4-(Hydroxymethyl)Phenyl] Carbamoyl]-2-Methyl-Propyl]Carbamoyl]Pyrrolidine-1-Carboxylate, II-1d

To a solution of II-1c (10 g, 22.9 mmol, 1.0 eq) and (4-aminophenyl)methanol (4.23 g, 34.4 mmol, 1.5 eq) in MeOH (80 mL) and DCM (80 mL) was added N-ethoxycarbonyl-2-ethoxy-1,2-dihydroquinoline, EEDQ (8.50 g, 34.36 mmol, 1.5 eq) and then stirred at 20° C. for 12 h. The mixture was concentrated in vacuum to give a residue and the residue was purified by flash silica gel chromatography (Silica Flash Column, Eluent of 0 to 40% Ethyl acetate/MeOH at 65 mL/min). The crude product was triturated with EtOAc at 20° C. for 20 min to give II-1d (13 g, 24.0 mmol, 52.38% yield) as yellow solid. ¹H NMR (MeOD, 400 MHz) δ 7.85-7.71 (m, 2H), 7.68-7.48 (m, 3H), 7.47-7.16 (m, 7H), 4.53 (d, J = 15.2 Hz, 2H), 4.49-4.41 (m, 1H), 4.40-4.33 (m, 2H), 4.32-4.27 (m, 1H), 4.26-4.17 (m, 1H), 4.16-4.07 (m, 1H), 3.69-3.38 (m, 2H), 2.40-2.05 (m, 2H), 1.99-1.82 (m, 2H), 1.08-0.88 (m, 6H)

### Preparation of (2s)-N-[(1s)-1-[[4-(Hydroxymethyl)Phenyl]Carbamoyl]-2-MethylPropyl]Pyrrolidine-2-Carboxamide, II-1e

To a solution of II-1d (13 g, 24.0 mmol, 1.0 eq) in DCM (130 mL) was added piperidine (10.22 g, 120 mmol, 11.85 mL, 5.0 eq) and then stirred at 20° C. for 2 h. The mixture was concentrated to give a residue and the residue was triturated with EtOAc at 20° C. for 20 min to give II-1e (8 g, crude) as white solid.

### Preparation of 9H-Fluoren-9-Ylmethyln-[(1r)-2-[(2s)-2-[[(1s)-1-[[4-(Hydroxymethyl) Phenyl]Carbamoyl]-2-Methyl-Propyl]Carbamoyl]Pyrrolidin-1-Yl]-1-Methyl-2-OxoEthyl]Carbamate, II-1f

To a solution of (2R)-2-(9H-fluoren-9-ylmethoxycarbonylamino)propanoic acid (2.73 g, 8.77 mmol, 1.4 eq) in DCM (30 mL) was added 1-[Bis(dimethylamino)methylene]-1H-,2,3-triazolo[4,5-b]pyridinium 3-oxide hexafluorophosphate; Hexafluorophosphate Azabenzotriazole Tetramethyl Uronium, HATU (2.50 g, 6.57 mmol, 1.05 eq), 4-methylmorpholine (1.90 g, 18.8 mmol, 2.07 mL, 3.0 eq) and II-1e (2 g, 6.26 mmol, 1.0 eq), and then stirred at 20° C. for 2 h. The mixture was diluted with water (40 mL) and extracted with DCM (30 mL × 3). The organic layer was washed with brine (30 mL), dried over Na₂SO₄, filtered and concentrated. The residue was purified by flash silica gel chromatography (ISCO®; 3.5 g SepaFlash® Silica Flash Column, Eluent of 0~100% Ethyl acetate/Petroleum ether gradient at 45 mL/min) to give II-1f (1.8 g, 2.94 mmol, 46.92% yield) as white solid. ¹H NMR (MeOD, 400 MHz) δ 7.83-7.71 (m, 4H), 7.56-7.45 (m, 2H), 7.39-7.20 (m, 6H), 4.53-4.41 (m, 4H), 4.05-3.93 (m, 2H), 3.82-3.64 (m, 2H), 3.18-3.08 (m, 1H), 2.53-2.42 (m, 1H), 2.38-2.26 (m, 1H), 2.10-2.04 (m, 2H), 1.37 (d, J = 6.8 Hz, 3H), 1.06-0.96 (m, 6H)

### Preparation of [4-[[(2s)-2-[[(2s)-1-[(2r)-2-(9h-Fluoren-9-Ylmethoxycarbonylamino)Propanoyl]Pyrrolidine-2-Carbonyl]Amino]-3-Methyl-Butanoyl]Amino]Phenyl]Methyl (4-Nitrophenyl) Carbonate, II-1g

To a solution of II-1f (1.8 g, 2.94 mmol, 1.0 eq) in DMF (15 mL) was added DIEA (569 mg, 4.41 mmol, 767 uL, 1.5 eq) and bis(4-nitrophenyl) carbonate (1.07 g, 3.53 mmol, 1.2 eq) and then stirred at 20° C. for 12 h. The mixture was diluted with water (50 mL) and extracted with EtOAc (30 mL × 3). The organic layer was washed with brine (30 mL), dried over Na₂SO₄, filtered and concentrated. The residue was purified by flash silica gel chromatography (ISCO®; 4 g SepaFlash® Silica Flash Column, Eluent of 0 to 100% Ethyl acetate/Petroleum ether gradient at 45 mL/min) to give II-1g (1.1 g, 1.41 mmol, 48.14% yield) as light yellow solid. ¹H NMR (MeOD, 400 MHz) δ 8.57 (s, 1H), 8.19-8.10 (m, 2H), 8.00 (d, J = 8.4 Hz, 2H), 7.74-7.63 (m, 2H), 7.38-7.25 (m, 7H), 7.23-7.14 (m, 3H), 7.08 (d, J = 10.0 Hz, 1H), 5.18-5.16 (m, 2H), 4.77 (dd, J = 10.0, 4.0 Hz, 1H), 4.64 (dd, J = 8.4, 3.6 Hz, 1H), 4.44-4.32 (m, 1H), 4.10-4.03 (m, 1H), 3.98-3.82 (m, 2H), 3.68-3.55 (m, 2H), 2.79-2.67 (m, 1H), 2.41-2.21 (m, 2H), 2.17-2.06 (m, 2H), 1.46 (d, J = 7.2 Hz, 3H), 0.97 (d, J = 7.2 Hz, 6H)

### Preparation of II-1l

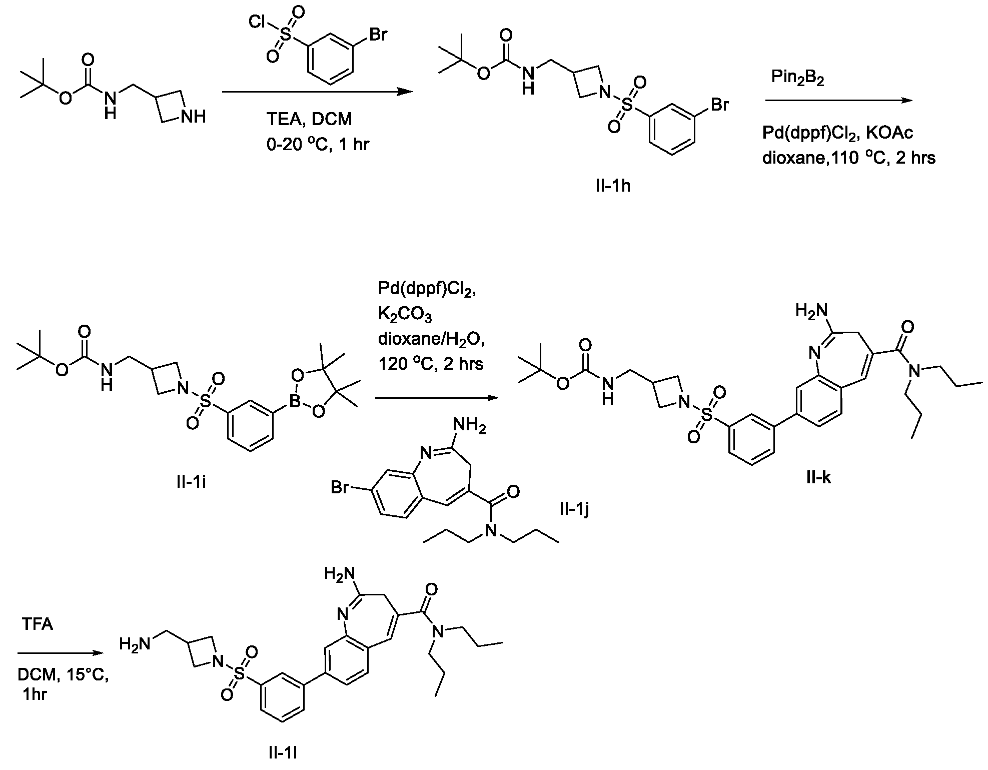

### Preparation of Tert-Butyl ((1-((3-Bromophenyl)Sulfonyl)Azetidin-3-Yl)Methyl)Carbamate, II-1h

To a mixture of tert-butyl N-(azetidin-3-ylmethyl)carbamate (1.6 g, 8.59 mmol, 1.2 eq) in DCM (5 mL) was added triethylamine, TEA (1.45 g, 14.32 mmol, 1.99 mL, 2 eq) and 3-bromobenzenesulfonyl chloride (1.83 g, 7.16 mmol, 1.03 mL, 1 eq) at 0° C. The mixture was stirred at 20° C. for 1 hr. The mixture was diluted with water (50 mL) and extracted with DCM (25 ml × 3). The organic layer was washed with brine (25 mL), dried over Na₂SO₄, filtered and concentrated. The residue was purified by flash silica gel chromatography (ISCO®; 4 g SepaFlash® Silica Flash Column, Eluent of 0 to 100% Ethyl acetate/Petroleum ether gradient at 35 mL/min). Compound II-1h (2.5 g, 6.17 mmol, 86.16% yield) was obtained as white solid. ¹H NMR (CDCh, 400 MHz) δ 7.99 (t, J= 4.0 Hz, 1H), 7.74-7.81 (m, 2H), 7.47 (t, J = 8.0 Hz, 1H), 4.61 (s, 1H), 3.86 (t, J = 8.0 Hz, 2H), 3.50-3.58 (m, 2H), 3.19 (t, J = 4.0 2H), 2.58-2.70 (m, 1H), 1.42 (s, 9H).

### Preparation of Tert-Butyl N-[[1-[3-(4,4,5,5-Tetramethyl-1,3,2-Dioxaborolan- 2-Yl)Phenyl]Sulfonylazetidin-3-Yl]Methyl]Carbamate, II-1i

To a mixture of II-1h (1 g, 2.47 mmol, 1 eq) in dioxane (10 mL) was added 4,4,4′,4′,5,5,5′,5′-octamethyl-2,2′-bi(1,3,2-dioxaborolane), Pin₂B₂ (939.80 mg, 3.70 mmol, 1.5 eq) and KOAc (484.29 mg, 4.93 mmol, 2 eq), [1,1′-bis(diphenylphosphino)ferrocene]dichloropalladium(II), Pd(dppf)Cl₂ (90.27 mg, 123.36 µmol, 0.05 eq) at 15° C. under N₂. The mixture was stirred at 110° C. for 2 hrs. The product II-1i was not isolated and used into next step.

### Preparation of Tert-Butyl ((1-((3-(2-Amino-4-(Dipropylcarbamoyl)-3h-Benzo[B]Azepin-8-Yl)Phenyl)Sulfonyl)Azetidin-3-Yl)Methyl)Carbamate, II-1k

To a mixture of II-1i (1.12 g, 2.48 mmol, 1 eq) and 2-amino-8-bromo-N,N-dipropyl-3H-1-benzazepine-4-carboxamide, II-Ij (901.90 mg, 2.48 mmol, 1 eq) in dioxane (3 mL) was added K₂CO₃ (684.35 mg, 4.95 mmol, 2 eq) and Pd(dppf)Cl₂ (90.58 mg, 123.79 µmol, 0.05 eq) at 15° C. under N₂. The mixture was stirred at 120° C. for 2 hrs. The mixture was filtered and concentrated. The residue was purified by flash silica gel chromatography (ISCO®; 2 g SepaFlash® Silica Flash Column, Eluent of 0 to 100% Ethyl acetate/Petroleum ether gradient @ 60 mL/min) to give II-1k (600 mg, 983.97 µmol, 39.74% yield, 100% purity) as yellow solid. ¹H NMR (MeOD-d₄, 400 MHz) 8 7.99-8.10 (m, 2H), 7.74-7.86 (m, 2H), 7.36-7.52 (m, 3H), 6.89 (s, 1H), 3.83 (t, J = 8.0 Hz, 2H), 3.54 (t, J = 8.0 Hz, 2H), 3.34-3.48 (m, 6H), 3.02 (d, J = 8.0 Hz, 2H), 2.48-2.64 (m, 1H), 1.59-1.76 (m, 4H), 1.37 (s, 9H), 0.96-0.89 (m, 6H). LC/MS [M+H] 610.31 (calculated); LC/MS [M+H] 610.40 (observed).

### Preparation of 2-Amino-8-[3-[3-(Aminomethyl)Azetidin-1-Yl]Sulfonylphenyl]-N,N-Dipropyl-3h-1-Benzazepine-4-Carboxamide, II-1l

To a solution of II-1k (0.15 g, 245.99 µmol, 1 eq) in DCM (20 mL) was added TFA (56.10 mg, 491.98 µmol, 36.43 µL, 2 eq) at 25° C. and stirred for 1 hour. The mixture was concentrated in reduced pressure at 40° C. The residue was purified by prep-HPLC (column: Nano-micro Kromasil C18 100 × 30 mm 5 um; mobile phase: [water (0.1%TFA)-ACN]; B%: 25%-50%, 10 min) to give, 11-11 (0.0546 g, 105.69 µmol, 42.97% yield, 98.66% purity) as a yellow solid. ¹H NMR (MeOD-d₄, 400 MHz) δ 8.16-8.07 (m, 2H), 7.92 (d, J = 8.0 Hz, 1H), 7.83 (t, J = 7.6 Hz, 1H), 7.79-7.72 (m, 2H), 7.68 (d, J = 8.4 Hz, 1H), 7.09 (s, 1H), 3.96 (t, J = 8.4 Hz, 2H), 3.67-3.63 (m, 2H), 3.50-3.42 (m, 4H), 3.37 (s, 2H), 3.05 (d, J = 7.4 Hz, 2H), 2.78-2.65 (m, 1H), 1.75-1.66 (m, 4H), 1.08-0.82 (m, 6H). LC/MS [M+H] 510.25 (calculated); LC/MS [M+H] 510.10 (observed).

### Preparation of [4-[[(2S)-2-[[(2S)-1-[(2R)-2-(9H- Fluoren-9-Ylmethoxycarbonylamino)Propanoyl]Pyrrolidine-2-Carbonyl]Amino]-3-Methyl-Butanoyl]Amino]Phenyl]Methyl N-[[1-[3-[2-Amino-4-(Dipropylcarbamoyl)-3h-1-Benzazepin-8-Yl] Phenyl]Sulfonylazetidin-3-Yl]Methyl]Carbamate, II-1m

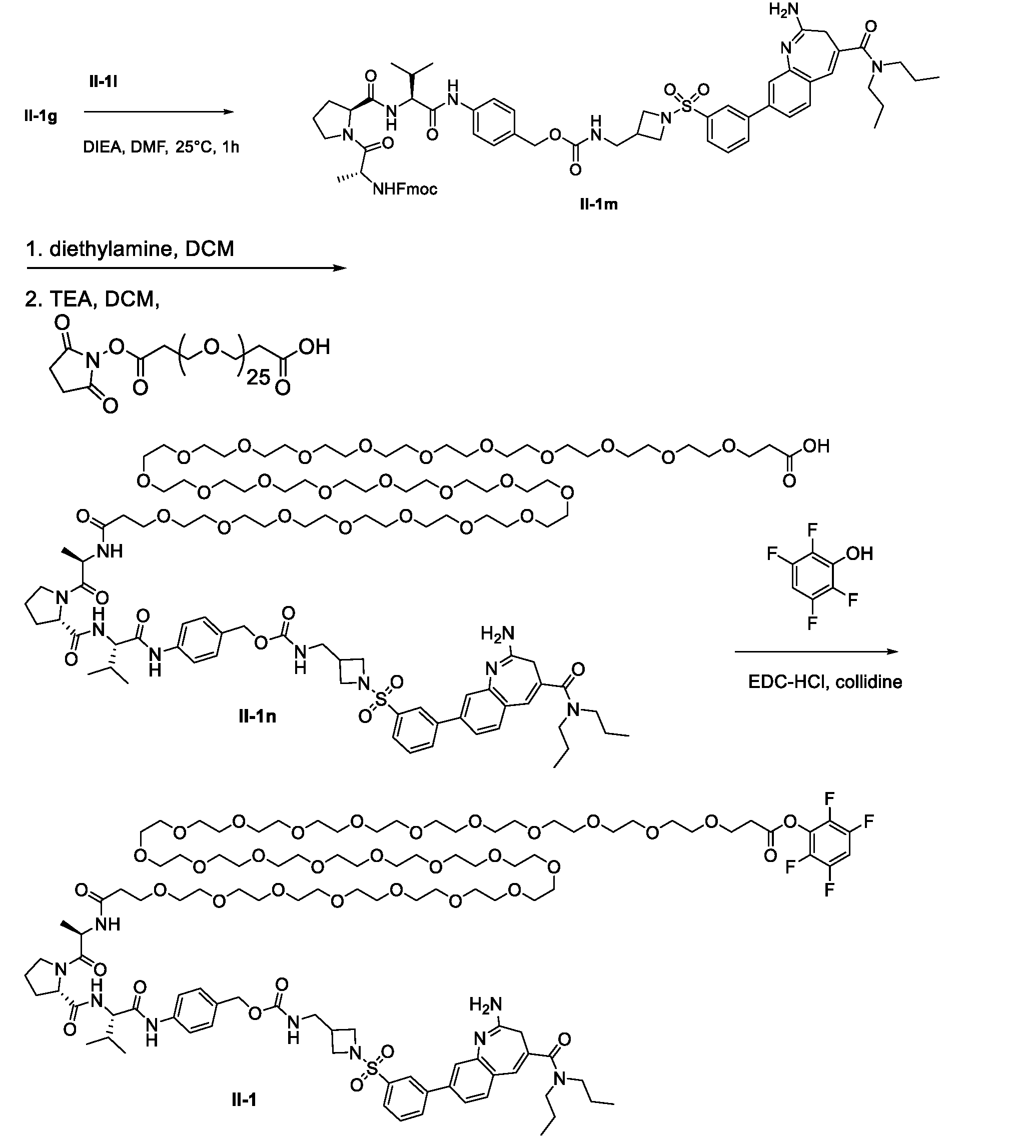

To a mixture of 11-11 (100 mg, 196 umol, 1.0 eq) and II-1g (153 mg, 196 umol, 1.0 eq) in DMF (0.5 mL) was added DIEA (50.7 mg, 392 umol, 68.3 uL, 2.0 eq) at 25° C. under N₂, and then stirred at 25° C. for 1 hour. The mixture was filtered and purified by prep-HPLC (column: Nano-micro Kromasil C18 100*30 mm 8 um; mobile phase: [water (0.1%TFA)-ACN]; B%: 35%-60%, 10 min) to afford II-1m (70 mg, 60.96 umol, 31.07% yield) as white solid. ¹H NMR (DMSO-d₆, 400 MHz) 612.16 (s, 1H), 10.10 (s, 1H), 9.82 (s, 2H), 9.55 (s, 1H), 9.23 (s, 2H), 8.33 (d, J = 8.4 Hz, 1H), 8.09-8.06 (m, 2H), 7.97 (s, 1H), 7.90-7.48 (m, 14H), 7.42-7.11 (m, 6H), 7.01 (s, 1H), 4.83-4.79 (m, 3H), 4.40-3.93 (m, 9H), 3.79-3.66 (m, 3H), 3.57-3.45 (m, 3H), 3.29 (s, 3H), 2.95-2.83 (m, 3H), 2.20-1.95 (m, 2H), 1.91-1.83 (m, 3H), 1.73-1.67 (m, 1H), 1.61-1.46 (m, 3H), 1.20 (d, J = 6.4 Hz, 3H), 1.05 (d, J = 6.4 Hz, 2H), 0.93-0.66 (m, 12H). LC/MS [M+H] 1148.5 (calculated); LC/MS [M+H] 1148.6 (observed).

### Preparation of (R)-1-((S)-2-(((S)-1-((4-(((((1-((3-(2-Amino-4-(Dipropylcarbamoyl)-3H-Benzo[B]Azepin-8-Yl)Phenyl)Sulfonyl)Azetidin-3-Yl)Methyl)Carbamoyl)Oxy)Methyl)Phenyl)Amino)-3-Methyl-1-Oxobutan-2-Yl)Carbamoyl)Pyrrolidin-1-Yl)-2-Methyl-1,4-Dioxo-7,10,13,16,19,22,25,28,31,34,37,40,43,46,49,52,55,58,61,64,67,70,73,76,79-Pentacosaoxa-3-Azadooctacontan-82-Oic Acid, II-1n

Fmoc-amine, II-1m (0.014 g, 0.015 mmol, 1 eq.) was dissolved in 50% diethylamine in DCM and allowed to sit at room temperature. Upon complete deprotection, the solution was concentrated overnight. To the crude material was added a solution of PEG25-NHS, 79-((2,5-dioxopyrrolidin-1-yl)oxy)-79-oxo-4,7,10,13,16,19,22,25,28,31,34,37,40,43,46,49,52,55,58,61,64,67,70,73,76-pentacosaoxanonaheptacontanoic acid (0.020 g, 0.015 mmol, 1 eq.) in DCM, followed by TEA (0.01 ml, 0.072 mmol, 3 eq.). The reaction mixture was concentrated and purified by HPLC to give II-1n (0.021 g, 0.01 mmol, 65%). LC/MS [M+2H] 1064.57 (calculated); LC/MS [M+2H] 1064.70 (observed).

Preparation of II-1: Acid, II-1n (0.021 g, 0.0097 mmol, 1 eq.) and 2,3,5,6-tetrafluorophenol, TFP (0.0032 g, 0.019 mmol, 2 eq.) were dissolved in 1 ml dimethylformamide, DMF. Collidine (trimethylpyridine, 0.006 ml, 0.048 mmol, 5 eq.) was added, followed by EDC-HCl (0.0056 g, 0.029 mmol, 3 eq.). The reaction was stirred at room temperature and monitored by LCMS, then purified by HPLC to give tetrafluorophenol, TFP ester, II-1 (0.0097 g, 0.0043 mmol, 44%). LC/MS [M+2H] 1138.57 (calculated); LC/MS [M+2H] 1138.73 (observed).

### Example 2 Synthesis of 2,3,5,6-Tetrafluorophenyl (R)-1-((S)-2-(((S)-1-((4-(((((1-((3-(2-Amino-4-((3-((Tert-Butoxycarbonyl)Amino)Propyl)(Propyl)Carbamoyl)-3h-Benzo[B]Azepin-8-Yl)Phenyl)Sulfonyl)Azetidin-3-Yl)Methyl)Carbamoyl)Oxy)Methyl)Phenyl)Amino)-3-Methyl-1-Oxobutan-2-Yl)Carbamoyl)Pyrrolidin-1-Yl)-2-Methyl-1,4-Dioxo-7,10,13,16,19,22,25,28,31,34,37,40,43,46,49,52,55,58,61,64,67,70,73,76,79-Pentacosaoxa-3-Azadooctacontan-82-Oate, II-2

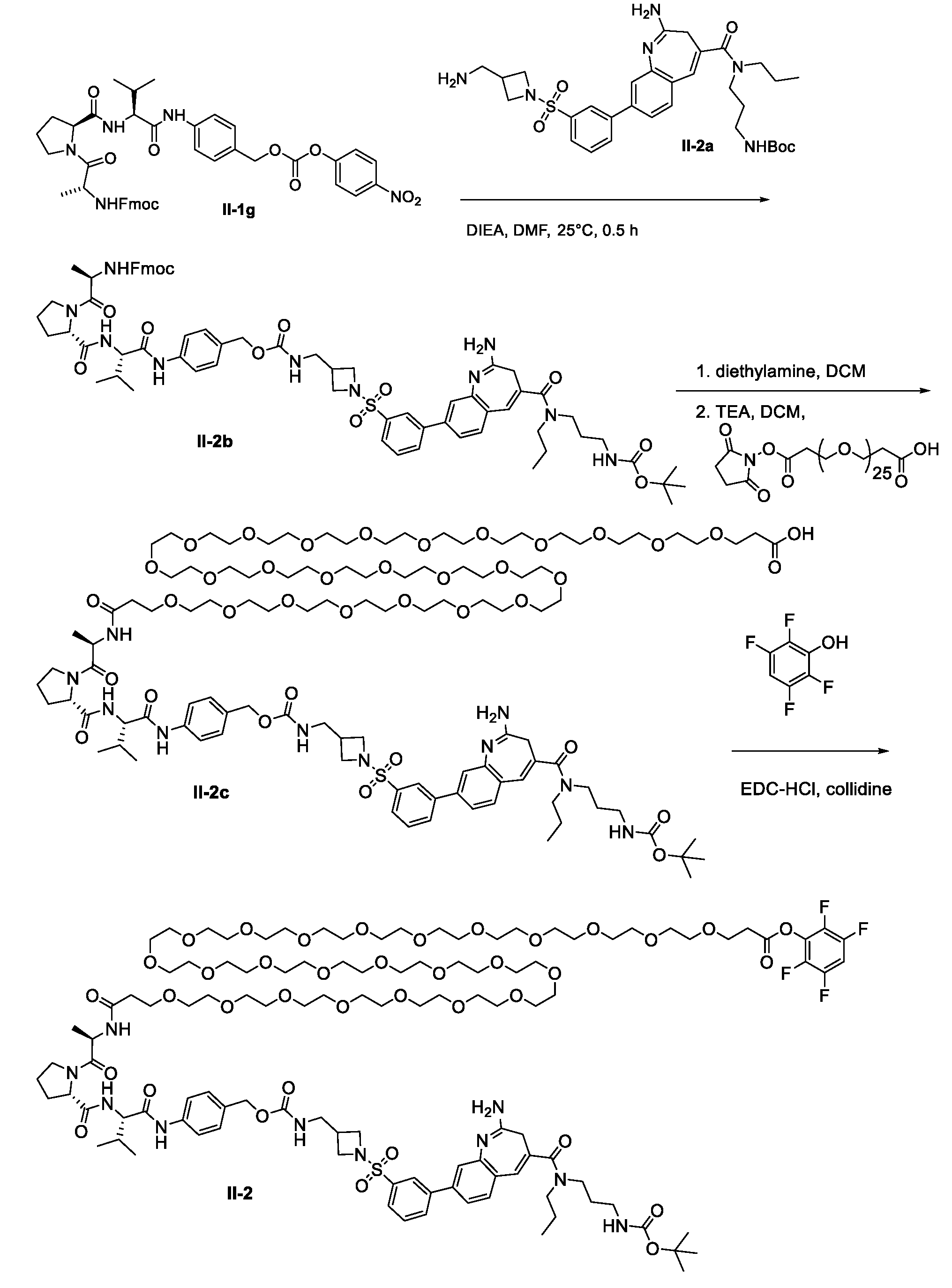

### Preparation of [4-[[(2S)-2-[[(2S)-1-[(2R)-2-(9H-Fluoren-9-Ylmethoxycarbonylamino) Propanoyl]Pyrrolidine-2-Carbonyl]Amino]-3-Methyl-Butanoyl]Amino]Phenyl]Methyl N-[[1-[3-[2-Amino-4-[3-(Tert-Butoxycarbonylamino)Propyl-Propyl-Carbamoyl]-3h-1-Benzazepin-8-Yl]Phenyl]Sulfonylazetidin-3-Yl]Methyl]Carbamate, II-2b

To a mixture of tert-butyl N-[3-[[2-amino-8-[3-[3-(aminomethyl)azetidin-1-yl] sulfonylphenyl]-3H-1-benzazepine-4-carbonyl]-propyl-amino]propyl]carbamate, II-2a (100 mg, 160 µmol, 1.0 eq) and [4-[[(2S)-2-[[(2S)-1-[(2R)-2-(9H-fluoren-9-ylmethoxycarbonylamino) propanoyl]pyrrolidine-2-carbonyl]amino]-3-methyl-butanoyl]amino]phenyl]methyl (4-nitrophenyl) carbonate, II-1g (125 mg, 160 µmol, 1.0 eq) in DMF (0.5 mL) was added DIEA (41.4 mg, 320 umol, 55.7 uL, 2.0 eq) at 25° C. under N₂, and then stirred at 25° C. for 0.5 hours. The mixture was filtered and purified by prep-HPLC (column: Nano-micro Kromasil C18 100*30 mm 8 um;mobile phase: [water(0.1%TFA)-ACN];B%: 35%-60%,10 min) to give II-2b (58 mg, 45.90 umol, 28.68% yield) as white solid. ¹H NMR (DMSO-d₆, 400 MHz) 812.14 (s, 2H), 10.09 (s, 1H), 9.82 (s, 1H), 9.55 (s, 1H), 9.20 (s, 2H), 8.32 (d, J = 8.8 Hz, 1H), 8.08 (s, 1H), 7.97 (s, 1H), 7.89-7.62 (m, 8H), 7.62-7.49 (m, 4H), 7.41-7.13 (m, 5H), 7.01 (s, 1H), 6.81 (s, 1H), 4.83-4.80 (m, 3H), 4.41-3.96 (m, 7H), 3.78-3.65 (m, 2H), 3.38-3.22 (m, 7H), 2.94-2.86 (m, 5H), 2.19-1.97 (m, 1H), 1.92-1.84 (m, 3H), 1.74-1.61 (m, 3H), 1.58-1.48 (m, 3H), 1.41-1.00 (m, 12H), 0.93-0.75 (m, 9H). LC/MS [M+H] 1263.6 (calculated); LC/MS [M+H] 1263.6 (observed).

### Preparation of (R)-1-((S)-2-(((S)-1-((4-(((((1-((3-(2-Amino-4-((3-((Tert-Butoxycarbonyl)Amino)Propyl)(Propyl)Carbamoyl)-3h-Benzo[B]Azepin-8-Yl)Phenyl)Sulfonyl)Azetidin-3-Yl)Methyl)Carbamoyl)Oxy)Methyl)Phenyl)Amino)-3-Methyl-1-Oxobutan-2-Yl)Carbamoyl)Pyrrolidin-1-Yl)-2-Methyl-1,4-Dioxo-7,10,13,16,19,22,25,28,31,34,37,40,43,46,49,52,55,58,61,64,67,70,73,76,79-Pentacosaoxa-3-Azadooctacontan-82-Oic Acid, II-2c

Fmoc-amine, II-2b (0.03 g, 0.024 mmol, 1 eq.) was dissolved in 50% diethylamine in DCM and allowed to sit at room temperature. Upon complete deprotection, the solution was concentrated overnight. To the crude material was added a solution of PEG25-NHS, 79-((2,5-dioxopyrrolidin-1-yl)oxy)-79-oxo-4,7,10,13,16,19,22,25,28,31,34,37,40,43,46,49,52,55,58,61,64,67,70,73,76-pentacosaoxanonaheptacontanoic acid (0.031 g, 0.024 mmol, 1 eq.) in DCM, followed by TEA (0.01 ml, 0.072 mmol, 3 eq.). The reaction mixture was concentrated and purified by HPLC to give, II-2c (0.015 g, 0.0066 mmol, 28%). LC/MS [M+2H] 1122.10 (calculated); LC/MS [M+2H] 1122.40 (observed).

Preparation of II-2: Acid II-2c (0.015 g, 0.0066 mmol, 1 eq.) and TFP (0.0022 g, 0.013 mmol, 2 eq.) were dissolved in 1 ml DMF. Collidine (0.004 ml, 0.033 mmol, 5 eq.) was added, followed by EDC-HCl; 1-(3-dimethylaminopropyl)-3-ethylcarbodiimide hydrochloride; CAS 25952-53-8 (0.0038 g, 0.020 mmol, 3 eq.). The reaction was stirred at room temperature and monitored by LCMS, then purified by HPLC to give TFP ester, II-2 (0.009 g, 0.0038 mmol, 57%). LC/MS [M+2H] 1196.10 (calculated); LC/MS [M+2H] 1196.40 (observed).

### Example 3 Synthesis of 2,3,5,6-tetrafluorophenyl 34-((S)-2-(((S)-1-((4-(((((1-((3-(2-Amino-4-((3-((Tert-Butoxycarbonyl)Amino)Propyl)(Propyl)Carbamoyl)-3h-Benzo[B]Azepin-8-Yl)Phenyl)Sulfonyl)Azetidin-3-Yl)Methyl)Carbamoyl)Oxy)Methyl)Phenyl)Amino)-3-Methyl-1-Oxobutan-2-Yl)Carbamoyl)Pyrrolidin-1-Yl)-34-Oxo-4,7,10,13,16,19,22,25,28,31-Decaoxatetratriacontanoate, II-3

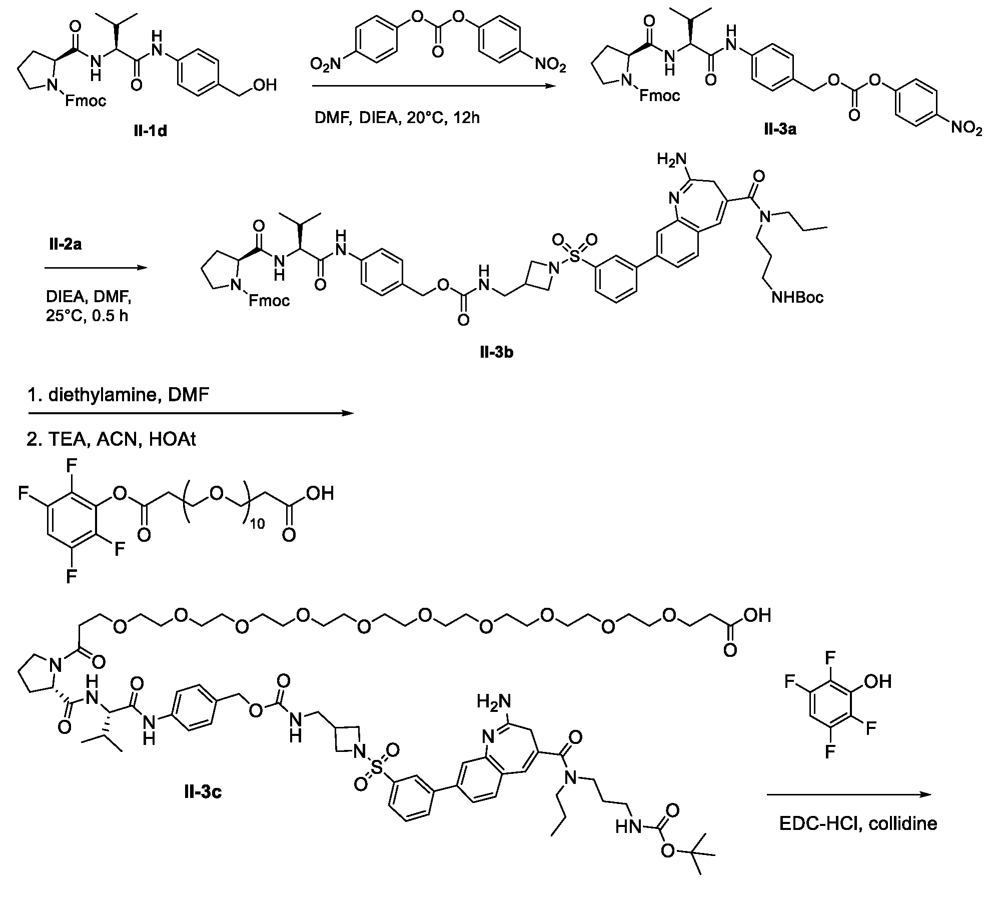

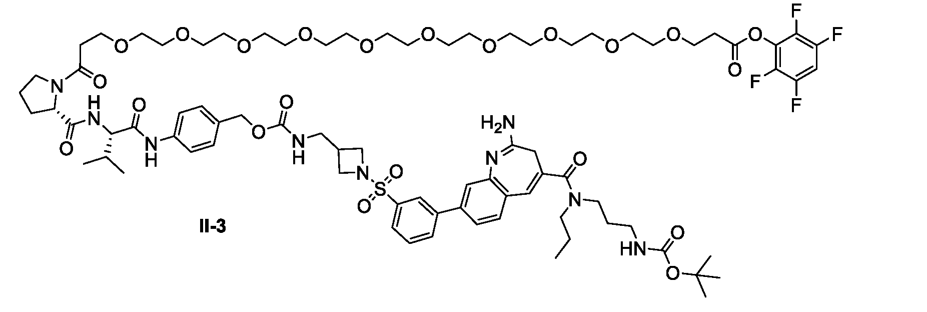

### Preparation of 9H-Fluoren-9-Ylmethyl(2s)-2-[[(1s)-2-Methyl-1-[[4-[(4-Nitrophenoxy) Carbonyloxymethyl]Phenyl]Carbamoyl]Propyl]Carbamoyl]Pyrrolidine-1-Carboxylate, II-3a

To a mixture of 9H-fluoren-9-ylmethyl(2S)-2-[[(1S)-1-[[4-(hydroxymethyl) phenyl]carbamoyl]-2-methyl-propyl]carbamoyl]pyrrolidine-1-carboxylate, II-1d (1 g, 1.85 mmol, 1.0 eq) in DMF (20 mL) was added DIEA (358 mg, 2.77 mmol, 482 uL, 1.5 eq) and bis(4-nitrophenyl) carbonate (674 mg, 2.22 mmol, 1.2 eq), and then stirred at 25° C. for 16 hr. The reaction mixture was partitioned between EtOAc (50 mL) and water (50 mL). The organic phase was separated, washed with brine (20 mL), dried over Na₂SO₄, filtered and concentrated. The crude product was purified by column chromatography (SiOz, Petroleum ether/Ethyl acetate=1:0-0:1) to give II-3a (550 mg, 778 umol, 42.2% yield).

### Preparation of 9H-Fluoren-9-Ylmethyl (2s)-2-[[(1s)-1-[[4-[[1-[3-[2-Amino-4-[3-(Tert-Butoxycarbonylamino)Propyl-Propyl-Carbamoyl]-3h-1-Benzazepin-8-Yl]Phenyl]Sulfonylazetidin-3-Yl]Methylcarbamoyloxymethyl]Phenyl]Carbamoyl]-2-MethylPropyl]Carbamoyl]Pyrrolidine-1-Carboxylate, II-3b

To a mixture of tert-butyl N-[3-[[2-amino-8-[3-[3-(aminomethyl)azetidin-1-yl] sulfonylphenyl]-3H-1-benzazepine-4-carbonyl]-propyl-amino]propyl]carbamate, II-2a (100 mg, 160 umol, 1.0 eq) and II-3a (113 mg, 160 umol, 1.0 eq) in DMF (0.5 mL) was added diisopropylethylamine, DIEA (41.4 mg, 320.1 umol, 55.7 uL, 2.0 eq) at 25° C. The mixture was stirred at 25° C. for 0.5 hours. Then it was filtered and purified by prep-HPLC (column: Nano-micro Kromasil C18 100*30 mm 8 um;mobile phase: [water (0.1%TFA)-ACN]; B%: 35%-65%, 10 min) to afford II-3b (70 mg, 58.70 umol, 36.68% yield) as white solid. ¹H NMR (DMSO-d₆, 400 MHz) 612.04 (s, 1H), 10.13-9.99 (m, 2H), 9.81 (s, 1H), 9.09 (s, 2H), 8.20 (d, J = 8.8 Hz, 1H), 8.09 (s, 1H), 7.98 (s, 1H), 7.89-7.70 (m, 5H), 7.68-7.60 (m, 2H), 7.51 (t, J = 8.8 Hz, 2H), 7.42-7.22 (m, 5H), 7.18 (t, J = 8.8 Hz, 2H), 7.02 (s, 1H), 6.81 (s, 1H), 4.82 (s, 2H), 4.56-4.53 (m, 1H), 4.36-4.14 (m, 4H), 4.08-3.92 (m, 2H), 3.74 (t, J = 8.0 Hz, 2H), 3.36-3.22 (m, 6H), 2.93-2.89 (m, 5H), 2.24-2.20 (m, 1H), 2.03-1.73 (m, 6H), 1.72-1.61 (m, 2H), 1.56-1.52 (m, 2H), 1.41-1.08 (m, 9H), 0.93-0.60 (m, 9H). LC/MS [M+H] 1192.6 (calculated); LC/MS [M+H] 1192.6 (observed).

### Preparation of 34-((S)-2-(((S)-1-((4-(((((1-((3-(2-Amino-4-((3-((Tert-Butoxycarbonyl)Amino)Propyl)(Propyl)Carbamoyl)-3h-Benzo[B]Azepin-8-Yl)Phenyl)Sulfonyl)Azetidin-3-Yl)Methyl)Carbamoyl)Oxy)Methyl)Phenyl)Amino)-3-Methyl-1-Oxobutan-2-Yl)Carbamoyl)Pyrrolidin-1-yl)-34-Oxo-4,7,10,13,16,19,22,25,28,31-Decaoxatetratriacontanoic Acid, II-3c

Fmoc-amine, II-3b (0.037 g, 0.031 mmol, 1 eq.) was dissolved in 0.5 ml DMF. Diethylamine (0.1 ml) was added, and the reaction allowed to sit at room temperature for one hour. The reaction mixture was concentrated and the product triturated three times with diethyl ether. To the crude material was added a solution of PEG10-TFP, 34-oxo-34-(2,3,5,6-tetrafluorophenoxy)-4,7,10,13,16,19,22,25,28,31-decaoxatetratriacontanoic acid (0.0188 g, 0.027 mmol, 0.86 eq.) in acetonitrile, ACN, followed by triethylamine, TEA (0.022 ml, 0.16 mmol, 5 eq.) and HOAt (0.0017 g, 0.012 mmol, 0.4 eq.). Upon completion, the reaction mixture was diluted with water and the product purified by HPLC to give II-3c (0.016 g, 0.01 mmol, 33%). LC/MS [M+H] 1510.76 (calculated); LC/MS [M+H] 1511.14 (observed).

Preparation of II-3: Acid II-3a (0.0156 g, 0.0103 mmol, 1 eq.) and TFP (0.004 g, 0.024 mmol, 2.3 eq.) were dissolved in 1 ml ACN. Collidine (0.0068 ml, 0.051 mmol, 5 eq.) was added, followed by EDC-HCl (0.0045 g, 0.023 mmol, 2.23 eq.). The reaction was stirred at room temperature and monitored by LCMS, then purified by HPLC to give TFP ester, II-3 (0.0097 g, 0.0058 mmol, 57%). LC/MS [M+H] 1658.56 (calculated); LC/MS [M+H] 1659.15 (observed).

### Example 4 Synthesis of [4-[[(2S)-2-[[(2S)-1-[(2R)-2-(9H-Fluoren-9-Ylmethoxycarbonylamino)Propanoyl]Pyrrolidine-2-Carbonyl]Amino]-3-Methyl-Butanoyl]Amino]Phenyl]Methyl 4-[[5-Amino-7-(Dipropylcarbamoyl)-6H-Thieno[3,2-b]Azepin-2-Yl]Methyl] Piperazine-1-Carboxylate, II-4

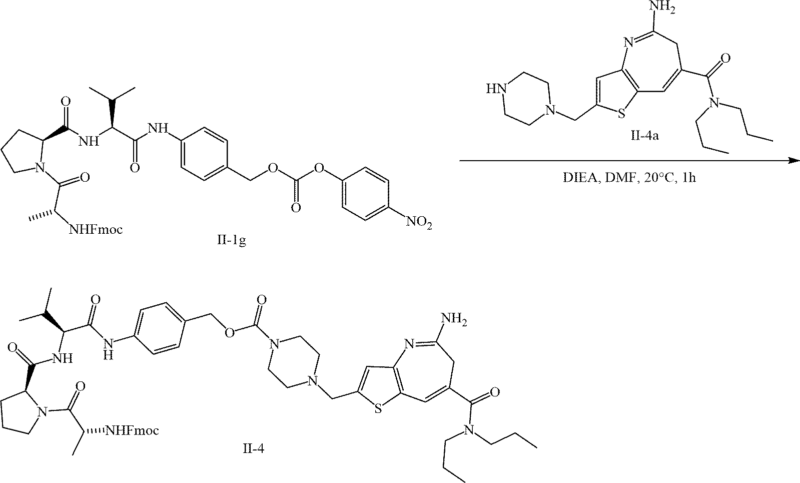

To a mixture of 5-amino-2-(piperazin-1-ylmethyl)-N,N-dipropyl-6H-thieno[3,2-b]azepine-7-carboxamide, II-4a (100 mg, 162 umol, 1.0 eq, 2 TFA) and [4-[[(2S)-2-[[(2S)-1-[(2R)-2- (9H-fluoren-9-ylmethoxycarbonylamino)propanoyl]pyrrolidine-2-carbonyl]amino]-3-methyl-butanoyl]amino]phenyl]methyl (4-nitrophenyl) carbonate, II-1g (139 mg, 178 umol, 1.1 eq) in DMF (1 mL) was added DIEA (62.8 mg, 486 umol, 84.6 uL, 3.0 eq) at 20° C. under N₂, and then stirred at 20° C. for 1 hour. The mixture was filtered and purified by prep-HPLC (column: Phenomenex Luna C18 150*30 mm*5 um; mobile phase: [water (0.1% TFA)-ACN]; B%: 35%-65%, 8 min) to afford II-4 (24.5 mg, 23.40 umol, 14.45% yield, 98.19% purity) as white solid. ¹H NMR (MeOD, 400 MHz) δ7.91 (d, J = 8.4 Hz, 2H), 7.75 (dd, J = 7.6, 3.6 Hz, 2H), 7.57 (d, J = 8.0 Hz, 2H), 7.43-7.31 (m, 6H), 7.30-7.20 (m, 3H), 7.12 (s, 1H), 5.03 (s, 2H), 4.58-4.50 (m, 2H), 4.46 (q, J = 6.8 Hz, 1H), 4.38 (s, 2H), 4.07-3.98 (m, 2H), 3.78-3.69 (m, 2H), 3.62-3.56 (m, 1H), 3.53-3.43 (m, 6H), 3.41 (s, 3H), 3.11-2.83 (m, 4H), 2.60-2.48 (m, 1H), 2.44-2.31 (m, 1H), 2.17-2.03 (m, 3H), 1.75-1.63 (m, 4H), 1.42 (d, J = 6.8 Hz, 3H), 1.03-0.87 (m, 12H). LC/MS [M+H] 1028.5 (calculated); LC/MS [M+H] 1028.5 (observed).

### Example 5 Synthesis of [4-[[(2S)-2-[[(2S)-1-[(2R)-2-(9H-Fluoren-9-Ylmethoxycarbonylamino)Propanoyl]Pyrrolidine-2-Carbonyl]Amino]-3-Methyl-Butanoyl]Amino]Phenyl]Methyl N-[4-[[(1S)-1-(2-Hydroxyethyl) Pentyl]Amino]Quinazolin-2-Yl]Carbamate, II-5

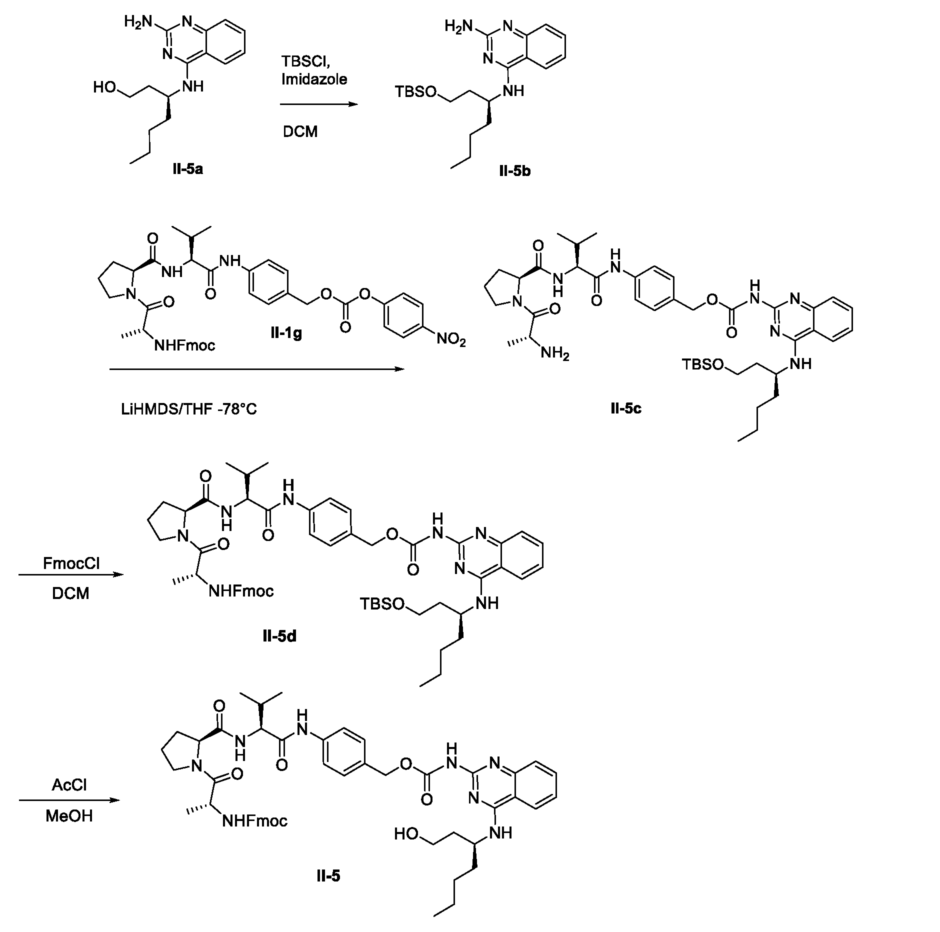

### Preparation of N4-[(1S)-1-[2-[Tert-Butyl(Dimethyl)Silyl]Oxyethyl] Pentyl]Quinazoline-2,4-Diamine, II-5b

To a mixture of (3S)-3-[(2-aminoquinazolin-4-yl)amino]heptan-1-ol, II-5a (0.69 g, 2.51 mmol, 1.0 eq) in DCM (10 mL) was added imidazole (514 mg, 7.54 mmol, 3.0 eq) and tertbutyldimethylsilyl chloride, TBSCI (379 mg, 2.51 mmol, 1.0 eq) at 0° C., and then stirred at 15° C. for 10 hr. The mixture was washed by water (10 mL). The organic layer was dried over Na₂SO₄, filtered and concentrated. The residue was purified by column chromatography (SiO2, Petroleum ether/Ethyl acetate=1:0 to 0/1) to give II-5b (400 mg, 1.03 mmol, 40.93% yield) as yellow solid. ¹H NMR (DMSO-d₆, 400 MHz) 68.08 (d, J = 8.8 Hz, 1H), 7.49 (t, J = 7.2 Hz, 1H), 7.40 (d, J = 8.8 Hz, 1H), 7.23 (d, J = 8.0 Hz, 1H), 7.05 (t, J = 7.2 Hz, 1H), 5.92 (s, 2H), 3.73-3.64 (m, 2H), 3.22-3.21 (m, 1H), 1.89-1.78 (m, 2H), 1.65-1.61 (m, 2H), 1.34-1.31 (m, 4H), 0.90-0.86 (m, 12H), 0.00 (s, 6H).

### Preparation of [4-[[(2S)-2-[[(2S)-1-[(2R)-2-Aminopropanoyl]Pyrrolidine-2-Carbonyl]Amino]-3-Methyl-Butanoyl]Amino]Phenyl]Methyl N-[4-[[(1s)-1-[2-[Tert-Butyl (Dimethyl)Silyl]Oxyethyl]Pentyl]Amino] Quinazolin-2-Yl]Carbamate, II-5c

To a mixture of 11-5b (115 mg, 296 umol, 1.0 eq) in THF (8 mL) was added LiHMDS (1 M, 2.96 mL, 10 eq) at -78° C., and then stirred at this temperature for 0.5 hr, [4-[[(2S)-2-[[(2S)-1-[(2R)-2-(9H-fluoren-9-ylmethoxycarbonylamino)propanoyl]pyrrolidine-2-carbonyl]amino]-3-methyl-butanoyl]amino]phenyl]methyl (4-nitrophenyl) carbonate, II-1g (230 mg, 296 umol, 1.0 eq) was added and it was stirred at -78° C. for 2 hr. The reaction was quenched by aq NH₄Cl (15 mL) and extracted with EtOAc (10 mL × 3), the organic layer was dried over Na₂SO₄ and concentrated to give II-5c (0.3 g, crude) as yellow solid.

### Preparation of [4-[[(2S)-2-[[(2S)-1-[(2R)-2-(9H-Fluoren-9-Ylmethoxycarbonylamino) Propanoyl]Pyrrolidine-2-Carbonyl]Amino]-3-Methyl-Butanoyl]Amino]Phenyl]Methyl N-[4-[[(1S)-1-[2-[Tert-Butyl(Dimethyl)Silyl]Oxyethyl]Pentyl]Amino]Quinazolin-2-Yl]Carbamate, II-5d

To a mixture of II-5c (290 mg, 360 umol, 1.0 eq) in DCM (10 mL) was added imidazole (73.6 mg, 1.08 mmol, 3.0 eq) and Fmoc-Cl; 9H-fluoren-9-ylmethyl carbonochloridate (93.2 mg, 360 umol, 1.0 eq) at 0° C., and then stirred at 15° C. for 10 hr. The reaction mixture was washed by water (5 mL), and the organic layer was dried over Na₂SO₄, filtered and concentrated to give 11-5d (0.37 g, 360 umol, 99% yield) as yellow oil.

Preparation of II-5: To a mixture of II-5d (300 mg, 292 umol, 1.0 eq) in MeOH (5 mL) was added acetyl chloride (22.9 mg, 292 umol, 20.8 uL, 1.0 eq) at 0° C., and then stirred at 15° C. for 2 hr. The mixture was concentrated to give a residue, the residue was purified by prep-HPLC (column: Phenomenex Synergi C18 150*25*10 um; mobile phase: [water(0.1%TFA)-ACN]; B%: 40%-56%, 10 min) to obtain (30.4 mg, 28.66 umol, 9.82% yield, 96.84% purity, TFA) as white solid ¹H NMR (MeOD, 400 MHz) b8.83 (s, 1H), 8.15 (d, J = 8.0 Hz, 1H), 7.91 (d, J = 8.4 Hz, 2H), 7.77-7.63 (m, 1H), 7.54-7.48 (m, 1H), 7.47-7.36 (m, 3H), 7.33-7.17 (m, 3H), 7.10-6.97 (m, 4H), 5.23-5.02 (m, 2H), 4.61-4.28 (m, 4H), 3.99-3.73 (m, 2H), 3.65-3.26 (m, 5H), 2.49-2.16 (m, 2H), 2.10-1.57 (m, 7H), 1.44-1.16 (m, 7H), 1.02-0.58 (m, 9H). LC/MS [M+H] 913.3 (calculated); LC/MS [M+H] 913.3 (observed).

### Example 6 Synthesis of [4-[[(2S)-2-[[(2S)-1-[(2R)-2-(9H-Fluoren-9-Ylmethoxycarbonylamino)Propanoyl]Pyrrolidine-2-Carbonyl]Amino]-3-Methyl-Butanoyl]Amino]Phenyl]Methyl 4-[[5-Amino-7-[3-(3,3-Dimethyl Butanoylamino)Propyl-PropylCarbamoyl]-6H-Thieno[3,2-B]Azepin-2-Yl]Methyl]Piperazine-1-Carboxylate, II-6

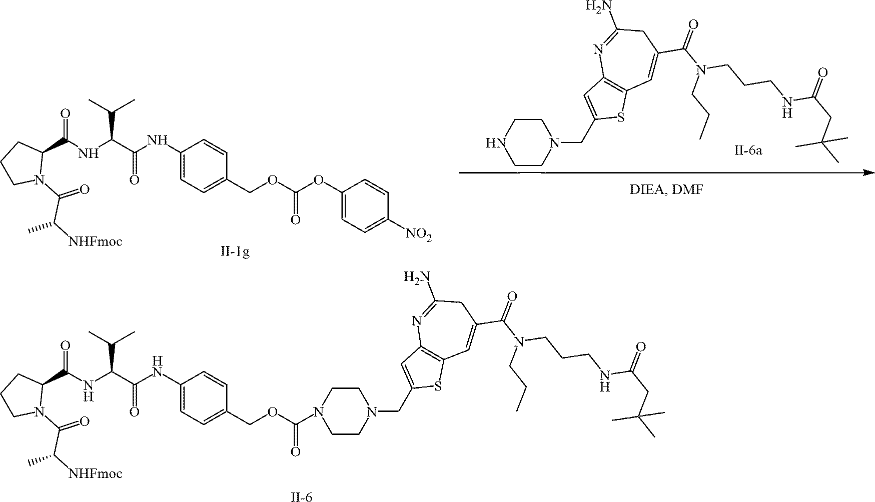

To a solution of 5-amino-N-[3-(3,3-dimethylbutanoylamino)propyl]-2-(piperazin-1-ylmethyl)-N-propyl-6H-thieno[3,2-b]azepine-7-carboxamide, II-6a (100 mg, 174 umol, 1.0 eq, 2HCl) and DIEA (89.8 mg, 695 umol, 121 uL, 4.0 eq) in DMF (0.5 mL) was added [4-[[(2S)-2-[[(2S)-1-[(2R)-2-(9H-fluoren-9-ylmethoxycarbonylamino)propanoyl]pyrrolidine-2-carbonyl]amino]-3-methyl-butanoyl]amino]phenyl]methyl (4-nitrophenyl) carbonate, II-1g (133 mg, 170 umol, 1.0 eq). The mixture was stirred at 20° C. for 1 hr under N₂ atmosphere. The reaction mixture was filtered and purified by prep-HPLC (column: Phenomenex Synergi C18 150*25*10 um; mobile phase: [water (0.1%TFA)-ACN]; B%: 35%-45%, 7 min) to give II-6 (58.3 mg, 51.08 umol, 29.40% yield) as white solid. ¹H NMR (MeOD, 400 MHz) b7.89 (d, J = 8.4 Hz, 2H), 7.71 (dd, J = 4.0, 7.6 Hz, 2H), 7.54 (d, J = 8.0 Hz, 1H), 7.38-7.28 (m, 5H), 7.27-7.18 (m, 2H), 7.13 (d, J = 5.2 Hz, 2H), 4.99 (s, 2H), 4.56-4.48 (m, 2H), 4.43 (q, J = 7.2 Hz, 1H), 4.21-4.05 (m, 2H), 4.04-3.94 (m, 2H), 3.75-3.65 (m, 2H), 3.59-3.47 (m, 4H), 3.46-3.35 (m, 6H), 3.21-3.17 (m, 2H), 2.86-2.62 (m, 2H), 2.55-2.49 (m, 1H), 2.40-2.28 (m, 1H), 2.15-1.97 (m, 5H), 1.88-1.78 (m, 2H), 1.71-1.59 (m, 2H), 1.39 (d, J = 6.8 Hz, 3H), 1.01-0.97 (m, 9H), 0.92-0.86 (m, 3H). LC/MS [M+H] 1141.6 (calculated); LC/MS [M+H] 1141.5 (observed).

### Example 7 Synthesis of [4-[[(2S)-2-[[(2S)-1-[(2S)-2-[3-[2-[2-[2-[2-[2-[2-[2-[2-[2-[2-[[2-(2,5-Dioxopyrrol-1-Yl)Acetyl]Amino]Ethoxy]Ethoxy]Ethoxy]Ethoxy]Ethoxy]Ethoxy]Ethoxy]Ethoxy]Ethoxy]Ethoxy]Pro Panoylamino]Propanoyl]Pyrrolidine-2-Carbonyl]Amino]-3-Methyl-Butanoyl]Amino]Phenyl]Methyl 4-[3-[[(2E)-6-Carbamoyl-3-[(E)-4-[(2E)-5-Carbamoyl-2-(2-Ethyl-5-Methyl-Pyrazole-3-Carbonyl)Imino-7-Methoxy-3H-Benzimidazol-1-Yl]But-2-Enyl]-2-(2-Ethyl-5-Methyl-Pyrazole-3-Carbonyl)Imino-1H-Benzimidazol-4-Yl]Oxy]Propyl]Piperazine-1-Carboxylate, II-7

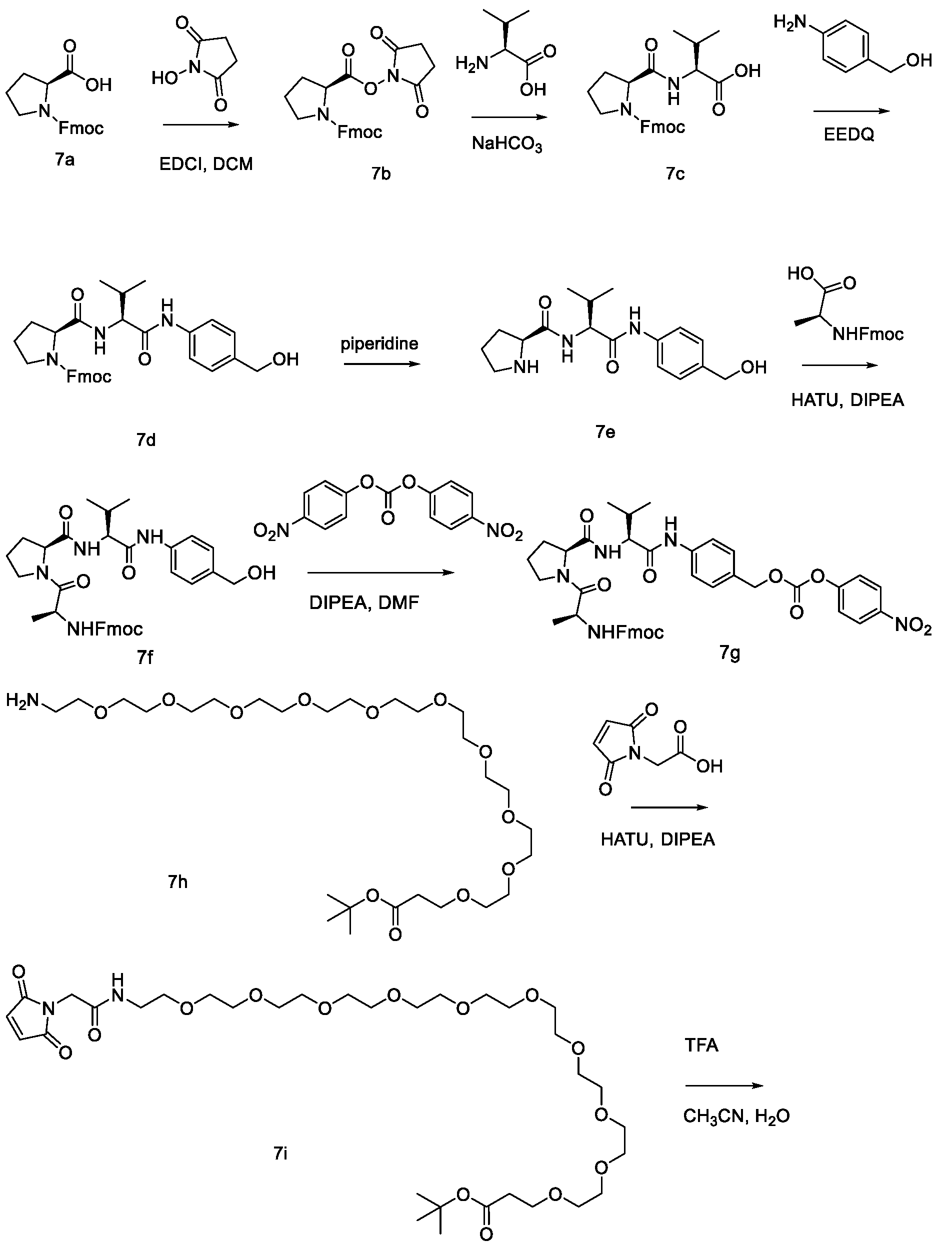

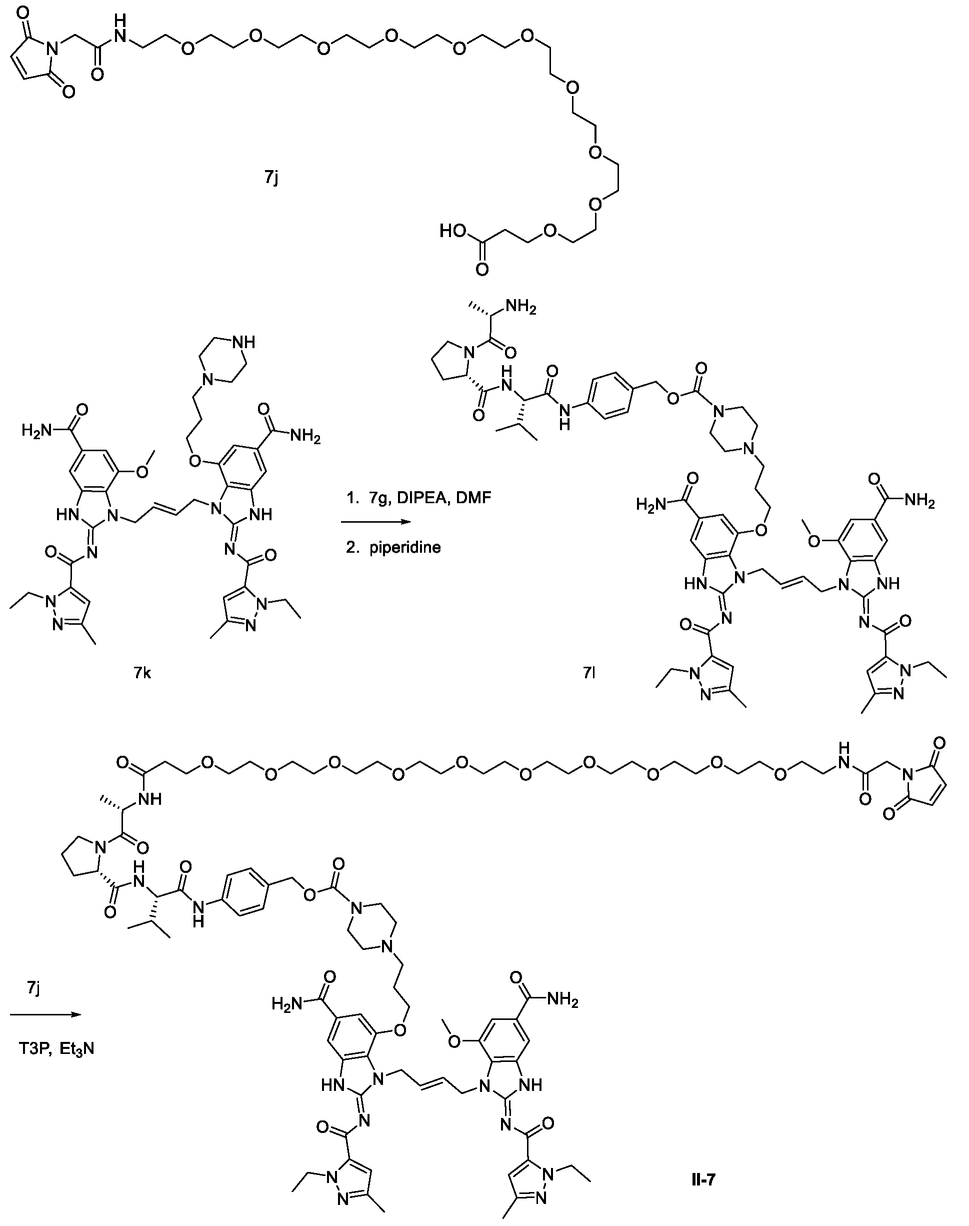

### Preparation of (S)-1-((9H-Fluoren-9-Yl)Methyl) 2-(2,5-Dioxopyrrolidin-1-Yl) Pyrrolidine-1,2-Dicarboxylate, 7b

To a solution of (2S)-1-(9H-fluoren-9-ylmethoxycarbonyl)pyrrolidine-2-carboxylic acid, 7a (15 g, 44.5 mmol, 1.0 eq) in DCM (200 mL) was added 1-hydroxypyrrolidine-2,5-dione (5.12 g, 44.5 mmol, 1.0 eq) and 1-ethyl-3-(3-dimethylaminopropyl)carbodiimide, EDCI, ED AC, EDC, CAS Reg. No. 25952-53-8 (10.2 g, 53.44 mmol, 1.2 eq), and it was stirred at 20° C. for 12 h. The mixture was washed by saturated aqueous solution of NaHCO₃ (70 mL × 3). The organic layer was dried over Na₂SO₄ and concentrated to give 7b (17.5 g, 40.28 mmol, 90.60% yield) as a white solid.

### Preparation of (2S)-2-[[(2S)-1-(9H-Fluoren-9-Ylmethoxycarbonyl)Pyrrolidine-2-Carbonyl]Amino]-3-Methyl-Butanoic Acid, 7c

To a solution of (2S)-2-amino-3-methyl-butanoic acid, L-valine (4.95 g, 42.3 mmol, 1.05 eq) in THF (200 mL) was added NaHC03 (3.55 g, 42.3 mmol, 1.64 mL, 1.05 eq) in H₂O (50 mL) and 7b (17.5 g, 40.28 mmol, 1.0 eq) and it was stirred at 20° C. for 12 h. The mixture was extracted with MTBE (2 × 100 mL) (discarded). The pH of aqueous layer was adjusted to 5-6 with HCl(6 M) and extracted with EtOAc (3 × 200 ml). The combined organic layer was dried over Na₂SO₄ and concentrated to give 7c (15 g, 34.36 mmol, 85.31% yield) as white solid. ¹H NMR (MeOD, 400 MHz) 67.80 (d, J = 7.2 Hz, 2H), 7.70-7.54 (m, 2H), 7.43-7.28 (m, 4H), 4.49-4.15 (m, 5H), 3.69-3.38 (m, 2H), 2.42-2.01 (m, 3H), 2.00-1.82 (m, 2H), 1.01-0.86 (m, 6H).

### Preparation of 9H-Fluoren-9-Ylmethyl(2S)-2-[[(1S)-1-[[4-(Hydroxymethyl)Phenyl] Carbamoyl]-2-Methyl-Propyl]Carbamoyl]Pyrrolidine-1-Carboxylate, 7d

To a solution 7c (10 g, 22.9 mmol, 1.0 eq) and (4-aminophenyl)methanol (4.23 g, 34.4 mmol, 1.5 eq) in MeOH (80 mL) and DCM (80 mL) was added 2-ethoxy-1-ethoxycarbonyl-1,2-dihydroquinoline, EEDQ, CAS Reg. No. 16357-59-8 (8.50 g, 34.36 mmol, 1.5 eq) and then stirred at 20° C. for 12 h. The mixture was concentrated in vacuum to give a residue and the residue was purified by flash silica gel chromatography (Silica Flash Column, Eluent of 0-40% Ethyl acetate/MeOH @ 65 mL/min) to give 7d (13 g, 24.0 mmol, 52.38% yield) as yellow solid. ¹H NMR (MeOD, 400 MHz) 67.85-7.71 (m, 2H), 7.68-7.48 (m, 3H), 7.47-7.16 (m, 7H), 4.53 (d, J = 15.2 Hz, 2H), 4.49-4.41 (m, 1H), 4.40-4.33 (m, 2H), 4.32-4.27 (m, 1H), 4.26-4.17 (m, 1H), 4.16-4.07 (m, 1H), 3.69-3.38 (m, 2H), 2.40-2.05 (m, 2H), 1.99-1.82 (m, 2H), 1.08-0.88 (m, 6H).

### Preparation of (2S)-N-[(1S)-1-[[4-(Hydroxymethyl)Phenyl]Carbamoyl]-2-MethylPropyl]Pyrrolidine-2-Carboxamide, 7e

To a solution of 7d (13 g, 24.0 mmol, 1.0 eq) in DCM (130 mL) was added piperidine (10.22 g, 120 mmol, 11.85 mL, 5.0 eq) and then stirred at 20° C. for 2 h. The mixture was concentrated to give a residue and the residue was triturated with EtOAc at 20° C. for 20 min to give 7e (8 g, crude) as white solid.

### Preparation of 9H-Fluoren-9-Ylmethyl N-[(1S)-2-[(2S)-2-[[(1S)-1-[[4-(Hydroxymethyl) Phenyl]Carbamoyl]-2-Methyl-Propyl]Carbamoyl]Pyrrolidin-1-Yl]-1-Methyl-2-OxoEthyl]Carbamate, 7f

To a solution of (2S)-2-(9H-fluoren-9-ylmethoxycarbonylamino)propanoic acid (3.95 g, 12.68 mmol, 1.5 eq) in DMF (30 mL) was added 1-bBis(dimethylamino)methylene]-1H-1,2,3-triazolo[4,5-b]pyridinium 3-oxide hexafluorophosphate, Hexafluorophosphate Azabenzotriazole Tetramethyl Uronium, HATU, CAS Reg. No. 148893-10-1 (4.82 g, 12.68 mmol, 1.5 eq) and DIPEA (3.28 g, 25.36 mmol, 4.42 mL, 3 eq) at 0° C. After addition, the mixture was stirred at this temperature for 5 min, and then 7e (2.7 g, 8.45 mmol, 1 eq) was added at 0° C. and then the resulting mixture was stirred at 0° C. for 25 min. The reaction mixture was quenched by addition of H₂O (150 mL) and then extracted with EtOAc (70 mL x 3). The combined organic layers were washed with brine (50 mL × 3), dried over Na₂SO₄, filtered and concentrated under reduced pressure. The residue was purified by column chromatography (SiO₂, Petroleum ether:Ethyl acetate = 1:0 to 0:1) and then (SiO₂, EtOAc:MeOH = 1:0 to 10:1) to give 7f (2.94 g, 4.80 mmol, 56.76% yield) as an off-white solid. ¹H NMR (MeOD-d₄, 400 MHz) 87.79 (d, J = 7.6 Hz, 2H), 7.66 (t, J = 6.4 Hz, 2H), 7.54 (d, J = 8.4 Hz, 2H), 7.39 (t, J = 7.2 Hz, 2H), 7.35-7.26 (m, 4H), 4.59-4.51 (m, 3H), 4.50-4.40 (m, 1H), 4.39-4.30 (m, 2H), 4.29-4.18 (m, 2H), 3.83-3.71 (m, 1H), 3.68-3.63 (m, 1H), 2.31-2.09 (m, 2H), 2.07-1.91 (m, 3H), 1.36 (dd, J = 6.4, 9.6 Hz, 4H), 1.03 (dd, J = 4.0, 6.8 Hz, 6H).

### Preparation of [4-[[(2S)-2-[[(2S)-1-[(2S)-2-(9H-Fluoren-9-Ylmethoxycarbonylamino) Propanoyl]Pyrrolidine-2-Carbonyl]Amino]-3-Methyl-Butanoyl]Amino]Phenyl]Methyl (4-Nitrophenyl) Carbonate, 7g

To a solution of 9H-fluoren-9-ylmethyl N-[(1S)-2-[(2S)-2-[[(1S)-1-[[4-(hydroxymethyl)phenyl]carbamoyl]-2-methyl-propyl]carbamoyl]pyrrolidin-1-yl]-1-methyl-2-oxo-ethyl]carbamate (2.4 g, 3.92 mmol, 1 eq) in DMF(20 mL) was added bis(4-nitrophenyl) carbonate (2.38 g, 7.83 mmol, 2 eq)and DIPEA (1.01 g, 7.83 mmol, 1.36 mL, 2 eq) and then stirred at 20° C. for 1 h. The reaction mixture was quenched by addition of H₂O (100 mL) at 0° C., and then extracted with EtOAc (80 mL × 3). The combined organic layers were washed with brine (50 mL × 3), dried over Na₂SO₄, filtered and concentrated under reduced pressure. The residue was purified by column chromatography (SiO₂, Petroleum ether:Ethyl acetate = 1:0 to 0:1) to give 7 g (2.7 g, 3.47 mmol, 88.62% yield) as a white solid. ¹H NMR (CDCl₃, 400 MHz) 6 8.32-8.22 (m, 3H), 7.78 (d, J = 7.6 Hz, 2H), 7.67 (br d, J = 8.4 Hz, 2H), 7.60 (br d, J = 7.6 Hz, 2H), 7.46-7.29 (m, 8H), 7.18 (br d, J = 8.4 Hz, 1H), 5.60 (br d, J = 7.6 Hz, 1H), 5.25 (s, 2H), 4.70-4.66 (m, 1H), 4.62-4.52 (m, 1H), 4.46-4.28 (m, 3H), 4.28-4.19 (m, 1H), 3.81-3.68 (m, 1H), 3.62-3.58 (m, 1H), 2.48-2.29 (m, 2H), 2.19-1.98 (m, 3H), 1.41 (d, J = 7.2 Hz, 3H), 1.10-0.94 (m, 6H). LC/MS [M+H] 778.3 (calculated); LC/MS [M+H] 778.2 (observed).

### Preparation Of Tert-Butyl 3-[2-[2-[2-[2-[2-[2-[2-[2-[2-[2-[[2-(2,5-Dioxopyrrol-1-Yl) Acetyl Amino] Ethoxy] Ethoxy] Ethoxy]Ethoxy] Ethoxy] Ethoxy]Ethoxy] Ethoxy] Ethoxy] Ethoxy]Propa Noate, 7i

To a solution of tert-butyl 3-[2-[2-[2-[2-[2-[2-[2-[2-[2-(2-aminoethoxy)ethoxy]ethoxy]ethoxy]ethoxy]ethoxy]ethoxy]ethoxy]ethoxy]ethoxy]propanoate, 7 h (11.3 g, 19.3 mmol, 1 eq), 2-(2,5-dioxopyrrol-1-yl)acetic acid (3 g, 19.3 mmol, 1 eq) and DIPEA (10.0 g, 77.4 mmol, 13.5 mL, 4 eq) in DCM (100 mL) was added HATU (8.09 g, 21.3 mmol, 1.1 eq) at 0° C. and then the mixture was stirred at 0° C. for 30 min. The reaction mixture was concentrated under reduced pressure. The residue was purified by prep-HPLC (TFA condition; column: Phenomenex luna c18 250 mm* 100 mm*10 um;mobile phase: [water(0.1%TFA)-ACN];B%: 25%-55%,25 min) to give 7i (4.5 g, 6.23 mmol, 32.2% yield) as a yellow oil. ¹H NMR (CDCl₃, 400 MHz) 66.88-6.80 (m, 1H), 6.78 (s, 2H), 4.22 (s, 2H), 3.77-3.54 (m, 40H), 3.47 (q, J = 5.2 Hz, 2H), 2.51 (t, J = 6.4 Hz, 2H), 1.46 (s, 9H).

### Preparation Of 3-[2-[2-[2-[2-[2-[2-[2-[2-[2-[2-[[2-(2,5-Dioxopyrrol-1-Yl) Acetyl Amino] Ethoxy] Ethoxy] Ethoxy]Ethoxy] Ethoxy] Ethoxy]Ethoxy] Ethoxy] Ethoxy] Ethoxy]Propa Noic Acid, 7j

To a solution of 7i (4.5 g, 6.23 mmol, 1 eq) in CH₃CN (25 mL) and H₂O (25 mL) was added TFA (5.68 g, 49.8 mmol, 3.69 mL, 8 eq) and then stirred at 80° C. for 1 h. The reaction mixture was concentrated under reduced pressure to remove CH₃CN. The residue was extracted with MTBE (10 mL × 3) and discarded. The water phase was concentrated under reduced pressure to give a residue. The residue was purified by prep-HPLC (TFA condition; column: Phenomenex Luna c18 250 mm × 100 mm × 10 um;mobile phase: [water(0.1%TFA)-ACN];B%: 0%-25%,24 min) to give 7j (1.6 g, 2.40 mmol, 38.6% yield) as a light yellow oil. ¹H NMR (CDCh, 400 MHz) 66.95 (br s, 1H), 6.78 (s, 2H), 4.22 (s, 2H), 3.78 (t, J = 6.4 Hz, 2H), 3.70-3.63 (m, 36H), 3.60-3.54 (m, 2H), 3.46 (q, J = 5.2 Hz, 2H), 2.61 (t, J = 6.0 Hz, 2H). LCMS (ESI): mass calcd. for C₄₂H₄₃N₅O₁₀ 667.3, m/z found 667.2 [M+H]⁺.

### Preparation Of [4-[[(2s)-2-[[(2s)-1-[(2s)-2-Aminopropanoyl]Pyrrolidine-2-Carbonyl]Amino]-3-Methyl-Butanoyl]Amino]Phenyl]Methyl 4-[3-[[(2e)-6-Carbamoyl-3-[(E)- 4-[(2e)-5-Carbamoyl-2-(2-Ethyl-5-Methyl-Pyrazole-3-Carbonyl)Imino-7-Methoxy-3h-Benzimidazol-1-Yl]But-2-Enyl]-2-(2-Ethyl-5-Methyl-Pyrazole-3-Carbonyl)Imino-1h-Benzimidazol-4-Yl]Oxy]Propyl]Piperazine-1-Carboxylate, 7l

To a solution of (2E)-1-[(E)-4-[(2E)-5-carbamoyl-2-(2-ethyl-5-methyl-pyrazole-3 -carbonyl)imino-7-(3-piperazin-1-ylpropoxy)-3H-benzimidazol-1-yl]but-2-enyl]-2-(2-ethyl-5-methyl-pyrazole-3-carbonyl)imino-7-methoxy-3H-benzimidazole-5-carboxamide, 7k (150 mg, 151 umol, 1 eq, 4HCl) and 7 g (129 mg, 166 umol, 1.1 eq) in DMF (3.00 mL) was added DIPEA (97.0 mg, 754 umol, 131 uL, 5 eq) at 20° C., the mixture was stirred at this temperature for 2 h, and then piperidine (39.0 mg, 452.35 umol, 45.0 uL, 3 eq) was added. The mixture was stirred at 20° C. for another 2 h. The mixture was filtered and the residue was purified by prep-HPLC (column: Phenomenex Luna 80*30 mm*3 um;mobile phase: [water(0.1%TFA)-ACN];B%: 20%-40%,8 min) to give 71 (90 mg, 60.26 umol, 39.97% yield, 2TFA) as light yellow solid. ¹H NMR (MeOD, 400 MHz) 67.67-7.53 (m, 4H), 7.37 (d, J = 8.4 Hz, 2H), 7.25 (dd, J = 1.2, 14.0 Hz, 2H), 6.60 (d, J = 7.6 Hz, 2H), 5.85-5.68 (m, 2H), 5.14 (s, 2H), 5.00 (br s, 5H), 4.64-4.54 (m, 4H), 4.33-4.18 (m, 2H), 3.95 (br t, J = 6.0 Hz, 2H), 3.74-3.58 (m, 5H), 3.25-3.13 (m, 4H), 2.21 (s, 3H), 2.19 (s, 3H), 2.16-1.92 (m, 6H), 1.52 (d, J = 7.2 Hz, 3H), 1.40-1.29 (m, 6H), 1.08-0.98 (m, 6H).

### Preparation of 7

To a solution 71 (50 mg, 33.5 umol, 1 eq, 2TFA) and 7j (22.0 mg, 33.5 umol, 1 eq) in DMF (1.00 mL) was added Et₃N (7.00 mg, 66.9 umol, 9.00 uL, 2 eq) and 1-Propanephosphonic anhydride, T₃P, CAS Reg. No. 68957-94-8 (32.0 mg, 50.2 umol, 30.0 uL, 50% purity, 1.5 eq), and then stirred at 20° C. for 2 h. The mixture was filtered and the residue was purified by prep-HPLC (column: Phenomenex Luna 80*30 mm*3 um;mobile phase: [water(0.1%TFA)-ACN];B%: 15%-40%,8 min) to give 7 (16 mg, 7.47 umol, 22.31% yield, 2TFA) as white solid. ¹H NMR (MeOD, 400 MHz) 67.68-7.53 (m, 4H), 7.37 (d, J = 8.4 Hz, 2H), 7.30-7.20 (m, 2H), 6.88 (s, 2H), 6.60 (d, J = 12 Hz, 2H), 5.78 (br s, 2H), 5.14 (s, 2H), 4.99 (br s, 4H), 4.68-4.47 (m, 8H), 4.29-4.25 (m, 1H), 4.16 (s, 3H), 3.96 (br t, J = 5.6 Hz, 2H), 3.80 (td, J = 6.8, 9.2 Hz, 2H), 3.74-3.71 (m, 2H), 3.70 (s, 4H), 3.67-3.56 (m, 36H), 3.53 (br t, J = 5.6 Hz, 2H), 3.48 (td, J = 1.6, 3.2 Hz, 1H), 3.36 (br t, J = 5.2 Hz, 2H), 3.23-3.13 (m, 4H), 2.54-2.43 (m, 2H), 2.20 (d, J = 10.4 Hz, 6H), 2.18-2.10 (m, 2H), 2.06-1.94 (m, 5H), 1.40-1.28 (m, 9H), 1.02 (dd, J = 5.2, 6.4 Hz, 6H). LCMS (ESI): mass calcd. for C₄₂H₄₃N₅O₁₀ 1913.9, m/z found 1914.0 [M+H]⁺.

### Example 201 Preparation of Immunoconjugates (IC)

In an exemplary procedure, an antibody is buffer exchanged into a conjugation buffer containing 100 mM boric acid, 50 mM sodium chloride, 1 mM ethylenediaminetetraacetic acid at pH 8.3, using G-25 SEPHADEX™ desalting columns (Sigma-Aldrich, St. Louis, MO). The eluates are then each adjusted to 6 mg/ml using the buffer and then sterile filtered. The antibody at 6 mg/ml is pre-warmed to 30° C. and rapidly mixed with 2-20 (e.g., 7-10) molar equivalents of immunostimulant-elastase substrate, peptide linker compound. The reaction is allowed to proceed for 16 hours at 30° C. and Immunoconjugate A is separated from reactants by running over two successive G-25 desalting columns equilibrated in phosphate buffered saline (PBS) at pH 7.2 to provide the Immunoconjugate (IC) of Table 3. Adjuvant-antibody ratio (DAR) is determined by liquid chromatography mass spectrometry analysis using a C4 reverse phase column on an ACQUITY™ UPLC H-class (Waters Corporation, Milford, Massachusetts) connected to a XEVO™ G2-XS TOF mass spectrometer (Waters Corporation).

For conjugation, the antibody may dissolved in a physiological buffer system known in the art that will not adversely impact the stability or antigen-binding specificity of the antibody. Phosphate buffered saline may be used. The immunostimulant-elastase substrate, peptide linker compound is dissolved in a solvent system comprising at least one polar aprotic solvent as described elsewhere herein. In some such aspects, immunostimulant-elastase substrate, peptide linker intermediate is dissolved to a concentration of about 5 mM, 10 mM, about 20 mM, about 30 mM, about 40 mM or about 50 mM, and ranges thereof such as from about 50 mM to about 50 mM or from about 10 mM to about 30 mM in pH 8 Tris buffer (e.g., 50 mM Tris). In some aspects, the immunostimulant-elastase substrate, peptide linker intermediate is dissolved in DMSO or acetonitrile, or in DMSO. In the conjugation reaction, an equivalent excess of immunostimulant-elastase substrate, peptide linker intermediate solution is diluted and combined with chilled antibody solution (e.g. from about 1° C. to about 10° C.). The immunostimulant-elastase substrate, peptide linker intermediate solution may suitably be diluted with at least one polar aprotic solvent and at least one polar protic solvent, examples of which include water, methanol, ethanol, n-propanol, and acetic acid. In some particular aspects the thienoazepine-linker intermediate is dissolved in DMSO and diluted with acetonitrile and water prior to admixture with the antibody solution. The molar equivalents of immunostimulant-elastase substrate, peptide linker intermediate to antibody may be about 1.5:1, about 3:1, about 5:1, about 10:1 about 15:1 or about 20:1, and ranges thereof, such as from about 1.5:1 to about 20:1 from about 1.5:1 to about 15:1, from about 1.5:1 to about 10:1, from about 3:1 to about 15:1, from about 3:1 to about 10:1, from about 5:1 to about 15:1 or from about 5:1 to about 10:1. The reaction may suitably be monitored for completion by methods known in the art, such as LC-MS, and the reaction is typically complete in from about 1 hour to about 24 hours. After the reaction is complete, a reagent may be added to the reaction mixture to quench the reaction and/or cap unreacted antibody thiol groups. An example of a suitable capping reagent is ethylmaleimide.

Following conjugation, the immunoconjugates may be purified and separated from unconjugated reactants and/or conjugate aggregates by purification methods known in the art such as, for example and not limited to, size exclusion chromatography, hydrophobic interaction chromatography, ion exchange chromatography, chromatofocusing, µLtrafiltration, centrifugal µLtrafiltration, and combinations thereof. For instance, purification may be preceded by diluting the immunoconjugate, such in 20 mM sodium succinate, pH 5. The diluted solution is applied to a cation exchange column followed by washing with, e.g., at least 10 column volumes of 20 mM sodium succinate, pH 5. The conjugate may be suitably eluted with a buffer such as PBS.

### Example 202 HEK Reporter Assay

HEK293 reporter cells expressing human TLR7 or human TLR8 were purchased from Invivogen and vendor protocols were followed for cellular propagation and experimentation. Briefly, cells were grown to 80-85% confluence at 5% CO₂ in DMEM supplemented with 10% FBS, Zeocin, and Blasticidin. Cells were then seeded in 96-well flat plates at 4x10⁴ cells/well with substrate containing HEK detection medium and immunostimulatory molecules. Activity of immunostimulatory compounds are measured using a plate reader at 620-655 nm wavelength.

### Example 203 Assessment of Immunoconjugate Activity In Vitro

The RAW 264.7 murine macrophage cell line was cultured according to vendor protocols (Invivogen). RAW 264.7 cells were co-cultured with the human HER2-expressing tumor cell line, HCC1954, and then stimulated with a range of concentrations of trastuzumab-based ISACs, ISAC1 and ISAC2 (FIG. 9). Stimulated cells were cultured for 18 hours and then assessed for cellular activation based on production of proinflammatory cytokine TNFa. Cell-free supernatant was collected and analyzed by ELISA. This example shows that Immunoconjugates of the invention are effective at eliciting myeloid activation, and therefore are useful for the treatment of cancer.

Isolation of Human Antigen Presenting Cells: Human myeloid antigen presenting cells (APCs) were negatively selected from human peripheral blood obtained from healthy blood donors (Stanford Blood Center, Palo Alto, California) by density gradient centrifugation using a ROSETTESEP™ Human Monocyte Enrichment Cocktail (Stem Cell Technologies, Vancouver, Canada) containing monoclonal antibodies against CD14, CD16, CD40, CD86, CD123, and HLA-DR. Immature APCs were subsequently purified to >90% purity via negative selection using an EASYSEP™ Human Monocyte Enrichment Kit (Stem Cell Technologies) without CD16 depletion containing monoclonal antibodies against CD14, CD16, CD40, CD86, CD123, and HLA-DR.

Myeloid APC Activation Assay: 2 × 10⁵ APCs were incubated in 96-well plates (Corning, Corning, NY) containing Gibco Iscove’s modified Dulbecco’s medium, IMDM (Lonza) supplemented with 10% FBS, 100 U/mL penicillin, 100 µg/mL (micrograms per milliliter) streptomycin, 2 mM L-glutamine, sodium pyruvate, non-essential amino acids, and where indicated, various concentrations of unconjugated (naked) PD-L1 or HER2 antibodies and immunoconjugates of the invention (as prepared according to the Example above). Trastuzumab and avelumab were used as the antibody constructs. Cell-free supernatants were analyzed after 18 hours via ELISA to measure TNFα secretion as a readout of a proinflammatory response.

Myeloid cell types for use in screening assays: Conventional myeloid cell types include Monocytes, M-CSF MΦ (M0), GM/IL4 DC, ex vivo cDC for TLR⅞ stimulation assays in a Cytokine read out to exclude inactive agonists. Polarized myeloid cells include Monocytes differentiated toward immunosuppressive state such as M2a MΦ (IL4/IL13), M2c MΦ (IL10/TGFb) and tumor-educated monocytes (TEM) - differentiation w/ tumor-conditioned media (786.O, MDA-MB-231, HCC1954) GM/IL6 “MDSC” in a cytokine read out to exclude agonists with limited activity across assays. Tumor-associated myeloid cells include Myeloid cells present in dissociated tumor cell suspensions (Discovery Life Sciences) in an assay for discovery of agonists.

Other useful cell lines for screening may include murine cell lines such as the RAW 264.7 described above, bone marrow derived monocytes, bone marrow derived dendritic cells or macrophages, splenic dendritic cells, TAMs (tumor associated macrophages), and myeloid-derived suppressor cells (MDSCs) in the murine setting.

All references, including publications, patent applications, and patents, cited herein are hereby incorporated by reference to the same extent as if each reference were individually and specifically indicated to be incorporated by reference and were set forth in its entirety herein.

1. An immunoconjugate comprising a cell-binding agent covalently attached to one or more immunostimulatory moieties by a linker comprising an elastase-substrate, peptide linker unit.

2. The immunoconjugate of claim 1 wherein the cell-binding agent is an antibody.

3. The immunoconjugate of claim 2 wherein the antibody is an antibody construct that has an antigen binding domain that binds to a target selected from PD-L1, HER2, CEA, and TROP2.

4. The immunoconjugate of claim 3 wherein the antibody is selected from the group consisting of atezolizumab, durvalumab, avelumab, trastuzumab, pertuzumab, labetuzumab, and sacituzumab or a biosimilar or a biobetter thereof.

5-10. (canceled)

11. The immunoconjugate of claim 1 wherein the one or more immunostimulatory moieties is a pattern-recognition receptor.

12. The immunoconjugate of claim 1 wherein the one or more immunostimulatory moieties interact with or modulate a receptor selected from the group consisting of TLR, STING, NOD2, RIG-1, and NLRP3.

13. The immunoconjugate of claim 1 having the Formula I: 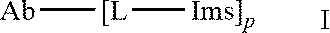
or a pharmaceutically acceptable salt thereof, wherein:
Ab is the antibody;
L is the linker comprising an elastase-substrate, peptide linker unit;
Ims is the immunostimulatory moiety; and
p is an integer from 1 to 8.

14. The immunoconjugate of claim 13 wherein Ims is a Toll-like receptor (TLR) agonist.

15. The immunoconjugate of claim 13 wherein Ims is selected from formulas Ia-f: 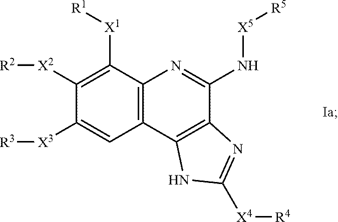 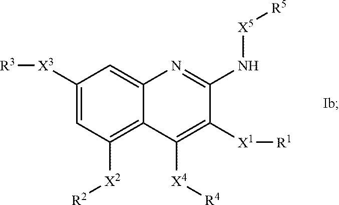 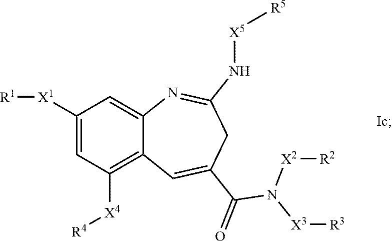 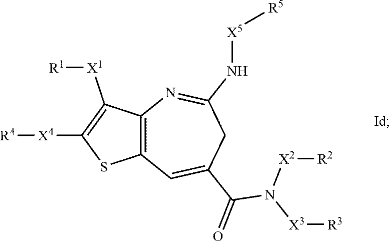 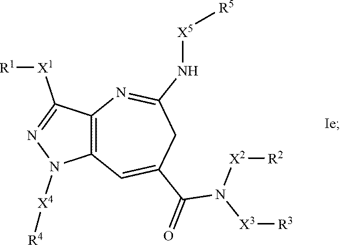 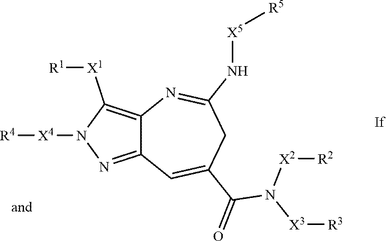
wherein R¹, R², R³, R⁴, and R⁵ are independently selected from the group consisting of H, C₁-C₁₂ alkyl, C₂-C₆ alkenyl, C₂-C₆ alkynyl, C₃-C₁₂ carbocyclyl, C₆-C₂₀ aryl, C₂-C₉ heterocyclyl, and C₁-C₂₀ heteroaryl, where alkyl, alkenyl, alkynyl, carbocyclyl, aryl, heterocyclyl, and heteroaryl are independently and optionally substituted with one or more groups selected from:
-(C₁-C₁₂ alkyldiyl)—N(R⁶)—*;
-(C₁-C₁₂ alkyldiyl)-N(R⁶)₂;
-(C₁-C₁₂ alkyldiyl)-OR⁶;
-(C₃-C₁₂ carbocyclyl);
-(C₃-C₁₂ carbocyclyl)-*;
-(C₃-C₁₂ carbocyclyl)-(C₁-C₁₂ alkyldiyl)—NR⁶—*;
-(C₃-C₁₂ carbocyclyl)-(C₁-C₁₂ alkyldiyl)-N(R⁶)₂;
-(C₃-C₁₂ carbocyclyl)-NR⁵-C(=NR^{6a})NR⁶-*;
-(C₆-C₂₀ aryl);
-(C₆-C₂₀ aryldiyl)-*;
-(C₆-C₂₀ aryldiyl)—N(R⁶)—*;
-(C₆-C₂₀ aryldiyl)-(C₁-C₁₂ alkyldiyl)—N(R⁶)—*;
-(C₆-C₂₀ aryldiyl)-(C₁-C₁₂ alkyldiyl)-(C₂-C₂₀ heterocyclyldiyl)-*;
-(C₆-C₂₀ aryldiyl)-(C₁-C₁₂ alkyldiyl)-N(R⁶)₂;
-(C₆-C₂₀ aryldiyl)-(C₁-C₁₂ alkyldiyl)-NR⁶-C(=NR^{6a})N(R⁶)-*;
-(C₂-C₂₀ heterocyclyl);
-(C₂-C₂₀ heterocyclyl)-*;
-(C₂-C₉ heterocyclyl)-(C₁-C₁₂ alkyldiyl)—NR⁶—*;
-(C₂-C₉ heterocyclyl)-(C₁-C₁₂ alkyldiyl)-N(R⁶)₂;
-(C₂-C₉ heterocyclyl)—C(═O)—(C₁-C₁₂ alkyldiyl)—N(R⁶)—*;
-(C₂-C₉ heterocyclyl)-NR⁵-C(=NR^{6a})NR⁶-*;
-(C₂-C₉ heterocyclyl)-NR⁶-(C₆-C₂₀ aryldiyl)-(C₁-C₁₂ alkyldiyl)—N(R⁶)—*;
-(C₂-C₉ heterocyclyl)-(C₆-C₂₀ aryldiyl)-*;
-(C₁-C₂₀ heteroaryl);
-(C₁-C₂₀ heteroaryldiyl)-*;
-(C₁-C₂₀ heteroaryldiyl)-(C₁-C₁₂ alkyldiyl)—N(R⁶)—*;
-(C₁-C₂₀ heteroaryldiyl)-(C₁-C₁₂ alkyldiyl)-N(R⁶)₂;
-(C₁-C₂₀ heteroaryldiyl)-NR⁶-C(=NR^{6a})N(R⁶)-*;
-(C₁-C₂₀ heteroaryldiyl)—N(R⁶)C(═O)—(C₁-C₁₂ alkyldiyl)—N(R⁶)—*;
—C(═O)—*;
—C(═O)—(C₁-C₁₂ alkyldiyl)—N(R⁶)—*;
—C(═O)—(C₂-C₂₀ heterocyclyldiyl)-*;
—C(═O)N(R⁶)₂;
—C(═O)N(R⁶)—*;
—C(═O)N(R⁶)—(C₁-C₁₂ alkyldiyl)—N(R⁶)C(═O)R⁵;
—C(═O)N(R⁶)—(C₁-C₁₂ alkyldiyl)—N(R⁶)C(═O)N(R⁶)₂;
—C(═O)NR⁶—(C₁-C₁₂ alkyldiyl)-N(R⁶)CO₂R⁶;
—C(═O)NR⁶—(C₁-C₁₂ alkyldiyl)-N(R⁶)C(=NR^{6a})N(R⁶)₂;
—C(═O)NR⁶—(C₁-C₁₂ alkyldiyl)-NR⁶C(=NR^{6a})R⁶;
—C(═O)NR⁶—(C₁-C₈ alkyldiyl)-NR⁶(C₂-C₅ heteroaryl);
—C(═O)NR⁶—(C₁-C₂₀ heteroaryldiyl)—N(R⁶)—*;
—C(═O)NR⁶—(C₁-C₂₀ heteroaryldiyl)-*;
-C(=O)NR^{6_}(C₁-C₂₀ heteroaryldiyl)-(C₁-C₁₂ alkyldiyl)-N(R⁶)₂;
—C(═O)NR⁶—(C₁-C₂₀ heteroaryldiyl)-(C₂-C₂₀ heterocyclyldiyl)—C(═O)NR⁶—(C₁-C₁₂ alkyldiyl)—NR⁶—*;
—N(R⁶)₂;
—N(R₆)—*;
—N(R⁶)C(═O)R⁶;
—N(R⁶)C(═O)—*;
—N(R⁶)C(═O)N(R⁶)₂;
—N(R⁶)C(═O)N(R⁶)—*;
—N(R⁶)CO₂R⁶;
—N(R⁶)CO₂(R⁶)—*;
-NR⁶C(=NR^{6a})N(R⁶)₂;
-NR⁶C(=NR^{6a})N(R⁶)-*;
-NR⁶C(=NR^{6a})R⁶;
—N(R⁶)C(═O)—(C₁-C₁₂ alkyldiyl)—N(R⁶)—*;
—N(R⁶)—(C₂-C₅ heteroaryl);
—N(R⁶)—S(═O)₂—(C₁-C₁₂ alkyl);
—O—(C₁-C₁₂ alkyl);
—O—(C₁-C₁₂ alkyldiyl)-N(R⁶)₂;
—O—(C₁-C₁₂ alkyldiyl)—N(R⁶)—*;
—OC(═O)N(R⁶)₂;
—OC(═O)N(R⁶)—*;
—S(═O)₂—(C₂-C₂₀ heterocyclyldiyl)-*;
—S(═O)₂—(C₂-C₂₀ heterocyclyldiyl)-(C₁-C₁₂ alkyldiyl)-N(R⁶)₂;
—S(═O)₂—(C₂-C₂₀ heterocyclyldiyl)-(C₁-C₁₂ alkyldiyl)—NR⁶—*; and
—S(═O)₂—(C₂-C₂₀ heterocyclyldiyl)-(C₁-C₁₂ alkyldiyl)-OH;
or R² and R³ of formulas Ic—If together form a 5- or 6-membered heterocyclyl ring;
X¹, X², X³, X⁴, and X⁵ are independently selected from the group consisting of a bond, C(═O), C(═O)N(R⁶), O, N(R⁶), S, S(O)₂, and S(O)₂N(R⁶);
R⁶ is selected from the group consisting of H, C₆-C₂₀ aryl, C₆-C₂₀ aryldiyl, C₁-C₁₂ alkyl, and C₁-C₁₂ alkyldiyl, or two R⁶ groups together form a 5- or 6-membered heterocyclyl ring;
R^{6a} is selected from the group consisting of C₆-C₂₀ aryl and C₁-C₂₀ heteroaryl;
where the asterisk * indicates the attachment site of L, and where one of R¹, R², R³, R⁴ and R⁵ is attached to L; and
alkyl, alkyldiyl, alkenyl, alkenyldiyl, alkynyl, alkynyldiyl, aryl, aryldiyl, carbocyclyl, carbocyclyldiyl, heterocyclyl, heterocyclyldiyl, heteroaryl, and heteroaryldiyl are independently and optionally substituted with one or more groups independently selected from F, Cl, Br, I, —CN, —CH₃, —CH₂CH₃, —CH═CH₂, —C═CH, —C≡CCH₃, —CH₂CH₂CH₃, —CH(CH₃)₂, —CH₂CH(CH₃)₂, —CH₂OH, —CH₂OCH₃, —CH₂CH₂OH, —C(CH₃)₂OH, —CH(OH)CH(CH₃)₂, —C(CH₃)₂CH₂OH, —CH₂CH₂SO₂CH₃, —CH₂OP(O)(OH)₂, —CH₂F, —CHF₂, —CF₃, —CH₂CF₃, —CH₂CHF₂, —CH(CH₃)CN, —C(CH₃)₂CN, —CH₂CN, —CH₂NH₂, —CH₂NHSO₂CH₃, —CH₂NHCH₃, —CH₂N(CH₃)₂, —CO₂H, —COCH3, —CO₂CH₃, —CO₂C(CH₃)₃, —COCH(OH)CH₃, —CONH₂, —CONHCH₃, —CON(CH₃)₂, —C(CH₃)₂CONH₂, —NH₂, —NHCH₃, —N(CH₃)₂, —NHCOCH₃, —N(CH₃)COCH₃, —NHS(O)₂CH₃, —N(CH₃)C(CH₃)₂CONH₂, —N(CH₃)CH₂CH₂S(O)₂CH₃, — NHC(═NH)H, —NHC(═NH)CH₃, —NHC(═NH)NH₂, —NHC(═O)NH₂, —NO₂, ═O, —OH, —OCH₃, —OCH₂CH₃, —OCH₂CH₂OCH₃, —OCH₂CH₂OH, —OCH₂CH₂N(CH₃)₂, —O(CH₂CH₂O)ₙ—(CH₂)ₘCO₂H, —O(CH₂CH₂O)ₙH, —OP(O)(OH)₂, —S(O)₂N(CH₃)₂, —SCH₃, —S(O)₂CH₃, and —S(O)₃H.

16. The immunoconjugate of claim 15 wherein one of R¹, R², R³, R⁴ and R⁵ is selected from the formulas: 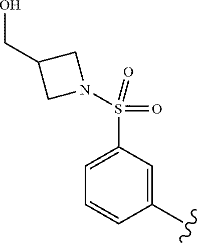 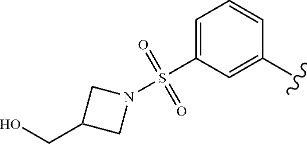 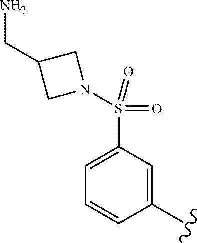 [SMILES: N=C(NCCC[C@H](N)C(=O)O)N[N+](=O)[O-]] 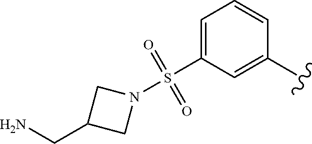
.

17. The immunoconjugate of claim 13 wherein Ims is a STING agonist.

18. The immunoconjugate of claim 13 wherein Ims has formula Ig: 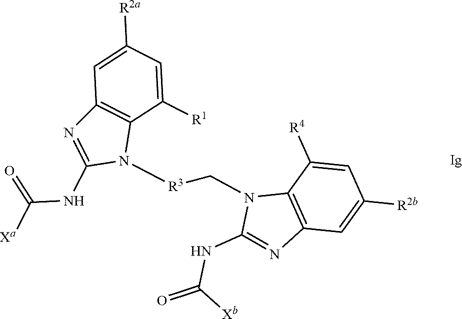
wherein X
^{a} and X^{b} are independently selected from a five-membered heteroaryl;
R¹ is selected from the group consisting of F, Cl, Br, I, —CN, —OH, and —O—(C₁-C₆ alkyldiyl).
R^{2a} and R^{2b} are independently selected from —C(═O)N(R⁵)₂;
R³ is selected from C₁-C₆ alkyldiyl, -(C₁-C₃ alkyldiyl)-O-(C₁-C₃ alkyldiyl)-, C₂-C₆ alkenyldiyl and C₂-C₆ alkynyldiyl, optionally substituted with one or more groups selected from F, Cl, —OH, —OCH₃, —OCH₂CH₃, —OCH₂CH₂OCH₃, —OCH₂CH₂OH, —OCH₂CH₂N(CH₃)₂;
R⁴ is selected from the group consisting of:
-(C₁-C₁₂ alkyldiyl)—N(R⁵)—*;
-(C₁-C₁₂ alkyldiyl)-(C₂-C₂₀ heterocyclyldiyl)-*;
—O—(C₁-C₁₂ alkyldiyl)—N(R⁵)—*;
—O—(C₁-C₁₂ alkyldiyl)-(C₂-C₂₀ heterocyclyldiyl)-*;
—O—(C₁-C₁₂ alkyldiyl)-(C₂-C₂₀ heterocyclyldiyl)—N(R⁵)—*;
—OC(═O)N(R⁵)—*;
—N(R⁵)—(C₁-C₁₂ alkyldiyl)—N(R⁵)—*;
—N(R⁵)—(C₁-C₁₂ alkyldiyl)-(C₂-C₂₀ heterocyclyldiyl)-*;
—C(═O)N(R⁵)—*;
-(C₂-C₂₀ heterocyclyldiyl)-*;
—S(═O)₂—(C₂-C₂₀ heterocyclyldiyl)-*;
—S(═O)₂—(C₂-C₂₀ heterocyclyldiyl)-(C₁-C₁₂ alkyldiyl)—NR⁵—*;
where the asterisk * indicates the attachment site of L;
R⁵ is independently H or C₁-C₆ alkyl, or two R⁵ groups together form a 5- or 6-membered heterocyclyl ring; and
alkyl, alkyldiyl, alkenyl, alkenyldiyl, alkynyl, alkynyldiyl, aryl, aryldiyl, carbocyclyl, carbocyclyldiyl, heterocyclyl, heterocyclyldiyl, heteroaryl, and heteroaryldiyl are independently and optionally substituted with one or more groups independently selected from F, Cl, Br, I, —CN, —CH₃, —CH₂CH₃, —CH═CH₂, —C≡CH, —C≡CCH₃, —CH₂CH₂CH₃, —CH(CH₃)₂, —CH₂CH(CH₃)₂, —CH₂OH, —CH₂OCH₃, —CH₂CH₂OH, —C(CH₃)₂OH, —CH(OH)CH(CH₃)₂, —C(CH₃)₂CH₂OH, —CH₂CH₂SO₂CH₃, —CH₂OP(O)(OH)₂, —CH₂F, —CHF₂, —CF₃, —CH₂CF₃, —CH₂CHF₂, —CH(CH₃)CN, —C(CH₃)₂CN, —CH₂CN, —CH₂NH₂, —CH₂NHSO₂CH₃, —CH₂NHCH₃, —CH₂N(CH₃)₂, —CO₂H, —COCH₃, —CO₂CH₃, —CO₂C(CH₃)₃, —COCH(OH)CH₃, —CONH₂, —CONHCH₃, —CON(CH₃)₂, —C(CH₃)₂CONH₂, —NH₂, —NHCH₃, —N(CH₃)₂, —NHCOCH₃, —N(CH₃)COCH₃, —NHS(O)₂CH₃, —N(CH₃)C(CH₃)₂CONH₂, —N(CH₃)CH₂CH₂S(O)₂CH₃, — NHC(═NH)H, —NHC(═NH)CH₃, —NHC(═NH)NH₂, —NHC(═O)NH₂, —NO₂, =O, —OH, —OCH₃, —OCH₂CH₃, —OCH₂CH₂OCH₃, —OCH₂CH₂OH, —OCH₂CH₂N(CH₃)₂, —O(CH₂CH₂O)ₙ—(CH₂)ₘCO₂H, —O(CH₂CH₂O)ₙH, —OCH₂F, —OCHF₂, —OCF₃, —OP(O)(OH)₂, —S(O)₂N(CH₃)₂, —SCH₃, —S(O)₂CH₃, and —S(O)₃H.

19. The immunoconjugate of claim 18 wherein X^{a} and X^{b} are independently selected from the group consisting of imidazolyl, pyrazolyl, triazolyl, tetrazolyl, furyl, thienyl, isoxazolyl, thiazolyl, oxadiazolyl, oxazolyl, isothiazolyl, pyrrolyl, oxadiazolyl, and thiadiazolyl.

20. The immunoconjugate of claim 19 wherein X^{a} and X^{b} are each pyrazolyl, substituted with one or more C₁-C₁₂ alkyl groups.

21. The immunoconjugate of claim 18 wherein R¹ is selected from the group consisting of —OCH₃, —OCH₂CH₃, —OCH₂CH₂OCH₃, —OCH₂CH₂OH, and -OCH₂CH₂N(CH₃)₂.

22. The immunoconjugate of claim 21 wherein R¹ is —OCH₃ or F.

23. (canceled)

24. The immunoconjugate of claim 18 wherein R^{2a} and R^{2b} are each —C(═O)NH₂.

25. The immunoconjugate of claim 18 wherein R³ is selected from —CH₂CH₂—, —CH═CH—, and —C═C—.

26. The immunoconjugate of claim 18 wherein R³ is C₂-C₄ alkenyldiyl, substituted with one or more groups selected from F, —OH, and —OCH3.

27. The immunoconjugate of claim 18 wherein R⁴ is —O—(C₁-C₁₂ alkyldiyl)-(C₂-C₂₀ heterocyclyldiyl)-*.

28. The immunoconjugate of claim 27 wherein C₁-C₁₂ alkyldiyl is propyldiyl and C₂-C₂₀ heterocyclyldiyl is piperidiyl.

29. The immunoconjugate of claim 13 wherein L is selected from the group consisting of:
—C(═O)—(PEG)—C(═O)—(EsPEP)—;
—C(═O)—(PEG)—C(═O)—(EsPEP)—N(R⁶)—;
—C(═O)—(PEG)—C(═O)—(EsPEP)—N(R⁶)—(C₁-C₁₂ alkyldiyl)-;
—C(═O)—(PEG)—C(═O)—(EsPEP)—N(R⁶)—(C₁-C₁₂ alkyldiyl)—N(R⁶)C(═O)—(C₂-C₅ monoheterocyclyldiyl)-;
—C(═O)—(PEG)—N(R⁶)—(PEG)—C(═O)—(EsPEP)—;
—C(═O)—(PEG)—N⁺(R⁶)₂—(PEG)—C(═O)—(EsPEP)—;
—C(═O)—(PEG)—C(═O)—N(R⁶)CH(AA₁)C(═O)—(PEG)—C(═O)—(EsPEP)—;
—C(═O)—(C₁-C₁₂ alkyldiyl)—C(═O)—(EsPEP)—;
—C(═O)—(C₁-C₁₂ alkyldiyl)—C(═O)—(EsPEP)—N(R⁶)—(C₁-C₁₂ alkyldiyl)-;
—C(═O)—(C₁-C₁₂ alkyldiyl)—C(═O)—(EsPEP)—N(R⁶)—(C₁-C₁₂ alkyldiyl)—N(R⁶)—C(═O);
—C(═O)—(C₁-C₁₂ alkyldiyl)—C(═O)—(EsPEP)—N(R⁶)—(C₁-C₁₂ alkyldiyl)—N(R⁶)C(═O)—(C₂-C₅ monoheterocyclyldiyl)-;
-succinimidyl—(CH₂)ₘ—C(═O)N(R⁶)—PEG—C(═O)—(EsPEP)—;
-succinimidyl—(CH₂)ₘ—C(═O)—(EsPEP)—N(R⁶)—(C₁-C₁₂ alkyldiyl)-;
-succinimidyl—(CH₂)ₘ—C(═O)—(EsPEP)—N(R⁶)—(C₁-C₁₂ alkyldiyl)N(R⁶)C(═O)—; and
-(succinimidyl)—(CH₂)ₘ—C(═O)—(EsPEP)—N(R⁶)—(C₁-C₁₂alkyldiyl)—N(R⁶)C(═O)—(C₂-C₅ monoheterocyclyldiyl)-;
PEG has the formula: —(CH₂CH₂O)ₙ—(CH₂)ₘ—; m is an integer from 1 to 5, and n is an integer from 2 to 50;
EsPEP is the elastase-substrate, peptide linker unit comprising 2 to 12 amino acid residues; and
R⁶ is selected from the group consisting of H, C₆-C₂₀ aryl, C₆-C₂₀ aryldiyl, C₁-C₁₂ alkyl, and C₁-C₁₂ alkyldiyl, or two R⁶ groups together form a 5- or 6-membered heterocyclyl ring;
alkyl, alkyldiyl, alkenyl, alkenyldiyl, alkynyl, alkynyldiyl, aryl, aryldiyl, carbocyclyl, carbocyclyldiyl, heterocyclyl, heterocyclyldiyl, heteroaryl, and heteroaryldiyl are independently and optionally substituted with one or more groups independently selected from F, Cl, Br, I, —CN, —CH₃, —CH₂CH₃, —CH═CH₂, —C≡CH, —C≡CCH₃, —CH₂CH₂CH₃, —CH(CH₃)₂, —CH₂CH(CH₃)₂, —CH₂OH, —CH₂OCH₃, —CH₂CH₂OH, —C(CH₃)₂OH, —CH(OH)CH(CH₃)₂, —C(CH₃)₂CH₂OH, —CH₂CH₂SO₂CH₃, —CH₂OP(O)(OH)₂, —CH₂F, —CHF₂, —CF₃, —CH₂CF₃, —CH₂CHF₂, —CH(CH₃)CN, —C(CH₃)₂CN, —CH₂CN, —CH₂NH₂, —CH₂NHSO₂CH₃, —CH₂NHCH₃, —CH₂N(CH₃)₂, —CO₂H, —COCH₃, —CO₂CH₃, —CO₂C(CH₃)₃, —COCH(OH)CH₃, —CONH₂, —CONHCH₃, —CON(CH₃)₂, —C(CH₃)₂CONH₂, —NH₂, —NHCH₃, —N(CH₃)₂, —NHCOCH₃, —N(CH₃)COCH₃, —NHS(O)₂CH₃, —N(CH₃)C(CH₃)₂CONH₂, —N(CH₃)CH₂CH₂S(O)₂CH₃, — NHC(═NH)H, —NHC(═NH)CH₃, —NHC(═NH)NH₂, —NHC(═O)NH₂, —NO₂, =O, —OH, —OCH₃, —OCH₂CH₃, —OCH₂CH₂OCH₃, —OCH₂CH₂OH, —OCH₂CH₂N(CH₃)₂, —O(CH₂CH₂O)ₙ—(CH₂)ₘCO₂H, —O(CH₂CH₂O)ₙH, —OP(O)(OH)₂, —S(O)₂N(CH₃)₂, —SCH₃, —S(O)₂CH₃, and —S(O)₃H.

30. The immunoconjugate of claim 29 wherein EsPEP is comprised of one or more unnatural amino acid residues.

31. The immunoconjugate of claim 29 wherein EsPEP is comprised of amino acid residues of amino acids selected from the group consisting of: 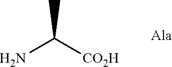 [SMILES: N[C@@H](Cc1ccc(C(=O)c2ccccc2)cc1)C(=O)O] 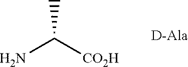 [SMILES: N[C@@H](CCCCOCc1ccccc1)C(=O)O] 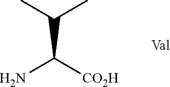 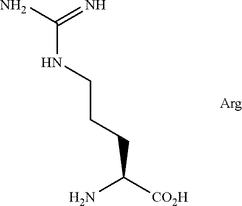 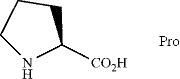 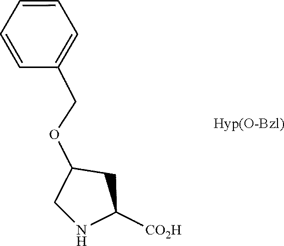 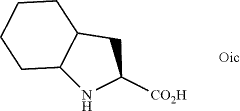 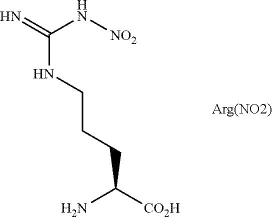 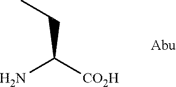 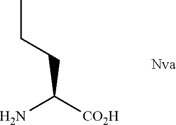 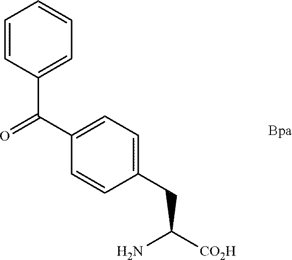 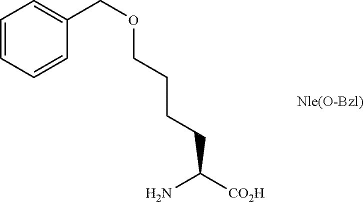 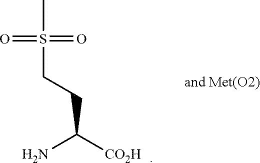
.

32. The immunoconjugate of claim 29 wherein EsPEP is selected from the group consisting of Ala—Pro—Val, Asn—Pro—Val, Ala—Ala—Val, Ala—Ala—Pro—Ala (SEQ ID NO— 639), Ala—Ala—Pro—Val (SEQ ID NO— 640), and Ala—Ala—Pro—Nva (SEQ ID NO: 641).

33. The immunoconjugate of claim 29 wherein EsPEP has the formula: 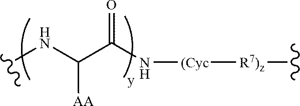
where AA is independently selected from a natural or unnatural amino acid side chain, or one or more of AA, and an adjacent nitrogen atom form a 5-membered ring proline amino acid, and the wavy line indicates a point of attachment;
Cyc is selected from C₆-C₂₀ aryldiyl and C₁-C₂₀ heteroaryldiyl, optionally substituted with one or more groups selected from F, Cl, NO₂, —OH, —OCH₃, and a glucuronic acid having the structure: 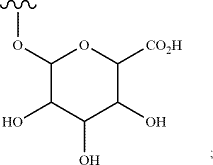
R⁷ is selected from the group consisting of —CH(R⁸)O—, —CH₂—, —CH₂N(R⁸)—, and —CH(R⁸)O—C(═O)—, where R⁸ is selected from H, C₁-C₆ alkyl, C(═O)—C₁-C₆ alkyl, and —C(═O)N(R⁹)₂, where R⁹ is independently selected from the group consisting of H, C₁-C₁₂ alkyl, and —(CH₂CH₂O)ₙ—(CH₂)ₘ—OH, where m is an integer from 1 to 5, and n is an integer from 2 to 50, or two R⁹ groups together form a 5- or 6-membered heterocyclyl ring;
y is an integer from 2 to 12; and
z is 0 or 1.

34. The immunoconjugate of claim 33 wherein y is selected from 2, 3, and 4.

35. The immunoconjugate of claim 29 wherein EsPEP is a tripeptide having the formula: 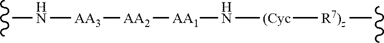
where AA₁, AA₂ and AA₃ are independently selected from a natural or unnatural amino acid, and the wavy line indicates a point of attachment;
Cyc is selected from C₆-C₂₀ aryldiyl and C₁-C₂₀ heteroaryldiyl, optionally substituted with one or more groups selected from F, Cl, NO₂, —OH, —OCH₃, and a glucuronic acid having the structure: 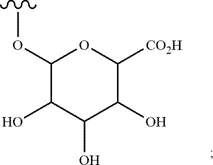
R⁷ is selected from the group consisting of —CH(R⁸)O—, —CH₂—, —CH₂N(R⁸)—, and —CH(R⁸)O—C(═O)—, where R⁸ is selected from H, C₁-C₆ alkyl, C(═O)—C₁-C₆ alkyl, and —C(═O)N(R⁹)₂, where R⁹ is independently selected from the group consisting of H, C₁-C₁₂ alkyl, and —(CH₂CH₂O)ₙ—(CH₂)ₘ—OH, where m is an integer from 1 to 5, and n is an integer from 2 to 50, or two R⁹ groups together form a 5- or 6-membered heterocyclyl ring; and
z is 0 or 1.

36. The immunoconjugate of claim 35 wherein AA₁ is methyl, AA₂ forms proline, and AA₃ is isopropyl.

37. The immunoconjugate of claim 35 wherein EsPEP has the formula: 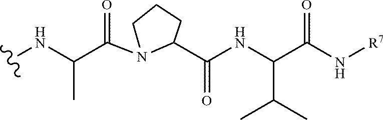
.

38. The immunoconjugate of claim 37 wherein EsPEP is selected from the formulas: 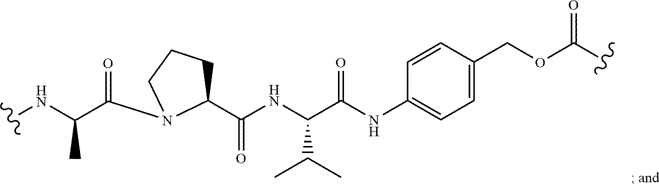 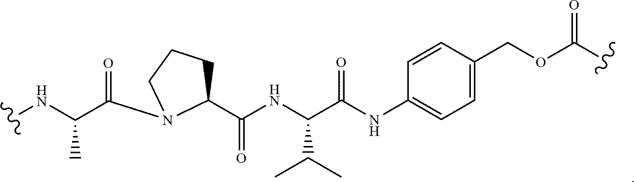
.

39. The immunoconjugate of claim 29 wherein L is: —C(═O)—(PEG)—C(═O)—(EsPEP)— .

40. The immunoconjugate of claim 39 wherein PEG is: —(CH₂CH₂O)₂₅—(CH₂)₂— or —(CH₂CH₂O)₁₀—(CH₂)₂— .

41. The immunoconjugate of claim 40 selected from the formulas: 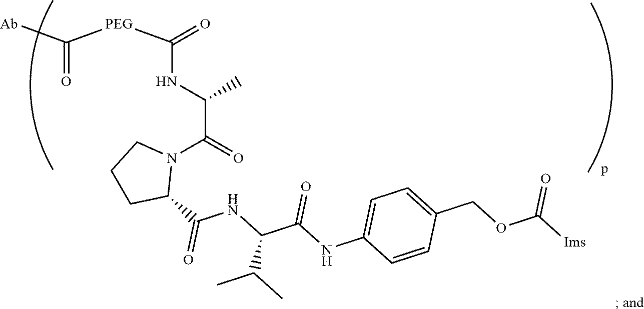 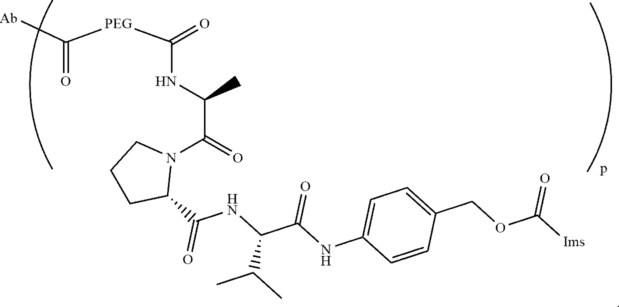
.

42. The immunoconjugate of claim 41 wherein Ims has the structure: 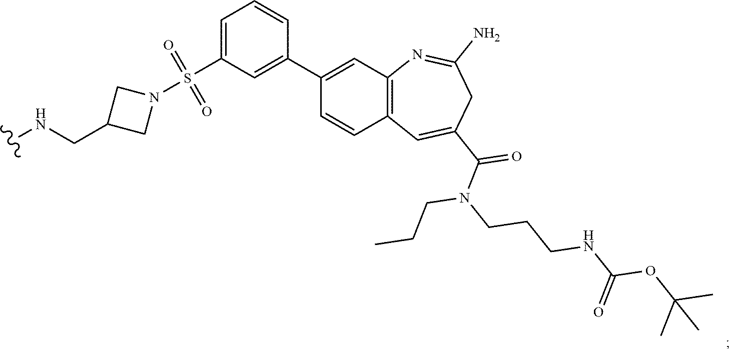
and the wavy line indicates the site of attachment.

43. The immunoconjugate of claim 29 wherein EsPEP is a tetrapeptide having the formula: 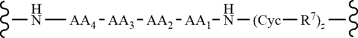
where AA₁, AA₂, AA₃ and AA₄ are independently selected from a natural or unnatural amino acid, and the wavy line indicates a point of attachment;
Cyc is selected from C₆-C₂₀ aryldiyl and C₁-C₂₀ heteroaryldiyl, optionally substituted with one or more groups selected from F, Cl, NO₂, —OH, —OCH₃, and a glucuronic acid having the structure: 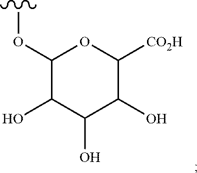
R⁷ is selected from the group consisting of —CH(R⁸)O—, —CH₂—, —CH₂N(R⁸)—, and —CH(R⁸)O—C(═O)—, where R⁸ is selected from H, C₁-C₆ alkyl, C(═O)—C₁-C₆ alkyl, and —C(═O)N(R⁹)₂, where R⁹ is independently selected from the group consisting of H, C₁-C₁₂ alkyl, and —(CH₂CH₂O)ₙ—(CH₂)ₘ—OH, where m is an integer from 1 to 5, and n is an integer from 2 to 50, or two R⁹ groups together form a 5- or 6-membered heterocyclyl ring; and
z is 0 or 1.

44. The immunoconjugate of claim 43 wherein
AA₁ is selected from the group consisting of Abu, Ala, and Val;
AA₂ is selected from the group consisting of Nle(O-Bzl), Oic and Pro;
AA₃ is selected from the group consisting of Ala and Met(O)₂; and
AA₄ is selected from the group consisting of Oic, Arg(NO₂), Bpa, and Nle(O-Bzl).

45. The immunoconjugate of claim 44 wherein EsPEP has the formula: 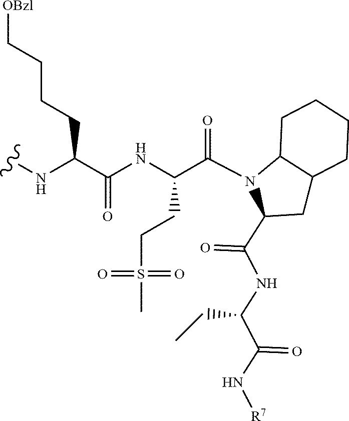
.

46. The immunoconjugate of claim 45 wherein EsPEP has the formula: 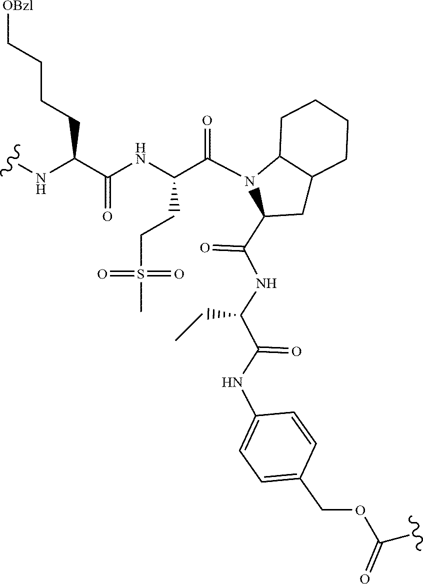
.

47. The immunoconjugate of claim 29 comprising a structure selected from IIe-h: 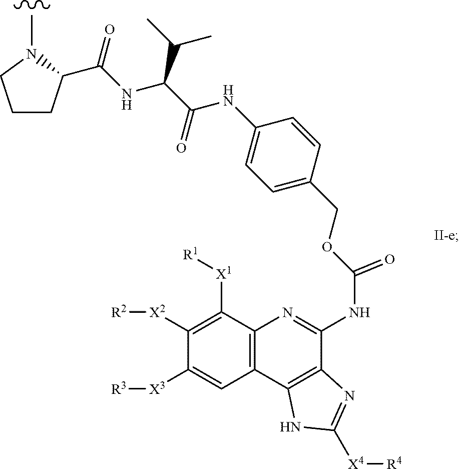 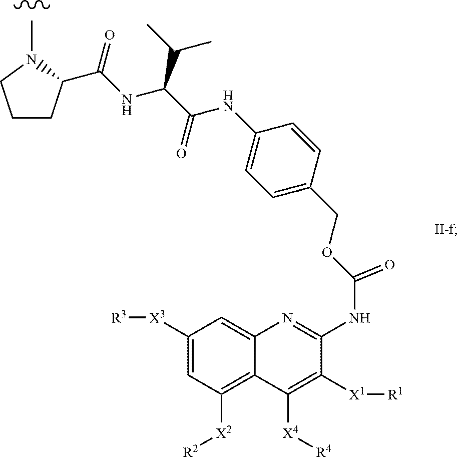 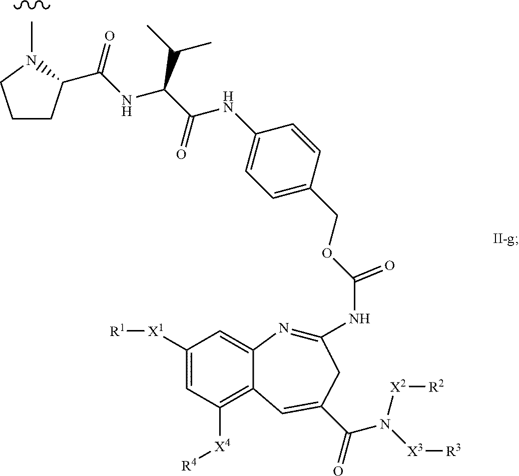 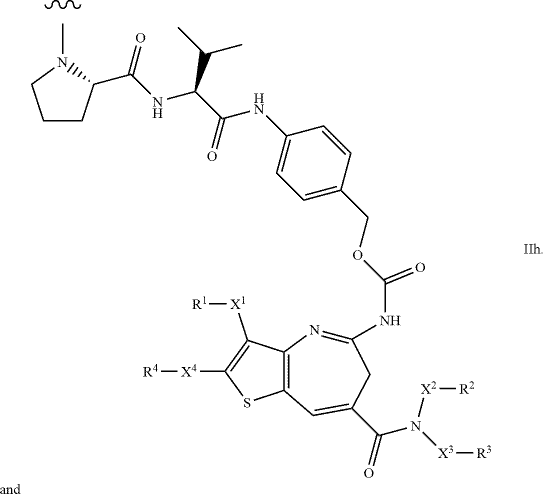
where the wavy line indicates the attachment through L to the antibody.

48. The immunoconjugate of claim 29 comprising a structure selected from IIi-1: 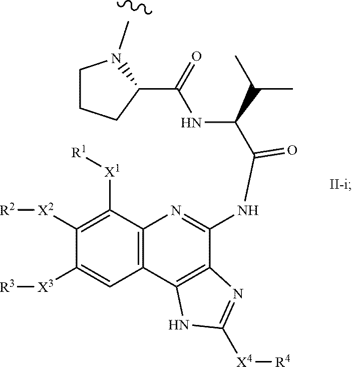 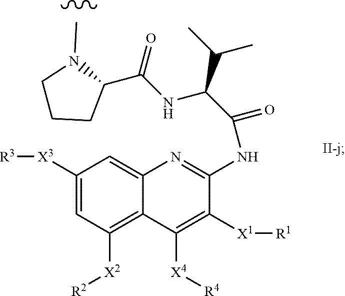 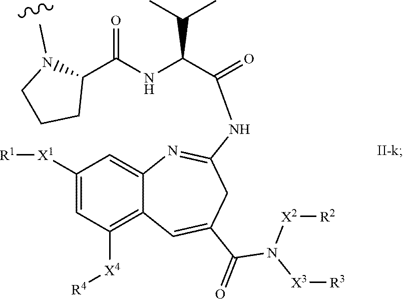 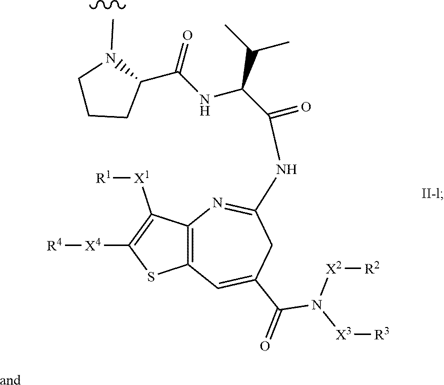
where the wavy line indicates the attachment through L to the antibody.

49. The immunoconjugate of claim 48 wherein R² and R³ are each C₁-C₈ alkyl.

50. The immunoconjugate of claim 49 wherein R² and R³ are each -CH₂CH₂CH₃.

51. The immunoconjugate of claim 48 wherein X² and X³ are each a bond, and R² or R³ is —O—(C₁-C₁₂ alkyl).

52. The immunoconjugate of claim 51 wherein R² or R³ is —OCH₂CH₃.

53. The immunoconjugate of claim 1 wherein the elastase-substrate, peptide linker is cleaved by elastase.

54. An immunostimulant-elastase substrate, peptide linker compound selected from formulas IIa-f: 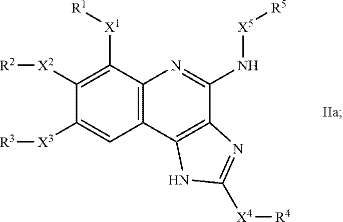 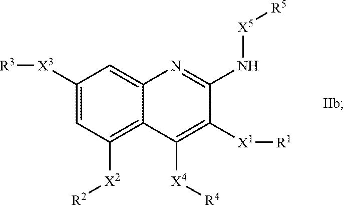 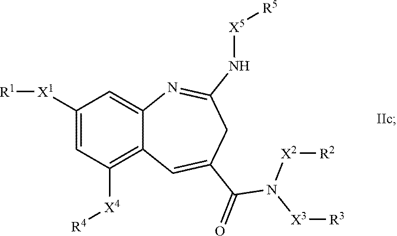 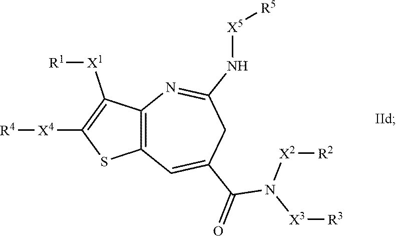 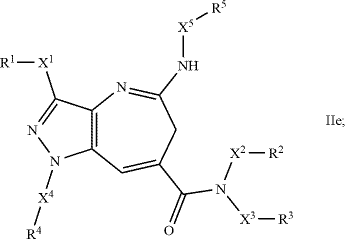 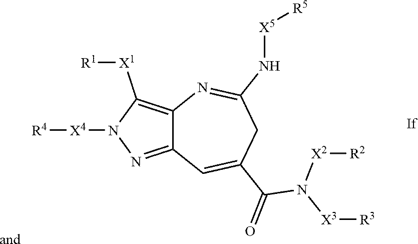
wherein R¹, R², R³, R⁴, and R⁵ are independently selected from the group consisting of H, C₁-C₁₂ alkyl, C₂-C₆ alkenyl, C₂-C₆ alkynyl, C₃-C₁₂ carbocyclyl, C₆-C₂₀ aryl, C₂-C₉ heterocyclyl, and C₁-C₂₀ heteroaryl, where alkyl, alkenyl, alkynyl, carbocyclyl, aryl, heterocyclyl, and heteroaryl are independently and optionally substituted with one or more groups selected from:
-(C₁-C₁₂ alkyldiyl)—N(R⁶)—*;
-(C₁-C₁₂ alkyldiyl)-N(R⁶)₂;
-(C₁-C₁₂ alkyldiyl)-OR⁶;
-(C₃-C₁₂ carbocyclyl);
-(C₃-C₁₂ carbocyclyl)-*;
-(C₃-C₁₂ carbocyclyl)-(C₁-C₁₂ alkyldiyl)—NR⁶—*;
-(C₃-C₁₂ carbocyclyl)-(C₁-C₁₂ alkyldiyl)-N(R⁶)₂;
-(C₃-C₁₂ carbocyclyl)-NR⁵-C(=NR^{6a})NR⁶-*;
-(C₆-C₂₀ aryl);
-(C₆-C₂₀ aryldiyl)-*;
-(C₆-C₂₀ aryldiyl)—N(R⁶)—*;
-(C₆-C₂₀ aryldiyl)-(C₁-C₁₂ alkyldiyl)—N(R⁶)—*;
-(C₆-C₂₀ aryldiyl)-(C₁-C₁₂ alkyldiyl)-(C₂-C₂₀ heterocyclyldiyl)-*;
-(C₆-C₂₀ aryldiyl)-(C₁-C₁₂ alkyldiyl)-N(R⁶)₂;
-(C₆-C₂₀ aryldiyl)-(C₁-C₁₂ alkyldiyl)-NR⁶-C(=NR^{6a})N(R⁶)-*;
-(C₂-C₂₀ heterocyclyl);
-(C₂-C₂₀ heterocyclyl)-*;
-(C₂-C₉ heterocyclyl)-(C₁-C₁₂ alkyldiyl)—NR⁶—*;
-(C₂-C₉ heterocyclyl)-(C₁-C₁₂ alkyldiyl)-N(R⁶)₂;
-(C₂-C₉ heterocyclyl)—C(═O)—(C₁-C₁₂ alkyldiyl)—N(R⁶)—*;
-(C₂-C₉ heterocyclyl)-NR⁵-C(=NR^{6a})NR⁶-*;
-(C₂-C₉ heterocyclyl)-NR⁶-(C₆-C₂₀ aryldiyl)-(C₁-C₁₂ alkyldiyl)—N(R⁶)—*;
-(C₂-C₉ heterocyclyl)-(C₆-C₂₀ aryldiyl)-*;
-(C₁-C₂₀ heteroaryl);
-(C₁-C₂₀ heteroaryldiyl)-*;
-(C₁-C₂₀ heteroaryldiyl)-(C₁-C₁₂ alkyldiyl)—N(R⁶)—*;
-(C₁-C₂₀ heteroaryldiyl)-(C₁-C₁₂ alkyldiyl)-N(R⁶)₂;
-(C₁-C₂₀ heteroaryldiyl)-NR⁶-C(=NR^{6a})N(R⁶)-*;
-(C₁-C₂₀ heteroaryldiyl)—N(R⁶)C(═O)—(C₁-C₁₂ alkyldiyl)—N(R⁶)—*;
—C(═O)—*;
—C(═O)—(C₁-C₁₂ alkyldiyl)—N(R⁶)—*;
—C(═O)—(C₂-C₂₀ heterocyclyldiyl)-*;
—C(═O)N(R⁶)₂;
—C(═O)N(R⁶)—*;
—C(═O)N(R⁶)—(C₁-C₁₂ alkyldiyl)—N(R⁶)C(═O)R⁵;
—C(═O)N(R⁶)—(C₁-C₁₂ alkyldiyl)—N(R⁶)C(═O)N(R⁶)₂;
—C(═O)NR⁶—(C₁-C₁₂ alkyldiyl)-N(R⁶)CO₂R⁶;
—C(═O)NR⁶—(C₁-C₁₂ alkyldiyl)-N(R⁶)C(=NR^{6a})N(R⁶)₂;
—C(═O)NR⁶—(C₁-C₁₂ alkyldiyl)-NR⁶C(=NR^{6a})R⁶;
—C(═O)NR⁶—(C₁-C₈ alkyldiyl)-NR⁶(C₂-C₅ heteroaryl);
—C(═O)NR⁶—(C₁-C₂₀ heteroaryldiyl)—N(R⁶)—*;
—C(═O)NR⁶—(C₁-C₂₀ heteroaryldiyl)-*;
—C(═O)NR⁶—(C₁-C₂₀ heteroaryldiyl)-(C₁-C₁₂ alkyldiyl)-N(R⁶)₂;
—C(═O)NR⁶—(C₁-C₂₀ heteroaryldiyl)-(C₂-C₂₀ heterocyclyldiyl)—C(═O)NR⁶—(C₁-C₁₂ alkyldiyl)—NR⁶—*;
—N(R⁶)₂;
—N(R⁶)—*;
—N(R⁶)C(═O)R⁶;
—N(R⁶)C(═O)—*;
—N(R⁶)C(═O)N(R⁶)₂;
—N(R⁶)C(═O)N(R⁶)—*;
—N(R⁶)CO₂R⁶;
—N(R⁶)CO₂(R⁶)—*;
-NR⁶C(=NR^{6a})N(R⁶)₂;
-NR⁶C(=NR^{6a})N(R⁶)-*;
-NR⁶C(=NR^{6a})R⁶;
—N(R⁶)C(═O)—(C₁-C₁₂ alkyldiyl)—N(R⁶)—*;
—N(R⁶)—(C₂-C₅ heteroaryl);
—N(R⁶)—S(═O)₂—(C₁-C₁₂ alkyl);
—O—(C₁-C₁₂ alkyl);
—O—(C₁-C₁₂ alkyldiyl)-N(R⁶)₂;
—O—(C₁-C₁₂ alkyldiyl)—N(R⁶)—*;
—OC(═O)N(R⁶)₂;
—OC(═O)N(R⁶)—*;
—S(═O)₂—(C₂-C₂₀ heterocyclyldiyl)-*;
—S(═O)₂—(C₂-C₂₀ heterocyclyldiyl)-(C₁-C₁₂ alkyldiyl)-N(R⁶)₂;
—S(═O)₂—(C₂-C₂₀ heterocyclyldiyl)-(C₁-C₁₂ alkyldiyl)—NR⁶—*; and
—S(═O)₂—(C₂-C₂₀ heterocyclyldiyl)-(C₁-C₁₂ alkyldiyl)-OH;
or R² and R³ of formulas Ic or Id together form a 5- or 6-membered heterocyclyl ring;
X¹, X², X³, X⁴, and X⁵ are independently selected from the group consisting of a bond, C(═O), C(═O)N(R⁶), O, N(R⁶), S, S(O)₂, and S(O)₂N(R⁶);
R⁶ is selected from the group consisting of H, C₆-C₂₀ aryl, C₆-C₂₀ aryldiyl, C₁-C₁₂ alkyl, and C₁-C₁₂ alkyldiyl, or two R⁶ groups together form a 5- or 6-membered heterocyclyl ring;
R^{6a} is selected from the group consisting of C₆-C₂₀ aryl and C₁-C₂₀ heteroaryl;
where the asterisk * indicates the attachment site of L, and where one of R¹, R², R³, R⁴ and R⁵ is attached to L;
L is selected from the group consisting of:
Q—C(═O)—(PEG)—C(═O)—(EsPEP)—;
Q—C(═O)—(PEG)—C(═O)—(EsPEP)—N(R⁶)—(C₁-C₁₂ alkyldiyl)-;
Q—C(═O)—(PEG)—C(═O)—(EsPEP)—N(R⁶)—(C₁-C₁₂ alkyldiyl)—N(R⁶)C(═O)—(C₂-C₅ monoheterocyclyldiyl)-;
Q—C(═O)—(PEG)—N(R⁶)—(PEG)—C(═O)—(EsPEP)—;
Q—C(═O)—(PEG)—N⁺(R⁶)₂—(PEG)—C(═O)—(EsPEP)—;
Q—C(═O)—(PEG)—C(═O)—N(R⁶)CH(AA₁)C(═O)—(PEG)—C(═O)—(EsPEP)—;
Q—C(═O)—(C₁-C₁₂ alkyldiyl)—C(═O)—(EsPEP)—;
Q—C(═O)—(C₁-C₁₂ alkyldiyl)—C(═O)—(EsPEP)—N(R⁶)—(C₁-C₁₂ alkyldiyl)-;
Q—C(═O)—(C₁-C₁₂ alkyldiyl)—C(═O)—(EsPEP)—N(R⁶)—(C₁-C₁₂ alkyldiyl)—N(R⁶)—C(═O); Q—C(═O)—(C₁-C₁₂ alkyldiyl)—C(═O)—(EsPEP)—N(R⁶)—(C₁-C₁₂
alkyldiyl)—N(R⁶)C(═O)—(C₂-C₅ monoheterocyclyldiyl)-;
Q—(CH₂)ₘ—C(═O)N(R⁶)—PEG—C(═O)—(EsPEP)—;
Q—(CH₂)ₘ—C(═O)—(EsPEP)—N(R⁶)—(C₁-C₁₂ alkyldiyl)-;
Q—(CH₂)ₘ—C(═O)—(EsPEP)—N(R⁶)—(C₁-C₁₂ alkyldiyl)N(R⁶)C(═O)—; and
Q—(CH₂)ₘ—C(═O)—(EsPEP)—N(R⁶)—(C₁-C₁₂ alkyldiyl)—N(R⁶)C(═O)—(C₂-C₅ monoheterocyclyldiyl)-;
PEG has the formula: —(CH₂CH₂O)ₙ—(CH₂)ₘ—; m is an integer from 1 to 5, and n is an integer from 2 to 50;
EsPEP is an elastase-substrate, peptide linker unit comprising 2 to 12 amino acid residues; and
Q is selected from the group consisting of N-hydroxysuccinimidyl, N-hydroxysulfosuccinimidyl, maleimide, and phenoxy substituted with one or more groups independently selected from F, Cl, NO₂, and SO₃⁻;
alkyl, alkyldiyl, alkenyl, alkenyldiyl, alkynyl, alkynyldiyl, aryl, aryldiyl, carbocyclyl, carbocyclyldiyl, heterocyclyl, heterocyclyldiyl, heteroaryl, and heteroaryldiyl are independently and optionally substituted with one or more groups independently selected from F, Cl, Br, I, —CN, —CH₃, —CH₂CH₃, —CH═CH₂, —C═CH, —C≡CCH₃, —CH₂CH₂CH₃, —CH(CH₃)₂, —CH₂CH(CH₃)₂, —CH₂OH, —CH₂OCH₃, —CH₂CH₂OH, —C(CH₃)₂OH, —CH(OH)CH(CH₃)₂, —C(CH₃)₂CH₂OH, —CH₂CH₂SO₂CH₃, —CH₂OP(O)(OH)₂, —CH₂F, —CHF₂, —CF₃, —CH₂CF₃, —CH₂CHF₂, —CH(CH₃)CN, —C(CH₃)₂CN, —CH₂CN, —CH₂NH₂, —CH₂NHSO₂CH₃, —CH₂NHCH₃, —CH₂N(CH₃)₂, —CO₂H, —COCH₃, —CO₂CH₃, —CO₂C(CH₃)₃, —COCH(OH)CH₃, —CONH₂, —CONHCH₃, —CON(CH₃)₂, —C(CH₃)₂CONH₂, —NH₂, —NHCH₃, —N(CH₃)₂, —NHCOCH₃, —N(CH₃)COCH₃, —NHS(O)₂CH₃, —N(CH₃)C(CH₃)₂CONH₂, —N(CH₃)CH₂CH₂S(O)₂CH₃, — NHC(═NH)H, —NHC(═NH)CH₃, —NHC(═NH)NH₂, —NHC(═O)NH₂, —NO₂, ═O, —OH, —OCH3, —OCH₂CH₃, —OCH₂CH₂OCH₃, —OCH₂CH₂OH, —OCH₂CH₂N(CH₃)₂, —O(CH₂CH₂O)ₙ—(CH₂)ₘCO2H, —O(CH₂CH₂O)ₙH, —OP(O)(OH)₂, —S(O)₂N(CH₃)₂, —SCH₃, —S(O)₂CH₃, and —S(O)₃H.

55. The immunostimulant-elastase substrate, peptide linker compound of claim 54 wherein PEG has the formula: —(CH₂CH₂O)₂₅—(CH₂)₂— or —(CH₂CH₂O)₁₀—(CH₂)₂—.

56. The immunostimulant-elastase substrate, peptide linker compound of claim 54 wherein R² and R³ are each C₁-C₈ alkyl.

57. The immunostimulant-elastase substrate, peptide linker compound of claim 56 wherein R² and R³ are each —CH₂CH₂CH₃.

58. The immunostimulant-elastase substrate, peptide linker compound of claim 54 wherein X² and X³ are each a bond, and R² or R³ is —O—(C₁-C₁₂ alkyl).

59. The immunostimulant-elastase substrate, peptide linker compound of claim 58 wherein R² or R³ is —OCH₂CH₃.

60. The immunostimulant-elastase substrate, peptide linker compound of claim 54 is selected from the formulas: 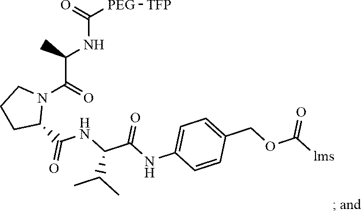 [SMILES: CCn1nc(C)cc1C(=O)/N=c1\[nH]c2cc(C(N)=O)cc(OC)c2n1C/C=C/Cn1/c(=N/C(=O)c2cc(C)nn2CC)[nH]c2cc(C(N)=O)cc(OCCCN3CCN(C(=O)OCc4ccc(NC(=O)[C@@H](NC(=O)[C@@H]5CCCN5C(=O)[C@H](C)NC(=O)CCOCCOCCOCCOCCOCCOCCOCCOCCOCCOCCNC(=O)CN5C(=O)C=CC5=O)C(C)C)cc4)CC3)c21] 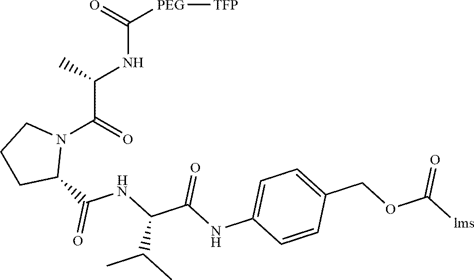
where TFP is 2,3,5,6-tetrafluorophenoxy.

61. The immunostimulant-elastase substrate, peptide linker compound of claim 54 comprising a structure selected from IIe-h: 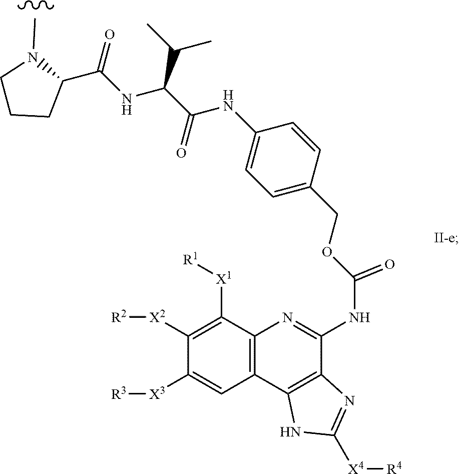 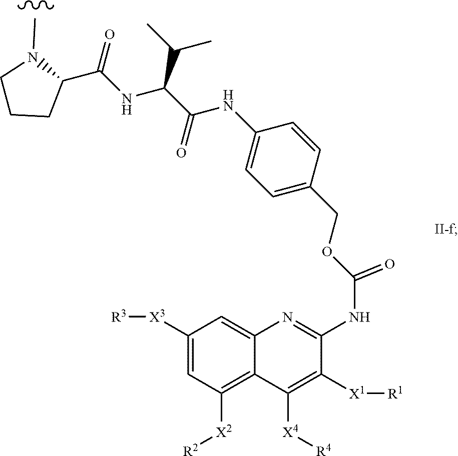 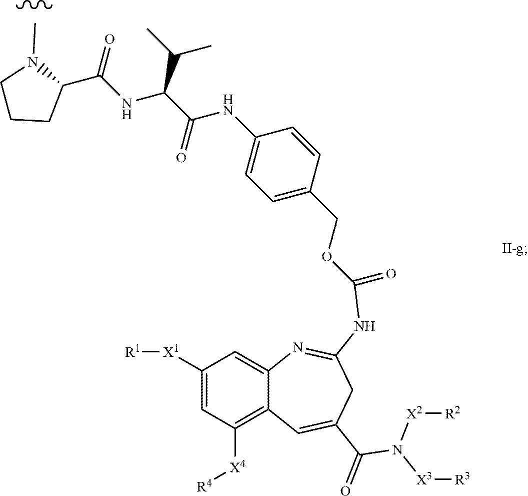 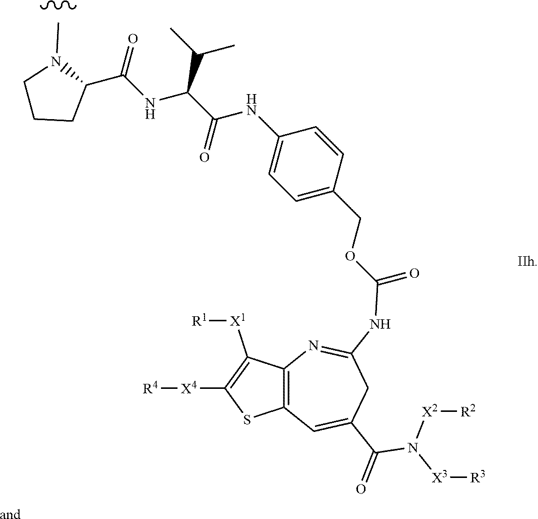
where the wavy line indicates the attachment through L to the antibody.

62. The immunostimulant-elastase substrate, peptide linker compound of claim 54 comprising a structure selected from IIi-1: 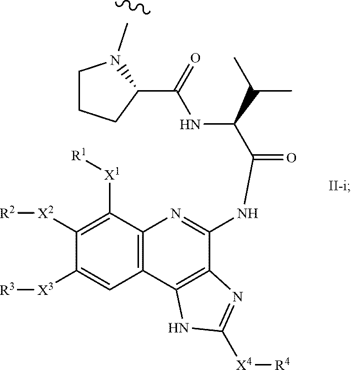 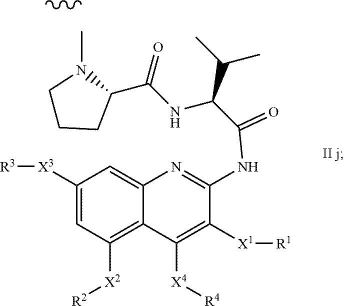 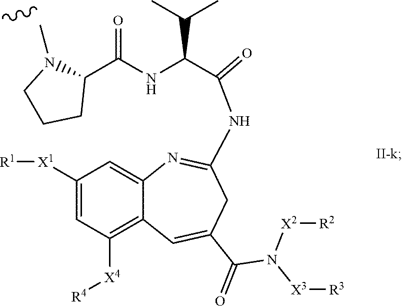 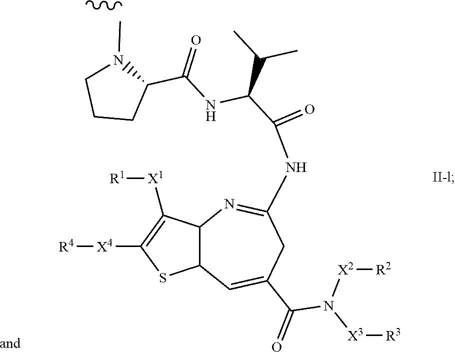
where the wavy line indicates the attachment through L to the antibody.

63. An immunoconjugate prepared by conjugation of a cell-binding agent with an immunostimulant-elastase substrate, peptide linker compound of claim 54.

64. An immunoconjugate prepared by conjugation of a cell-binding agent with an immunostimulant-elastase substrate, peptide linker compound having the structure: 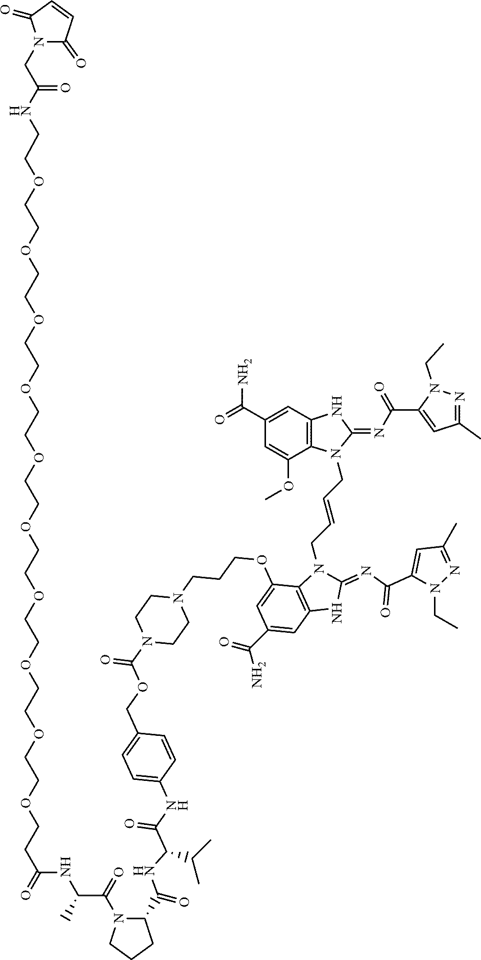
wherein the cell-binding agent is an antibody.

65. (canceled)

66. A pharmaceutical composition comprising a therapeutically effective amount of an immunoconjugate claim 1 and one or more pharmaceutically acceptable diluent, vehicle, carrier or excipient.

67. (canceled)

68. A method of treatment comprising administering a therapeutically-effective dose of the pharmaceutical composition of claim 66 to a patient with an immune-related disorder.

69. The method of claim 68 wherein the elastase-substrate, peptide linker of the immunoconjugate is cleaved by elastase.

70. (canceled)

71. A method for treating cancer comprising administering a therapeutically effective amount of the pharmaceutical composition according to claim 66 to a patient in need thereof wherein the cancer is selected from bladder cancer, urinary tract cancer, urothelial carcinoma, lung cancer, non-small cell lung cancer, Merkel cell carcinoma, colon cancer, colorectal cancer, gastric cancer, and breast cancer, and is susceptible to a pro-inflammatory response induced by TLR7 and/or TLR8 agonism, or by STING agonism.

72. (canceled)

73. (canceled)

74. The method of claim 71, wherein the cancer is selected from a PD-L1-expressing cancer, a HER2-expressing cancer, a CEA-expressing cancer, and a TROP2-expressing cancer.

75-83. (canceled)


SEQUENCE LISTING (641 sequences)

SEQ ID NO: 1 - protein, 5 aa, Artificial Sequence
  source: /note="Description of Artificial Sequence: Synthetic peptide"
SYAIS

SEQ ID NO: 2 - protein, 5 aa, Artificial Sequence
  source: /note="Description of Artificial Sequence: Synthetic peptide"
SYYMH

SEQ ID NO: 3 - protein, 5 aa, Artificial Sequence
  source: /note="Description of Artificial Sequence: Synthetic peptide"
RHLLH

SEQ ID NO: 4 - protein, 5 aa, Artificial Sequence
  source: /note="Description of Artificial Sequence: Synthetic peptide"
SHHMH

SEQ ID NO: 5 - protein, 4 aa, Artificial Sequence
  source: /note="Description of Artificial Sequence: Synthetic peptide"
RFMH

SEQ ID NO: 6 - protein, 5 aa, Artificial Sequence
  source: /note="Description of Artificial Sequence: Synthetic peptide"
SYYIH

SEQ ID NO: 7 - protein, 5 aa, Artificial Sequence
  source: /note="Description of Artificial Sequence: Synthetic peptide"
NYYIH

SEQ ID NO: 8 - protein, 5 aa, Artificial Sequence
  source: /note="Description of Artificial Sequence: Synthetic peptide"
HYYMH

SEQ ID NO: 9 - protein, 5 aa, Artificial Sequence
  source: /note="Description of Artificial Sequence: Synthetic peptide"
GYYMH

SEQ ID NO: 10 - protein, 5 aa, Artificial Sequence
  source: /note="Description of Artificial Sequence: Synthetic peptide"
NYMYH

SEQ ID NO: 11 - protein, 5 aa, Artificial Sequence
  source: /note="Description of Artificial Sequence: Synthetic peptide"
TYYVH

SEQ ID NO: 12 - protein, 5 aa, Artificial Sequence
  source: /note="Description of Artificial Sequence: Synthetic peptide"
SYALS

SEQ ID NO: 13 - protein, 5 aa, Artificial Sequence
  source: /note="Description of Artificial Sequence: Synthetic peptide"
RHYVH

SEQ ID NO: 14 - protein, 5 aa, Artificial Sequence
  source: /note="Description of Artificial Sequence: Synthetic peptide"
SHYMH

SEQ ID NO: 15 - protein, 5 aa, Artificial Sequence
  source: /note="Description of Artificial Sequence: Synthetic peptide"
DYYMH

SEQ ID NO: 16 - protein, 5 aa, Artificial Sequence
  source: /note="Description of Artificial Sequence: Synthetic peptide"
GYTLH

SEQ ID NO: 17 - protein, 5 aa, Artificial Sequence
  source: /note="Description of Artificial Sequence: Synthetic peptide"
NHYMH

SEQ ID NO: 18 - protein, 5 aa, Artificial Sequence
  source: /note="Description of Artificial Sequence: Synthetic peptide"
NSYMH

SEQ ID NO: 19 - protein, 5 aa, Artificial Sequence
  source: /note="Description of Artificial Sequence: Synthetic peptide"
TYYMH

SEQ ID NO: 20 - protein, 5 aa, Artificial Sequence
  source: /note="Description of Artificial Sequence: Synthetic peptide"
RHFIH

SEQ ID NO: 21 - protein, 5 aa, Artificial Sequence
  source: /note="Description of Artificial Sequence: Synthetic peptide"
TFGIS

SEQ ID NO: 22 - protein, 5 aa, Artificial Sequence
  source: /note="Description of Artificial Sequence: Synthetic peptide"
SYGIN

SEQ ID NO: 23 - protein, 5 aa, Artificial Sequence
  source: /note="Description of Artificial Sequence: Synthetic peptide"
NHYVH

SEQ ID NO: 24 - protein, 17 aa, Artificial Sequence
  source: /note="Description of Artificial Sequence: Synthetic peptide"
VINPSAGSTDYAQKFQG

SEQ ID NO: 25 - protein, 17 aa, Artificial Sequence
  source: /note="Description of Artificial Sequence: Synthetic peptide"
WMNPNSDIAGYAQKFQG

SEQ ID NO: 26 - protein, 17 aa, Artificial Sequence
  source: /note="Description of Artificial Sequence: Synthetic peptide"
WISPQHGVRNYAQKFQG

SEQ ID NO: 27 - protein, 17 aa, Artificial Sequence
  source: /note="Description of Artificial Sequence: Synthetic peptide"
WVSPSHGLTGYAQKFQG

SEQ ID NO: 28 - protein, 17 aa, Artificial Sequence
  source: /note="Description of Artificial Sequence: Synthetic peptide"
WMSLNSGLTGYAQKFQG

SEQ ID NO: 29 - protein, 17 aa, Artificial Sequence
  source: /note="Description of Artificial Sequence: Synthetic peptide"
WMKPSSGTTGYAQKFQG

SEQ ID NO: 30 - protein, 17 aa, Artificial Sequence
  source: /note="Description of Artificial Sequence: Synthetic peptide"
WMNPNGDVAGYAQKFQG

SEQ ID NO: 31 - protein, 17 aa, Artificial Sequence
  source: /note="Description of Artificial Sequence: Synthetic peptide"
GIDPNSGGTNYAQKFQG

SEQ ID NO: 32 - protein, 17 aa, Artificial Sequence
  source: /note="Description of Artificial Sequence: Synthetic peptide"
WMNPDSGSTGYAQKFQG

SEQ ID NO: 33 - protein, 17 aa, Artificial Sequence
  source: /note="Description of Artificial Sequence: Synthetic peptide"
WMSLNSGLTGYAQKFQG

SEQ ID NO: 34 - protein, 17 aa, Artificial Sequence
  source: /note="Description of Artificial Sequence: Synthetic peptide"
WMNPNGDVAGYADSFQG

SEQ ID NO: 35 - protein, 17 aa, Artificial Sequence
  source: /note="Description of Artificial Sequence: Synthetic peptide"
WISTYHGSTNYAQKFQG

SEQ ID NO: 36 - protein, 17 aa, Artificial Sequence
  source: /note="Description of Artificial Sequence: Synthetic peptide"
WMNPNTVYTGSAQKFQG

SEQ ID NO: 37 - protein, 17 aa, Artificial Sequence
  source: /note="Description of Artificial Sequence: Synthetic peptide"
RIIPAVGSVTYAQKFQG

SEQ ID NO: 38 - protein, 17 aa, Artificial Sequence
  source: /note="Description of Artificial Sequence: Synthetic peptide"
GIIPIFGTANYAQKFQG

SEQ ID NO: 39 - protein, 17 aa, Artificial Sequence
  source: /note="Description of Artificial Sequence: Synthetic peptide"
WMSPSSGITGYAQKFQG

SEQ ID NO: 40 - protein, 17 aa, Artificial Sequence
  source: /note="Description of Artificial Sequence: Synthetic peptide"
WMTPSTGNAGYAQKFQG

SEQ ID NO: 41 - protein, 17 aa, Artificial Sequence
  source: /note="Description of Artificial Sequence: Synthetic peptide"
WMNPNSGNTGYAQKFQG

SEQ ID NO: 42 - protein, 17 aa, Artificial Sequence
  source: /note="Description of Artificial Sequence: Synthetic peptide"
WMHPNSGHTGYAQKFQG

SEQ ID NO: 43 - protein, 17 aa, Artificial Sequence
  source: /note="Description of Artificial Sequence: Synthetic peptide"
WMNPNSGHTGNAQKFQG

SEQ ID NO: 44 - protein, 17 aa, Artificial Sequence
  source: /note="Description of Artificial Sequence: Synthetic peptide"
WIDPNSGVTSSAQKFQG

SEQ ID NO: 45 - protein, 17 aa, Artificial Sequence
  source: /note="Description of Artificial Sequence: Synthetic peptide"
WISPNSGVTDFTQKFQG

SEQ ID NO: 46 - protein, 17 aa, Artificial Sequence
  source: /note="Description of Artificial Sequence: Synthetic peptide"
WMNPNSGHTGYAQRFQG

SEQ ID NO: 47 - protein, 17 aa, Artificial Sequence
  source: /note="Description of Artificial Sequence: Synthetic peptide"
WMSPNGGNTGYAQKFQG

SEQ ID NO: 48 - protein, 17 aa, Artificial Sequence
  source: /note="Description of Artificial Sequence: Synthetic peptide"
WMDPSSGYTGSAHKFQG

SEQ ID NO: 49 - protein, 17 aa, Artificial Sequence
  source: /note="Description of Artificial Sequence: Synthetic peptide"
WMNPHSADTGYAEKFQG

SEQ ID NO: 50 - protein, 17 aa, Artificial Sequence
  source: /note="Description of Artificial Sequence: Synthetic peptide"
WLTPSTGHAGYAQKFQG

SEQ ID NO: 51 - protein, 17 aa, Artificial Sequence
  source: /note="Description of Artificial Sequence: Synthetic peptide"
WMNPNSGHTGYAQKFQG

SEQ ID NO: 52 - protein, 17 aa, Artificial Sequence
  source: /note="Description of Artificial Sequence: Synthetic peptide"
WISPQHGVRNYAHKFQG

SEQ ID NO: 53 - protein, 17 aa, Artificial Sequence
  source: /note="Description of Artificial Sequence: Synthetic peptide"
MINPSGGSTSYAQKFQG

SEQ ID NO: 54 - protein, 17 aa, Artificial Sequence
  source: /note="Description of Artificial Sequence: Synthetic peptide"
WISPRSGVTSYAQKFQG

SEQ ID NO: 55 - protein, 17 aa, Artificial Sequence
  source: /note="Description of Artificial Sequence: Synthetic peptide"
WMDPNSGNTGYAQKFQG

SEQ ID NO: 56 - protein, 17 aa, Artificial Sequence
  source: /note="Description of Artificial Sequence: Synthetic peptide"
WMNPTGGITGYAQKFQG

SEQ ID NO: 57 - protein, 17 aa, Artificial Sequence
  source: /note="Description of Artificial Sequence: Synthetic peptide"
WVSPIHGLTGYAPRFQG

SEQ ID NO: 58 - protein, 18 aa, Artificial Sequence
  source: /note="Description of Artificial Sequence: Synthetic peptide"
DLYPYVVVVAAGSYGMDV

SEQ ID NO: 59 - protein, 12 aa, Artificial Sequence
  source: /note="Description of Artificial Sequence: Synthetic peptide"
PSIVGAYDAFDI

SEQ ID NO: 60 - protein, 9 aa, Artificial Sequence
  source: /note="Description of Artificial Sequence: Synthetic peptide"
ESVEGYFDL

SEQ ID NO: 61 - protein, 11 aa, Artificial Sequence
  source: /note="Description of Artificial Sequence: Synthetic peptide"
DNWNVHDAFDI

SEQ ID NO: 62 - protein, 9 aa, Artificial Sequence
  source: /note="Description of Artificial Sequence: Synthetic peptide"
GTYNDAFDI

SEQ ID NO: 63 - protein, 11 aa, Artificial Sequence
  source: /note="Description of Artificial Sequence: Synthetic peptide"
EQWLVNDAFDI

SEQ ID NO: 64 - protein, 13 aa, Artificial Sequence
  source: /note="Description of Artificial Sequence: Synthetic peptide"
DSSGWMRNDAFDI

SEQ ID NO: 65 - protein, 14 aa, Artificial Sequence
  source: /note="Description of Artificial Sequence: Synthetic peptide"
SMFPTIFGDNAFDI

SEQ ID NO: 66 - protein, 14 aa, Artificial Sequence
  source: /note="Description of Artificial Sequence: Synthetic peptide"
ALFPYPFYYYYMDV

SEQ ID NO: 67 - protein, 7 aa, Artificial Sequence
  source: /note="Description of Artificial Sequence: Synthetic peptide"
DRGWFDP

SEQ ID NO: 68 - protein, 10 aa, Artificial Sequence
  source: /note="Description of Artificial Sequence: Synthetic peptide"
DARGYSGYDL

SEQ ID NO: 69 - protein, 9 aa, Artificial Sequence
  source: /note="Description of Artificial Sequence: Synthetic peptide"
EGRHGEYLY

SEQ ID NO: 70 - protein, 11 aa, Artificial Sequence
  source: /note="Description of Artificial Sequence: Synthetic peptide"
EGWGSSGYFDY

SEQ ID NO: 71 - protein, 15 aa, Artificial Sequence
  source: /note="Description of Artificial Sequence: Synthetic peptide"
HLFPTVFDDYYGMDV

SEQ ID NO: 72 - protein, 10 aa, Artificial Sequence
  source: /note="Description of Artificial Sequence: Synthetic peptide"
GGYSYGSFQH

SEQ ID NO: 73 - protein, 10 aa, Artificial Sequence
  source: /note="Description of Artificial Sequence: Synthetic peptide"
VRWSSDAFDI

SEQ ID NO: 74 - protein, 9 aa, Artificial Sequence
  source: /note="Description of Artificial Sequence: Synthetic peptide"
EEWLGHFQH

SEQ ID NO: 75 - protein, 9 aa, Artificial Sequence
  source: /note="Description of Artificial Sequence: Synthetic peptide"
ERFLGGMDV

SEQ ID NO: 76 - protein, 9 aa, Artificial Sequence
  source: /note="Description of Artificial Sequence: Synthetic peptide"
GNWVDAFDI

SEQ ID NO: 77 - protein, 11 aa, Artificial Sequence
  source: /note="Description of Artificial Sequence: Synthetic peptide"
ESEVMMAYFQH

SEQ ID NO: 78 - protein, 9 aa, Artificial Sequence
  source: /note="Description of Artificial Sequence: Synthetic peptide"
ESWSGEFDY

SEQ ID NO: 79 - protein, 9 aa, Artificial Sequence
  source: /note="Description of Artificial Sequence: Synthetic peptide"
EAVAGPMDV

SEQ ID NO: 80 - protein, 10 aa, Artificial Sequence
  source: /note="Description of Artificial Sequence: Synthetic peptide"
DAWELLAFDI

SEQ ID NO: 81 - protein, 10 aa, Artificial Sequence
  source: /note="Description of Artificial Sequence: Synthetic peptide"
DRWDGDYYSA

SEQ ID NO: 82 - protein, 10 aa, Artificial Sequence
  source: /note="Description of Artificial Sequence: Synthetic peptide"
ESWELTGFDY

SEQ ID NO: 83 - protein, 9 aa, Artificial Sequence
  source: /note="Description of Artificial Sequence: Synthetic peptide"
ERFAGGMDA

SEQ ID NO: 84 - protein, 8 aa, Artificial Sequence
  source: /note="Description of Artificial Sequence: Synthetic peptide"
DSGGAFDI

SEQ ID NO: 85 - protein, 9 aa, Artificial Sequence
  source: /note="Description of Artificial Sequence: Synthetic peptide"
EVFEGGMDV

SEQ ID NO: 86 - protein, 9 aa, Artificial Sequence
  source: /note="Description of Artificial Sequence: Synthetic peptide"
EGYGGNYGN

SEQ ID NO: 87 - protein, 9 aa, Artificial Sequence
  source: /note="Description of Artificial Sequence: Synthetic peptide"
EDFYGDFDY

SEQ ID NO: 88 - protein, 9 aa, Artificial Sequence
  source: /note="Description of Artificial Sequence: Synthetic peptide"
ELSRWGFDY

SEQ ID NO: 89 - protein, 14 aa, Artificial Sequence
  source: /note="Description of Artificial Sequence: Synthetic peptide"
DIFPTMIAGGGFDL

SEQ ID NO: 90 - protein, 10 aa, Artificial Sequence
  source: /note="Description of Artificial Sequence: Synthetic peptide"
GGYSYGSFDY

SEQ ID NO: 91 - protein, 15 aa, Artificial Sequence
  source: /note="Description of Artificial Sequence: Synthetic peptide"
GSFPLVFTIFGVGDV

SEQ ID NO: 92 - protein, 10 aa, Artificial Sequence
  source: /note="Description of Artificial Sequence: Synthetic peptide"
DLDYVRAFDI

SEQ ID NO: 93 - protein, 9 aa, Artificial Sequence
  source: /note="Description of Artificial Sequence: Synthetic peptide"
ESWGGYFDL

SEQ ID NO: 94 - protein, 9 aa, Artificial Sequence
  source: /note="Description of Artificial Sequence: Synthetic peptide"
DRTTYAFDI

SEQ ID NO: 95 - protein, 11 aa, Artificial Sequence
  source: /note="Description of Artificial Sequence: Synthetic peptide"
VHGSGSDGMDV

SEQ ID NO: 96 - protein, 11 aa, Artificial Sequence
  source: /note="Description of Artificial Sequence: Synthetic peptide"
RASQGIDSYLA

SEQ ID NO: 97 - protein, 11 aa, Artificial Sequence
  source: /note="Description of Artificial Sequence: Synthetic peptide"
RASQSISSWLA

SEQ ID NO: 98 - protein, 11 aa, Artificial Sequence
  source: /note="Description of Artificial Sequence: Synthetic peptide"
RASQSISSYLN

SEQ ID NO: 99 - protein, 11 aa, Artificial Sequence
  source: /note="Description of Artificial Sequence: Synthetic peptide"
RASQGISSYLA

SEQ ID NO: 100 - protein, 11 aa, Artificial Sequence
  source: /note="Description of Artificial Sequence: Synthetic peptide"
RASQTISNYLN

SEQ ID NO: 101 - protein, 12 aa, Artificial Sequence
  source: /note="Description of Artificial Sequence: Synthetic peptide"
RASQSVDRNYVT

SEQ ID NO: 102 - protein, 11 aa, Artificial Sequence
  source: /note="Description of Artificial Sequence: Synthetic peptide"
RASQGISQYLA

SEQ ID NO: 103 - protein, 11 aa, Artificial Sequence
  source: /note="Description of Artificial Sequence: Synthetic peptide"
QASQDIGNYLN

SEQ ID NO: 104 - protein, 11 aa, Artificial Sequence
  source: /note="Description of Artificial Sequence: Synthetic peptide"
RASQGIRNDLG

SEQ ID NO: 105 - protein, 11 aa, Artificial Sequence
  source: /note="Description of Artificial Sequence: Synthetic peptide"
RASQIIGNYLA

SEQ ID NO: 106 - protein, 11 aa, Artificial Sequence
  source: /note="Description of Artificial Sequence: Synthetic peptide"
RASQIISSYLN

SEQ ID NO: 107 - protein, 11 aa, Artificial Sequence
  source: /note="Description of Artificial Sequence: Synthetic peptide"
QASQDISNYLN

SEQ ID NO: 108 - protein, 11 aa, Artificial Sequence
  source: /note="Description of Artificial Sequence: Synthetic peptide"
RASQGISNYLA

SEQ ID NO: 109 - protein, 11 aa, Artificial Sequence
  source: /note="Description of Artificial Sequence: Synthetic peptide"
RASQGISNNLN

SEQ ID NO: 110 - protein, 11 aa, Artificial Sequence
  source: /note="Description of Artificial Sequence: Synthetic peptide"
RASQGISNGLS

SEQ ID NO: 111 - protein, 11 aa, Artificial Sequence
  source: /note="Description of Artificial Sequence: Synthetic peptide"
RASQSITGWLA

SEQ ID NO: 112 - protein, 11 aa, Artificial Sequence
  source: /note="Description of Artificial Sequence: Synthetic peptide"
RASQGIRNDLA

SEQ ID NO: 113 - protein, 11 aa, Artificial Sequence
  source: /note="Description of Artificial Sequence: Synthetic peptide"
QASQDISSYLN

SEQ ID NO: 114 - protein, 11 aa, Artificial Sequence
  source: /note="Description of Artificial Sequence: Synthetic peptide"
RASQSITTYLN

SEQ ID NO: 115 - protein, 11 aa, Artificial Sequence
  source: /note="Description of Artificial Sequence: Synthetic peptide"
RASQSVSTWLA

SEQ ID NO: 116 - protein, 11 aa, Artificial Sequence
  source: /note="Description of Artificial Sequence: Synthetic peptide"
RASQSISNWLA

SEQ ID NO: 117 - protein, 11 aa, Artificial Sequence
  source: /note="Description of Artificial Sequence: Synthetic peptide"
QASQGISNYLA

SEQ ID NO: 118 - protein, 12 aa, Artificial Sequence
  source: /note="Description of Artificial Sequence: Synthetic peptide"
RASQSLSSSSLA

SEQ ID NO: 119 - protein, 11 aa, Artificial Sequence
  source: /note="Description of Artificial Sequence: Synthetic peptide"
RASEHIANWLA

SEQ ID NO: 120 - protein, 11 aa, Artificial Sequence
  source: /note="Description of Artificial Sequence: Synthetic peptide"
RASQSVGSWVA

SEQ ID NO: 121 - protein, 11 aa, Artificial Sequence
  source: /note="Description of Artificial Sequence: Synthetic peptide"
RASQSISPWLA

SEQ ID NO: 122 - protein, 11 aa, Artificial Sequence
  source: /note="Description of Artificial Sequence: Synthetic peptide"
RASQGISRYLA

SEQ ID NO: 123 - protein, 12 aa, Artificial Sequence
  source: /note="Description of Artificial Sequence: Synthetic peptide"
RASQTVSSNYLA

SEQ ID NO: 124 - protein, 11 aa, Artificial Sequence
  source: /note="Description of Artificial Sequence: Synthetic peptide"
RSSQGIRNDLS

SEQ ID NO: 125 - protein, 11 aa, Artificial Sequence
  source: /note="Description of Artificial Sequence: Synthetic peptide"
RDSHSITTWLA

SEQ ID NO: 126 - protein, 11 aa, Artificial Sequence
  source: /note="Description of Artificial Sequence: Synthetic peptide"
RASQSISRWLA

SEQ ID NO: 127 - protein, 11 aa, Artificial Sequence
  source: /note="Description of Artificial Sequence: Synthetic peptide"
RASQVIRNDLA

SEQ ID NO: 128 - skipped

SEQ ID NO: 129 - protein, 7 aa, Artificial Sequence
  source: /note="Description of Artificial Sequence: Synthetic peptide"
AASSLQS

SEQ ID NO: 130 - protein, 7 aa, Artificial Sequence
  source: /note="Description of Artificial Sequence: Synthetic peptide"
GASNLQS

SEQ ID NO: 131 - protein, 7 aa, Artificial Sequence
  source: /note="Description of Artificial Sequence: Synthetic peptide"
GASTRAT

SEQ ID NO: 132 - protein, 7 aa, Artificial Sequence
  source: /note="Description of Artificial Sequence: Synthetic peptide"
GASNLHS

SEQ ID NO: 133 - protein, 7 aa, Artificial Sequence
  source: /note="Description of Artificial Sequence: Synthetic peptide"
AASSLES

SEQ ID NO: 134 - protein, 7 aa, Artificial Sequence
  source: /note="Description of Artificial Sequence: Synthetic peptide"
SASNLQS

SEQ ID NO: 135 - protein, 7 aa, Artificial Sequence
  source: /note="Description of Artificial Sequence: Synthetic peptide"
AASTLES

SEQ ID NO: 136 - protein, 7 aa, Artificial Sequence
  source: /note="Description of Artificial Sequence: Synthetic peptide"
HASILET

SEQ ID NO: 137 - protein, 7 aa, Artificial Sequence
  source: /note="Description of Artificial Sequence: Synthetic peptide"
AASTLQS

SEQ ID NO: 138 - protein, 7 aa, Artificial Sequence
  source: /note="Description of Artificial Sequence: Synthetic peptide"
AATTLQS

SEQ ID NO: 139 - protein, 7 aa, Artificial Sequence
  source: /note="Description of Artificial Sequence: Synthetic peptide"
DATHLET

SEQ ID NO: 140 - protein, 7 aa, Artificial Sequence
  source: /note="Description of Artificial Sequence: Synthetic peptide"
AASSLQT

SEQ ID NO: 141 - protein, 7 aa, Artificial Sequence
  source: /note="Description of Artificial Sequence: Synthetic peptide"
AASSLQG

SEQ ID NO: 142 - protein, 7 aa, Artificial Sequence
  source: /note="Description of Artificial Sequence: Synthetic peptide"
AASNLES

SEQ ID NO: 143 - protein, 7 aa, Artificial Sequence
  source: /note="Description of Artificial Sequence: Synthetic peptide"
DVSHLES

SEQ ID NO: 144 - protein, 7 aa, Artificial Sequence
  source: /note="Description of Artificial Sequence: Synthetic peptide"
DASSLQS

SEQ ID NO: 145 - protein, 7 aa, Artificial Sequence
  source: /note="Description of Artificial Sequence: Synthetic peptide"
GVSSLES

SEQ ID NO: 146 - protein, 7 aa, Artificial Sequence
  source: /note="Description of Artificial Sequence: Synthetic peptide"
PASTLQS

SEQ ID NO: 147 - protein, 7 aa, Artificial Sequence
  source: /note="Description of Artificial Sequence: Synthetic peptide"
DASNLET

SEQ ID NO: 148 - protein, 7 aa, Artificial Sequence
  source: /note="Description of Artificial Sequence: Synthetic peptide"
GASTRAS

SEQ ID NO: 149 - protein, 7 aa, Artificial Sequence
  source: /note="Description of Artificial Sequence: Synthetic peptide"
DSSSLQT

SEQ ID NO: 150 - protein, 7 aa, Artificial Sequence
  source: /note="Description of Artificial Sequence: Synthetic peptide"
ATSTLQS

SEQ ID NO: 151 - protein, 7 aa, Artificial Sequence
  source: /note="Description of Artificial Sequence: Synthetic peptide"
LASNSHS

SEQ ID NO: 152 - protein, 9 aa, Artificial Sequence
  source: /note="Description of Artificial Sequence: Synthetic peptide"
QQSYSTPIT

SEQ ID NO: 153 - protein, 9 aa, Artificial Sequence
  source: /note="Description of Artificial Sequence: Synthetic peptide"
QQSYTTPIT

SEQ ID NO: 154 - protein, 9 aa, Artificial Sequence
  source: /note="Description of Artificial Sequence: Synthetic peptide"
QQIFSTPLT

SEQ ID NO: 155 - protein, 9 aa, Artificial Sequence
  source: /note="Description of Artificial Sequence: Synthetic peptide"
QQSYSTPLT

SEQ ID NO: 156 - protein, 9 aa, Artificial Sequence
  source: /note="Description of Artificial Sequence: Synthetic peptide"
QQSYTTPYT

SEQ ID NO: 157 - protein, 9 aa, Artificial Sequence
  source: /note="Description of Artificial Sequence: Synthetic peptide"
QQTFTTPLT

SEQ ID NO: 158 - protein, 9 aa, Artificial Sequence
  source: /note="Description of Artificial Sequence: Synthetic peptide"
QQANSFPFT

SEQ ID NO: 159 - protein, 9 aa, Artificial Sequence
  source: /note="Description of Artificial Sequence: Synthetic peptide"
QQSYTTPYS

SEQ ID NO: 160 - protein, 9 aa, Artificial Sequence
  source: /note="Description of Artificial Sequence: Synthetic peptide"
QQTFITPLT

SEQ ID NO: 161 - protein, 8 aa, Artificial Sequence
  source: /note="Description of Artificial Sequence: Synthetic peptide"
QQSYSTPT

SEQ ID NO: 162 - protein, 9 aa, Artificial Sequence
  source: /note="Description of Artificial Sequence: Synthetic peptide"
QQGFSTPFT

SEQ ID NO: 163 - protein, 9 aa, Artificial Sequence
  source: /note="Description of Artificial Sequence: Synthetic peptide"
QQSFTNPVT

SEQ ID NO: 164 - protein, 9 aa, Artificial Sequence
  source: /note="Description of Artificial Sequence: Synthetic peptide"
QQSYSAPYT

SEQ ID NO: 165 - protein, 9 aa, Artificial Sequence
  source: /note="Description of Artificial Sequence: Synthetic peptide"
QQSYSTPYT

SEQ ID NO: 166 - protein, 9 aa, Artificial Sequence
  source: /note="Description of Artificial Sequence: Synthetic peptide"
QQSHSTPLT

SEQ ID NO: 167 - protein, 9 aa, Artificial Sequence
  source: /note="Description of Artificial Sequence: Synthetic peptide"
QQSYSTPFT

SEQ ID NO: 168 - protein, 9 aa, Artificial Sequence
  source: /note="Description of Artificial Sequence: Synthetic peptide"
QQYGSSPFT

SEQ ID NO: 169 - protein, 9 aa, Artificial Sequence
  source: /note="Description of Artificial Sequence: Synthetic peptide"
QQTYSTPIT

SEQ ID NO: 170 - protein, 9 aa, Artificial Sequence
  source: /note="Description of Artificial Sequence: Synthetic peptide"
QQYYTTPLT

SEQ ID NO: 171 - protein, 9 aa, Artificial Sequence
  source: /note="Description of Artificial Sequence: Synthetic peptide"
QQSFSTPLT

SEQ ID NO: 172 - protein, 9 aa, Artificial Sequence
  source: /note="Description of Artificial Sequence: Synthetic peptide"
QQSYSTPPT

SEQ ID NO: 173 - protein, 9 aa, Artificial Sequence
  source: /note="Description of Artificial Sequence: Synthetic peptide"
LQHNSYPLT

SEQ ID NO: 174 - protein, 9 aa, Artificial Sequence
  source: /note="Description of Artificial Sequence: Synthetic peptide"
QQSYSTPVT

SEQ ID NO: 175 - protein, 9 aa, Artificial Sequence
  source: /note="Description of Artificial Sequence: Synthetic peptide"
QHFYNTQYT

SEQ ID NO: 176 - protein, 10 aa, Artificial Sequence
  source: /note="Description of Artificial Sequence: Synthetic peptide"
QQSLQYPSHF

SEQ ID NO: 177 - protein, 30 aa, Artificial Sequence
  source: /note="Description of Artificial Sequence: Synthetic polypeptide"
QVQLVQSGAEVKKPGASVKVSCKASGGTFS

SEQ ID NO: 178 - protein, 30 aa, Artificial Sequence
  source: /note="Description of Artificial Sequence: Synthetic polypeptide"
QVQLVQSGAEVKKPGASVKVSCKASGYTFT

SEQ ID NO: 179 - protein, 30 aa, Artificial Sequence
  source: /note="Description of Artificial Sequence: Synthetic polypeptide"
QVQLVQSGAEVKKPGASVKVSCKASGYTFN

SEQ ID NO: 180 - protein, 30 aa, Artificial Sequence
  source: /note="Description of Artificial Sequence: Synthetic polypeptide"
QVQLVQSGAEVKKPGASVKVSCKASGNTFT

SEQ ID NO: 181 - protein, 30 aa, Artificial Sequence
  source: /note="Description of Artificial Sequence: Synthetic polypeptide"
QVQLVQSGAEVKKPGASVKVSCKASGHSFT

SEQ ID NO: 182 - protein, 30 aa, Artificial Sequence
  source: /note="Description of Artificial Sequence: Synthetic polypeptide"
QVQLVQSGAEVKKPGASVKVSCKASGYPFT

SEQ ID NO: 183 - protein, 30 aa, Artificial Sequence
  source: /note="Description of Artificial Sequence: Synthetic polypeptide"
QVQLVQSGAEVKKPGASVKVSCKASGYRFT

SEQ ID NO: 184 - protein, 30 aa, Artificial Sequence
  source: /note="Description of Artificial Sequence: Synthetic polypeptide"
QVQLVQSGAEVKKPGSSVKVSCKASGGTFS

SEQ ID NO: 185 - protein, 30 aa, Artificial Sequence
  source: /note="Description of Artificial Sequence: Synthetic polypeptide"
QVQLVQSGAEVKKPGASVKVSCKASGDTFT

SEQ ID NO: 186 - protein, 30 aa, Artificial Sequence
  source: /note="Description of Artificial Sequence: Synthetic polypeptide"
QVQLAQSGAEVKKPGASVKVSCKASGYTFT

SEQ ID NO: 187 - protein, 30 aa, Artificial Sequence
  source: /note="Description of Artificial Sequence: Synthetic polypeptide"
QVQLVQSGAEVKKPGASVKVSCKASGYTFS

SEQ ID NO: 188 - protein, 30 aa, Artificial Sequence
  source: /note="Description of Artificial Sequence: Synthetic polypeptide"
QVQLVQSGAEVKKPGASVKVSCKASGYPFS

SEQ ID NO: 189 - protein, 30 aa, Artificial Sequence
  source: /note="Description of Artificial Sequence: Synthetic polypeptide"
QVQLVQSGAEVKKPGASVKVSCKASGYSFT

SEQ ID NO: 190 - protein, 30 aa, Artificial Sequence
  source: /note="Description of Artificial Sequence: Synthetic polypeptide"
QVQLVQSGAEVKKPGASVKVSCKASGYTFI

SEQ ID NO: 191 - protein, 30 aa, Artificial Sequence
  source: /note="Description of Artificial Sequence: Synthetic polypeptide"
QVQLVQSGAEVKKPGSSVKVSCKASGYTFT

SEQ ID NO: 192 - protein, 14 aa, Artificial Sequence
  source: /note="Description of Artificial Sequence: Synthetic peptide"
WVRQAPGQGLEWMG

SEQ ID NO: 193 - protein, 14 aa, Artificial Sequence
  source: /note="Description of Artificial Sequence: Synthetic peptide"
WVRQAPGQGLEWLG

SEQ ID NO: 194 - protein, 14 aa, Artificial Sequence
  source: /note="Description of Artificial Sequence: Synthetic peptide"
WVRQAPGQGLEWIG

SEQ ID NO: 195 - protein, 14 aa, Artificial Sequence
  source: /note="Description of Artificial Sequence: Synthetic peptide"
WVRQAPGQGLEWVG

SEQ ID NO: 196 - protein, 32 aa, Artificial Sequence
  source: /note="Description of Artificial Sequence: Synthetic polypeptide"
RVTMTRDTSTSTVYMELSSLRSEDTAVYYCAR

SEQ ID NO: 197 - protein, 32 aa, Artificial Sequence
  source: /note="Description of Artificial Sequence: Synthetic polypeptide"
RVTMTRDTSTSTVYMELSSLRSEDTAVYYCAI

SEQ ID NO: 198 - protein, 32 aa, Artificial Sequence
  source: /note="Description of Artificial Sequence: Synthetic polypeptide"
RVTMTRDTSTSTVYMELSSLRSEDTAVYYCTR

SEQ ID NO: 199 - protein, 32 aa, Artificial Sequence
  source: /note="Description of Artificial Sequence: Synthetic polypeptide"
RVTITADESTSTAYMELSSLRSEDTAVYYCAR

SEQ ID NO: 200 - protein, 32 aa, Artificial Sequence
  source: /note="Description of Artificial Sequence: Synthetic polypeptide"
RVTMTRDTSTSTVNMELSSLRSEDTAVYYCAR

SEQ ID NO: 201 - protein, 32 aa, Artificial Sequence
  source: /note="Description of Artificial Sequence: Synthetic polypeptide"
RVTMTRDTSTSTVYMELSSLRSEDTAVYYCAE

SEQ ID NO: 202 - protein, 11 aa, Artificial Sequence
  source: /note="Description of Artificial Sequence: Synthetic peptide"
WGQGTLVTVSS

SEQ ID NO: 203 - protein, 11 aa, Artificial Sequence
  source: /note="Description of Artificial Sequence: Synthetic peptide"
WGQGTTVTVSS

SEQ ID NO: 204 - protein, 11 aa, Artificial Sequence
  source: /note="Description of Artificial Sequence: Synthetic peptide"
WGQGTMVTVSS

SEQ ID NO: 205 - protein, 11 aa, Artificial Sequence
  source: /note="Description of Artificial Sequence: Synthetic peptide"
WGPGTMVTVSS

SEQ ID NO: 206 - protein, 11 aa, Artificial Sequence
  source: /note="Description of Artificial Sequence: Synthetic peptide"
WGRGTLVTVSS

SEQ ID NO: 207 - protein, 23 aa, Artificial Sequence
  source: /note="Description of Artificial Sequence: Synthetic peptide"
DIQMTQSPSSLSASVGDRVTITC

SEQ ID NO: 208 - protein, 23 aa, Artificial Sequence
  source: /note="Description of Artificial Sequence: Synthetic peptide"
EIVMTQSPATLSVSPGERATLSC

SEQ ID NO: 209 - protein, 23 aa, Artificial Sequence
  source: /note="Description of Artificial Sequence: Synthetic peptide"
DIQITHSPSSLSASVGYRLTITC

SEQ ID NO: 210 - protein, 15 aa, Artificial Sequence
  source: /note="Description of Artificial Sequence: Synthetic peptide"
WYQQKPGKAPKLLIY

SEQ ID NO: 211 - protein, 15 aa, Artificial Sequence
  source: /note="Description of Artificial Sequence: Synthetic peptide"
WYQQKPGQAPRLLIY

SEQ ID NO: 212 - protein, 15 aa, Artificial Sequence
  source: /note="Description of Artificial Sequence: Synthetic peptide"
WYHQKPWNAPKLMIY

SEQ ID NO: 213 - protein, 32 aa, Artificial Sequence
  source: /note="Description of Artificial Sequence: Synthetic polypeptide"
GVPSRFSGSGSGTDFTLTISSLQPEDFATYYC

SEQ ID NO: 214 - protein, 32 aa, Artificial Sequence
  source: /note="Description of Artificial Sequence: Synthetic polypeptide"
GIPARFSGSGSGTEFTLTISSLQSEDFAVYYC

SEQ ID NO: 215 - protein, 32 aa, Artificial Sequence
  source: /note="Description of Artificial Sequence: Synthetic polypeptide"
GVPSRFSGSGSGTYFTLTISSLQPEDFATYYC

SEQ ID NO: 216 - protein, 32 aa, Artificial Sequence
  source: /note="Description of Artificial Sequence: Synthetic polypeptide"
GVPSRFSGSGSGTDFTLTISSLQPEDFAPYYC

SEQ ID NO: 217 - protein, 10 aa, Artificial Sequence
  source: /note="Description of Artificial Sequence: Synthetic peptide"
FGGGTKVEIK

SEQ ID NO: 218 - protein, 10 aa, Artificial Sequence
  source: /note="Description of Artificial Sequence: Synthetic peptide"
FGQGTKVEIK

SEQ ID NO: 219 - protein, 10 aa, Artificial Sequence
  source: /note="Description of Artificial Sequence: Synthetic peptide"
FGQGTRLEIK

SEQ ID NO: 220 - protein, 10 aa, Artificial Sequence
  source: /note="Description of Artificial Sequence: Synthetic peptide"
FGGGTKLEIK

SEQ ID NO: 221 - protein, 10 aa, Artificial Sequence
  source: /note="Description of Artificial Sequence: Synthetic peptide"
FGQGTKLEIK

SEQ ID NO: 222 - protein, 10 aa, Artificial Sequence
  source: /note="Description of Artificial Sequence: Synthetic peptide"
FGPGTKVDIK

SEQ ID NO: 223 - protein, 127 aa, Artificial Sequence
  source: /note="Description of Artificial Sequence: Synthetic polypeptide"
QVQLVQSGAEVKKPGASVKVSCKASGGTFSSYAISWVRQAPGQGLEWMGVINPSAGSTDYAQKFQGRVTMTRDTSTSTVYMELSSLRSEDTAVYYCARDLYPYVVVVAAGSYGMDVWGQGTLVTVSS

SEQ ID NO: 224 - protein, 121 aa, Artificial Sequence
  source: /note="Description of Artificial Sequence: Synthetic polypeptide"
QVQLVQSGAEVKKPGASVKVSCKASGYTFTSYYMHWVRQAPGQGLEWMGWMNPNSDIAGYAQKFQGRVTMTRDTSTSTVYMELSSLRSEDTAVYYCAIPSIVGAYDAFDIWGQGTLVTVSS

SEQ ID NO: 225 - protein, 118 aa, Artificial Sequence
  source: /note="Description of Artificial Sequence: Synthetic polypeptide"
QVQLVQSGAEVKKPGASVKVSCKASGYTFTRHLLHWVRQAPGQGLEWMGWISPQHGVRNYAQKFQGRVTMTRDTSTSTVYMELSSLRSEDTAVYYCARESVEGYFDLWGQGTLVTVSS

SEQ ID NO: 226 - protein, 120 aa, Artificial Sequence
  source: /note="Description of Artificial Sequence: Synthetic polypeptide"
QVQLVQSGAEVKKPGASVKVSCKASGYTFTSHHMHWVRQAPGQGLEWMGWVSPSHGLTGYAQKFQGRVTMTRDTSTSTVYMELSSLRSEDTAVYYCARDNWNVHDAFDIWGQGTLVTVSS

SEQ ID NO: 227 - protein, 117 aa, Artificial Sequence
  source: /note="Description of Artificial Sequence: Synthetic polypeptide"
QVQLVQSGAEVKKPGASVKVSCKASGYTFNRFMHWVRQAPGQGLEWMGWMSLNSGLTGYAQKFQGRVTMTRDTSTSTVYMELSSLRSEDTAVYYCTRGTYNDAFDIWGQGTLVTVSS

SEQ ID NO: 228 - protein, 120 aa, Artificial Sequence
  source: /note="Description of Artificial Sequence: Synthetic polypeptide"
QVQLVQSGAEVKKPGASVKVSCKASGYTFTSYYIHWVRQAPGQGLEWMGWMKPSSGTTGYAQKFQGRVTMTRDTSTSTVYMELSSLRSEDTAVYYCAREQWLVNDAFDIWGQGTLVTVSS

SEQ ID NO: 229 - protein, 120 aa, Artificial Sequence; identical to SEQ ID NO: 228
  source: /note="Description of Artificial Sequence: Synthetic polypeptide"

SEQ ID NO: 230 - protein, 123 aa, Artificial Sequence
  source: /note="Description of Artificial Sequence: Synthetic polypeptide"
QVQLVQSGAEVKKPGASVKVSCKASGNTFTSYYMHWVRQAPGQGLEWMGGIDPNSGGTNYAQKFQGRVTMTRDTSTSTVYMELSSLRSEDTAVYYCARSMFPTIFGDNAFDIWGQGTLVTVSS

SEQ ID NO: 231 - protein, 123 aa, Artificial Sequence
  source: /note="Description of Artificial Sequence: Synthetic polypeptide"
QVQLVQSGAEVKKPGASVKVSCKASGHSFTHYYMHWVRQAPGQGLEWMGWMNPDSGSTGYAQKFQGRVTMTRDTSTSTVYMELSSLRSEDTAVYYCARALFPYPFYYYYMDVWGQGTLVTVSS

SEQ ID NO: 232 - protein, 116 aa, Artificial Sequence
  source: /note="Description of Artificial Sequence: Synthetic polypeptide"
QVQLVQSGAEVKKPGASVKVSCKASGYTFTGYYMHWVRQAPGQGLEWIGWMSLNSGLTGYAQKFQGRVTMTRDTSTSTVYMELSSLRSEDTAVYYCARDRGWFDPWGQGTLVTVSS

SEQ ID NO: 233 - protein, 122 aa, Artificial Sequence
  source: /note="Description of Artificial Sequence: Synthetic polypeptide"
QVQLVQSGAEVKKPGASVKVSCKASGYTFTNYYIHWVRQAPGQGLEWLGWMNPNGDVAGYADSFQGRVTMTRDTSTSTVYMELSSLRSEDTAVYYCAIDSSGWMRNDAFDIWGQGTLVTVSS

SEQ ID NO: 234 - protein, 119 aa, Artificial Sequence
  source: /note="Description of Artificial Sequence: Synthetic polypeptide"
QVQLVQSGAEVKKPGASVKVSCKASGGTFSNYMYHWVRQAPGQGLEWMGWISTYHGSTNYAQKFQGRVTMTRDTSTSTVYMELSSLRSEDTAVYYCARDARGYSGYDLWGQGTLVTVSS

SEQ ID NO: 235 - protein, 118 aa, Artificial Sequence
  source: /note="Description of Artificial Sequence: Synthetic polypeptide"
QVQLVQSGAEVKKPGASVKVSCKASGYPFTSYYMHWVRQAPGQGLEWMGWMNPNSDIAGYAQKFQGRVTMTRDTSTSTVYMELSSLRSEDTAVYYCAREGRHGEYLYWGQGTLVTVSS

SEQ ID NO: 236 - protein, 120 aa, Artificial Sequence
  source: /note="Description of Artificial Sequence: Synthetic polypeptide"
QVQLVQSGAEVKKPGASVKVSCKASGYRFTTYYVHWVRQAPGQGLEWMGWMNPNTVYTGSAQKFQGRVTMTRDTSTSTVYMELSSLRSEDTAVYYCAREGWGSSGYFDYWGQGTLVTVSS

SEQ ID NO: 237 - protein, 124 aa, Artificial Sequence
  source: /note="Description of Artificial Sequence: Synthetic polypeptide"
QVQLVQSGAEVKKPGSSVKVSCKASGGTFSSYALSWVRQAPGQGLEWMGRIIPAVGSVTYAQKFQGRVTITADESTSTAYMELSSLRSEDTAVYYCARHLFPTVFDDYYGMDVWGQGTLVTVSS

SEQ ID NO: 238 - protein, 119 aa, Artificial Sequence
  source: /note="Description of Artificial Sequence: Synthetic polypeptide"
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGGYSYGSFQHWGQGTLVTVSS

SEQ ID NO: 239 - protein, 119 aa, Artificial Sequence
  source: /note="Description of Artificial Sequence: Synthetic polypeptide"
QVQLVQSGAEVKKPGASVKVSCKASGDTFTRHYVHWVRQAPGQGLEWMGWMSPSSGITGYAQKFQGRVTMTRDTSTSTVYMELSSLRSEDTAVYYCARVRWSSDAFDIWGQGTLVTVSS

SEQ ID NO: 240 - protein, 118 aa, Artificial Sequence
  source: /note="Description of Artificial Sequence: Synthetic polypeptide"
QVQLVQSGAEVKKPGASVKVSCKASGYTFTSYYMHWVRQAPGQGLEWVGWMTPSTGNAGYAQKFQGRVTMTRDTSTSTVYMELSSLRSEDTAVYYCAREEWLGHFQHWGQGTLVTVSS

SEQ ID NO: 241 - protein, 118 aa, Artificial Sequence
  source: /note="Description of Artificial Sequence: Synthetic polypeptide"
QVQLVQSGAEVKKPGASVKVSCKASGYTFTSHYMHWVRQAPGQGLEWMGWMNPNSGNTGYAQKFQGRVTMTRDTSTSTVYMELSSLRSEDTAVYYCARERFLGGMDVWGQGTTVTVSS

SEQ ID NO: 242 - protein, 118 aa, Artificial Sequence
  source: /note="Description of Artificial Sequence: Synthetic polypeptide"
QVQLVQSGAEVKKPGASVKVSCKASGYTFTDYYMHWVRQAPGQGLEWMGWMHPNSGHTGYAQKFQGRVTMTRDTSTSTVNMELSSLRSEDTAVYYCAREEWLGHFQHWGQGTLVTVSS

SEQ ID NO: 243 - protein, 118 aa, Artificial Sequence
  source: /note="Description of Artificial Sequence: Synthetic polypeptide"
QVQLVQSGAEVKKPGASVKVSCKASGYTFTSHYMHWVRQAPGQGLEWMGWMNPNSGHTGNAQKFQGRVTMTRDTSTSTVYMELSSLRSEDTAVYYCARGNWVDAFDIWGQGTMVTVSS

SEQ ID NO: 244 - protein, 120 aa, Artificial Sequence
  source: /note="Description of Artificial Sequence: Synthetic polypeptide"
QVQLVQSGAEVKKPGASVKVSCKASGYTFTGYTLHWVRQAPGQGLEWMGWIDPNSGVTSSAQKFQGRVTMTRDTSTSTVYMELSSLRSEDTAVYYCARESEVMMAYFQHWGQGTLVTVSS

SEQ ID NO: 245 - protein, 118 aa, Artificial Sequence
  source: /note="Description of Artificial Sequence: Synthetic polypeptide"
QVQLVQSGAEVKKPGASVKVSCKASGYTFTGYYMHWVRQAPGQGLEWMGWISPNSGVTDFTQKFQGRVTMTRDTSTSTVYMELSSLRSEDTAVYYCARESWSGEFDYWGQGTLVTVSS

SEQ ID NO: 246 - protein, 118 aa, Artificial Sequence
  source: /note="Description of Artificial Sequence: Synthetic polypeptide"
QVQLVQSGAEVKKPGASVKVSCKASGDTFTNHYMHWVRQAPGQGLEWMGWMNPNSGHTGYAQRFQGRVTMTRDTSTSTVYMELSSLRSEDTAVYYCAREAVAGPMDVWGQGTTVTVSS

SEQ ID NO: 247 - protein, 119 aa, Artificial Sequence
  source: /note="Description of Artificial Sequence: Synthetic polypeptide"
QVQLVQSGAEVKKPGASVKVSCKASGYTFTGYYMHWVRQAPGQGLEWMGWMNPNSDIAGYAQKFQGRVTMTRDTSTSTVYMELSSLRSEDTAVYYCARDAWELLAFDIWGQGTLVTVSS

SEQ ID NO: 248 - protein, 119 aa, Artificial Sequence
  source: /note="Description of Artificial Sequence: Synthetic polypeptide"
QVQLAQSGAEVKKPGASVKVSCKASGYTFTNHYMHWVRQAPGQGLEWMGWMNPNSGNTGYAQKFQGRVTMTRDTSTSTVYMELSSLRSEDTAVYYCARDRWDGDYYSAWGQGTLVTVSS

SEQ ID NO: 249 - protein, 119 aa, Artificial Sequence
  source: /note="Description of Artificial Sequence: Synthetic polypeptide"
QVQLVQSGAEVKKPGASVKVSCKASGYTFTNYYIHWVRQAPGQGLEWMGWMSPNGGNTGYAQKFQGRVTMTRDTSTSTVYMELSSLRSEDTAVYYCARESWELTGFDYWGQGTLVTVSS

SEQ ID NO: 250 - protein, 118 aa, Artificial Sequence
  source: /note="Description of Artificial Sequence: Synthetic polypeptide"
QVQLVQSGAEVKKPGASVKVSCKASGYTFTSYYMHWVRQAPGQGLEWMGWMNPNSGNTGYAQKFQGRVTMTRDTSTSTVYMELSSLRSEDTAVYYCARERFAGGMDAWGQGTTVTVSS

SEQ ID NO: 251 - protein, 117 aa, Artificial Sequence
  source: /note="Description of Artificial Sequence: Synthetic polypeptide"
QVQLVQSGAEVKKPGASVKVSCKASGYTFSNSYMHWVRQAPGQGLEWMGWMDPSSGYTGSAHKFQGRVTMTRDTSTSTVYMELSSLRSEDTAVYYCAEDSGGAFDIWGQGTMVTVSS

SEQ ID NO: 252 - protein, 118 aa, Artificial Sequence
  source: /note="Description of Artificial Sequence: Synthetic polypeptide"
QVQLVQSGAEVKKPGASVKVSCKASGYPFSTYYMHWVRQAPGQGLEWMGWMNPHSADTGYAEKFQGRVTMTRDTSTSTVYMELSSLRSEDTAVYYCAREVFEGGMDVWGQGTTVTVSS

SEQ ID NO: 253 - protein, 118 aa, Artificial Sequence
  source: /note="Description of Artificial Sequence: Synthetic polypeptide"
QVQLVQSGAEVKKPGASVKVSCKASGYTFTSYYMHWVRQAPGQGLEWMGWLTPSTGHAGYAQKFQGRVTMTRDTSTSTVYMELSSLRSEDTAVYYCAREGYGGNYGNWGQGTLVTVSS

SEQ ID NO: 254 - protein, 118 aa, Artificial Sequence
  source: /note="Description of Artificial Sequence: Synthetic polypeptide"
QVQLVQSGAEVKKPGASVKVSCKASGYTFTSYYMHWVRQAPGQGLEWMGWMNPNSGHTGYAQKFQGRVTMTRDTSTSTVYMELSSLRSEDTAVYYCAREDFYGDFDYWGQGTLVTVSS

SEQ ID NO: 255 - protein, 118 aa, Artificial Sequence
  source: /note="Description of Artificial Sequence: Synthetic polypeptide"
QVQLVQSGAEVKKPGASVKVSCKASGYTFTRHFIHWVRQAPGQGLEWMGWIDPNSGVTSSAQKFQGRVTMTRDTSTSTVYMELSSLRSEDTAVYYCARELSRWGFDYWGPGTMVTVSS

SEQ ID NO: 256 - protein, 118 aa, Artificial Sequence
  source: /note="Description of Artificial Sequence: Synthetic polypeptide"
QVQLVQSGAEVKKPGASVKVSCKASGYTFTRHLLHWVRQAPGQGLEWMGWISPQHGVRNYAHKFQGRVTMTRDTSTSTVYMELSSLRSEDTAVYYCARESVEGYFDLWGRGTLVTVSS

SEQ ID NO: 257 - protein, 123 aa, Artificial Sequence
  source: /note="Description of Artificial Sequence: Synthetic polypeptide"
QVQLVQSGAEVKKPGASVKVSCKASGYPFSSYYMHWVRQAPGQGLEWMGMINPSGGSTSYAQKFQGRVTMTRDTSTSTVYMELSSLRSEDTAVYYCARDIFPTMIAGGGFDLWGRGTLVTVSS

SEQ ID NO: 258 - protein, 119 aa, Artificial Sequence
  source: /note="Description of Artificial Sequence: Synthetic polypeptide"
QVQLVQSGAEVKKPGSSVKVSCKASGGTFSTFGISWVRQAPGQGLEWMGGIIPIFGTANYAQKFQGRVTITADESTSTAYMELSSLRSEDTAVYYCARGGYSYGSFDYWGQGTLVTVSS

SEQ ID NO: 259 - protein, 124 aa, Artificial Sequence
  source: /note="Description of Artificial Sequence: Synthetic polypeptide"
QVQLVQSGAEVKKPGASVKVSCKASGYTFTSYGINWVRQAPGQGLEWMGWMNPNSGNTGYAQKFQGRVTMTRDTSTSTVYMELSSLRSEDTAVYYCARGSFPLVFTIFGVGDVWGQGTMVTVSS

SEQ ID NO: 260 - protein, 119 aa, Artificial Sequence
  source: /note="Description of Artificial Sequence: Synthetic polypeptide"
QVQLVQSGAEVKKPGASVKVSCKASGYTFTSYYMHWVRQAPGQGLEWMGWISPRSGVTSYAQKFQGRVTMTRDTSTSTVYMELSSLRSEDTAVYYCARDLDYVRAFDIWGQGTTVTVSS

SEQ ID NO: 261 - protein, 118 aa, Artificial Sequence
  source: /note="Description of Artificial Sequence: Synthetic polypeptide"
QVQLVQSGAEVKKPGASVKVSCKASGYSFTSYYMHWVRQAPGQGLEWMGWMDPNSGNTGYAQKFQGRVTMTRDTSTSTVYMELSSLRSEDTAVYYCARESWGGYFDLWGRGTLVTVSS

SEQ ID NO: 262 - protein, 118 aa, Artificial Sequence
  source: /note="Description of Artificial Sequence: Synthetic polypeptide"
QVQLVQSGAEVKKPGASVKVSCKASGYTFINHYVHWVRQAPGQGLEWMGWMNPTGGITGYAQKFQGRVTMTRDTSTSTVYMELSSLRSEDTAVYYCARDRTTYAFDIWGQGTMVTVSS

SEQ ID NO: 263 - protein, 118 aa, Artificial Sequence; identical to SEQ ID NO: 243
  source: /note="Description of Artificial Sequence: Synthetic polypeptide"

SEQ ID NO: 264 - protein, 120 aa, Artificial Sequence
  source: /note="Description of Artificial Sequence: Synthetic polypeptide"
QVQLVQSGAEVKKPGSSVKVSCKASGYTFTRHLLHWVRQAPGQGLEWMGWVSPIHGLTGYAPRFQGRVTITADESTSTAYMELSSLRSEDTAVYYCARVHGSGSDGMDVWGQGTTVTVSS

SEQ ID NO: 265 - protein, 107 aa, Artificial Sequence
  source: /note="Description of Artificial Sequence: Synthetic polypeptide"
DIQMTQSPSSLSASVGDRVTITCRASQGIDSYLAWYQQKPGKAPKLLIYAASSLQSGVPSRFSGSGSGTDFTLTISSLQPEDFATYYCQQSYSTPITFGGGTKVEIK

SEQ ID NO: 266 - protein, 107 aa, Artificial Sequence
  source: /note="Description of Artificial Sequence: Synthetic polypeptide"
DIQMTQSPSSLSASVGDRVTITCRASQSISSWLAWYQQKPGKAPKLLIYAASSLQSGVPSRFSGSGSGTDFTLTISSLQPEDFATYYCQQSYTTPITFGGGTKVEIK

SEQ ID NO: 267 - protein, 107 aa, Artificial Sequence
  source: /note="Description of Artificial Sequence: Synthetic polypeptide"
DIQMTQSPSSLSASVGDRVTITCRASQSISSYLNWYQQKPGKAPKLLIYAASSLQSGVPSRFSGSGSGTDFTLTISSLQPEDFATYYCQQIFSTPLTFGGGTKVEIK

SEQ ID NO: 268 - protein, 107 aa, Artificial Sequence
  source: /note="Description of Artificial Sequence: Synthetic polypeptide"
DIQMTQSPSSLSASVGDRVTITCRASQGISSYLAWYQQKPGKAPKLLIYGASNLQSGVPSRFSGSGSGTDFTLTISSLQPEDFATYYCQQSYSTPLTFGGGTKVEIK

SEQ ID NO: 269 - protein, 107 aa, Artificial Sequence
  source: /note="Description of Artificial Sequence: Synthetic polypeptide"
DIQMTQSPSSLSASVGDRVTITCRASQTISNYLNWYQQKPGKAPKLLIYAASSLQSGVPSRFSGSGSGTDFTLTISSLQPEDFATYYCQQSYTTPITFGGGTKVEIK

SEQ ID NO: 270 - protein, 108 aa, Artificial Sequence
  source: /note="Description of Artificial Sequence: Synthetic polypeptide"
EIVMTQSPATLSVSPGERATLSCRASQSVDRNYVTWYQQKPGQAPRLLIYGASTRATGIPARFSGSGSGTEFTLTISSLQSEDFAVYYCQQSYTTPYTFGQGTKVEIK

SEQ ID NO: 271 - protein, 107 aa, Artificial Sequence
  source: /note="Description of Artificial Sequence: Synthetic polypeptide"
DIQMTQSPSSLSASVGDRVTITCRASQGISQYLAWYQQKPGKAPKLLIYGASNLHSGVPSRFSGSGSGTDFTLTISSLQPEDFATYYCQQTFTTPLTFGGGTKVEIK

SEQ ID NO: 272 - protein, 107 aa, Artificial Sequence
  source: /note="Description of Artificial Sequence: Synthetic polypeptide"
DIQMTQSPSSLSASVGDRVTITCQASQDIGNYLNWYQQKPGKAPKLLIYAASSLESGVPSRFSGSGSGTDFTLTISSLQPEDFATYYCQQSYSTPLTFGGGTKVEIK

SEQ ID NO: 273 - protein, 107 aa, Artificial Sequence
  source: /note="Description of Artificial Sequence: Synthetic polypeptide"
DIQMTQSPSSLSASVGDRVTITCRASQGIRNDLGWYQQKPGKAPKLLIYSASNLQSGVPSRFSGSGSGTDFTLTISSLQPEDFATYYCQQANSFPFTFGGGTKVEIK

SEQ ID NO: 274 - protein, 107 aa, Artificial Sequence
  source: /note="Description of Artificial Sequence: Synthetic polypeptide"
DIQMTQSPSSLSASVGDRVTITCRASQSISSWLAWYQQKPGKAPKLLIYAASTLESGVPSRFSGSGSGTDFTLTISSLQPEDFATYYCQQSYTTPYSFGGGTKVEIK

SEQ ID NO: 275 - protein, 107 aa, Artificial Sequence
  source: /note="Description of Artificial Sequence: Synthetic polypeptide"
DIQMTQSPSSLSASVGDRVTITCRASQGISQYLAWYQQKPGKAPKLLIYGASNLHSGVPSRFSGSGSGTDFTLTISSLQPEDFATYYCQQTFITPLTFGGGTKVEIK

SEQ ID NO: 276 - protein, 106 aa, Artificial Sequence
  source: /note="Description of Artificial Sequence: Synthetic polypeptide"
DIQMTQSPSSLSASVGDRVTITCRASQIIGNYLAWYQQKPGKAPKLLIYHASILETGVPSRFSGSGSGTDFTLTISSLQPEDFATYYCQQSYSTPTFGGGTKVEIK

SEQ ID NO: 277 - protein, 107 aa, Artificial Sequence
  source: /note="Description of Artificial Sequence: Synthetic polypeptide"
DIQMTQSPSSLSASVGDRVTITCRASQIISSYLNWYQQKPGKAPKLLIYAASSLQSGVPSRFSGSGSGTDFTLTISSLQPEDFATYYCQQGFSTPFTFGGGTKVEIK

SEQ ID NO: 278 - protein, 107 aa, Artificial Sequence
  source: /note="Description of Artificial Sequence: Synthetic polypeptide"
DIQMTQSPSSLSASVGDRVTITCQASQDISNYLNWYQQKPGKAPKLLIYAASSLQSGVPSRFSGSGSGTDFTLTISSLQPEDFATYYCQQSFTNPVTFGGGTKVEIK

SEQ ID NO: 279 - protein, 107 aa, Artificial Sequence
  source: /note="Description of Artificial Sequence: Synthetic polypeptide"
DIQMTQSPSSLSASVGDRVTITCRASQGISNYLAWYQQKPGKAPKLLIYAASTLQSGVPSRFSGSGSGTDFTLTISSLQPEDFATYYCQQSYSAPYTFGGGTKVEIK

SEQ ID NO: 280 - protein, 107 aa, Artificial Sequence
  source: /note="Description of Artificial Sequence: Synthetic polypeptide"
DIQMTQSPSSLSASVGDRVTITCRASQGISNNLNWYQQKPGKAPKLLIYAATTLQSGVPSRFSGSGSGTDFTLTISSLQPEDFATYYCQQSYSTPYTFGGGTKVEIK

SEQ ID NO: 281 - protein, 107 aa, Artificial Sequence [sequence omitted]

SEQ ID NO: 282 - protein, 107 aa, Artificial Sequence
  source: /note="Description of Artificial Sequence: Synthetic polypeptide"
DIQMTQSPSSLSASVGDRVTITCRASQGISNGLSWYQQKPGKAPKLLIYAASTLQSGVPSRFSGSGSGTDFTLTISSLQPEDFATYYCQQSHSTPLTFGGGTKVEIK

SEQ ID NO: 283 - protein, 107 aa, Artificial Sequence
  source: /note="Description of Artificial Sequence: Synthetic polypeptide"
DIQMTQSPSSLSASVGDRVTITCRASQSITGWLAWYQQKPGKAPKLLIYAASSLQSGVPSRFSGSGSGTDFTLTISSLQPEDFATYYCQQSYSTPYTFGQGTKVEIK

SEQ ID NO: 284 - protein, 107 aa, Artificial Sequence
  source: /note="Description of Artificial Sequence: Synthetic polypeptide"
DIQMTQSPSSLSASVGDRVTITCRASQSISSWLAWYQQKPGKAPKLLIYDATHLETGVPSRFSGSGSGTDFTLTISSLQPEDFATYYCQQSYSTPITFGQGTRLEIK

SEQ ID NO: 285 - protein, 107 aa, Artificial Sequence
  source: /note="Description of Artificial Sequence: Synthetic polypeptide"
DIQMTQSPSSLSASVGDRVTITCRASQGIRNDLAWYQQKPGKAPKLLIYAASTLQSGVPSRFSGSGSGTDFTLTISSLQPEDFATYYCQQSYSTPLTFGGGTKVEIK

SEQ ID NO: 286 - protein, 107 aa, Artificial Sequence
  source: /note="Description of Artificial Sequence: Synthetic polypeptide"
DIQMTQSPSSLSASVGDRVTITCQASQDISSYLNWYQQKPGKAPKLLIYAASSLQTGVPSRFSGSGSGTDFTLTISSLQPEDFATYYCQQSYSTPYTFGQGTKLEIK

SEQ ID NO: 287 - protein, 107 aa, Artificial Sequence
  source: /note="Description of Artificial Sequence: Synthetic polypeptide"
DIQMTQSPSSLSASVGDRVTITCRASQSITTYLNWYQQKPGKAPKLLIYAASSLQGGVPSRFSGSGSGTDFTLTISSLQPEDFATYYCQQSYSTPYTFGQGTKVEIK

SEQ ID NO: 288 - protein, 107 aa, Artificial Sequence
  source: /note="Description of Artificial Sequence: Synthetic polypeptide"
DIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKLEIK

SEQ ID NO: 289 - protein, 107 aa, Artificial Sequence
  source: /note="Description of Artificial Sequence: Synthetic polypeptide"
DIQMTQSPSSLSASVGDRVTITCRASQSVSTWLAWYQQKPGKAPKLLIYAASNLESGVPSRFSGSGSGTDFTLTISSLQPEDFATYYCQQSYSTPYTFGQGTKLEIK

SEQ ID NO: 290 - protein, 107 aa, Artificial Sequence
  source: /note="Description of Artificial Sequence: Synthetic polypeptide"
DIQMTQSPSSLSASVGDRVTITCRASQSISNWLAWYQQKPGKAPKLLIYDVSHLESGVPSRFSGSGSGTDFTLTISSLQPEDFATYYCQQSYSTPFTFGPGTKVDIK

SEQ ID NO: 291 - protein, 107 aa, Artificial Sequence
  source: /note="Description of Artificial Sequence: Synthetic polypeptide"
DIQMTQSPSSLSASVGDRVTITCQASQGISNYLAWYQQKPGKAPKLLIYDASSLQSGVPSRFSGSGSGTDFTLTISSLQPEDFATYYCQQSYSTPLTFGGGTKVEIK

SEQ ID NO: 292 - protein, 108 aa, Artificial Sequence
  source: /note="Description of Artificial Sequence: Synthetic polypeptide"
EIVMTQSPATLSVSPGERATLSCRASQSLSSSSLAWYQQKPGQAPRLLIYGASTRATGIPARFSGSGSGTEFTLTISSLQSEDFAVYYCQQYGSSPFTFGPGTKVDIK

SEQ ID NO: 293 - protein, 107 aa, Artificial Sequence
  source: /note="Description of Artificial Sequence: Synthetic polypeptide"
DIQMTQSPSSLSASVGDRVTITCRASEHIANWLAWYQQKPGKAPKLLIYGVSSLESGVPSRFSGSGSGTDFTLTISSLQPEDFATYYCQQSYSTPYTFGQGTKLEIK

SEQ ID NO: 294 - protein, 107 aa, Artificial Sequence
  source: /note="Description of Artificial Sequence: Synthetic polypeptide"
DIQMTQSPSSLSASVGDRVTITCRASQSVGSWVAWYQQKPGKAPKLLIYPASTLQSGVPSRFSGSGSGTDFTLTISSLQPEDFATYYCQQSYSTPLTFGGGTKVEIK

SEQ ID NO: 295 - protein, 107 aa, Artificial Sequence
  source: /note="Description of Artificial Sequence: Synthetic polypeptide"
DIQMTQSPSSLSASVGDRVTITCRASQSISPWLAWYQQKPGKAPKLLIYDASNLETGVPSRFSGSGSGTDFTLTISSLQPEDFATYYCQQTYSTPITFGQGTRLEIK

SEQ ID NO: 296 - protein, 107 aa, Artificial Sequence
  source: /note="Description of Artificial Sequence: Synthetic polypeptide"
DIQMTQSPSSLSASVGDRVTITCRASQGISRYLAWYQQKPGKAPKLLIYAASTLQSGVPSRFSGSGSGTDFTLTISSLQPEDFATYYCQQSYSTPLTFGQGTKVEIK

SEQ ID NO: 297 - protein, 108 aa, Artificial Sequence
  source: /note="Description of Artificial Sequence: Synthetic polypeptide"
EIVMTQSPATLSVSPGERATLSCRASQTVSSNYLAWYQQKPGQAPRLLIYGASTRASGIPARFSGSGSGTEFTLTISSLQSEDFAVYYCQQYYTTPLTFGGGTKVEIK

SEQ ID NO: 298 - protein, 107 aa, Artificial Sequence
  source: /note="Description of Artificial Sequence: Synthetic polypeptide"
DIQMTQSPSSLSASVGDRVTITCRASQSISSYLNWYQQKPGKAPKLLIYAASSLQSGVPSRFSGSGSGTDFTLTISSLQPEDFATYYCQQSFSTPLTFGQGTRLEIK

SEQ ID NO: 299 - protein, 107 aa, Artificial Sequence
  source: /note="Description of Artificial Sequence: Synthetic polypeptide"
DIQMTQSPSSLSASVGDRVTITCRASQSISSWLAWYQQKPGKAPKLLIYDASNLETGVPSRFSGSGSGTDFTLTISSLQPEDFATYYCQQSYSTPPTFGQGTKVEIK

SEQ ID NO: 300 - protein, 107 aa, Artificial Sequence
  source: /note="Description of Artificial Sequence: Synthetic polypeptide"
DIQMTQSPSSLSASVGDRVTITCRASQGISNNLNWYQQKPGKAPKLLIYATSTLQSGVPSRFSGSGSGTDFTLTISSLQPEDFATYYCQQSYSTPYTFGQGTKVEIK

SEQ ID NO: 301 - protein, 107 aa, Artificial Sequence
  source: /note="Description of Artificial Sequence: Synthetic polypeptide"
DIQMTQSPSSLSASVGDRVTITCRSSQGIRNDLSWYQQKPGKAPKLLIYLASNSHSGVPSRFSGSGSGTDFTLTISSLQPEDFATYYCLQHNSYPLTFGGGTKVEIK

SEQ ID NO: 302 - protein, 107 aa, Artificial Sequence
  source: /note="Description of Artificial Sequence: Synthetic polypeptide"
DIQMTQSPSSLSASVGDRVTITCRASQSISRWLAWYQQKPGKAPKLLIYAASSLQSGVPSRFSGSGSGTDFTLTISSLQPEDFATYYCQQSYSTPYTFGQGTKLEIK

SEQ ID NO: 303 - protein, 107 aa, Artificial Sequence
  source: /note="Description of Artificial Sequence: Synthetic polypeptide"
DIQMTQSPSSLSASVGDRVTITCRASQSISSWLAWYQQKPGKAPKLLIYDSSSLQTGVPSRFSGSGSGTDFTLTISSLQPEDFATYYCQQSYSTPVTFGQGTKVEIK

SEQ ID NO: 304 - protein, 107 aa, Artificial Sequence
  source: /note="Description of Artificial Sequence: Synthetic polypeptide"
DIQITHSPSSLSASVGYRLTITCRDSHSITTWLAWYHQKPWNAPKLMIYAASNLESGVPSRFSGSGSGTYFTLTISSLQPEDFATYYCQHFYNTQYTFGQGTKVEIK

SEQ ID NO: 305 - protein, 108 aa, Artificial Sequence
  source: /note="Description of Artificial Sequence: Synthetic polypeptide"
DIQMTQSPSSLSASVGDRVTITCRASQVIRNDLAWYQQKPGKAPKLLIYAASTLQSGVPSRFSGSGSGTDFTLTISSLQPEDFATYYCQQSLQYPSHFFGQGTKVEIK

SEQ ID NO: 306 - protein, 107 aa, Artificial Sequence
  source: /note="Description of Artificial Sequence: Synthetic polypeptide"
DIQMTQSPSSLSASVGDRVTITCRASQSISRYLNWYQQKPGKAPKLLIYAASTLQSGVPSRFSGSGSGTDFTLTISSLQPEDFAPYYCQQSYSTPLTFGPGTKVDIK

SEQ ID NO: 307 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 308 - protein, 5 aa, Artificial Sequence
  source: /note="Description of Artificial Sequence: Synthetic peptide"
SDYMH

SEQ ID NO: 309 - protein, 5 aa, Artificial Sequence
  source: /note="Description of Artificial Sequence: Synthetic peptide"
GYYMH

SEQ ID NO: 310 - protein, 5 aa, Artificial Sequence
  source: /note="Description of Artificial Sequence: Synthetic peptide"
SYYMH

SEQ ID NO: 311 - protein, 5 aa, Artificial Sequence
  source: /note="Description of Artificial Sequence: Synthetic peptide"
GYYIH

SEQ ID NO: 312 - protein, 5 aa, Artificial Sequence
  source: /note="Description of Artificial Sequence: Synthetic peptide"
THYMH

SEQ ID NO: 313 - protein, 5 aa, Artificial Sequence
  source: /note="Description of Artificial Sequence: Synthetic peptide"
SHDIN

SEQ ID NO: 314 - protein, 5 aa, Artificial Sequence
  source: /note="Description of Artificial Sequence: Synthetic peptide"
DHYLH

SEQ ID NO: 315 - protein, 5 aa, Artificial Sequence
  source: /note="Description of Artificial Sequence: Synthetic peptide"
NYYMH

SEQ ID NO: 316 - protein, 5 aa, Artificial Sequence
  source: /note="Description of Artificial Sequence: Synthetic peptide"
AYYVH

SEQ ID NO: 317 - protein, 5 aa, Artificial Sequence
  source: /note="Description of Artificial Sequence: Synthetic peptide"
RHYVH

SEQ ID NO: 318 - protein, 4 aa, Artificial Sequence
  source: /note="Description of Artificial Sequence: Synthetic peptide"
NYIH

SEQ ID NO: 319 - protein, 5 aa, Artificial Sequence
  source: /note="Description of Artificial Sequence: Synthetic peptide"
NHYVH

SEQ ID NO: 320 - protein, 5 aa, Artificial Sequence
  source: /note="Description of Artificial Sequence: Synthetic peptide"
SHYMH

SEQ ID NO: 321 - protein, 5 aa, Artificial Sequence
  source: /note="Description of Artificial Sequence: Synthetic peptide"
RHLLH

SEQ ID NO: 322 - protein, 17 aa, Artificial Sequence
  source: /note="Description of Artificial Sequence: Synthetic peptide"
WMSPYNGITGYAQKFQG

SEQ ID NO: 323 - protein, 17 aa, Artificial Sequence
  source: /note="Description of Artificial Sequence: Synthetic peptide"
WMSPSSGITGYAQKFQG

SEQ ID NO: 324 - protein, 17 aa, Artificial Sequence
  source: /note="Description of Artificial Sequence: Synthetic peptide"
WMTTNSGITGYAQKFQG

SEQ ID NO: 325 - protein, 17 aa, Artificial Sequence
  source: /note="Description of Artificial Sequence: Synthetic peptide"
GIIPIFGTASYAQKFQG

SEQ ID NO: 326 - protein, 17 aa, Artificial Sequence
  source: /note="Description of Artificial Sequence: Synthetic peptide"
WMNPNSGHAGSAQKFQG

SEQ ID NO: 327 - protein, 17 aa, Artificial Sequence
  source: /note="Description of Artificial Sequence: Synthetic peptide"
WMNPNSGNTGYAQKFQG

SEQ ID NO: 328 - protein, 17 aa, Artificial Sequence
  source: /note="Description of Artificial Sequence: Synthetic peptide"
WMNPNSGNTGYSQKFQG

SEQ ID NO: 329 - protein, 17 aa, Artificial Sequence
  source: /note="Description of Artificial Sequence: Synthetic peptide"
WMNPNIGNTGYAQKFQG

SEQ ID NO: 330 - protein, 17 aa, Artificial Sequence
  source: /note="Description of Artificial Sequence: Synthetic peptide"
WMNPNGGTTGYAQNFQG

SEQ ID NO: 331 - protein, 17 aa, Artificial Sequence
  source: /note="Description of Artificial Sequence: Synthetic peptide"
WMNPNRGITDSAQKFQG

SEQ ID NO: 332 - protein, 17 aa, Artificial Sequence
  source: /note="Description of Artificial Sequence: Synthetic peptide"
WMNPNSGSAGYAQKFQG

SEQ ID NO: 333 - protein, 17 aa, Artificial Sequence
  source: /note="Description of Artificial Sequence: Synthetic peptide"
WIHPRSGATGYAPKFQG

SEQ ID NO: 334 - protein, 17 aa, Artificial Sequence
  source: /note="Description of Artificial Sequence: Synthetic peptide"
WISPRSGVTSYAQKFQG

SEQ ID NO: 335 - protein, 17 aa, Artificial Sequence
  source: /note="Description of Artificial Sequence: Synthetic peptide"
WMDPNSGNTGYAQKFQG

SEQ ID NO: 336 - protein, 17 aa, Artificial Sequence
  source: /note="Description of Artificial Sequence: Synthetic peptide"
WMNPTGGITGYAQKFQG

SEQ ID NO: 337 - protein, 17 aa, Artificial Sequence
  source: /note="Description of Artificial Sequence: Synthetic peptide"
WMNPNSGHTGNAQKFQG

SEQ ID NO: 338 - protein, 17 aa, Artificial Sequence
  source: /note="Description of Artificial Sequence: Synthetic peptide"
WVSPIHGLTGYAPRFQG

SEQ ID NO: 339 - protein, 9 aa, Artificial Sequence
  source: /note="Description of Artificial Sequence: Synthetic peptide"
DRFSGSYDY

SEQ ID NO: 340 - protein, 7 aa, Artificial Sequence
  source: /note="Description of Artificial Sequence: Synthetic peptide"
DRGWFDP

SEQ ID NO: 341 - protein, 9 aa, Artificial Sequence
  source: /note="Description of Artificial Sequence: Synthetic peptide"
EGYSSGLDY

SEQ ID NO: 342 - protein, 11 aa, Artificial Sequence
  source: /note="Description of Artificial Sequence: Synthetic peptide"
DGRFWSGYPDY

SEQ ID NO: 343 - protein, 10 aa, Artificial Sequence
  source: /note="Description of Artificial Sequence: Synthetic peptide"
ESIAVAGYDY

SEQ ID NO: 344 - protein, 10 aa, Artificial Sequence
  source: /note="Description of Artificial Sequence: Synthetic peptide"
DRWYMGSADY

SEQ ID NO: 345 - protein, 10 aa, Artificial Sequence
  source: /note="Description of Artificial Sequence: Synthetic peptide"
DDWGGDWFDP

SEQ ID NO: 346 - protein, 10 aa, Artificial Sequence
  source: /note="Description of Artificial Sequence: Synthetic peptide"
ERLSVAGFDY

SEQ ID NO: 347 - protein, 10 aa, Artificial Sequence
  source: /note="Description of Artificial Sequence: Synthetic peptide"
EPLQLGGFDY

SEQ ID NO: 348 - protein, 10 aa, Artificial Sequence
  source: /note="Description of Artificial Sequence: Synthetic peptide"
EGFGPNAFDI

SEQ ID NO: 349 - protein, 10 aa, Artificial Sequence
  source: /note="Description of Artificial Sequence: Synthetic peptide"
DSWYGDWFDP

SEQ ID NO: 350 - protein, 9 aa, Artificial Sequence
  source: /note="Description of Artificial Sequence: Synthetic peptide"
EVIEVGMDV

SEQ ID NO: 351 - protein, 9 aa, Artificial Sequence
  source: /note="Description of Artificial Sequence: Synthetic peptide"
EAWFGELST

SEQ ID NO: 352 - protein, 9 aa, Artificial Sequence
  source: /note="Description of Artificial Sequence: Synthetic peptide"
EAYVAAFDI

SEQ ID NO: 353 - protein, 9 aa, Artificial Sequence
  source: /note="Description of Artificial Sequence: Synthetic peptide"
ERGYNAFDY

SEQ ID NO: 354 - protein, 8 aa, Artificial Sequence
  source: /note="Description of Artificial Sequence: Synthetic peptide"
DSVFGLDY

SEQ ID NO: 355 - protein, 10 aa, Artificial Sequence
  source: /note="Description of Artificial Sequence: Synthetic peptide"
DLDYVRAFDI

SEQ ID NO: 356 - protein, 9 aa, Artificial Sequence
  source: /note="Description of Artificial Sequence: Synthetic peptide"
ESWGGYFDL

SEQ ID NO: 357 - protein, 9 aa, Artificial Sequence
  source: /note="Description of Artificial Sequence: Synthetic peptide"
DRTTYAFDI

SEQ ID NO: 358 - protein, 9 aa, Artificial Sequence
  source: /note="Description of Artificial Sequence: Synthetic peptide"
GNWVDAFDI

SEQ ID NO: 359 - protein, 11 aa, Artificial Sequence
  source: /note="Description of Artificial Sequence: Synthetic peptide"
VHGSGSDGMDV

SEQ ID NO: 360 - protein, 11 aa, Artificial Sequence
  source: /note="Description of Artificial Sequence: Synthetic peptide"
RASQSISSWLA

SEQ ID NO: 361 - protein, 11 aa, Artificial Sequence
  source: /note="Description of Artificial Sequence: Synthetic peptide"
RASQSVGTWLA

SEQ ID NO: 362 - protein, 11 aa, Artificial Sequence
  source: /note="Description of Artificial Sequence: Synthetic peptide"
RASQGISNYLA

SEQ ID NO: 363 - protein, 11 aa, Artificial Sequence
  source: /note="Description of Artificial Sequence: Synthetic peptide"
RASQSISTWLA

SEQ ID NO: 364 - protein, 11 aa, Artificial Sequence
  source: /note="Description of Artificial Sequence: Synthetic peptide"
QASQDISNHLN

SEQ ID NO: 365 - protein, 11 aa, Artificial Sequence
  source: /note="Description of Artificial Sequence: Synthetic peptide"
RASQGISSWLA

SEQ ID NO: 366 - protein, 11 aa, Artificial Sequence
  source: /note="Description of Artificial Sequence: Synthetic peptide"
RASESISSWLA

SEQ ID NO: 367 - protein, 11 aa, Artificial Sequence
  source: /note="Description of Artificial Sequence: Synthetic peptide"
RASQSVGSWLA

SEQ ID NO: 368 - protein, 11 aa, Artificial Sequence
  source: /note="Description of Artificial Sequence: Synthetic peptide"
RASQNISNFLN

SEQ ID NO: 369 - protein, 12 aa, Artificial Sequence
  source: /note="Description of Artificial Sequence: Synthetic peptide"
RASQSLSSSYLA

SEQ ID NO: 370 - protein, 11 aa, Artificial Sequence
  source: /note="Description of Artificial Sequence: Synthetic peptide"
RASQSISSYLN

SEQ ID NO: 371 - protein, 11 aa, Artificial Sequence
  source: /note="Description of Artificial Sequence: Synthetic peptide"
RASQSISRWLA

SEQ ID NO: 372 - protein, 11 aa, Artificial Sequence
  source: /note="Description of Artificial Sequence: Synthetic peptide"
RDSHSITTWLA

SEQ ID NO: 373 - protein, 11 aa, Artificial Sequence
  source: /note="Description of Artificial Sequence: Synthetic peptide"
RASQVIRNDLA

SEQ ID NO: 374 - protein, 11 aa, Artificial Sequence
  source: /note="Description of Artificial Sequence: Synthetic peptide"
RASQSISRYLN

SEQ ID NO: 375 - protein, 7 aa, Artificial Sequence
  source: /note="Description of Artificial Sequence: Synthetic peptide"
AASSLQS

SEQ ID NO: 376 - protein, 7 aa, Artificial Sequence
  source: /note="Description of Artificial Sequence: Synthetic peptide"
AASTLEN

SEQ ID NO: 377 - protein, 7 aa, Artificial Sequence
  source: /note="Description of Artificial Sequence: Synthetic peptide"
RASNLES

SEQ ID NO: 378 - protein, 7 aa, Artificial Sequence
  source: /note="Description of Artificial Sequence: Synthetic peptide"
AASTLQR

SEQ ID NO: 379 - protein, 7 aa, Artificial Sequence
  source: /note="Description of Artificial Sequence: Synthetic peptide"
AASTLQS

SEQ ID NO: 380 - protein, 7 aa, Artificial Sequence
  source: /note="Description of Artificial Sequence: Synthetic peptide"
GASNLQR

SEQ ID NO: 381 - protein, 7 aa, Artificial Sequence
  source: /note="Description of Artificial Sequence: Synthetic peptide"
AASNLQS

SEQ ID NO: 382 - protein, 7 aa, Artificial Sequence
  source: /note="Description of Artificial Sequence: Synthetic peptide"
GASSLQS

SEQ ID NO: 383 - protein, 7 aa, Artificial Sequence
  source: /note="Description of Artificial Sequence: Synthetic peptide"
AASHLQS

SEQ ID NO: 384 - skipped

SEQ ID NO: 385 - protein, 7 aa, Artificial Sequence
  source: /note="Description of Artificial Sequence: Synthetic peptide"
DSSSLQT

SEQ ID NO: 386 - protein, 7 aa, Artificial Sequence
  source: /note="Description of Artificial Sequence: Synthetic peptide"
AASNLES

SEQ ID NO: 387 - protein, 9 aa, Artificial Sequence
  source: /note="Description of Artificial Sequence: Synthetic peptide"
QQSYSTPYT

SEQ ID NO: 388 - protein, 9 aa, Artificial Sequence
  source: /note="Description of Artificial Sequence: Synthetic peptide"
QQSFSTPYT

SEQ ID NO: 389 - protein, 9 aa, Artificial Sequence
  source: /note="Description of Artificial Sequence: Synthetic peptide"
QQSYSTPLT

SEQ ID NO: 390 - protein, 9 aa, Artificial Sequence
  source: /note="Description of Artificial Sequence: Synthetic peptide"
QQSYSTPFT

SEQ ID NO: 391 - protein, 9 aa, Artificial Sequence
  source: /note="Description of Artificial Sequence: Synthetic peptide"
QQSYSTPIT

SEQ ID NO: 392 - protein, 9 aa, Artificial Sequence
  source: /note="Description of Artificial Sequence: Synthetic peptide"
QQYYSTPYT

SEQ ID NO: 393 - protein, 9 aa, Artificial Sequence
  source: /note="Description of Artificial Sequence: Synthetic peptide"
QQSYSLPYT

SEQ ID NO: 394 - protein, 9 aa, Artificial Sequence
  source: /note="Description of Artificial Sequence: Synthetic peptide"
HQYFTTPLT

SEQ ID NO: 395 - protein, 9 aa, Artificial Sequence
  source: /note="Description of Artificial Sequence: Synthetic peptide"
QQSYSMPYT

SEQ ID NO: 396 - protein, 9 aa, Artificial Sequence
  source: /note="Description of Artificial Sequence: Synthetic peptide"
QQSYSTPVT

SEQ ID NO: 397 - protein, 9 aa, Artificial Sequence
  source: /note="Description of Artificial Sequence: Synthetic peptide"
QHFYNTQYT

SEQ ID NO: 398 - protein, 10 aa, Artificial Sequence
  source: /note="Description of Artificial Sequence: Synthetic peptide"
QQSLQYPSHF

SEQ ID NO: 399 - protein, 30 aa, Artificial Sequence; identical to SEQ ID NO: 187
  source: /note="Description of Artificial Sequence: Synthetic polypeptide"

SEQ ID NO: 400 - protein, 30 aa, Artificial Sequence; identical to SEQ ID NO: 178
  source: /note="Description of Artificial Sequence: Synthetic polypeptide"

SEQ ID NO: 401 - protein, 30 aa, Artificial Sequence; identical to SEQ ID NO: 191
  source: /note="Description of Artificial Sequence: Synthetic polypeptide"

SEQ ID NO: 402 - protein, 30 aa, Artificial Sequence
  source: /note="Description of Artificial Sequence: Synthetic polypeptide"
QVQLVQSGAEVKEPGASVKVSCKASGYTFT

SEQ ID NO: 403 - protein, 30 aa, Artificial Sequence
  source: /note="Description of Artificial Sequence: Synthetic polypeptide"
QVQLVQSGAEVKKPGASVKVSCKASGYNFS

SEQ ID NO: 404 - skipped

SEQ ID NO: 405 - protein, 30 aa, Artificial Sequence; identical to SEQ ID NO: 189
  source: /note="Description of Artificial Sequence: Synthetic polypeptide"

SEQ ID NO: 406 - protein, 30 aa, Artificial Sequence; identical to SEQ ID NO: 190
  source: /note="Description of Artificial Sequence: Synthetic polypeptide"

SEQ ID NO: 407 - protein, 14 aa, Artificial Sequence
  source: /note="Description of Artificial Sequence: Synthetic peptide"
WVRQAPGQGLEWMG

SEQ ID NO: 408 - protein, 14 aa, Artificial Sequence
  source: /note="Description of Artificial Sequence: Synthetic peptide"
WMRQAPGQGLEWIG

SEQ ID NO: 409 - protein, 32 aa, Artificial Sequence; identical to SEQ ID NO: 196
  source: /note="Description of Artificial Sequence: Synthetic polypeptide"

SEQ ID NO: 410 - protein, 32 aa, Artificial Sequence; identical to SEQ ID NO: 199
  source: /note="Description of Artificial Sequence: Synthetic polypeptide"

SEQ ID NO: 411 - protein, 11 aa, Artificial Sequence
  source: /note="Description of Artificial Sequence: Synthetic peptide"
WGQGTLVTVSS

SEQ ID NO: 412 - protein, 11 aa, Artificial Sequence
  source: /note="Description of Artificial Sequence: Synthetic peptide"
WGQGTTVTVSS

SEQ ID NO: 413 - protein, 11 aa, Artificial Sequence
  source: /note="Description of Artificial Sequence: Synthetic peptide"
WGRGTLVTVSS

SEQ ID NO: 414 - protein, 11 aa, Artificial Sequence
  source: /note="Description of Artificial Sequence: Synthetic peptide"
WGQGTMVTVSS

SEQ ID NO: 415 - protein, 23 aa, Artificial Sequence
  source: /note="Description of Artificial Sequence: Synthetic peptide"
DIQMTQSPSSLSASVGDRVTITC

SEQ ID NO: 416 - protein, 23 aa, Artificial Sequence
  source: /note="Description of Artificial Sequence: Synthetic peptide"
EIVMTQSPATLSVSPGERATLSC

SEQ ID NO: 417 - protein, 23 aa, Artificial Sequence
  source: /note="Description of Artificial Sequence: Synthetic peptide"
DIQITHSPSSLSASVGYRLTITC

SEQ ID NO: 418 - protein, 15 aa, Artificial Sequence
  source: /note="Description of Artificial Sequence: Synthetic peptide"
WYQQKPGKAPKLLIY

SEQ ID NO: 419 - protein, 15 aa, Artificial Sequence
  source: /note="Description of Artificial Sequence: Synthetic peptide"
WYQQKPGQAPRLLIY

SEQ ID NO: 420 - protein, 15 aa, Artificial Sequence
  source: /note="Description of Artificial Sequence: Synthetic peptide"
WYHQKPWNAPKLMIY

SEQ ID NO: 421 - protein, 32 aa, Artificial Sequence; identical to SEQ ID NO: 213
  source: /note="Description of Artificial Sequence: Synthetic polypeptide"

SEQ ID NO: 422 - protein, 32 aa, Artificial Sequence; identical to SEQ ID NO: 214
  source: /note="Description of Artificial Sequence: Synthetic polypeptide"

SEQ ID NO: 423 - protein, 32 aa, Artificial Sequence; identical to SEQ ID NO: 215
  source: /note="Description of Artificial Sequence: Synthetic polypeptide"

SEQ ID NO: 424 - protein, 32 aa, Artificial Sequence; identical to SEQ ID NO: 216
  source: /note="Description of Artificial Sequence: Synthetic polypeptide"

SEQ ID NO: 425 - protein, 10 aa, Artificial Sequence
  source: /note="Description of Artificial Sequence: Synthetic peptide"
FGGGTKVEIK

SEQ ID NO: 426 - protein, 10 aa, Artificial Sequence
  source: /note="Description of Artificial Sequence: Synthetic peptide"
FGQGTKLEIK

SEQ ID NO: 427 - protein, 10 aa, Artificial Sequence
  source: /note="Description of Artificial Sequence: Synthetic peptide"
FGQGTKVEIK

SEQ ID NO: 428 - protein, 10 aa, Artificial Sequence
  source: /note="Description of Artificial Sequence: Synthetic peptide"
FGQGTRLEIK

SEQ ID NO: 429 - protein, 10 aa, Artificial Sequence
  source: /note="Description of Artificial Sequence: Synthetic peptide"
FGPGTKVDIK

SEQ ID NO: 430 - protein, 118 aa, Artificial Sequence
  source: /note="Description of Artificial Sequence: Synthetic polypeptide"
QVQLVQSGAEVKKPGASVKVSCKASGYTFSSDYMHWVRQAPGQGLEWMGWMSPYNGITGYAQKFQGRVTMTRDTSTSTVYMELSSLRSEDTAVYYCARDRFSGSYDYWGQGTLVTVSS

SEQ ID NO: 431 - protein, 116 aa, Artificial Sequence
  source: /note="Description of Artificial Sequence: Synthetic polypeptide"
QVQLVQSGAEVKKPGASVKVSCKASGYTFTGYYMHWVRQAPGQGLEWMGWMSPSSGITGYAQKFQGRVTMTRDTSTSTVYMELSSLRSEDTAVYYCARDRGWFDPWGQGTLVTVSS

SEQ ID NO: 432 - protein, 118 aa, Artificial Sequence
  source: /note="Description of Artificial Sequence: Synthetic polypeptide"
QVQLVQSGAEVKKPGASVKVSCKASGYTFSSYYMHWVRQAPGQGLEWMGWMTTNSGITGYAQKFQGRVTMTRDTSTSTVYMELSSLRSEDTAVYYCAREGYSSGLDYWGQGTLVTVSS

SEQ ID NO: 433 - protein, 120 aa, Artificial Sequence
  source: /note="Description of Artificial Sequence: Synthetic polypeptide"
QVQLVQSGAEVKKPGSSVKVSCKASGYTFTGYYIHWVRQAPGQGLEWMGGIIPIFGTASYAQKFQGRVTITADESTSTAYMELSSLRSEDTAVYYCARDGRFWSGYPDYWGQGTLVTVSS

SEQ ID NO: 434 - protein, 119 aa, Artificial Sequence
  source: /note="Description of Artificial Sequence: Synthetic polypeptide"
QVQLVQSGAEVKKPGASVKVSCKASGYTFSTHYMHWVRQAPGQGLEWMGWMNPNSGHAGSAQKFQGRVTMTRDTSTSTVYMELSSLRSEDTAVYYCARESIAVAGYDYWGQGTLVTVSS

SEQ ID NO: 435 - protein, 119 aa, Artificial Sequence
  source: /note="Description of Artificial Sequence: Synthetic polypeptide"
QVQLVQSGAEVKKPGASVKVSCKASGYTFTSHDINWVRQAPGQGLEWMGWMNPNSGNTGYAQKFQGRVTMTRDTSTSTVYMELSSLRSEDTAVYYCARDRWYMGSADYWGQGTLVTVSS

SEQ ID NO: 436 - protein, 119 aa, Artificial Sequence
  source: /note="Description of Artificial Sequence: Synthetic polypeptide"
QVQLVQSGAEVKKPGASVKVSCKASGYTFTSYYMHWVRQAPGQGLEWMGWMNPNSGNTGYAQKFQGRVTMTRDTSTSTVYMELSSLRSEDTAVYYCARDDWGGDWFDPWGQGTLVTVSS

SEQ ID NO: 437 - protein, 119 aa, Artificial Sequence
  source: /note="Description of Artificial Sequence: Synthetic polypeptide"
QVQLVQSGAEVKKPGASVKVSCKASGYTFSTHYMHWVRQAPGQGLEWMGWMNPNSGNTGYSQKFQGRVTMTRDTSTSTVYMELSSLRSEDTAVYYCARERLSVAGFDYWGQGTLVTVSS

SEQ ID NO: 438 - protein, 119 aa, Artificial Sequence
  source: /note="Description of Artificial Sequence: Synthetic polypeptide"
QVQLVQSGAEVKEPGASVKVSCKASGYTFTDHYLHWVRQAPGQGLEWMGWMNPNIGNTGYAQKFQGRVTMTRDTSTSTVYMELSSLRSEDTAVYYCAREPLQLGGFDYWGQGTLVTVSS

SEQ ID NO: 439 - protein, 119 aa, Artificial Sequence
  source: /note="Description of Artificial Sequence: Synthetic polypeptide"
QVQLVQSGAEVKKPGASVKVSCKASGYTFTGYYMHWVRQAPGQGLEWMGWMNPNGGTTGYAQNFQGRVTMTRDTSTSTVYMELSSLRSEDTAVYYCAREGFGPNAFDIWGQGTTVTVSS

SEQ ID NO: 440 - protein, 119 aa, Artificial Sequence
  source: /note="Description of Artificial Sequence: Synthetic polypeptide"
QVQLVQSGAEVKKPGASVKVSCKASGYTFTGYYMHWVRQAPGQGLEWMGWMNPNSGNTGYAQKFQGRVTMTRDTSTSTVYMELSSLRSEDTAVYYCARDSWYGDWFDPWGQGTLVTVSS

SEQ ID NO: 441 - protein, 118 aa, Artificial Sequence
  source: /note="Description of Artificial Sequence: Synthetic polypeptide"
QVQLVQSGAEVKKPGASVKVSCKASGYTFTGYYMHWMRQAPGQGLEWIGWMSPYNGITGYAQKFQGRVTMTRDTSTSTVYMELSSLRSEDTAVYYCAREVIEVGMDVWGQGTTVTVSS

SEQ ID NO: 442 - protein, 118 aa, Artificial Sequence
  source: /note="Description of Artificial Sequence: Synthetic polypeptide"
QVQLVQSGAEVKKPGASVKVSCKASGYTFTNYYMHWVRQAPGQGLEWMGWMSPSSGITGYAQKFQGRVTMTRDTSTSTVYMELSSLRSEDTAVYYCAREAWFGELSTWGQGTLVTVSS

SEQ ID NO: 443 - protein, 118 aa, Artificial Sequence
  source: /note="Description of Artificial Sequence: Synthetic polypeptide"
QVQLVQSGAEVKKPGASVKVSCKASGYNFSAYYVHWVRQAPGQGLEWMGWMNPNRGITDSAQKFQGRVTMTRDTSTSTVYMELSSLRSEDTAVYYCAREAYVAAFDIWGQGTTVTVSS

SEQ ID NO: 444 - protein, 118 aa, Artificial Sequence
  source: /note="Description of Artificial Sequence: Synthetic polypeptide"
QVQLVQSGAEVKKPGASVKVSCKASGYTFTRHYVHWVRQAPGQGLEWMGWMNPNSGSAGYAQKFQGRVTMTRDTSTSTVYMELSSLRSEDTAVYYCARERGYNAFDYWGQGTLVTVSS

SEQ ID NO: 445 - protein, 116 aa, Artificial Sequence
  source: /note="Description of Artificial Sequence: Synthetic polypeptide"
QVQLVQSGAEVKKPGASVKVSCKASGYTLPNYIHWVRQAPGQGLEWMGWIHPRSGATGYAPKFQGRVTMTRDTSTSTVYMELSSLRSEDTAVYYCARDSVFGLDYWGQGTLVTVSS

SEQ ID NO: 446 - protein, 119 aa, Artificial Sequence; identical to SEQ ID NO: 260
  source: /note="Description of Artificial Sequence: Synthetic polypeptide"

SEQ ID NO: 447 - protein, 118 aa, Artificial Sequence; identical to SEQ ID NO: 261
  source: /note="Description of Artificial Sequence: Synthetic polypeptide"

SEQ ID NO: 448 - protein, 118 aa, Artificial Sequence; identical to SEQ ID NO: 262
  source: /note="Description of Artificial Sequence: Synthetic polypeptide"

SEQ ID NO: 449 - protein, 118 aa, Artificial Sequence; identical to SEQ ID NO: 243
  source: /note="Description of Artificial Sequence: Synthetic polypeptide"

SEQ ID NO: 450 - protein, 120 aa, Artificial Sequence; identical to SEQ ID NO: 264
  source: /note="Description of Artificial Sequence: Synthetic polypeptide"

SEQ ID NO: 451 - protein, 107 aa, Artificial Sequence
  source: /note="Description of Artificial Sequence: Synthetic polypeptide"
DIQMTQSPSSLSASVGDRVTITCRASQSISSWLAWYQQKPGKAPKLLIYAASSLQSGVPSRFSGSGSGTDFTLTISSLQPEDFATYYCQQSYSTPYTFGGGTKVEIK

SEQ ID NO: 452 - protein, 107 aa, Artificial Sequence
  source: /note="Description of Artificial Sequence: Synthetic polypeptide"
DIQMTQSPSSLSASVGDRVTITCRASQSVGTWLAWYQQKPGKAPKLLIYAASTLENGVPSRFSGSGSGTDFTLTISSLQPEDFATYYCQQSFSTPYTFGGGTKVEIK

SEQ ID NO: 453 - protein, 107 aa, Artificial Sequence; identical to SEQ ID NO: 451
  source: /note="Description of Artificial Sequence: Synthetic polypeptide"

SEQ ID NO: 454 - protein, 107 aa, Artificial Sequence
  source: /note="Description of Artificial Sequence: Synthetic polypeptide"
DIQMTQSPSSLSASVGDRVTITCRASQGISNYLAWYQQKPGKAPKLLIYRASNLESGVPSRFSGSGSGTDFTLTISSLQPEDFATYYCQQSYSTPLTFGGGTKVEIK

SEQ ID NO: 455 - protein, 107 aa, Artificial Sequence
  source: /note="Description of Artificial Sequence: Synthetic polypeptide"
DIQMTQSPSSLSASVGDRVTITCRASQSISSWLAWYQQKPGKAPKLLIYAASTLQRGVPSRFSGSGSGTDFTLTISSLQPEDFATYYCQQSYSTPYTFGQGTKLEIK

SEQ ID NO: 456 - protein, 107 aa, Artificial Sequence
  source: /note="Description of Artificial Sequence: Synthetic polypeptide"
DIQMTQSPSSLSASVGDRVTITCRASQSISTWLAWYQQKPGKAPKLLIYAASTLQSGVPSRFSGSGSGTDFTLTISSLQPEDFATYYCQQSYSTPFTFGQGTKVEIK

SEQ ID NO: 457 - protein, 107 aa, Artificial Sequence
  source: /note="Description of Artificial Sequence: Synthetic polypeptide"
DIQMTQSPSSLSASVGDRVTITCQASQDISNHLNWYQQKPGKAPKLLIYGASNLQRGVPSRFSGSGSGTDFTLTISSLQPEDFATYYCQQSYSTPITFGQGTRLEIK

SEQ ID NO: 458 - protein, 107 aa, Artificial Sequence
  source: /note="Description of Artificial Sequence: Synthetic polypeptide"
DIQMTQSPSSLSASVGDRVTITCRASQGISSWLAWYQQKPGKAPKLLIYAASSLQSGVPSRFSGSGSGTDFTLTISSLQPEDFATYYCQQSYSTPYTFGQGTKVEIK

SEQ ID NO: 459 - protein, 107 aa, Artificial Sequence
  source: /note="Description of Artificial Sequence: Synthetic polypeptide"
DIQMTQSPSSLSASVGDRVTITCRASESISSWLAWYQQKPGKAPKLLIYAASSLQSGVPSRFSGSGSGTDFTLTISSLQPEDFATYYCQQSYSTPLTFGQGTKLEIK

SEQ ID NO: 460 - protein, 107 aa, Artificial Sequence
  source: /note="Description of Artificial Sequence: Synthetic polypeptide"
DIQMTQSPSSLSASVGDRVTITCRASQSISSWLAWYQQKPGKAPKLLIYAASNLQSGVPSRFSGSGSGTDFTLTISSLQPEDFATYYCQQYYSTPYTFGQGTKVEIK

SEQ ID NO: 461 - protein, 107 aa, Artificial Sequence
  source: /note="Description of Artificial Sequence: Synthetic polypeptide"
DIQMTQSPSSLSASVGDRVTITCRASQSVGSWLAWYQQKPGKAPKLLIYGASSLQSGVPSRFSGSGSGTDFTLTISSLQPEDFATYYCQQSYSTPLTFGGGTKVEIK

SEQ ID NO: 462 - protein, 107 aa, Artificial Sequence
  source: /note="Description of Artificial Sequence: Synthetic polypeptide"
DIQMTQSPSSLSASVGDRVTITCRASQSISSWLAWYQQKPGKAPKLLIYAASHLQSGVPSRFSGSGSGTDFTLTISSLQPEDFATYYCQQSYSTPYTFGQGTRLEIK

SEQ ID NO: 463 - protein, 107 aa, Artificial Sequence
  source: /note="Description of Artificial Sequence: Synthetic polypeptide"
DIQMTQSPSSLSASVGDRVTITCRASQNISNFLNWYQQKPGKAPKLLIYAASSLQSGVPSRFSGSGSGTDFTLTISSLQPEDFATYYCQQSYSLPYTFGQGTKVEIK

SEQ ID NO: 464 - protein, 107 aa, Artificial Sequence
  source: /note="Description of Artificial Sequence: Synthetic polypeptide"
DIQMTQSPSSLSASVGDRVTITCRASQGISSWLAWYQQKPGKAPKLLIYAASSLQSGVPSRFSGSGSGTDFTLTISSLQPEDFATYYCQQSYSTPLTFGGGTKVEIK

SEQ ID NO: 465 - protein, 108 aa, Artificial Sequence
  source: /note="Description of Artificial Sequence: Synthetic polypeptide"
EIVMTQSPATLSVSPGERATLSCRASQSLSSSYLAWYQQKPGQAPRLLIYGASTRATGIPARFSGSGSGTEFTLTISSLQSEDFAVYYCHQYFTTPLTFGQGTRLEIK

SEQ ID NO: 466 - protein, 107 aa, Artificial Sequence
  source: /note="Description of Artificial Sequence: Synthetic polypeptide"
DIQMTQSPSSLSASVGDRVTITCRASQSISSYLNWYQQKPGKAPKLLIYAASTLQSGVPSRFSGSGSGTDFTLTISSLQPEDFATYYCQQSYSMPYTFGQGTKLEIK

SEQ ID NO: 467 - protein, 107 aa, Artificial Sequence; identical to SEQ ID NO: 302
  source: /note="Description of Artificial Sequence: Synthetic polypeptide"

SEQ ID NO: 468 - protein, 107 aa, Artificial Sequence; identical to SEQ ID NO: 303
  source: /note="Description of Artificial Sequence: Synthetic polypeptide"

SEQ ID NO: 469 - protein, 107 aa, Artificial Sequence; identical to SEQ ID NO: 304
  source: /note="Description of Artificial Sequence: Synthetic polypeptide"

SEQ ID NO: 470 - protein, 108 aa, Artificial Sequence; identical to SEQ ID NO: 305
  source: /note="Description of Artificial Sequence: Synthetic polypeptide"

SEQ ID NO: 471 - protein, 107 aa, Artificial Sequence; identical to SEQ ID NO: 306
  source: /note="Description of Artificial Sequence: Synthetic polypeptide"

SEQ ID NO: 472 - protein, 106 aa, Artificial Sequence
  source: /note="Description of Artificial Sequence: Synthetic polypeptide"
DIQLTQSPSSLSASVGDRVTITCKASQDVGTSVAWYQQKPGKAPKLLIYWTSTRHTGVPSRFSGSGSGTDFTFTISSLQPEDIATYYCQQYSLYRSFGQGTKVEIK

SEQ ID NO: 473 - protein, 23 aa, Artificial Sequence
  source: /note="Description of Artificial Sequence: Synthetic peptide"
DIQLTQSPSSLSASVGDRVTITC

SEQ ID NO: 474 - protein, 11 aa, Artificial Sequence
  source: /note="Description of Artificial Sequence: Synthetic peptide"
KASQDVGTSVA

SEQ ID NO: 475 - protein, 15 aa, Artificial Sequence
  source: /note="Description of Artificial Sequence: Synthetic peptide"
WYQQKPGKAPKLLIY

SEQ ID NO: 476 - protein, 7 aa, Artificial Sequence
  source: /note="Description of Artificial Sequence: Synthetic peptide"
WTSTRHT

SEQ ID NO: 477 - protein, 32 aa, Artificial Sequence
  source: /note="Description of Artificial Sequence: Synthetic polypeptide"
GVPSRFSGSGSGTDFTFTISSLQPEDIATYYC

SEQ ID NO: 478 - protein, 8 aa, Artificial Sequence
  source: /note="Description of Artificial Sequence: Synthetic peptide"
QQYSLYRS

SEQ ID NO: 479 - protein, 10 aa, Artificial Sequence
  source: /note="Description of Artificial Sequence: Synthetic peptide"
FGQGTKVEIK

SEQ ID NO: 480 - protein, 119 aa, Artificial Sequence
  source: /note="Description of Artificial Sequence: Synthetic polypeptide"
EVQLVESGGGVVQPGRSLRLSCSSSGFDFTTYWMSWVRQAPGKGLEWVAEIHPDSSTINYAPSLKDRFTISRDNSKNTLFLQMDSLRPEDTGVYFCASLYFGFPWFAYWGQGTPVTVSS

SEQ ID NO: 481 - protein, 30 aa, Artificial Sequence
  source: /note="Description of Artificial Sequence: Synthetic polypeptide"
EVQLVESGGGVVQPGRSLRLSCSSSGFDFT

SEQ ID NO: 482 - protein, 5 aa, Artificial Sequence
  source: /note="Description of Artificial Sequence: Synthetic peptide"
TYWMS

SEQ ID NO: 483 - protein, 14 aa, Artificial Sequence
  source: /note="Description of Artificial Sequence: Synthetic peptide"
WVRQAPGKGLEWVA

SEQ ID NO: 484 - protein, 17 aa, Artificial Sequence
  source: /note="Description of Artificial Sequence: Synthetic peptide"
EIHPDSSTINYAPSLKD

SEQ ID NO: 485 - protein, 32 aa, Artificial Sequence
  source: /note="Description of Artificial Sequence: Synthetic polypeptide"
RFTISRDNSKNTLFLQMDSLRPEDTGVYFCAS

SEQ ID NO: 486 - protein, 10 aa, Artificial Sequence
  source: /note="Description of Artificial Sequence: Synthetic peptide"
LYFGFPWFAY

SEQ ID NO: 487 - protein, 11 aa, Artificial Sequence
  source: /note="Description of Artificial Sequence: Synthetic peptide"
WGQGTPVTVSS

SEQ ID NO: 488 - protein, 108 aa, Artificial Sequence
  source: /note="Description of Artificial Sequence: Synthetic polypeptide"
DIQMTQSPSSLSASVGDRVTITCKASAAVGTYVAWYQQKPGKAPKLLIYSASYRKRGVPSRFSGSGSGTDFTLTISSLQPEDFATYYCHQYYTYPLFTFGQGTKLEIK

SEQ ID NO: 489 - protein, 23 aa, Artificial Sequence
  source: /note="Description of Artificial Sequence: Synthetic peptide"
DIQMTQSPSSLSASVGDRVTITC

SEQ ID NO: 490 - protein, 11 aa, Artificial Sequence
  source: /note="Description of Artificial Sequence: Synthetic peptide"
KASAAVGTYVA

SEQ ID NO: 491 - protein, 15 aa, Artificial Sequence
  source: /note="Description of Artificial Sequence: Synthetic peptide"
WYQQKPGKAPKLLIY

SEQ ID NO: 492 - protein, 7 aa, Artificial Sequence
  source: /note="Description of Artificial Sequence: Synthetic peptide"
SASYRKR

SEQ ID NO: 493 - protein, 32 aa, Artificial Sequence; identical to SEQ ID NO: 213
  source: /note="Description of Artificial Sequence: Synthetic polypeptide"

SEQ ID NO: 494 - protein, 10 aa, Artificial Sequence
  source: /note="Description of Artificial Sequence: Synthetic peptide"
HQYYTYPLFT

SEQ ID NO: 495 - protein, 10 aa, Artificial Sequence
  source: /note="Description of Artificial Sequence: Synthetic peptide"
FGQGTKLEIK

SEQ ID NO: 496 - protein, 30 aa, Artificial Sequence; identical to SEQ ID NO: 178
  source: /note="Description of Artificial Sequence: Synthetic polypeptide"

SEQ ID NO: 497 - protein, 5 aa, Artificial Sequence
  source: /note="Description of Artificial Sequence: Synthetic peptide"
EFGMN

SEQ ID NO: 498 - protein, 14 aa, Artificial Sequence
  source: /note="Description of Artificial Sequence: Synthetic peptide"
WVRQAPGQGLEWMG

SEQ ID NO: 499 - protein, 17 aa, Artificial Sequence
  source: /note="Description of Artificial Sequence: Synthetic peptide"
WINTKTGEATYVEEFKG

SEQ ID NO: 500 - protein, 32 aa, Artificial Sequence
  source: /note="Description of Artificial Sequence: Synthetic polypeptide"
RVTFTTDTSTSTAYMELRSLRSDDTAVYYCAR

SEQ ID NO: 501 - protein, 12 aa, Artificial Sequence
  source: /note="Description of Artificial Sequence: Synthetic peptide"
WDFAYYVEAMDY

SEQ ID NO: 502 - protein, 11 aa, Artificial Sequence
  source: /note="Description of Artificial Sequence: Synthetic peptide"
WGQGTTVTVSS

SEQ ID NO: 503 - protein, 106 aa, Artificial Sequence
  source: /note="Description of Artificial Sequence: Synthetic polypeptide"
ENVLTQSPSSMSASVGDRVNIACSASSSVSYMHWFQQKPGKSPKLWIYSTSNLASGVPSRFSGSGSGTDYSLTISSMQPEDAATYYCQQRSSYPLTFGGGTKLEIK

SEQ ID NO: 504 - protein, 23 aa, Artificial Sequence
  source: /note="Description of Artificial Sequence: Synthetic peptide"
ENVLTQSPSSMSASVGDRVNIAC

SEQ ID NO: 505 - protein, 10 aa, Artificial Sequence
  source: /note="Description of Artificial Sequence: Synthetic peptide"
SASSSVSYMH

SEQ ID NO: 506 - protein, 15 aa, Artificial Sequence
  source: /note="Description of Artificial Sequence: Synthetic peptide"
WFQQKPGKSPKLWIY

SEQ ID NO: 507 - protein, 7 aa, Artificial Sequence
  source: /note="Description of Artificial Sequence: Synthetic peptide"
STSNLAS

SEQ ID NO: 508 - protein, 32 aa, Artificial Sequence
  source: /note="Description of Artificial Sequence: Synthetic polypeptide"
GVPSRFSGSGSGTDYSLTISSMQPEDAATYYC

SEQ ID NO: 509 - protein, 9 aa, Artificial Sequence
  source: /note="Description of Artificial Sequence: Synthetic peptide"
QQRSSYPLT

SEQ ID NO: 510 - protein, 10 aa, Artificial Sequence
  source: /note="Description of Artificial Sequence: Synthetic peptide"
FGGGTKLEIK

SEQ ID NO: 511 - protein, 120 aa, Artificial Sequence
  source: /note="Description of Artificial Sequence: Synthetic polypeptide"
QVKLEQSGAEVVKPGASVKLSCKASGFNIKDSYMHWLRQGPGQRLEWIGWIDPENGDTEYAPKFQGKATFTTDTSANTAYLGLSSLRPEDTAVYYCNEGTPTGPYYFDYWGQGTLVTVSS

SEQ ID NO: 512 - protein, 30 aa, Artificial Sequence
  source: /note="Description of Artificial Sequence: Synthetic polypeptide"
QVKLEQSGAEVVKPGASVKLSCKASGFNIK

SEQ ID NO: 513 - protein, 5 aa, Artificial Sequence
  source: /note="Description of Artificial Sequence: Synthetic peptide"
DSYMH

SEQ ID NO: 514 - protein, 14 aa, Artificial Sequence
  source: /note="Description of Artificial Sequence: Synthetic peptide"
WLRQGPGQRLEWIG

SEQ ID NO: 515 - protein, 17 aa, Artificial Sequence
  source: /note="Description of Artificial Sequence: Synthetic peptide"
WIDPENGDTEYAPKFQG

SEQ ID NO: 516 - protein, 32 aa, Artificial Sequence
  source: /note="Description of Artificial Sequence: Synthetic polypeptide"
KATFTTDTSANTAYLGLSSLRPEDTAVYYCNE

SEQ ID NO: 517 - protein, 11 aa, Artificial Sequence
  source: /note="Description of Artificial Sequence: Synthetic peptide"
GTPTGPYYFDY

SEQ ID NO: 518 - protein, 11 aa, Artificial Sequence
  source: /note="Description of Artificial Sequence: Synthetic peptide"
WGQGTLVTVSS

SEQ ID NO: 519 - protein, 106 aa, Artificial Sequence
  source: /note="Description of Artificial Sequence: Synthetic polypeptide"
ENVLTQSPSSMSVSVGDRVTIACSASSSVPYMHWLQQKPGKSPKLLIYLTSNLASGVPSRFSGSGSGTDYSLTISSVQPEDAATYYCQQRSSYPLTFGGGTKLEIK

SEQ ID NO: 520 - protein, 23 aa, Artificial Sequence
  source: /note="Description of Artificial Sequence: Synthetic peptide"
ENVLTQSPSSMSVSVGDRVTIAC

SEQ ID NO: 521 - protein, 10 aa, Artificial Sequence
  source: /note="Description of Artificial Sequence: Synthetic peptide"
SASSSVPYMH

SEQ ID NO: 522 - protein, 15 aa, Artificial Sequence
  source: /note="Description of Artificial Sequence: Synthetic peptide"
WLQQKPGKSPKLLIY

SEQ ID NO: 523 - protein, 7 aa, Artificial Sequence
  source: /note="Description of Artificial Sequence: Synthetic peptide"
LTSNLAS

SEQ ID NO: 524 - protein, 32 aa, Artificial Sequence
  source: /note="Description of Artificial Sequence: Synthetic polypeptide"
GVPSRFSGSGSGTDYSLTISSVQPEDAATYYC

SEQ ID NO: 525 - protein, 9 aa, Artificial Sequence
  source: /note="Description of Artificial Sequence: Synthetic peptide"
QQRSSYPLT

SEQ ID NO: 526 - protein, 10 aa, Artificial Sequence
  source: /note="Description of Artificial Sequence: Synthetic peptide"
FGGGTKLEIK

SEQ ID NO: 527 - protein, 120 aa, Artificial Sequence; identical to SEQ ID NO: 511
  source: /note="Description of Artificial Sequence: Synthetic polypeptide"

SEQ ID NO: 528 - protein, 30 aa, Artificial Sequence; identical to SEQ ID NO: 512
  source: /note="Description of Artificial Sequence: Synthetic polypeptide"

SEQ ID NO: 529 - protein, 5 aa, Artificial Sequence
  source: /note="Description of Artificial Sequence: Synthetic peptide"
DSYMH

SEQ ID NO: 530 - protein, 14 aa, Artificial Sequence
  source: /note="Description of Artificial Sequence: Synthetic peptide"
WLRQGPGQRLEWIG

SEQ ID NO: 531 - protein, 17 aa, Artificial Sequence
  source: /note="Description of Artificial Sequence: Synthetic peptide"
WIDPENGDTEYAPKFQG

SEQ ID NO: 532 - protein, 32 aa, Artificial Sequence; identical to SEQ ID NO: 516
  source: /note="Description of Artificial Sequence: Synthetic polypeptide"

SEQ ID NO: 533 - protein, 11 aa, Artificial Sequence
  source: /note="Description of Artificial Sequence: Synthetic peptide"
GTPTGPYYFDY

SEQ ID NO: 534 - protein, 11 aa, Artificial Sequence
  source: /note="Description of Artificial Sequence: Synthetic peptide"
WGQGTLVTVSS

SEQ ID NO: 535 - protein, 23 aa, Artificial Sequence
  source: /note="Description of Artificial Sequence: Synthetic peptide"
QTVLSQSPAILSASPGEKVTMTC

SEQ ID NO: 536 - protein, 10 aa, Artificial Sequence
  source: /note="Description of Artificial Sequence: Synthetic peptide"
RASSSVTYIH

SEQ ID NO: 537 - protein, 15 aa, Artificial Sequence
  source: /note="Description of Artificial Sequence: Synthetic peptide"
WYQQKPGSSPKSWIY

SEQ ID NO: 538 - protein, 7 aa, Artificial Sequence
  source: /note="Description of Artificial Sequence: Synthetic peptide"
ATSNLAS

SEQ ID NO: 539 - protein, 32 aa, Artificial Sequence
  source: /note="Description of Artificial Sequence: Synthetic polypeptide"
GVPARFSGSGSGTSYSLTISRVEAEDAATYYC

SEQ ID NO: 540 - protein, 9 aa, Artificial Sequence
  source: /note="Description of Artificial Sequence: Synthetic peptide"
QHWSSKPPT

SEQ ID NO: 541 - protein, 10 aa, Artificial Sequence
  source: /note="Description of Artificial Sequence: Synthetic peptide"
FGGGTKLEIK

SEQ ID NO: 542 - protein, 121 aa, Artificial Sequence
  source: /note="Description of Artificial Sequence: Synthetic polypeptide"
EVKLVESGGGLVQPGGSLRLSCATSGFTFTDYYMNWVRQPPGKALEWLGFIGNKANGYTTEYSASVKGRFTISRDKSQSILYLQMNTLRAEDSATYYCTRDRGLRFYFDYWGQGTTLTVSS

SEQ ID NO: 543 - protein, 30 aa, Artificial Sequence
  source: /note="Description of Artificial Sequence: Synthetic polypeptide"
EVKLVESGGGLVQPGGSLRLSCATSGFTFT

SEQ ID NO: 544 - protein, 5 aa, Artificial Sequence
  source: /note="Description of Artificial Sequence: Synthetic peptide"
DYYMN

SEQ ID NO: 545 - protein, 14 aa, Artificial Sequence
  source: /note="Description of Artificial Sequence: Synthetic peptide"
WVRQPPGKALEWLG

SEQ ID NO: 546 - protein, 19 aa, Artificial Sequence
  source: /note="Description of Artificial Sequence: Synthetic peptide"
FIGNKANGYTTEYSASVKG

SEQ ID NO: 547 - protein, 32 aa, Artificial Sequence
  source: /note="Description of Artificial Sequence: Synthetic polypeptide"
RFTISRDKSQSILYLQMNTLRAEDSATYYCTR

SEQ ID NO: 548 - protein, 10 aa, Artificial Sequence
  source: /note="Description of Artificial Sequence: Synthetic peptide"
DRGLRFYFDY

SEQ ID NO: 549 - protein, 11 aa, Artificial Sequence
  source: /note="Description of Artificial Sequence: Synthetic peptide"
WGQGTTLTVSS

SEQ ID NO: 550 - protein, 111 aa, Artificial Sequence
  source: /note="Description of Artificial Sequence: Synthetic polypeptide"
DIQLTQSPSSLSASVGDRVTITCRAGESVDIFGVGFLHWYQQKPGKAPKLLIYRASNLESGVPSRFSGSGSRTDFTLTISSLQPEDFATYYCQQTNEDPYTFGQGTKVEIK

SEQ ID NO: 551 - protein, 23 aa, Artificial Sequence
  source: /note="Description of Artificial Sequence: Synthetic peptide"
DIQLTQSPSSLSASVGDRVTITC

SEQ ID NO: 552 - protein, 15 aa, Artificial Sequence
  source: /note="Description of Artificial Sequence: Synthetic peptide"
RAGESVDIFGVGFLH

SEQ ID NO: 553 - protein, 15 aa, Artificial Sequence
  source: /note="Description of Artificial Sequence: Synthetic peptide"
WYQQKPGKAPKLLIY

SEQ ID NO: 554 - protein, 7 aa, Artificial Sequence
  source: /note="Description of Artificial Sequence: Synthetic peptide"
RASNLES

SEQ ID NO: 555 - protein, 32 aa, Artificial Sequence
  source: /note="Description of Artificial Sequence: Synthetic polypeptide"
GVPSRFSGSGSRTDFTLTISSLQPEDFATYYC

SEQ ID NO: 556 - protein, 9 aa, Artificial Sequence
  source: /note="Description of Artificial Sequence: Synthetic peptide"
QQTNEDPYT

SEQ ID NO: 557 - protein, 10 aa, Artificial Sequence
  source: /note="Description of Artificial Sequence: Synthetic peptide"
FGQGTKVEIK

SEQ ID NO: 558 - protein, 121 aa, Artificial Sequence
  source: /note="Description of Artificial Sequence: Synthetic polypeptide"
EVQLVESGGGLVQPGGSLRLSCAASGFNIKDTYMHWVRQAPGKGLEWVARIDPANGNSKYADSVKGRFTISADTSKNTAYLQMNSLRAEDTAVYYCAPFGYYVSDYAMAYWGQGTLVTVSS

SEQ ID NO: 559 - protein, 30 aa, Artificial Sequence
  source: /note="Description of Artificial Sequence: Synthetic polypeptide"
EVQLVESGGGLVQPGGSLRLSCAASGFNIK

SEQ ID NO: 560 - protein, 5 aa, Artificial Sequence
  source: /note="Description of Artificial Sequence: Synthetic peptide"
DTYMH

SEQ ID NO: 561 - protein, 14 aa, Artificial Sequence
  source: /note="Description of Artificial Sequence: Synthetic peptide"
WVRQAPGKGLEWVA

SEQ ID NO: 562 - protein, 17 aa, Artificial Sequence
  source: /note="Description of Artificial Sequence: Synthetic peptide"
RIDPANGNSKYADSVKG

SEQ ID NO: 563 - protein, 32 aa, Artificial Sequence
  source: /note="Description of Artificial Sequence: Synthetic polypeptide"
RFTISADTSKNTAYLQMNSLRAEDTAVYYCAP

SEQ ID NO: 564 - protein, 12 aa, Artificial Sequence
  source: /note="Description of Artificial Sequence: Synthetic peptide"
FGYYVSDYAMAY

SEQ ID NO: 565 - protein, 11 aa, Artificial Sequence
  source: /note="Description of Artificial Sequence: Synthetic peptide"
WGQGTLVTVSS

SEQ ID NO: 566 - protein, 107 aa, Artificial Sequence [sequence omitted]

SEQ ID NO: 567 - protein, 23 aa, Artificial Sequence
  source: /note="Description of Artificial Sequence: Synthetic peptide"
DIQMTQSPASLSASVGDRVTITC

SEQ ID NO: 568 - protein, 11 aa, Artificial Sequence
  source: /note="Description of Artificial Sequence: Synthetic peptide"
RASENIFSYLA

SEQ ID NO: 569 - protein, 15 aa, Artificial Sequence
  source: /note="Description of Artificial Sequence: Synthetic peptide"
WYQQKPGKSPKLLVY

SEQ ID NO: 570 - protein, 7 aa, Artificial Sequence
  source: /note="Description of Artificial Sequence: Synthetic peptide"
NTRTLAE

SEQ ID NO: 571 - protein, 32 aa, Artificial Sequence
  source: /note="Description of Artificial Sequence: Synthetic polypeptide"
GVPSRFSGSGSGTDFSLTISSLQPEDFATYYC

SEQ ID NO: 572 - protein, 9 aa, Artificial Sequence
  source: /note="Description of Artificial Sequence: Synthetic peptide"
QHHYGTPFT

SEQ ID NO: 573 - protein, 10 aa, Artificial Sequence
  source: /note="Description of Artificial Sequence: Synthetic peptide"
FGSGTKLEIK

SEQ ID NO: 574 - protein, 120 aa, Artificial Sequence [sequence omitted]

SEQ ID NO: 575 - protein, 30 aa, Artificial Sequence
  source: /note="Description of Artificial Sequence: Synthetic polypeptide"
EVQLQESGPGLVKPGGSLSLSCAASGFVFS

SEQ ID NO: 576 - protein, 5 aa, Artificial Sequence
  source: /note="Description of Artificial Sequence: Synthetic peptide"
SYDMS

SEQ ID NO: 577 - protein, 14 aa, Artificial Sequence
  source: /note="Description of Artificial Sequence: Synthetic peptide"
WVRQTPERGLEWVA

SEQ ID NO: 578 - protein, 17 aa, Artificial Sequence
  source: /note="Description of Artificial Sequence: Synthetic peptide"
YISSGGGITYAPSTVKG

SEQ ID NO: 579 - protein, 32 aa, Artificial Sequence
  source: /note="Description of Artificial Sequence: Synthetic polypeptide"
RFTVSRDNAKNTLYLQMNSLTSEDTAVYYCAA

SEQ ID NO: 580 - protein, 11 aa, Artificial Sequence
  source: /note="Description of Artificial Sequence: Synthetic peptide"
HYFGSSGPFAY

SEQ ID NO: 581 - protein, 11 aa, Artificial Sequence
  source: /note="Description of Artificial Sequence: Synthetic peptide"
WGQGTLVTVSS

SEQ ID NO: 582 - protein, 116 aa, Artificial Sequence
  source: /note="Description of Artificial Sequence: Synthetic polypeptide"
QAVLTQPASLSASPGASASLTCTLRRGINVGAYSIYWYQQKPGSPPQYLLRYKSDSDKQQGSGVSSRFSASKDASANAGILLISGLQSEDEADYYCMIWHSGASAVFGGGTKLTVL

SEQ ID NO: 583 - protein, 22 aa, Artificial Sequence
  source: /note="Description of Artificial Sequence: Synthetic peptide"
QAVLTQPASLSASPGASASLTC

SEQ ID NO: 584 - protein, 14 aa, Artificial Sequence
  source: /note="Description of Artificial Sequence: Synthetic peptide"
TLRRGINVGAYSIY

SEQ ID NO: 585 - protein, 15 aa, Artificial Sequence
  source: /note="Description of Artificial Sequence: Synthetic peptide"
WYQQKPGSPPQYLLR

SEQ ID NO: 586 - protein, 11 aa, Artificial Sequence
  source: /note="Description of Artificial Sequence: Synthetic peptide"
YKSDSDKQQGS

SEQ ID NO: 587 - protein, 34 aa, Artificial Sequence
  source: /note="Description of Artificial Sequence: Synthetic polypeptide"
GVSSRFSASKDASANAGILLISGLQSEDEADYYC

SEQ ID NO: 588 - protein, 10 aa, Artificial Sequence
  source: /note="Description of Artificial Sequence: Synthetic peptide"
MIWHSGASAV

SEQ ID NO: 589 - protein, 10 aa, Artificial Sequence
  source: /note="Description of Artificial Sequence: Synthetic peptide"
FGGGTKLTVL

SEQ ID NO: 590 - protein, 121 aa, Artificial Sequence
  source: /note="Description of Artificial Sequence: Synthetic polypeptide"
EVQLVESGGGLVQPGRSLRLSCAASGFTVSSYWMHWVRQAPGKGLEWVGFIRNKANGGTTEYAASVKGRFTISRDDSKNTLYLQMNSLRAEDTAVYYCARDRGLRFYFDYWGQGTTVTVSS

SEQ ID NO: 591 - protein, 30 aa, Artificial Sequence
  source: /note="Description of Artificial Sequence: Synthetic polypeptide"
EVQLVESGGGLVQPGRSLRLSCAASGFTVS

SEQ ID NO: 592 - protein, 5 aa, Artificial Sequence
  source: /note="Description of Artificial Sequence: Synthetic peptide"
SYWMH

SEQ ID NO: 593 - protein, 14 aa, Artificial Sequence
  source: /note="Description of Artificial Sequence: Synthetic peptide"
WVRQAPGKGLEWVG

SEQ ID NO: 594 - protein, 19 aa, Artificial Sequence
  source: /note="Description of Artificial Sequence: Synthetic peptide"
FIRNKANGGTTEYAASVKG

SEQ ID NO: 595 - protein, 32 aa, Artificial Sequence
  source: /note="Description of Artificial Sequence: Synthetic polypeptide"
RFTISRDDSKNTLYLQMNSLRAEDTAVYYCAR

SEQ ID NO: 596 - protein, 10 aa, Artificial Sequence
  source: /note="Description of Artificial Sequence: Synthetic peptide"
DRGLRFYFDY

SEQ ID NO: 597 - protein, 11 aa, Artificial Sequence
  source: /note="Description of Artificial Sequence: Synthetic peptide"
WGQGTTVTVSS

SEQ ID NO: 598 - protein, 121 aa, Artificial Sequence
  source: /note="Description of Artificial Sequence: Synthetic polypeptide"
EVQLVESGGGLVQPGRSLRLSCAASGFTVSSYWMHWVRQAPGKGLEWVGFILNKANGGTTEYAASVKGRFTISRDDSKNTLYLQMNSLRAEDTAVYYCARDRGLRFYFDYWGQGTTVTVSS

SEQ ID NO: 599 - protein, 30 aa, Artificial Sequence; identical to SEQ ID NO: 591
  source: /note="Description of Artificial Sequence: Synthetic polypeptide"

SEQ ID NO: 600 - protein, 5 aa, Artificial Sequence
  source: /note="Description of Artificial Sequence: Synthetic peptide"
SYWMH

SEQ ID NO: 601 - protein, 14 aa, Artificial Sequence
  source: /note="Description of Artificial Sequence: Synthetic peptide"
WVRQAPGKGLEWVG

SEQ ID NO: 602 - protein, 19 aa, Artificial Sequence
  source: /note="Description of Artificial Sequence: Synthetic peptide"
FILNKANGGTTEYAASVKG

SEQ ID NO: 603 - protein, 32 aa, Artificial Sequence; identical to SEQ ID NO: 595
  source: /note="Description of Artificial Sequence: Synthetic polypeptide"

SEQ ID NO: 604 - protein, 10 aa, Artificial Sequence
  source: /note="Description of Artificial Sequence: Synthetic peptide"
DRGLRFYFDY

SEQ ID NO: 605 - protein, 11 aa, Artificial Sequence
  source: /note="Description of Artificial Sequence: Synthetic peptide"
WGQGTTVTVSS

SEQ ID NO: 606 - protein, 30 aa, Artificial Sequence
  source: /note="Description of Artificial Sequence: Synthetic polypeptide"
QVQLVQSGAEVKKPGASVKVSCKASGYTLP

SEQ ID NO: 607 - protein, 11 aa, Artificial Sequence
  source: /note="Description of Artificial Sequence: Synthetic peptide"
KASQDVSIAVA

SEQ ID NO: 608 - protein, 7 aa, Artificial Sequence
  source: /note="Description of Artificial Sequence: Synthetic peptide"
SASYRYT

SEQ ID NO: 609 - protein, 9 aa, Artificial Sequence
  source: /note="Description of Artificial Sequence: Synthetic peptide"
QQHYITPLT

SEQ ID NO: 610 - protein, 5 aa, Artificial Sequence
  source: /note="Description of Artificial Sequence: Synthetic peptide"
NYGMN

SEQ ID NO: 611 - protein, 17 aa, Artificial Sequence
  source: /note="Description of Artificial Sequence: Synthetic peptide"
WINTYTGEPTYTDDFKG

SEQ ID NO: 612 - protein, 12 aa, Artificial Sequence
  source: /note="Description of Artificial Sequence: Synthetic peptide"
GGFGSSYWYFDV

SEQ ID NO: 613 - protein, 17 aa, Artificial Sequence
  source: /note="Description of Artificial Sequence: Synthetic peptide"
WINTKTGEPTYAEEFKG

SEQ ID NO: 614 - protein, 12 aa, Artificial Sequence
  source: /note="Description of Artificial Sequence: Synthetic peptide"
GGYGSSYWYFDV

SEQ ID NO: 615 - protein, 17 aa, Artificial Sequence
  source: /note="Description of Artificial Sequence: Synthetic peptide"
KSSQSLLNSGNQQNYLA

SEQ ID NO: 616 - protein, 7 aa, Artificial Sequence
  source: /note="Description of Artificial Sequence: Synthetic peptide"
GASTRES

SEQ ID NO: 617 - protein, 9 aa, Artificial Sequence
  source: /note="Description of Artificial Sequence: Synthetic peptide"
QSDHIYPYT

SEQ ID NO: 618 - protein, 5 aa, Artificial Sequence
  source: /note="Description of Artificial Sequence: Synthetic peptide"
IYWLG

SEQ ID NO: 619 - protein, 17 aa, Artificial Sequence
  source: /note="Description of Artificial Sequence: Synthetic peptide"
NIFPGSAYINYNEKFKG

SEQ ID NO: 620 - protein, 7 aa, Artificial Sequence
  source: /note="Description of Artificial Sequence: Synthetic peptide"
EGSNSGY

SEQ ID NO: 621 - protein, 11 aa, Artificial Sequence
  source: /note="Description of Artificial Sequence: Synthetic peptide"
KASQDVSTAVA

SEQ ID NO: 622 - protein, 5 aa, Artificial Sequence
  source: /note="Description of Artificial Sequence: Synthetic peptide"
TAGMQ

SEQ ID NO: 623 - protein, 17 aa, Artificial Sequence
  source: /note="Description of Artificial Sequence: Synthetic peptide"
WINTHSGVPKYAEDFKG

SEQ ID NO: 624 - protein, 12 aa, Artificial Sequence
  source: /note="Description of Artificial Sequence: Synthetic peptide"
SGFGSSYWYFDV

SEQ ID NO: 625 - protein, 15 aa, Artificial Sequence
  source: /note="Description of Artificial Sequence: Synthetic peptide"
  VARIANT (10)..(10): /replace="L" or "N"
  SITE (1)..(15): /note="Variant residues given in the sequence have no preference with respect to those in the annotations for variant positions"
RASKSVSTSGYSYMH

SEQ ID NO: 626 - protein, 7 aa, Artificial Sequence
  source: /note="Description of Artificial Sequence: Synthetic peptide"
LASNLES

SEQ ID NO: 627 - protein, 9 aa, Artificial Sequence
  source: /note="Description of Artificial Sequence: Synthetic peptide"
QHSRELPYT

SEQ ID NO: 628 - protein, 5 aa, Artificial Sequence
  source: /note="Description of Artificial Sequence: Synthetic peptide"
SYGVH

SEQ ID NO: 629 - protein, 7 aa, Artificial Sequence
  source: /note="Description of Artificial Sequence: Synthetic peptide"
GGSISSY

SEQ ID NO: 630 - protein, 10 aa, Artificial Sequence
  source: /note="Description of Artificial Sequence: Synthetic peptide"
GGSISSYGVH

SEQ ID NO: 631 - protein, 16 aa, Artificial Sequence
  source: /note="Description of Artificial Sequence: Synthetic peptide"
  VARIANT (5)..(5): /replace="S"
  VARIANT (7)..(7): /replace="V"
  VARIANT (16)..(16): /replace="G"
  SITE (1)..(16): /note="Variant residues given in the sequence have no preference with respect to those in the annotations for variant positions"
VIWTGGSTDYNSALMS

SEQ ID NO: 632 - protein, 5 aa, Artificial Sequence
  source: /note="Description of Artificial Sequence: Synthetic peptide"
  VARIANT (3)..(3): /replace="S"
  VARIANT (5)..(5): /replace="V"
  SITE (1)..(5): /note="Variant residues given in the sequence have no preference with respect to those in the annotations for variant positions"
WTGGS

SEQ ID NO: 633 - protein, 11 aa, Artificial Sequence
  source: /note="Description of Artificial Sequence: Synthetic peptide"
DGDYDRYTMDY

SEQ ID NO: 634 - protein, 15 aa, Artificial Sequence
  source: /note="Description of Artificial Sequence: Synthetic peptide"
RASKSVSTSGYSYMH

SEQ ID NO: 635 - protein, 16 aa, Artificial Sequence
  source: /note="Description of Artificial Sequence: Synthetic peptide"
VIWTSGVTDYNSALMG

SEQ ID NO: 636 - protein, 5 aa, Artificial Sequence
  source: /note="Description of Artificial Sequence: Synthetic peptide"
WTSGV

SEQ ID NO: 637 - protein, 5 aa, Artificial Sequence
  source: /note="Description of Artificial Sequence: Synthetic peptide"
  MOD_RES (1)..(1): Any peptide amino capping group
XAAPV

SEQ ID NO: 638 - protein, 5 aa, Artificial Sequence
  source: /note="Description of Artificial Sequence: Synthetic peptide"
  MOD_RES (1)..(1): Any peptide amino capping group
  MOD_RES (5)..(5): Norvaline
XAAPX

SEQ ID NO: 639 - protein, 4 aa, Artificial Sequence
  source: /note="Description of Artificial Sequence: Synthetic peptide"
AAPA

SEQ ID NO: 640 - protein, 4 aa, Artificial Sequence
  source: /note="Description of Artificial Sequence: Synthetic peptide"
AAPV

SEQ ID NO: 641 - protein, 4 aa, Artificial Sequence
  source: /note="Description of Artificial Sequence: Synthetic peptide"
  MOD_RES (4)..(4): Norvaline
AAPX